SEQUENCE LISTING (399 sequences)

SEQ ID NO: 1 - protein, 525 aa, Homo sapiens
  misc_feature (1)..(525): Human LAG3
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 2 - protein, 533 aa, Macaca fascicularis
  misc_feature (1)..(533): Cynomolgus LAG3
  misc_feature (74)..(74): Xaa can be any naturally occurring amino acid
MWEAQFLGLLFLQPLWVAPVKPPQPGAEISVVWAQEGAPAQLPCSPTIPLQDLSLLRRAGVTWQHQPDSGPPAXAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHLPLFLILGVLFLLLLVTGAFGFHLWRRQWRPRRFSALEQGIHPPQAQSKIEELEQEPELEPEPELERELGPEPEPGPEPEPEQL

SEQ ID NO: 3 - protein, 521 aa, Mus musculus
  misc_feature (1)..(521): Mouse LAG3
MREDLLLGFLLLGLLWEAPVVSSGPGKELPVVWAQEGAPVHLPCSLKSPNLDPNFLRRGGVIWQHQPDSGQPTPIPALDLHQGMPSPRQPAPGRYTVLSVAPGGLRSGRQPLHPHVQLEERGLQRGDFSLWLRPALRTDAGEYHATVRLPNRALSCSLRLRVGQASMIASPSGVLKLSDWVLLNCSFSRPDRPVSVHWFQGQNRVPVYNSPRHFLAETFLLLPQVSPLDSGTWGCVLTYRDGFNVSITYNLKVLGLEPVAPLTVYAAEGSRVELPCHLPPGVGTPSLLIAKWTPPGGGPELPVAGKSGNFTLHLEAVGLAQAGTYTCSIHLQGQQLNATVTLAVITVTPKSFGLPGSRGKLLCEVTPASGKERFVWRPLNNLSRSCPGPVLEIQEARLLAERWQCQLYEGQRLLGATVYAAESSSGAHSARRISGDLKGGHLVLVLILGALSLFLLVAGAFGFHWWRKQLLLRRFSALEHGIQPFPAQRKIEELERELETEMGQEPEPEPEPQLEPEPRQL

SEQ ID NO: 4 - protein, 6 aa, Artificial Sequence
  Synthetic 26H10, CDR-H1
GFTSSY

SEQ ID NO: 5 - protein, 7 aa, Artificial Sequence
  Synthetic SRP1496-A03-VH, CDR-H1
GFNINDT

SEQ ID NO: 6 - protein, 7 aa, Artificial Sequence
  Synthetic SRP1496-A04-VH, CDR-H1
GFNINDT

SEQ ID NO: 7 - protein, 7 aa, Artificial Sequence
  Synthetic SRP1496-B08-VH, CDR-H1
GFNINDT

SEQ ID NO: 8 - protein, 7 aa, Artificial Sequence
  Synthetic SRP1648-B07-VH, CDR-H1
GFNIADT

SEQ ID NO: 9 - protein, 7 aa, Artificial Sequence
  Synthetic SRP1648-E02-VH, CDR-H1
GFNINDN

SEQ ID NO: 10 - protein, 7 aa, Artificial Sequence
  Synthetic SRP1449-B03-VH, CDR-H1
GFTFSSY

SEQ ID NO: 11 - protein, 7 aa, Artificial Sequence
  Synthetic SRP1449-F01-VH, CDR-H1
GFTFSSY

SEQ ID NO: 12 - protein, 7 aa, Artificial Sequence
  Synthetic SRP1449-B07-VH, CDR-H1
GFTFSSY

SEQ ID NO: 13 - protein, 7 aa, Artificial Sequence
  Synthetic 1449-G09.2-VH, CDR-H1
GFTFSSY

SEQ ID NO: 14 - protein, 7 aa, Artificial Sequence
  Synthetic SRP1449-D05-VH, CDR-H1
GFTFRSF

SEQ ID NO: 15 - protein, 7 aa, Artificial Sequence
  Synthetic SRP1558-F01-VH, CDR-H1
GFTFPDS

SEQ ID NO: 16 - protein, 7 aa, Artificial Sequence
  Synthetic SRP1448-D09-VH, CDR-H1
GFTFTDS

SEQ ID NO: 17 - protein, 7 aa, Artificial Sequence
  Synthetic SRP1558-A06-VH, CDR-H1
GFTFSES

SEQ ID NO: 18 - protein, 7 aa, Artificial Sequence
  Synthetic SRP1558-E11-VH, CDR-H1
GFTFTSS

SEQ ID NO: 19 - protein, 7 aa, Artificial Sequence
  Synthetic SRP1627-A02-VH, CDR-H1
GFNINDY

SEQ ID NO: 20 - protein, 7 aa, Artificial Sequence
  Synthetic SRP1627-A11-VH, CDR-H1
GFNINDY

SEQ ID NO: 21 - protein, 7 aa, Artificial Sequence
  Synthetic h5G11-2-VH, CDR-H1
GFNIKDY

SEQ ID NO: 22 - protein, 7 aa, Artificial Sequence
  Synthetic SRP1627-B01-VH, CDR-H1
GFNITDL

SEQ ID NO: 23 - protein, 7 aa, Artificial Sequence
  Synthetic 421.61.4.5G11-VH, CDR-H1
GFNIKDY

SEQ ID NO: 24 - protein, 7 aa, Artificial Sequence
  Synthetic 1353-G10-wt, CDR-H1
GYRFPHY

SEQ ID NO: 25 - protein, 7 aa, Artificial Sequence
  Synthetic 1353-G10 Y27D, CDR-H1
GDRFPHY

SEQ ID NO: 26 - protein, 7 aa, Artificial Sequence
  Synthetic 1353-G10 R28D, CDR-H1
GYDFPHY

SEQ ID NO: 27 - protein, 7 aa, Artificial Sequence
  Synthetic 1353-G10 F29D, CDR-H1
GYRDPHY

SEQ ID NO: 28 - protein, 7 aa, Artificial Sequence
  Synthetic 1353-G10 P30D, CDR-H1
GYRFDHY

SEQ ID NO: 29 - protein, 7 aa, Artificial Sequence
  Synthetic 1353-G10 H31D, CDR-H1
GYRFPDY

SEQ ID NO: 30 - protein, 7 aa, Artificial Sequence
  Synthetic 1353-G10 Y32D, CDR-H1
GYRFPHD

SEQ ID NO: 31 - protein, 7 aa, Artificial Sequence
  Synthetic 1353-G10 R28T/P30D, CDR-H1
GYTFDHY

SEQ ID NO: 32 - protein, 7 aa, Artificial Sequence
  Synthetic 1353-G10 R28D/P30D, CDR-H1
GYDFDHY

SEQ ID NO: 33 - protein, 7 aa, Artificial Sequence
  Synthetic 1353-G10 R28D/H31D, CDR-H1
GYDFPDY

SEQ ID NO: 34 - protein, 7 aa, Artificial Sequence
  Synthetic 1353-G10 R28T/H31D, CDR-H1
GYTFPDY

SEQ ID NO: 35 - protein, 7 aa, Artificial Sequence
  Synthetic 1353-G10 R28D/P30T/H31S, CDR-H1
GYDFTSY

SEQ ID NO: 36 - protein, 7 aa, Artificial Sequence
  Synthetic 1353-G10 R28T/P30T/H31D, CDR-H1
GYTFTDY

SEQ ID NO: 37 - protein, 7 aa, Artificial Sequence
  Synthetic 1353-G10 R28T/P30T/H31S, CDR-H1
GYTFTSY

SEQ ID NO: 38 - protein, 7 aa, Artificial Sequence
  Synthetic 1353-G10-R28T/P30D/H31S, CDR-H1
GYTFDSY

SEQ ID NO: 39 - protein, 5 aa, Artificial Sequence
  Synthetic 26H10, CDR-H1
SYGMH

SEQ ID NO: 40 - protein, 5 aa, Artificial Sequence
  Synthetic SRP1496-A03-VH, CDR-H1
DTYIH

SEQ ID NO: 41 - protein, 5 aa, Artificial Sequence
  Synthetic SRP1496-A04-VH, CDR-H1
DTYIH

SEQ ID NO: 42 - protein, 5 aa, Artificial Sequence
  Synthetic SRP1496-B08-VH, CDR-H1
DTYIH

SEQ ID NO: 43 - protein, 5 aa, Artificial Sequence
  Synthetic SRP1648-B07-VH, CDR-H1
DTFIH

SEQ ID NO: 44 - protein, 5 aa, Artificial Sequence
  Synthetic SRP1648-E02-VH, CDR-H1
DNYIH

SEQ ID NO: 45 - protein, 5 aa, Artificial Sequence
  Synthetic SRP1449-B03-VH, CDR-H1
SYGMH

SEQ ID NO: 46 - protein, 5 aa, Artificial Sequence
  Synthetic SRP1449-F01-VH, CDR-H1
SYGMH

SEQ ID NO: 47 - protein, 5 aa, Artificial Sequence
  Synthetic SRP1449-B07-VH, CDR-H1
SYGMH

SEQ ID NO: 48 - protein, 5 aa, Artificial Sequence
  Synthetic 1449-G09.2-VH, CDR-H1
SYGMH

SEQ ID NO: 49 - protein, 5 aa, Artificial Sequence
  Synthetic SRP1449-D05-VH, CDR-H1
SFGMH

SEQ ID NO: 50 - protein, 5 aa, Artificial Sequence
  Synthetic SRP1558-F01-VH, CDR-H1
DSSMS

SEQ ID NO: 51 - protein, 5 aa, Artificial Sequence
  Synthetic SRP1448-D09-VH, CDR-H1
DSSMS

SEQ ID NO: 52 - protein, 5 aa, Artificial Sequence
  Synthetic SRP1558-A06-VH, CDR-H1
ESTMS

SEQ ID NO: 53 - protein, 5 aa, Artificial Sequence
  Synthetic SRP1558-E11-VH, CDR-H1
SSSMS

SEQ ID NO: 54 - protein, 5 aa, Artificial Sequence
  Synthetic SRP1627-A02-VH, CDR-H1
DYFMH

SEQ ID NO: 55 - protein, 5 aa, Artificial Sequence
  Synthetic SRP1627-A11-VH, CDR-H1
DYFMH

SEQ ID NO: 56 - protein, 5 aa, Artificial Sequence
  Synthetic h5G11-2-VH, CDR-H1
DYYMH

SEQ ID NO: 57 - protein, 5 aa, Artificial Sequence
  Synthetic SRP1627-B01-VH, CDR-H1
DLYMH

SEQ ID NO: 58 - protein, 5 aa, Artificial Sequence
  Synthetic 421.61.4.5G11-VH, CDR-H1
DYYMH

SEQ ID NO: 59 - protein, 5 aa, Artificial Sequence
  Synthetic 1353-G10-wt, CDR-H1
HYGIS

SEQ ID NO: 60 - protein, 5 aa, Artificial Sequence
  Synthetic 1353-G10 H31D, CDR-H1
DYGIS

SEQ ID NO: 61 - protein, 5 aa, Artificial Sequence
  Synthetic 1353-G10 Y32D, CDR-H1
HDGIS

SEQ ID NO: 62 - protein, 5 aa, Artificial Sequence
  Synthetic 1353-G10 G33D, CDR-H1
HYDIS

SEQ ID NO: 63 - protein, 5 aa, Artificial Sequence
  Synthetic 1353-G10 S35D, CDR-H1
HYGID

SEQ ID NO: 64 - protein, 5 aa, Artificial Sequence
  Synthetic 1353-G10 R28D/P30T/H31S, CDR-H1
SYGIS

SEQ ID NO: 65 - protein, 5 aa, Artificial Sequence
  Synthetic 1353-G10-R28T/P30D/H31S, CDR-H1
SYGIS

SEQ ID NO: 66 - protein, 6 aa, Artificial Sequence
  Synthetic 26H10, CDR-H2
WYDGSN

SEQ ID NO: 67 - protein, 6 aa, Artificial Sequence
  Synthetic SRP1496-A03-VH, CDR-H2
DPYDGA

SEQ ID NO: 68 - protein, 6 aa, Artificial Sequence
  Synthetic SRP1496-A04-VH, CDR-H2
DPYDGA

SEQ ID NO: 69 - protein, 6 aa, Artificial Sequence
  Synthetic SRP1496-B08-VH, CDR-H2
DPYDGA

SEQ ID NO: 70 - protein, 6 aa, Artificial Sequence
  Synthetic SRP1648-B07-VH, CDR-H2
DPYDGD

SEQ ID NO: 71 - protein, 6 aa, Artificial Sequence
  Synthetic SRP1648-E02-VH, CDR-H2
DPYDGF

SEQ ID NO: 72 - protein, 6 aa, Artificial Sequence
  Synthetic SRP1449-B03-VH, CDR-H2
WYDASY

SEQ ID NO: 73 - protein, 6 aa, Artificial Sequence
  Synthetic SRP1449-F01-VH, CDR-H2
WYDGSY

SEQ ID NO: 74 - protein, 6 aa, Artificial Sequence
  Synthetic SRP1449-B07-VH, CDR-H2
WYDGSN

SEQ ID NO: 75 - protein, 6 aa, Artificial Sequence
  Synthetic 1449-G09.2-VH, CDR-H2
WYDGSY

SEQ ID NO: 76 - protein, 6 aa, Artificial Sequence
  Synthetic SRP1449-D05-VH, CDR-H2
WYDGSV

SEQ ID NO: 77 - protein, 6 aa, Artificial Sequence
  Synthetic SRP1558-F01-VH, CDR-H2
TDNSGN

SEQ ID NO: 78 - protein, 6 aa, Artificial Sequence
  Synthetic SRP1448-D09-VH, CDR-H2
TGNSGT

SEQ ID NO: 79 - protein, 6 aa, Artificial Sequence
  Synthetic SRP1558-A06-VH, CDR-H2
TSDSGT

SEQ ID NO: 80 - protein, 6 aa, Artificial Sequence
  Synthetic SRP1558-E11-VH, CDR-H2
SDDTGS

SEQ ID NO: 81 - protein, 6 aa, Artificial Sequence
  Synthetic SRP1627-A02-VH, CDR-H2
DPWNGD

SEQ ID NO: 82 - protein, 6 aa, Artificial Sequence
  Synthetic SRP1627-A11-VH, CDR-H2
DPWNGD

SEQ ID NO: 83 - protein, 6 aa, Artificial Sequence
  Synthetic h5G11-2-VH, CDR-H2
DPENGD

SEQ ID NO: 84 - protein, 6 aa, Artificial Sequence
  Synthetic SRP1627-B01-VH, CDR-H2
DPWNGD

SEQ ID NO: 85 - protein, 6 aa, Artificial Sequence
  Synthetic 421.61.4.5G11-VH, CDR-H2
DPENGD

SEQ ID NO: 86 - protein, 6 aa, Artificial Sequence
  Synthetic 1353-G10-wt, CDRH2
SAYNGN

SEQ ID NO: 87 - protein, 6 aa, Artificial Sequence
  Synthetic 1353-G10-R28T/P30D/H31S, CDRH2
SAYNGN

SEQ ID NO: 88 - protein, 17 aa, Artificial Sequence
  Synthetic 26H10, CDR-H2
VIWYDGSNKYYADSVKG

SEQ ID NO: 89 - protein, 17 aa, Artificial Sequence
  Synthetic SRP1496-A03-VH, CDR-H2
IIDPYDGATDYADSVKG

SEQ ID NO: 90 - protein, 17 aa, Artificial Sequence
  Synthetic SRP1496-A04-VH, CDR-H2
IIDPYDGATDYADSVKG

SEQ ID NO: 91 - protein, 17 aa, Artificial Sequence
  Synthetic SRP1496-B08-VH, CDR-H2
IIDPYDGATDYADSVKG

SEQ ID NO: 92 - protein, 17 aa, Artificial Sequence
  Synthetic SRP1648-B07-VH, CDR-H2
IIDPYDGDTDYADSVKG

SEQ ID NO: 93 - protein, 17 aa, Artificial Sequence
  Synthetic SRP1648-E02-VH, CDR-H2
IIDPYDGFTAYADSVKG

SEQ ID NO: 94 - protein, 17 aa, Artificial Sequence
  Synthetic SRP1449-B03-VH, CDR-H2
AIWYDASYKYYADSVKG

SEQ ID NO: 95 - protein, 17 aa, Artificial Sequence
  Synthetic SRP1449-F01-VH, CDR-H2
VIWYDGSYKYYADSVKG

SEQ ID NO: 96 - protein, 17 aa, Artificial Sequence
  Synthetic SRP1449-B07-VH, CDR-H2
VIWYDGSNKYYADSVKG

SEQ ID NO: 97 - protein, 17 aa, Artificial Sequence
  Synthetic 1449-G09.2-VH, CDR-H2
VIWYDGSYKYYADSVKG

SEQ ID NO: 98 - protein, 17 aa, Artificial Sequence
  Synthetic SRP1449-D05-VH, CDR-H2
VIWYDGSVKYYADSVKG

SEQ ID NO: 99 - protein, 17 aa, Artificial Sequence
  Synthetic SRP1558-F01-VH, CDR-H2
VITDNSGNTDYADSVKG

SEQ ID NO: 100 - protein, 17 aa, Artificial Sequence
  Synthetic SRP1448-D09-VH, CDR-H2
VITGNSGTTDYADSVKG

SEQ ID NO: 101 - protein, 17 aa, Artificial Sequence
  Synthetic SRP1558-A06-VH, CDR-H2
FITSDSGTTDYADSVKG

SEQ ID NO: 102 - protein, 17 aa, Artificial Sequence
  Synthetic SRP1558-E11-VH, CDR-H2
VISDDTGSTDYADSVKG

SEQ ID NO: 103 - protein, 17 aa, Artificial Sequence
  Synthetic SRP1627-A02-VH, CDR-H2
RIDPWNGDTEYAPKFQG

SEQ ID NO: 104 - protein, 17 aa, Artificial Sequence
  Synthetic SRP1627-A11-VH, CDR-H2
RIDPWNGDTEYAPKFQG

SEQ ID NO: 105 - protein, 17 aa, Artificial Sequence
  Synthetic h5G11-2-VH, CDR-H2
WIDPENGDTEYAPKFQG

SEQ ID NO: 106 - protein, 17 aa, Artificial Sequence
  Synthetic SRP1627-B01-VH, CDR-H2
RIDPWNGDTEYAPKFQG

SEQ ID NO: 107 - protein, 17 aa, Artificial Sequence
  Synthetic 421.61.4.5G11-VH, CDR-H2
WIDPENGDTEYAPKFQG

SEQ ID NO: 108 - protein, 17 aa, Artificial Sequence
  Synthetic 1353-G10-wt, CDRH2
WISAYNGNTNYAQKLQG

SEQ ID NO: 109 - protein, 17 aa, Artificial Sequence
  Synthetic 1353-G10-R28T/P30D/H31S, CDRH2
WISAYNGNTNYAQKLQG

SEQ ID NO: 110 - protein, 13 aa, Artificial Sequence
  Synthetic 26H10, CDR-H3
EWAVASWDYGMDV

SEQ ID NO: 111 - protein, 12 aa, Artificial Sequence
  Synthetic SRP1496-A03-VH, CDR-H3
EIFGFYWNPFDY

SEQ ID NO: 112 - protein, 12 aa, Artificial Sequence
  Synthetic SRP1496-A04-VH, CDR-H3
EIFGFYWNPFDY

SEQ ID NO: 113 - protein, 12 aa, Artificial Sequence
  Synthetic SRP1496-B08-VH, CDR-H3
EIFGFYWNPFDY

SEQ ID NO: 114 - protein, 12 aa, Artificial Sequence
  Synthetic SRP1648-B07-VH, CDR-H3
EILGFYWNPFDY

SEQ ID NO: 115 - protein, 12 aa, Artificial Sequence
  Synthetic SRP1648-E02-VH, CDR-H3
ESIGFYLNPFDY

SEQ ID NO: 116 - protein, 13 aa, Artificial Sequence
  Synthetic SRP1449-B03-VH, CDR-H3
EWAVASWDYALDV

SEQ ID NO: 117 - protein, 13 aa, Artificial Sequence
  Synthetic SRP1449-F01-VH, CDR-H3
ESEVASWDYGLDV

SEQ ID NO: 118 - protein, 13 aa, Artificial Sequence
  Synthetic SRP1449-B07-VH, CDR-H3
EWAVSSWDYGMDV

SEQ ID NO: 119 - protein, 13 aa, Artificial Sequence
  Synthetic 1449-G09.2-VH, CDR-H3
EEAPENWDYALDV

SEQ ID NO: 120 - protein, 13 aa, Artificial Sequence
  Synthetic SRP1449-D05-VH, CDR-H3
EWADVSWDAGLDV

SEQ ID NO: 121 - protein, 12 aa, Artificial Sequence
  Synthetic SRP1558-F01-VH, CDR-H3
VFEGGVRPYSDY

SEQ ID NO: 122 - protein, 12 aa, Artificial Sequence
  Synthetic SRP1448-D09-VH, CDR-H3
VYEGGVRPYSDY

SEQ ID NO: 123 - protein, 12 aa, Artificial Sequence
  Synthetic SRP1558-A06-VH, CDR-H3
VFEGGVRPFSDY

SEQ ID NO: 124 - protein, 12 aa, Artificial Sequence
  Synthetic SRP1558-E11-VH, CDR-H3
VDNGGVRPYSDY

SEQ ID NO: 125 - protein, 6 aa, Artificial Sequence
  Synthetic SRP1627-A02-VH, CDR-H3
SDALDY

SEQ ID NO: 126 - protein, 6 aa, Artificial Sequence
  Synthetic SRP1627-A11-VH, CDR-H3
SDALDY

SEQ ID NO: 127 - protein, 6 aa, Artificial Sequence
  Synthetic h5G11-2-VH, CDR-H3
PDALDY

SEQ ID NO: 128 - protein, 6 aa, Artificial Sequence
  Synthetic SRP1627-B01-VH, CDR-H3
SEMVDY

SEQ ID NO: 129 - protein, 6 aa, Artificial Sequence
  Synthetic 421.61.4.5G11-VH, CDR-H3
PDALDY

SEQ ID NO: 130 - protein, 10 aa, Artificial Sequence
  Synthetic 1353-G10-wt, CDRH3
DVDYGTGSGY

SEQ ID NO: 131 - protein, 10 aa, Artificial Sequence
  Synthetic 1353-G10-R28T/P30D/H31S, CDRH3
DVDYGTGSGY

SEQ ID NO: 132 - protein, 12 aa, Artificial Sequence
  Synthetic 26H10, CDR-L1
RASQSVSSSYLA

SEQ ID NO: 133 - protein, 12 aa, Artificial Sequence
  Synthetic SRP1449-D05-VL, CDR-L1
RASQSVSSSYLA

SEQ ID NO: 134 - protein, 12 aa, Artificial Sequence
  Synthetic SRP1449-F01-VL, CDR-L1
RASRSVSSSYLA

SEQ ID NO: 135 - protein, 12 aa, Artificial Sequence
  Synthetic 1449-G09.2-VL, CDR-L1
RASQSVSSSYLA

SEQ ID NO: 136 - protein, 12 aa, Artificial Sequence
  Synthetic SRP1449-B07-VL, CDR-L1
RASQSVSSSYLA

SEQ ID NO: 137 - protein, 12 aa, Artificial Sequence
  Synthetic SRP1449-B03-VL, CDR-L1
RASQSVSSSYLA

SEQ ID NO: 138 - protein, 12 aa, Artificial Sequence
  Synthetic SRP1558-E11-VL, CDR-L1
RASQSVSSSYLA

SEQ ID NO: 139 - protein, 12 aa, Artificial Sequence
  Synthetic SRP1558-A06-VL, CDR-L1
RASQSVSSNPLA

SEQ ID NO: 140 - protein, 12 aa, Artificial Sequence
  Synthetic SRP1558-F01-VL, CDR-L1
RASQSVSSGNPA

SEQ ID NO: 141 - protein, 12 aa, Artificial Sequence
  Synthetic SRP1448-D09-VL, CDR-L1
RASQSVSSSYLA

SEQ ID NO: 142 - protein, 11 aa, Artificial Sequence
  Synthetic trastuzumab-VL, CDR-L1
RASQDVNTAVA

SEQ ID NO: 143 - protein, 16 aa, Artificial Sequence
  Synthetic SRP1627-A02-VL, CDR-L1
KSSQSLLDSDGKTYLN

SEQ ID NO: 144 - protein, 16 aa, Artificial Sequence
  Synthetic SRP1627-A11-VL, CDR-L1
KSSQSLLDSDGKTYLN

SEQ ID NO: 145 - protein, 16 aa, Artificial Sequence
  Synthetic SRP1627-B01-VL, CDR-L1
KSSQSLLDSDGKTYLN

SEQ ID NO: 146 - protein, 16 aa, Artificial Sequence
  Synthetic h5G11-2-VL, CDR-L1
KSSQSLLDSDGKTYLN

SEQ ID NO: 147 - protein, 16 aa, Artificial Sequence
  Synthetic 421.61.4.5G11-VL, CDR-L1
KSSQSLLDSDGKTYLN

SEQ ID NO: 148 - protein, 11 aa, Artificial Sequence
  Synthetic 1353-G10-wt, CDRL1
SGDALPKQYAY

SEQ ID NO: 149 - protein, 11 aa, Artificial Sequence
  Synthetic 1353-G10-kappa, CDRL1
SGDALPKQYAY

SEQ ID NO: 150 - protein, 7 aa, Artificial Sequence
  Synthetic 26H10, CDR-L2
GASSRAT

SEQ ID NO: 151 - protein, 7 aa, Artificial Sequence
  Synthetic SRP1449-D05-VL, CDR-L2
GASSRAT

SEQ ID NO: 152 - protein, 7 aa, Artificial Sequence
  Synthetic SRP1449-F01-VL, CDR-L2
GASSRAT

SEQ ID NO: 153 - protein, 7 aa, Artificial Sequence
  Synthetic 1449-G09.2-VL, CDR-L2
GASSRAT

SEQ ID NO: 154 - protein, 7 aa, Artificial Sequence
  Synthetic SRP1449-B07-VL, CDR-L2
GASSRAT

SEQ ID NO: 155 - protein, 7 aa, Artificial Sequence
  Synthetic SRP1449-B03-VL, CDR-L2
GASSRAT

SEQ ID NO: 156 - protein, 7 aa, Artificial Sequence
  Synthetic SRP1558-E11-VL, CDR-L2
GASSRAT

SEQ ID NO: 157 - protein, 7 aa, Artificial Sequence
  Synthetic SRP1558-A06-VL, CDR-L2
GASSRAT

SEQ ID NO: 158 - protein, 7 aa, Artificial Sequence
  Synthetic SRP1558-F01-VL, CDR-L2
GASSRAT

SEQ ID NO: 159 - protein, 7 aa, Artificial Sequence
  Synthetic SRP1448-D09-VL, CDR-L2
GASSRAT

SEQ ID NO: 160 - protein, 7 aa, Artificial Sequence
  Synthetic trastuzumab-VL, CDR-L2
SASFLYS

SEQ ID NO: 161 - protein, 7 aa, Artificial Sequence
  Synthetic SRP1627-A02-VL, CDR-L2
LVSKLDS

SEQ ID NO: 162 - protein, 7 aa, Artificial Sequence
  Synthetic SRP1627-A11-VL, CDR-L2
LVSKLDS

SEQ ID NO: 163 - protein, 7 aa, Artificial Sequence
  Synthetic SRP1627-B01-VL, CDR-L2
LVSKLDS

SEQ ID NO: 164 - protein, 7 aa, Artificial Sequence
  Synthetic h5G11-2-VL, CDR-L2
LVSKLDS

SEQ ID NO: 165 - protein, 7 aa, Artificial Sequence
  Synthetic 421.61.4.5G11-VL, CDR-L2
LVSKLDS

SEQ ID NO: 166 - protein, 7 aa, Artificial Sequence
  Synthetic 1353-G10-wt, CDRL2
KDTERPS

SEQ ID NO: 167 - protein, 7 aa, Artificial Sequence
  Synthetic 1353-G10 K50D, CDRL2
DDTERPS

SEQ ID NO: 168 - protein, 7 aa, Artificial Sequence
  Synthetic 1353-G10 T52D, CDRL2
KDDERPS

SEQ ID NO: 169 - protein, 7 aa, Artificial Sequence
  Synthetic 1353-G10 E53D, CDRL2
KDTDRPS

SEQ ID NO: 170 - protein, 7 aa, Artificial Sequence
  Synthetic 1353-G10 P55D, CDRL2
KDTERDS

SEQ ID NO: 171 - protein, 7 aa, Artificial Sequence
  Synthetic 1353-G10 S56D, CDRL2
KDTERPD

SEQ ID NO: 172 - protein, 7 aa, Artificial Sequence
  Synthetic 1353-G10-kappa, CDRL2
KDTERPS

SEQ ID NO: 173 - protein, 9 aa, Artificial Sequence
  Synthetic 26H10, CDR-L3
QQYGSSPFT

SEQ ID NO: 174 - protein, 9 aa, Artificial Sequence
  Synthetic SRP1449-D05-VL, CDR-L3
QQYGSTPFK

SEQ ID NO: 175 - protein, 9 aa, Artificial Sequence
  Synthetic SRP1449-F01-VL, CDR-L3
QQYGSSPFT

SEQ ID NO: 176 - protein, 9 aa, Artificial Sequence
  Synthetic 1449-G09.2-VL, CDR-L3
QQYGRSPFS

SEQ ID NO: 177 - protein, 9 aa, Artificial Sequence
  Synthetic SRP1449-B07-VL, CDR-L3
QQYGASPFT

SEQ ID NO: 178 - protein, 9 aa, Artificial Sequence
  Synthetic SRP1449-B03-VL, CDR-L3
QQYDRSPLT

SEQ ID NO: 179 - protein, 9 aa, Artificial Sequence
  Synthetic SRP1558-E11-VL, CDR-L3
QQYSLAPPT

SEQ ID NO: 180 - protein, 9 aa, Artificial Sequence
  Synthetic SRP1558-A06-VL, CDR-L3
QQYMAGPPT

SEQ ID NO: 181 - protein, 9 aa, Artificial Sequence
  Synthetic SRP1558-F01-VL, CDR-L3
QQYTAGPPT

SEQ ID NO: 182 - protein, 9 aa, Artificial Sequence
  Synthetic SRP1448-D09-VL, CDR-L3
QQDTAGPPT

SEQ ID NO: 183 - protein, 9 aa, Artificial Sequence
  Synthetic trastuzumab-VL, CDR-L3
QQHYTTPPT

SEQ ID NO: 184 - protein, 9 aa, Artificial Sequence
  Synthetic SRP1627-A02-VL, CDR-L3
SHGNPVPQT

SEQ ID NO: 185 - protein, 9 aa, Artificial Sequence
  Synthetic SRP1627-A11-VL, CDR-L3
WHGINFPQT

SEQ ID NO: 186 - protein, 9 aa, Artificial Sequence
  Synthetic SRP1627-B01-VL, CDR-L3
STYSHFPQT

SEQ ID NO: 187 - protein, 9 aa, Artificial Sequence
  Synthetic h5G11-2-VL, CDR-L3
WQGSHFPQT

SEQ ID NO: 188 - protein, 9 aa, Artificial Sequence
  Synthetic 421.61.4.5G11-VL, CDR-L3
WQGSHFPQT

SEQ ID NO: 189 - protein, 11 aa, Artificial Sequence
  Synthetic 1353-G10-wt, CDRL3
QSADNSITYRV

SEQ ID NO: 190 - protein, 11 aa, Artificial Sequence
  Synthetic 1353-G10-kappa, CDRL3
QSADNSITYRV

SEQ ID NO: 191 - protein, 122 aa, Artificial Sequence
  Synthetic 26H10, VH
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSS

SEQ ID NO: 192 - protein, 121 aa, Artificial Sequence
  Synthetic SRP1496-A03-VH, VH
EVQLVESGGGLVQPGGSLRLSCAASGFNINDTYIHWVRQAPGKGLEWVGIIDPYDGATDYADSVKGRFTISADTSKNTAYLQMNSLRAEDTAVYYCAREIFGFYWNPFDYWGQGTLVTVSS

SEQ ID NO: 193 - protein, 121 aa, Artificial Sequence; identical to SEQ ID NO: 192
  Synthetic SRP1496-A04-VH, VH

SEQ ID NO: 194 - protein, 121 aa, Artificial Sequence; identical to SEQ ID NO: 192
  Synthetic SRP1496-B08-VH, VH

SEQ ID NO: 195 - protein, 121 aa, Artificial Sequence
  Synthetic SRP1648-B07-VH, VH
EVQLVESGGGLVQPGGSLRLSCAASGFNIADTFIHWVRQAPGKGLEWVGIIDPYDGDTDYADSVKGRFTISADTSKNTAYLQMNSLRAEDTAVYYCAREILGFYWNPFDYWGQGTLVTVSS

SEQ ID NO: 196 - protein, 121 aa, Artificial Sequence
  Synthetic SRP1648-E02-VH, VH
EVQLVESGGGLVQPGGSLRLSCAASGFNINDNYIHWVRQAPGKGLEWVGIIDPYDGFTAYADSVKGRFTISADTSKNTAYLQMNSLRAEDTAVYYCARESIGFYLNPFDYWGQGTLVTVSS

SEQ ID NO: 197 - protein, 122 aa, Artificial Sequence
  Synthetic SRP1449-B03-VH, VH
QVQLVESGGGVVQPGRSLRLSCAASGFTFSSYGMHWVRQAPGKGLEWVAAIWYDASYKYYADSVKGRFTISRDNSKNTLYLQMNSLRAEDTAVYYCAREWAVASWDYALDVWGQGTTVTVSS

SEQ ID NO: 198 - protein, 122 aa, Artificial Sequence
  Synthetic SRP1449-F01-VH, VH
QVQLVESGGGVVQPGRSLRLSCAASGFTFSSYGMHWVRQAPGKGLEWVAVIWYDGSYKYYADSVKGRFAISRDNSKNTLYLQMNSLRAEDTAVYYCARESEVASWDYGLDVWGQGTTVTVSS

SEQ ID NO: 199 - protein, 122 aa, Artificial Sequence
  Synthetic SRP1449-B07-VH, VH
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVSSWDYGMDVWGQGTTVTVSS

SEQ ID NO: 200 - protein, 122 aa, Artificial Sequence
  Synthetic 1449-G09.2-VH, VH
QVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSS

SEQ ID NO: 201 - protein, 122 aa, Artificial Sequence
  Synthetic SRP1449-D05-VH, VH
QVQLVESGGGVVQPGRSLRLSCAASGFTFRSFGMHWVRQAPGKGLEWVAVIWYDGSVKYYADSVKGRFTISRDNSKNTLYLQMNSLRAEDTAVYYCAREWADVSWDAGLDVWGQGTTVTVSS

SEQ ID NO: 202 - protein, 121 aa, Artificial Sequence
  Synthetic SRP1558-F01-VH, VH
EVQLLESGGGLVQPGGSLRLSCAASGFTFPDSSMSWVRQAPGKGLEWVGVITDNSGNTDYADSVKGRFTISRDNSKNTLYLQMNSLRAEDTAVYYCAKVFEGGVRPYSDYWGQGTLVTVSS

SEQ ID NO: 203 - protein, 121 aa, Artificial Sequence
  Synthetic SRP1448-D09-VH, VH
EVQLLESGGGLVQPGGSLRLSCAASGFTFTDSSMSWVRQAPGKGLEWVGVITGNSGTTDYADSVKGRFTISRDNSKNTLYLQMNSLRAEDTAVYYCAKVYEGGVRPYSDYWGQGTLVTVSS

SEQ ID NO: 204 - protein, 121 aa, Artificial Sequence
  Synthetic SRP1558-A06-VH, VH
EVQLLESGGGLVQPGGSLRLSCAASGFTFSESTMSWVRQAPGKGLEWVGFITSDSGTTDYADSVKGRFTISRDNSKNTLYLQMNSLRAEDTAVYYCAKVFEGGVRPFSDYWGQGTLVTVSS

SEQ ID NO: 205 - protein, 121 aa, Artificial Sequence
  Synthetic SRP1558-E11-VH, VH
EVQLLESGGGLVQPGGSLRLSCAASGFTFTSSSMSWVRQAPGKGLEWVGVISDDTGSTDYADSVKGRFTISRDNSKNTLYLQMNSLRAEDTAVYYCAKVDNGGVRPYSDYWGQGTLVTVSS

SEQ ID NO: 206 - protein, 115 aa, Artificial Sequence
  Synthetic SRP1627-A02-VH, VH
QVQLVQSGAEVKKPGSSVKVSCKASGFNINDYFMHWVRQAPGQGLEWIARIDPWNGDTEYAPKFQGRVTITADESTSTAYMELSSLRSEDTAVYYCGMSDALDYWGQGTLVTVSS

SEQ ID NO: 207 - protein, 115 aa, Artificial Sequence; identical to SEQ ID NO: 206
  Synthetic SRP1627-A11-VH, VH

SEQ ID NO: 208 - protein, 115 aa, Artificial Sequence
  Synthetic h5G11-2-VH, VH
QVQLVQSGAEVKKPGSSVKVSCKASGFNIKDYYMHWVRQAPGQGLEWIAWIDPENGDTEYAPKFQGRVTITADESTSTAYMELSSLRSEDTAVYYCNAPDALDYWGQGTLVTVSS

SEQ ID NO: 209 - protein, 115 aa, Artificial Sequence
  Synthetic SRP1627-B01-VH, VH
QVQLVQSGAEVKKPGSSVKVSCKASGFNITDLYMHWVRQAPGQGLEWIARIDPWNGDTEYAPKFQGRATITADESTSTAYMELSSLRSEDTAVYYCIASEMVDYWGQGTLVTVSS

SEQ ID NO: 210 - protein, 115 aa, Artificial Sequence
  Synthetic 421.61.4.5G11-VH, VH
EVQLQQSGAELVRSGASVKLSCTASGFNIKDYYMHWVKQRPEQGLEWIAWIDPENGDTEYAPKFQGRATLTADTSSNTAYLHLSSLTSEDTAVYYCNAPDALDYWGQGTSVTVSS

SEQ ID NO: 211 - protein, 119 aa, Artificial Sequence
  Synthetic 1353-G10-wt, VH
EVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 212 - protein, 119 aa, Artificial Sequence
  Synthetic 1353-G10 Y27D, VH
EVQLVQSGAEVKKPGASVKVSCKASGDRFPHYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 213 - protein, 119 aa, Artificial Sequence
  Synthetic 1353-G10 R28D, VH
EVQLVQSGAEVKKPGASVKVSCKASGYDFPHYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 214 - protein, 119 aa, Artificial Sequence
  Synthetic 1353-G10 F29D, VH
EVQLVQSGAEVKKPGASVKVSCKASGYRDPHYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 215 - protein, 119 aa, Artificial Sequence
  Synthetic 1353-G10 P30D, VH
EVQLVQSGAEVKKPGASVKVSCKASGYRFDHYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 216 - protein, 119 aa, Artificial Sequence
  Synthetic 1353-G10 H31D, VH
EVQLVQSGAEVKKPGASVKVSCKASGYRFPDYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 217 - protein, 119 aa, Artificial Sequence
  Synthetic 1353-G10 Y32D, VH
EVQLVQSGAEVKKPGASVKVSCKASGYRFPHDGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 218 - protein, 119 aa, Artificial Sequence
  Synthetic 1353-G10 G33D, VH
EVQLVQSGAEVKKPGASVKVSCKASGYRFPHYDISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 219 - protein, 119 aa, Artificial Sequence
  Synthetic 1353-G10 S35D, VH
EVQLVQSGAEVKKPGASVKVSCKASGYRFPHYGIDWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 220 - protein, 119 aa, Artificial Sequence
  Synthetic 1353-G10 R28T/P30D, VH
EVQLVQSGAEVKKPGASVKVSCKASGYTFDHYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 221 - protein, 119 aa, Artificial Sequence
  Synthetic 1353-G10 R28D/P30D, VH
EVQLVQSGAEVKKPGASVKVSCKASGYDFDHYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 222 - protein, 119 aa, Artificial Sequence
  Synthetic 1353-G10 R28D/H31D, VH
EVQLVQSGAEVKKPGASVKVSCKASGYDFPDYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 223 - protein, 119 aa, Artificial Sequence
  Synthetic 1353-G10 R28T/H31D, VH
EVQLVQSGAEVKKPGASVKVSCKASGYTFPDYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 224 - protein, 119 aa, Artificial Sequence
  Synthetic 1353-G10 R28D/P30T/H31S, VH
EVQLVQSGAEVKKPGASVKVSCKASGYDFTSYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 225 - protein, 119 aa, Artificial Sequence
  Synthetic 1353-G10 R28T/P30T/H31D, VH
EVQLVQSGAEVKKPGASVKVSCKASGYTFTDYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 226 - protein, 119 aa, Artificial Sequence
  Synthetic 1353-G10 R28T/P30T/H31S, VH
EVQLVQSGAEVKKPGASVKVSCKASGYTFTSYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 227 - protein, 119 aa, Artificial Sequence
  Synthetic 1353-G10-R28T/P30D/H31S, VH
EVQLVQSGAEVKKPGASVKVSCKASGYTFDSYGISWVRQAPGQGLEWMGWISAYNGNTNYAQKLQGRVTMTTDTSTNTAYMELRSLRSDDTAVYYCARDVDYGTGSGYWGQGTLVTVSS

SEQ ID NO: 228 - protein, 119 aa, Artificial Sequence
  Synthetic 10B4, VH
QVQLQQSGAELMKPGASVKMSCKTTGYIFSSYWIGWVKQRPGHGLEWIGKIFPGSGSADYNENFKGKATFTVDTSSNTAYMQLSSLTSEDSAVYYCARGYGNYLYFDVWGAGTTVTVSS

SEQ ID NO: 229 - protein, 119 aa, Artificial Sequence
  Synthetic 1353-A09, VH
EVQLVQSGAEVKKPGASVKVSCKASGYRFTWYGISWVRQAPGQGLEWMGWISAYNGNTNYAQKLQGRVTMTTDTSTNTAYMELRSLRSDDTAVYYCARDSEYSSGSGYWGQGTLVTVSS

SEQ ID NO: 230 - protein, 119 aa, Artificial Sequence
  Synthetic 1353-C07, VH
EVQLVQSGAEVKKPGASVKVSCKASGYRFSTFGISWVRQAPGQGLEWMGWISAYNGNTNYAQKLQGRVTMTTDTSTNTAYMELRSLRSDDTAVYYCARDVDYSSGSGYWGQGTLVTVSS

SEQ ID NO: 231 - protein, 119 aa, Artificial Sequence
  Synthetic 1353-E07, VH
EVQLVQSGAEVKKPGASVKVSCKASGYRFETYGISWVRQAPGQGLEWMGWISAYNGNTNYAQKLQGRVTMTTDTSTNTAYMELRSLRSDDTAVYYCARDAEYSLGSGYWGQGTLVTVSS

SEQ ID NO: 232 - protein, 119 aa, Artificial Sequence
  Synthetic 1353-F09, VH
EVQLVQSGAEVKKPGASVKVSCKASGYRFRQYGISWVRQAPGQGLEWMGWISAYNGNTNYAQKLQGRVTMTTDTSTNTAYMELRSLRSDDTAVYYCARDAEYGSGSGYWGQGTLVTVSS

SEQ ID NO: 233 - protein, 119 aa, Artificial Sequence
  Synthetic 1353-G08, VH
EVQLVQSGAEVKKPGASVKVSCKASGYRFTRYGISWVRQAPGQGLEWMGWVSAHNGNTNYAQKLQGRVTMTTDTSTNTAYMELRSLRSDDTAVYYCARDADYGSGSGYWGQGTLVTVSS

SEQ ID NO: 234 - protein, 119 aa, Artificial Sequence
  Synthetic 1353-H08, VH
EVQLVQSGAEVKKPGASVKVSCKASGYRFTRQGISWVRQAPGQGLEWMGWISAYNGNTKYAQKLQGRVTMTTDTSTNTAYMELRSLRSDDTAVYYCARDVDYGSGSGYWGQGTLVTVSS

SEQ ID NO: 235 - protein, 119 aa, Artificial Sequence
  Synthetic 1353-H09, VH
EVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDAEYGSGSGYWGQGTLVTVSS

SEQ ID NO: 236 - protein, 115 aa, Artificial Sequence
  Synthetic 1B10, VH
DVQLQESGPGLVKPSQSLSLTCTVTGHSITSDYAWNWIRQFPGNKLEWMGYISYSGRTSYNPSLTSRISITRDTSKNQFFLQLNSVTTEDTATYYCARGYALDYWGQGTSVTVSS

SEQ ID NO: 237 - protein, 120 aa, Artificial Sequence
  Synthetic 1E9, VH
EVKLVESGGGLVSPGGSLKLSCAASGFTFSTFGMSWVRQTPEKRLEWVATISGGGSDTYYPDSVQGRFIISRYNAKNNLYLQMNSLRPEDTALYYCARQGYDVYSWFAYWGQGTLVTVSA

SEQ ID NO: 238 - protein, 118 aa, Artificial Sequence
  Synthetic 4B10, VH
EVKLVESGGGLVKPGGSLKLSCAASGFTFSTYGMSWVRQTPEKRLQWVATISGGGSNTYYSDSVKGRFTISRDNAKNNLYLQMSSLRSEDTALYYCARQRDSAWFASWGQGTLVTVSA

SEQ ID NO: 239 - protein, 120 aa, Artificial Sequence
  Synthetic h1E9-1, VH
EVQLVESGGGLVKPGGSLRLSCAASGFTFSTFGMSWVRQAPGKGLEWVSTISGGGSDTYYPDSVQGRFTISRDNAKNSLYLQMNSLRAEDTAVYYCARQGYDVYSWFAYWGQGTLVTVSS

SEQ ID NO: 240 - protein, 120 aa, Artificial Sequence; identical to SEQ ID NO: 239
  Synthetic h1E9-2, VH

SEQ ID NO: 241 - protein, 120 aa, Artificial Sequence
  Synthetic h1E9-4, VH
EVQLVESGGGLVQPGGSLRLSCAASGFTFSTFGMSWVRQAPGKGLEWVATISGGGSDTYYPDSVQGRFTISRDNAKNSLYLQMNSLRAEDTAVYYCARQGYDVYSWFAYWGQGTLVTVSS

SEQ ID NO: 242 - protein, 120 aa, Artificial Sequence; identical to SEQ ID NO: 241
  Synthetic h1E9-5, VH

SEQ ID NO: 243 - protein, 118 aa, Artificial Sequence
  Synthetic h4B10-1, VH
EVQLVESGGGLVKPGGSLRLSCAASGFTFSTYGMSWVRQAPGKGLEWVATISGGGSNTYYSDSVKGRFTISRDDSKNTLYLQMNSLKTEDTAVYYCARQRDSAWFASWGQGTLVTVSS

SEQ ID NO: 244 - protein, 118 aa, Artificial Sequence; identical to SEQ ID NO: 243
  Synthetic h4B10-2, VH

SEQ ID NO: 245 - protein, 118 aa, Artificial Sequence; identical to SEQ ID NO: 243
  Synthetic h4B10-3, VH

SEQ ID NO: 246 - protein, 128 aa, Artificial Sequence
  Synthetic PD1-17, VH
QVQLQESGPGVVKPSGTLSLTCAISGGSIGSGGSIRSTRWWSWVRQSPGKGLEWIGEIYHSGSTNYNPSLKSRVTISLDKSRNHFSLRLNSVTAADTAVYYCARQDYGDSGDWYFDLWGKGTMVTVSS

SEQ ID NO: 247 - protein, 119 aa, Artificial Sequence
  Synthetic PD1-28, VH
EVQLVQSGAEVKKPGASVKVSCKASGYRFTSYGISWVRQAPGQGLEWMGWISAYNGNTNYAQKLQGRVTMTTDTSTNTAYMELRSLRSDDTAVYYCARDADYSSGSGYWGQGTLVTVSS

SEQ ID NO: 248 - protein, 119 aa, Artificial Sequence
  Synthetic PD1-33, VH
QVQLVQSGAEVKKPGASVRVSCKASGYTLTSYYIHWVRQAPGQGLEWMGIINPRGATISYAQKFQGRVTMTRDTSTSTVYMELRNLKSEDTALYYCATAGIYGFDFDYWGRGTLVTVSS

SEQ ID NO: 249 - protein, 127 aa, Artificial Sequence
  Synthetic PD1-35, VH
QVQLQESGPGLVKPSQTLSLTCTVSGGSISSGAYYWSWIRQHPGKGLEWIGYIYYNGNTYYNPSLRSLVTISVDASKNQFSLKLSSVTAADTAVYYCARASDYVWGGYRYMDAFDIWGRGTLITVSS

SEQ ID NO: 250 - protein, 121 aa, Artificial Sequence
  Synthetic PD1-F2, VH
EVQLVQSGGGVVQPGRSLRLSCAASGFTFSSYWCDRMSWVRQAPGKGLEWVSAISGSGGSTYYADSVKGRFTISRDNSKNTLYLQMNSLRAEDTAVYYCAKENWGSYFDLWGQGTTVTVSS

SEQ ID NO: 251 - protein, 108 aa, Artificial Sequence
  Synthetic 26H10, VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 252 - protein, 108 aa, Artificial Sequence
  Synthetic SRP1449-D05-VL, VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSTPFKFGPGTKVDIK

SEQ ID NO: 253 - protein, 108 aa, Artificial Sequence
  Synthetic SRP1449-F01-VL, VL
EIALTQSPGTLSLSPGERATLSCRASRSVSSSYLAWYQQKPGQAPRLLIYGASSRATGIPDRFSGSGSGTDFTLTISRLEPEDFAVYYCQQYGSSPFTFGPGTKVDIK

SEQ ID NO: 254 - protein, 108 aa, Artificial Sequence
  Synthetic 1449-G09.2-VL, VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIK

SEQ ID NO: 255 - protein, 108 aa, Artificial Sequence
  Synthetic SRP1449-B07-VL, VL
EIVLTQSPGTLSLSPGERATLSCRASQSVSSSYLAWYQQKPGQAPRLLIYGASSRATGIPNRFSGSGSGTDFTLTISRLEPEDFAVYYCQQYGASPFTFGPGTKVDIK

SEQ ID NO: 256 - protein, 108 aa, Artificial Sequence
  Synthetic SRP1449-B03-VL, VL
EIVLTQSPGTMSLSPGERATLSCRASQSVSSSYLAWYQQKPGQAPRLLIYGASSRATGIPDRFSGSGSGTDFTLTISRLEPEDFAVYYCQQYDRSPLTFGPGTKVDIK

SEQ ID NO: 257 - protein, 108 aa, Artificial Sequence
  Synthetic SRP1558-E11-VL, VL
EIVLTQSPGTLSLSPGERATLSCRASQSVSSSYLAWYQQKPGQAPRLLIYGASSRATGIPDRFSGSGSGTDFTLTISRLEPEDFAVYYCQQYSLAPPTLGQGTKVEIK

SEQ ID NO: 258 - protein, 108 aa, Artificial Sequence
  Synthetic SRP1558-A06-VL, VL
EIVLTQSPGTLSLSPGERATLSCRASQSVSSNPLAWYQQKPGQAPRLLIYGASSRATGIPDRFSGSGSGTDFTLTISRLEPEDFAVYYCQQYMAGPPTFGQGTKVEIK

SEQ ID NO: 259 - protein, 108 aa, Artificial Sequence
  Synthetic SRP1558-F01-VL, VL
  misc_feature (82)..(82): Xaa can be any naturally occurring amino acid
EIVLTQSPGTLSLSPGERATLSCRASQSVSSGNPAWYQQKPGQAPRLLIYGASSRATGIPDRFSGSGSGTDFTLTISRLEPXDFAVYYCQQYTAGPPTFGQGTKVEIK

SEQ ID NO: 260 - protein, 108 aa, Artificial Sequence
  Synthetic SRP1448-D09-VL, VL
EIVLTQSPGTLSLSPGERATLSCRASQSVSSSYLAWYQQKPGQAPRLLIYGASSRATGIPDRFSGSGSGTDFTLTISRLEPEDFAVYYCQQDTAGPPTFGQGTKVEIK

SEQ ID NO: 261 - protein, 107 aa, Artificial Sequence [sequence omitted]

SEQ ID NO: 262 - protein, 112 aa, Artificial Sequence
  Synthetic SRP1627-A02-VL, VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCSHGNPVPQTFGQGTKVEIK

SEQ ID NO: 263 - protein, 112 aa, Artificial Sequence
  Synthetic SRP1627-A11-VL, VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWHGINFPQTFGQGTKVEIK

SEQ ID NO: 264 - protein, 112 aa, Artificial Sequence
  Synthetic SRP1627-B01-VL, VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCSTYSHFPQTFGQGTKVEIK

SEQ ID NO: 265 - protein, 112 aa, Artificial Sequence
  Synthetic h5G11-2-VL, VL
DVVMTQSPLSLPVTLGQPASISCKSSQSLLDSDGKTYLNWFQQRPGQSPRRLIYLVSKLDSGVPDRFSGSGSGTDFTLKISRVEAEDVGVYYCWQGSHFPQTFGQGTKVEIK

SEQ ID NO: 266 - protein, 112 aa, Artificial Sequence
  Synthetic 421.61.4.5G11-VL, VL
DVVMTQTPLTLSVTIGQIASISCKSSQSLLDSDGKTYLNWLLQRPGQSPKRLIYLVSKLDSGVPDRFTGSGSGTDFTLKISRVEAEDLGVYYCWQGSHFPQTFGGGTKLEIK

SEQ ID NO: 267 - protein, 108 aa, Artificial Sequence
  Synthetic 1353-G10-wt (lambda), VL
SYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVL

SEQ ID NO: 268 - protein, 108 aa, Artificial Sequence
  Synthetic 1353-G10 K50D (lambda), VL
SYELTQPPSVSVSPGQTARITCSGDALPKQYAYWYQQKPGQAPVMVIYDDTERPSGIPERFSGSSSGTKVTLTISGVQAEDEADYYCQSADNSITYRVFGGGTKVTVL

SEQ ID NO: 269 - protein, 108 aa, Artificial Sequence
  Synthetic 1353-G10 T52D (lambda), VL
SYELTQPPSVSVSPGQTARITCSGDALPKQYAYWYQQKPGQAPVMVIYKDDERPSGIPERFSGSSSGTKVTLTISGVQAEDEADYYCQSADNSITYRVFGGGTKVTVL

SEQ ID NO: 270 - protein, 108 aa, Artificial Sequence
  Synthetic 1353-G10 E53D (lambda), VL
SYELTQPPSVSVSPGQTARITCSGDALPKQYAYWYQQKPGQAPVMVIYKDTDRPSGIPERFSGSSSGTKVTLTISGVQAEDEADYYCQSADNSITYRVFGGGTKVTVL

SEQ ID NO: 271 - protein, 108 aa, Artificial Sequence
  Synthetic 1353-G10 P55D (lambda), VL
SYELTQPPSVSVSPGQTARITCSGDALPKQYAYWYQQKPGQAPVMVIYKDTERDSGIPERFSGSSSGTKVTLTISGVQAEDEADYYCQSADNSITYRVFGGGTKVTVL

SEQ ID NO: 272 - protein, 108 aa, Artificial Sequence
  Synthetic 1353-G10 S56D (lambda), VL
SYELTQPPSVSVSPGQTARITCSGDALPKQYAYWYQQKPGQAPVMVIYKDTERPDGIPERFSGSSSGTKVTLTISGVQAEDEADYYCQSADNSITYRVFGGGTKVTVL

SEQ ID NO: 273 - protein, 108 aa, Artificial Sequence
  Synthetic 1353-G10 VL1c (kappa), VL
SYELTQPPSVSVSPGQTARITCSGDALPKQYAYWYQQKPGQAPVMVIYKDTERPSGIPERFSGSSSGTKVTLTISGVQAEDFATYYCQSADNSITYRVFGGGTKVEIK

SEQ ID NO: 274 - protein, 108 aa, Artificial Sequence
  Synthetic 1353-G10 VL1d (kappa), VL
SYELTQPPSVSVSPGQTARITCSGDALPKQYAYWYQRKPGQAPVMVIYKDTERPSGIPERFSGSSSGTKVTLTISGVQAEDFATYYCQSADNSITYRVFGGGTKVEIK

SEQ ID NO: 275 - protein, 109 aa, Artificial Sequence
  Synthetic 1353-G10 Vk1-39 (kappa), VL
DIQLTQSPSSLSASVGDRVTITCSGDALPKQYAYWYQQKPGKAPKLLIYKDTERPSGVPSRFSGSSSGTKVTLTISSLQPEDFATYYCQSADNSITYRVFGGGTKVEIK

SEQ ID NO: 276 - protein, 109 aa, Artificial Sequence
  Synthetic 1353-G10 Vk3-20 (kappa), VL
EIVLTQSPGTLSLSPGERATLSCSGDALPKQYAYWYQQKPGQAPRLLIYKDTERPSGIPDRFSGSSSGTKVTLTISRLEPEDFAVYYCQSADNSITYRVFGGGTKVEIK

SEQ ID NO: 277 - protein, 108 aa, Artificial Sequence
  Synthetic 10B4, VL
NIVMTQTPKFLLVSAGDRITITCKASQSVSDDVAWYQQKPGQSPKLLISYAFKRYIGVPDRFTGSGYGTDFTFTISTVQAEDLAVYFCQQNYNSPYTFGGGTKLELKR

SEQ ID NO: 278 - protein, 108 aa, Artificial Sequence
  Synthetic 1353-A09, VL
SYELTQPPSVSVSPGQTARITCSGDALTTQYAYWYQQKPGQAPVMVIYKDTERPSGIPERFSGSSSGTKVTLTISGVQAEDEADYYCQSADNSITYRVFGGGTKVTVL

SEQ ID NO: 279 - protein, 108 aa, Artificial Sequence
  Synthetic 1353-C07, VL
SYELTQPPSVSVSPGQTARITCSGDALSEQYAYWYQQKPGQAPVMVIYKDTERPSGIPERFSGSSSGTKVTLTISGVQAEDEADYYCQSADNSITYRVFGGGTKVTVL

SEQ ID NO: 280 - protein, 108 aa, Artificial Sequence; identical to SEQ ID NO: 267
  Synthetic 1353-E07, VL

SEQ ID NO: 281 - protein, 108 aa, Artificial Sequence
  Synthetic 1353-F09, VL
SYELTQPPSVSVSPGQTARITCSGDALPKQYAYWYQQKPGQAPVMVLYKDTERPSGIPERFSGSSSGTKVTLTISGVQAEDEADYYCQSADNSITYRVFGGGTKVTVL

SEQ ID NO: 282 - protein, 108 aa, Artificial Sequence
  Synthetic 1353-G08, VL
SYELTQPPSVSVSPGQTARITCSGDALPMQYGYWYQQKPGQAPVMVIYKDTERPSGIPERFSGSSSGTKVTLTISGVQAEDEADYYCQSADNSITYRVFGGGTKVTVL

SEQ ID NO: 283 - protein, 108 aa, Artificial Sequence; identical to SEQ ID NO: 267
  Synthetic 1353-H08, VL

SEQ ID NO: 284 - protein, 108 aa, Artificial Sequence; identical to SEQ ID NO: 267
  Synthetic 1353-H09, VL

SEQ ID NO: 285 - protein, 106 aa, Artificial Sequence
  Synthetic 1B10, VL
QIVLSQSPAILSASPGEKVTMTCRTSSSVNYMHWFQQKPGSSPKPWIYATSKLASGVPARFSGSGSGTSYSLTISRVEAEDAATYFCQQWISDPWTFGGGTKLEIK

SEQ ID NO: 286 - protein, 111 aa, Artificial Sequence
  Synthetic 1E9, VL
DIILTQSPASLAVSLGQRAAISCRASESVDNSGISFMSWFQQKPGQPPKLLIYTASNQGSGVPARFSGSGSGTEFSLNIHPMEEDDTAMYFCQQSKEVPWTFGGGTKLEIR

SEQ ID NO: 287 - protein, 111 aa, Artificial Sequence
  Synthetic 4B10, VL
DIVLTQSPASLAVSLGQRATISCRASENVDDYGVSFMNWFQQKPGQPPKLLIYPASNQGSGVPARFSGSGSGTDFSLNIHPMEEDDTAMYFCQQSKEVPWTFGGGTKLEIK

SEQ ID NO: 288 - protein, 111 aa, Artificial Sequence
  Synthetic h1E9-1, VL
DIQLTQSPSFLSASVGDRVTITCRASESVDNSGISFMSWYQQKPGKAPKLLIYTASNQGSGVPSRFSGSGSGTEFTLTISSLQPEDFATYYCQQSKEVPWTFGQGTKVEIK

SEQ ID NO: 289 - protein, 111 aa, Artificial Sequence
  Synthetic h1E9-2, VL
EIVLTQSPATLSLSPGERATLSCRASESVDNSGISFMSWYQQKPGQAPRLLIYTASNQGSGIPARFSGSGSGTDFTLTISSLEPEDFAVYYCQQSKEVPWTFGQGTKVEIK

SEQ ID NO: 290 - protein, 111 aa, Artificial Sequence; identical to SEQ ID NO: 288
  Synthetic h1E9-4, VL

SEQ ID NO: 291 - protein, 111 aa, Artificial Sequence; identical to SEQ ID NO: 289
  Synthetic h1E9-5, VL

SEQ ID NO: 292 - protein, 111 aa, Artificial Sequence
  Synthetic h4B10-1, VL
EIVLTQSPATLSLSPGERATLSCRASENVDDYGVSFMNWYQQKPGQAPRLLIYPASNQGSGIPARFSGSGSGTDFTLTISSLEPEDFAVYYCQQSKEVPWTFGQGTKVEIK

SEQ ID NO: 293 - protein, 111 aa, Artificial Sequence
  Synthetic h4B10-2, VL
EIVLTQSPGTLSLSPGERATLSCRASENVDDYGVSFMNWYQQKPGQAPRLLIYPASNQGSGIPDRFSGSGSGTDFTLTISRLEPEDFAVYYCQQSKEVPWTFGQGTKVEIK

SEQ ID NO: 294 - protein, 111 aa, Artificial Sequence
  Synthetic h4B10-3, VL
DIVMTQSPDSLAVSLGERATINCRASENVDDYGVSFMNWYQQKPGQPPKLLIYPASNQGSGVPDRFSGSGSGTDFTLTISSLQAEDVAVYYCQQSKEVPWTFGGGTKLEIK

SEQ ID NO: 295 - protein, 110 aa, Artificial Sequence
  Synthetic PD1-17, VL
NFMLTQPHSVSESPGKTVTISCTRSSGSIASNSVQWYQQRPGSSPTTVIYEDNQRPSGVPDRFSGSIDSSSNSASLTVSGLKTEDEADYYCQSSDSSAVVFGSGTKLTVL

SEQ ID NO: 296 - protein, 108 aa, Artificial Sequence; identical to SEQ ID NO: 267
  Synthetic PD1-28, VL

SEQ ID NO: 297 - protein, 111 aa, Artificial Sequence
  Synthetic PD1-33, VL
QSALTQPASVSGSPGQSITISCTGTSNDVGGYNYVSWYQHHPGKAPKLIIYDVTNRPSGVSDRFSGSKSGNTASLTISGLLAEDEGDYYCSSYTIVTNFEVLFGGGTKLTV

SEQ ID NO: 298 - protein, 110 aa, Artificial Sequence
  Synthetic PD1-35, VL
QSVLTQPPSASGTPGQRVTISCSGSNSNIGSNSVNWYQQLPGTAPKLLIYGNNQRPSGVPDRFSGSKSGTSASLAISGLQSENEADYYCAAWDDSLNGPVFGRGTKVTVL

SEQ ID NO: 299 - protein, 107 aa, Artificial Sequence
  Synthetic PD1-F2, VL
DIVMTQSPSTLSASVGDRVTITCRASQGISSWLAWYQQKPGRAPKVLIYKASTLESGVPSRFSGSGSGTDFTLTISSLQPEDFATYYCQQSYSTPWTFGQGTKLEIK

SEQ ID NO: 300 - protein, 330 aa, Homo sapiens
  misc_feature (1)..(330): Human IgG1 HC Constant
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 301 - protein, 107 aa, Homo sapiens [sequence omitted]

SEQ ID NO: 302 - protein, 323 aa, Mus musculus
  misc_feature (1)..(323): Mouse IgG1 HC Constant
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPG

SEQ ID NO: 303 - protein, 107 aa, Mus musculus
  misc_feature (1)..(107): Mouse IgG LC Constant Ckappa
RADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 304 - protein, 108 aa, Artificial Sequence
  Synthetic Kappa LC
HMTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 305 - protein, 106 aa, Artificial Sequence
  Synthetic Lambda LC
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 306 - protein, 252 aa, Artificial Sequence
  Synthetic IgG1 Fc from scFvFc
AAGSDQEPKSSDKTHTCPPCSAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGDYKDDDDKGSG

SEQ ID NO: 307 - protein, 20 aa, Artificial Sequence
  Synthetic FlagHis Tag
GSGDYKDDDDKGSGHHHHHH

SEQ ID NO: 308 - protein, 15 aa, Artificial Sequence
  Synthetic Linker (GGGGS)3
GGGGSGGGGSGGGGS

SEQ ID NO: 309 - protein, 20 aa, Artificial Sequence
  Synthetic Linker (GGGGS)4
GGGGSGGGGSGGGGSGGGGS

SEQ ID NO: 310 - protein, 25 aa, Artificial Sequence
  Synthetic Linker (GGGGS)5
GGGGSGGGGSGGGGSGGGGSGGGGS

SEQ ID NO: 311 - protein, 30 aa, Artificial Sequence
  Synthetic Linker (GGGGS)6
GGGGSGGGGSGGGGSGGGGSGGGGSGGGGS

SEQ ID NO: 312 - protein, 11 aa, Artificial Sequence
  Synthetic Linker
AAGSDQEPKSS

SEQ ID NO: 313 - protein, 6 aa, Artificial Sequence
  Synthetic Linker
AAGSDQ

SEQ ID NO: 314 - protein, 18 aa, Artificial Sequence
  Synthetic Linker
APGPSAPSHRSLPSRAFG

SEQ ID NO: 315 - protein, 21 aa, Artificial Sequence
  Synthetic Linker-hinge
AAGSDQEPKSSDKTHTCPPCP

SEQ ID NO: 316 - protein, 10 aa, Artificial Sequence
  Synthetic Hinge - wt
DKTHTCPPCP

SEQ ID NO: 317 - protein, 245 aa, Artificial Sequence
  Synthetic 26H10 , scFv
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCAREWAVASWDYGMDVWGQGTTVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPFTFGPGTKVDIK

SEQ ID NO: 318 - protein, 288 aa, Artificial Sequence
  Synthetic hPD-1
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 319 - protein, 288 aa, Mus musculus
  misc_feature (1)..(288): murine PD-1
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALAVFCSTSMSEARGAGSKDDTLKEEPSAAPVPSVAYEELDFQGREKTPELPTACVHTEYATIVFTEGLGASAMGRRGSADGLQGPRPPRHEDGHCSWPL

SEQ ID NO: 320 - protein, 288 aa, Macaca fascicularis; identical to SEQ ID NO: 319
  misc_feature (1)..(288): cyno PD-1

SEQ ID NO: 321 - protein, 217 aa, Artificial Sequence
  Synthetic CH2-CH3, Fc-knob
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 217 aa, Artificial Sequence
  Synthetic CH2-CH3, Fc-knob-V262E
APELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 323 - protein, 217 aa, Artificial Sequence
  Synthetic CH2-CH3, Fc-knob-V264S
APELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 324 - protein, 217 aa, Artificial Sequence
  Synthetic CH2-CH3, Fc-knob-D399C
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLCSDGSFFLYSKLTVDKSRWQQGNVFSCSVMHEALHNHYTQKSLSLSPGK

SEQ ID NO: 325 - protein, 217 aa, Artificial Sequence
  Synthetic CH2-CH3, Fc-knob-S354C
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 326 - protein, 217 aa, Artificial Sequence
  Synthetic CH2-CH3, Fc-hole
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 327 - protein, 217 aa, Artificial Sequence
  Synthetic CH2-CH3, Fc-hole-V262E
APELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 328 - protein, 217 aa, Artificial Sequence
  Synthetic CH2-CH3, Fc-hole-V264S
APELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 329 - protein, 217 aa, Artificial Sequence
  Synthetic CH2-CH3, Fc-hole-Y349C
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 330 - protein, 217 aa, Artificial Sequence
  Synthetic CH2-CH3, Fc-hole-K392C
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYCTTPPVLDSDGSFFLVSKLTVDKSRWQQGNVFSCSVMHEALHNHYTQKSLSLSPGK

SEQ ID NO: 331 - protein, 217 aa, Artificial Sequence
  Synthetic Fc-zwA
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 332 - protein, 217 aa, Artificial Sequence
  Synthetic Fc-zwA-V262E
APELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 333 - protein, 217 aa, Artificial Sequence
  Synthetic Fc-zwA-V264S
APELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 334 - protein, 217 aa, Artificial Sequence
  Synthetic Fc-zwB
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 335 - protein, 217 aa, Artificial Sequence
  Synthetic Fc-zwB-V262E
APELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 336 - protein, 217 aa, Artificial Sequence
  Synthetic Fc-zwB-V264S
APELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 337 - protein, 227 aa, Artificial Sequence
  Synthetic hinge-CH2-CH3-zwA
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 338 - protein, 227 aa, Artificial Sequence
  Synthetic hinge-CH2-CH3-zwA V262E
DKTHTCPPCPAPELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 339 - protein, 227 aa, Artificial Sequence
  Synthetic hinge-CH2-CH3-zwA V264S
DKTHTCPPCPAPELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 340 - protein, 227 aa, Artificial Sequence
  Synthetic hinge-CH2-CH3-zwB
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 341 - protein, 227 aa, Artificial Sequence
  Synthetic hinge-CH2-CH3-zwB V262E
DKTHTCPPCPAPELLGGPSVFLFPPKPKDTLMISRTPEVTCEVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 342 - protein, 227 aa, Artificial Sequence
  Synthetic hinge-CH2-CH3-zwB V264S
DKTHTCPPCPAPELLGGPSVFLFPPKPKDTLMISRTPEVTCVVSDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 343 - protein, 103 aa, Artificial Sequence
  Synthetic CH1-(a)1
ASTKGPSVFPEAPSSKSTSGGTAALGCLVTDYFPEPVTVSWNSGALTSGVHTFPAVLESSGLYSLSSVVTVPSSSLGTQTYICNVNHKPSNTKVDKKVEPKSC

SEQ ID NO: 344 - protein, 103 aa, Artificial Sequence
  Synthetic CH1-(b)1
ASTKGPSVFPRAPSSKSTSGGTAALGCLVKDYFPEPVTVSWNSGALTSGVHTFPAVLQSSGLYKLSSVVTVPSSSLGTQTYICNVNHKPSNTKVDKKVEPKSC

SEQ ID NO: 345 - protein, 103 aa, Artificial Sequence
  Synthetic CH1-(c)1
ASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKVEPKSC

SEQ ID NO: 346 - protein, 103 aa, Artificial Sequence
  Synthetic CH1-(d)1
ASTKGPSVFPLAPSSKSTSGGTAALGCEVTDYFPEPVTVSWNSGALTSGVHTFPAVLESSGLYSLSSVVTVPSSSLGTQTYICNVNHKPSNTKVDKKVEPKSC

SEQ ID NO: 347 - protein, 103 aa, Artificial Sequence
  Synthetic CH1-(e)(f)1
ASTKGPSVFPLAPSSKSTSGGTAALGCLVKGYFPEPVTVSWNSGALTSGVHTFPAVLKSSGLYSLSSVVTVPSSSLGTQTYICNVNHKPSNTKVDKKVEPKSC

SEQ ID NO: 348 - protein, 103 aa, Artificial Sequence
  Synthetic CH1(a)2
ASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSC

SEQ ID NO: 349 - protein, 103 aa, Artificial Sequence; identical to SEQ ID NO: 346
  Synthetic CH1(b)2

SEQ ID NO: 350 - protein, 103 aa, Artificial Sequence
  Synthetic CH1(c)2
ASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKKVEPKSC

SEQ ID NO: 351 - protein, 103 aa, Artificial Sequence
  Synthetic CH1(d)2
ASTKGPSVFPLAPSSKSTSGGTAALGCLVKDYFPEPVTVSWNSGALTSGVRTFPAVLKSSGLYSLSSVVTVPSSSLGTQTYICNVNHKPSNTKVDKKVEPKSC

SEQ ID NO: 352 - protein, 103 aa, Artificial Sequence
  Synthetic CH1-(e)(f)2
ASTKGPSVFPLAPSSKSTSGGTAALGCEVTDYFPEPVTVSWNSGALTSGVHTFPAVLQSSGLYSLSSVVTVPSSSLGTQTYICNVNHKPSNTKVDKKVEPKSC

SEQ ID NO: 353 - protein, 107 aa, Artificial Sequence
  Synthetic Ck-(a)1
RTVAAPSVFIFPPSDEQLKSGTARVGCLLNNFYPREAKVQWKVDNALQSGNSQESVTEQDSKDSTYSLRSTLTLSKADYEKHKVYACEVTHQGLSSPVTKSFNRGEC

SEQ ID NO: 354 - protein, 107 aa, Artificial Sequence
  Synthetic Ck-(b)1
RTVAAPSVFIFPPSDEQLKSGTASVGCLLNNFYPREAKVQWKVDNALQSGNSQESVTEQDSKDSTYSLDSELTLSKADYEKHKVYACEVTHQGLSSPVTKSFNRGEC

SEQ ID NO: 355 - protein, 107 aa, Artificial Sequence
  Synthetic Ck-(c)1
RTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 356 - protein, 107 aa, Artificial Sequence
  Synthetic Ck-(d)1
RTVAAPSVFIFPPSDERLKSGTASVVCLLNNFYPREAKVQWKVDNALQSGNSKESVTEQDSKDSTYSLSSRLTLSKADYEKHKVYACEVTHQGLSSPVTKSFNRGEC

SEQ ID NO: 357 - protein, 107 aa, Artificial Sequence
  Synthetic Ck-(e)(f)1
RTVAAPSVFIFPPSDEELKSGTASVVCLLNNFYPREAKVQWKVDNALQSGNSEESVTEQDSKDSTYSLSSTLELSKADYEKHKVYACEVTHQGLSSPVTKSFNRGEC

SEQ ID NO: 358 - protein, 106 aa, Artificial Sequence
  Synthetic Cl-(a)2
GQPKAAPSVTLFPPSSEELQANKATLVCLISDFYPGAVTVAWKADSSPVKAGVETTTPSKQSNNKYAAESELSLTPEQWKSHRSYSCQVTHEGSTVEKTVAPTECS

SEQ ID NO: 359 - protein, 106 aa, Artificial Sequence
  Synthetic Cl-(b)2
GQPKAAPSVTLFPPSSEQLQANKARLVCLISDFYPGAVTVAWKADSSPVKAGVETTTPSKQSNNKYAASSYLSLTPEQWKSHRSYSCQVTHEGSTVEKTVAPTECS

SEQ ID NO: 360 - protein, 107 aa, Artificial Sequence
  Synthetic Ck-(c)2
RTVAAPSVFIFPPSDEELKSGTASVVCWLNNFYPREAKVQWKVDNALQSGNSEESVTEQDSKDSTYSLSSTLELSKADYEKHKVYACEVTHQGLSSPVTKSFNRGEC

SEQ ID NO: 361 - protein, 107 aa, Artificial Sequence; identical to SEQ ID NO: 357
  Synthetic Ck-(d)2

SEQ ID NO: 362 - protein, 107 aa, Artificial Sequence; identical to SEQ ID NO: 356
  Synthetic Ck-(e)2

SEQ ID NO: 363 - protein, 107 aa, Artificial Sequence
  Synthetic Ck-(f)2
RTVAAPSVFIFPPSDERLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 364 - protein, 243 aa, Artificial Sequence
  Synthetic aPD-1-1353-G10_R28T/P30D/H31S_Vk1-39_LC, scFv
EVQLVQSGAEVKKPGASVKVSCKASGYTFDSYGISWVRQAPGQGLEWMGWISAYNGNTNYAQKLQGRVTMTTDTSTNTAYMELRSLRSDDTAVYYCARDVDYGTGSGYWGQGTLVTVSSGGGGSGGGGSGGGGSDIQLTQSPSSLSASVGDRVTITCSGDALPKQYAYWYQQKPGKAPKLLIYKDTERPSGVPSRFSGSSSGTKVTLTISSLQPEDFATYYCQSADNSITYRVFGGGTKVEIK

SEQ ID NO: 365 - protein, 242 aa, Artificial Sequence
  Synthetic aPD1 1353-G10 scFvFc zwB R28T/P30D/H31S, scFv
EVQLVQSGAEVKKPGASVKVSCKASGYTFDSYGISWVRQAPGQGLEWMGWISAYNGNTNYAQKLQGRVTMTTDTSTNTAYMELRSLRSDDTAVYYCARDVDYGTGSGYWGQGTLVTVSSGGGGSGGGGSGGGGSSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVL

SEQ ID NO: 366 - protein, 243 aa, Artificial Sequence
  Synthetic aPD1 1353-G10 Vk139, scFv
EVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSGGGGSGGGGSGGGGSDIQLTQSPSSLSASVGDRVTITCSGDALPKQYAYWYQQKPGKAPKLLIYKDTERPSGVPSRFSGSSSGTKVTLTISSLQPEDFATYYCQSADNSITYRVFGGGTKVEIK

SEQ ID NO: 367 - protein, 512 aa, Artificial Sequence
  Synthetic 1353-G10, scFv-Fc
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSGGGGSGGGGSGGGGSSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLAAGSDQEPKKLAAGSDQEPKSSDKTHTCPPCSAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSGDYKDDDDKGSGHHHHHH

SEQ ID NO: 368 - protein, 481 aa, Artificial Sequence
  Synthetic 1353-G10 scFv-Fc hole, scFv-Fc
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSGGGGSGGGGSGGGGSSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 369 - protein, 481 aa, Artificial Sequence
  Synthetic 1353-G10 scFv-Fc knob, scFv-Fc
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSGGGGSGGGGSGGGGSSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 370 - protein, 481 aa, Artificial Sequence
  Synthetic 1353-G10 scFv-Fc zwA, scFv-Fc
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSGGGGSGGGGSGGGGSSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 371 - protein, 487 aa, Artificial Sequence
  Synthetic 1449-G09.2 scFv-Fc knob, scFv-Fc
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSAPGPSAPSHRSLPSRAFGEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIKAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 372 - protein, 453 aa, Artificial Sequence
  Synthetic 1449-G09.2 HC hole, HC
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 373 - protein, 453 aa, Artificial Sequence
  Synthetic 1449-G09.2 HC zwB, HC
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 374 - protein, 453 aa, Artificial Sequence
  Synthetic 1449-G09.2 HC1a zwA, HC
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSASTKGPSVFPEAPSSKSTSGGTAALGCLVTDYFPEPVTVSWNSGALTSGVHTFPAVLESSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 375 - protein, 450 aa, Artificial Sequence
  Synthetic 1449-G09.2 HC2a zwB, HC
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSASTKGPSVFPLAPSSKSTSGGTAALGCLVKDYFPEPVTVSWNSGALTSGVHTFPAVLQSSGLYSLKSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 376 - protein, 216 aa, Artificial Sequence
  Synthetic 1449-G09.2 LC, LC
MEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 377 - protein, 216 aa, Artificial Sequence
  Synthetic 1449-G09.2 LC1a, LC
MEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIKRTVAAPSVFIFPPSDEQLKSGTARVGCLLNNFYPREAKVQWKVDNALQSGNSQESVTEQDSKDSTYSLRSTLTLSKADYEKHKVYACEVTHQGLSSPVTKSFNRGEC

SEQ ID NO: 378 - protein, 215 aa, Artificial Sequence
  Synthetic 1449-G09.2 LC2a, LC
MSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLGQPKAAPSVTLFPPSSEELQANKATLVCLISDFYPGAVTVAWKADSSPVKAGVETTTPSKQSNNKYAAESELSLTPEQWKSHRSYSCQVTHEGSTVEKTVAPTECS

SEQ ID NO: 379 - protein, 185 aa, Artificial Sequence
  Synthetic LAG3 scFvFc, scFv
QVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSGGGGSGGGGSGGGGSEIVLTQSPGTLSLSPGERATLSCRASQSVSSSYLAWYQQKPGQKVDIK

SEQ ID NO: 380 - protein, 487 aa, Artificial Sequence
  Synthetic 1449-G09.2 scFv-Fc hole, scFv-Fc
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSAPGPSAPSHRSLPSRAFGEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIKAAGSDQEPKSSDKTHTCPPCSAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 381 - protein, 450 aa, Artificial Sequence
  Synthetic Anti-PD1 1353-G10 HC knob, HC
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 382 - protein, 450 aa, Artificial Sequence
  Synthetic Anti-PD1 1353-G10 HC hole, HC
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 383 - protein, 215 aa, Artificial Sequence
  Synthetic Anti-PD1 1353-G10 LC, LC
MSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 384 - protein, 487 aa, Artificial Sequence; identical to SEQ ID NO: 380
  Synthetic Anti-LAG3 1449-G09.2 scFvFc hole, scFv-Fc

SEQ ID NO: 385 - protein, 103 aa, Artificial Sequence
  Synthetic CH1-wt
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 386 - protein, 107 aa, Artificial Sequence [sequence omitted]

SEQ ID NO: 387 - protein, 106 aa, Artificial Sequence; identical to SEQ ID NO: 305
  Synthetic C-lambda-wt

SEQ ID NO: 388 - protein, 481 aa, Artificial Sequence
  Synthetic 1353-G10 scFv-Fc zwB, scFv-Fc
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSGGGGSGGGGSGGGGSSYELTQPPSVSVSPGQTARITCSGDALPKQYAYWYQQKPGQAPVMVIYKDTERPSGIPERFSGSSSGTKVTLTISGVQAEDEADYYCQSADNSITYRVFGGGTKVTVLAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 389 - protein, 450 aa, Artificial Sequence
  Synthetic 1353-G10 HC zwA, HC
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 390 - protein, 450 aa, Artificial Sequence
  Synthetic 1353-G10 HC zwB, HC
MEVQLVQSGAEVKKPGASVKVSCKASGYRFPHYGISWVRQAPGQGLEWMGWISAYNGNTNYAQKLQGRVTMTTDTSTNTAYMELRSLRSDDTAVYYCARDVDYGTGSGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 391 - protein, 487 aa, Artificial Sequence
  Synthetic 1449-G09.2 scFv-Fc zwA, scFv-Fc
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSAPGPSAPSHRSLPSRAFGEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIKAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 392 - protein, 487 aa, Artificial Sequence
  Synthetic 1449-G09.2 scFv-Fc zwB, scFv-Fc
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSAPGPSAPSHRSLPSRAFGEIVLTQSPGTLSLSPGERATLSCRASQSVSSSYLAWYQQKPGQAPRLLIYGASSRATGIPDRFSGSGSGTDFTLTISRLEPEDFAVYYCQQYGRSPFSFGPGTKVDIKAAGSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 393 - protein, 453 aa, Artificial Sequence
  Synthetic 1449-G09.2 HC zwA, HC
MQVQLVESGGGVVQPGRSLRLSCAASGFTFSSYGMHWVRQAPGKGLEWVAVIWYDGSYKYYADSVKGRFTISRDNSKNTLYLQMNSLRAEDTAVYYCAREEAPENWDYALDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 394 - protein, 216 aa, Artificial Sequence
  Synthetic 1353-G10 LC1c, LC
MSYELTQPPSVSVSPGQTARITCSGDALPKQYAYWYQQKPGQAPVMVIYKDTERPSGIPERFSGSSSGTKVTLTISGVQAEDFATYYCQSADNSITYRVFGGGTKVEIKRTVAAPSVAIFPPSDERLKSGTASVVCVLNNFYPREAKVQWKVDNALQSGNSQESVTEQDSKDSTYSLSSRLTLSKADYEKHKVYACEVTHQGLSSPVTKSFNRGEC

SEQ ID NO: 395 - protein, 216 aa, Artificial Sequence
  Synthetic 1353-G10 LC1d, LC
MSYELTQPPSVSVSPGQTARITCSGDALPKQYAYWYQRKPGQAPVMVIYKDTERPSGIPERFSGSSSGTKVTLTISGVQAEDFATYYCQSADNSITYRVFGGGTKVEIKRTVAAPSVFIFPPSDERLKSGTASVVCLLNNFYPREAKVQWKVDNALQSGNSKESVTEQDSKDSTYSLSSRLTLSKADYEKHKVYACEVTHQGLSSPVTKSFNRGEC

SEQ ID NO: 396 - protein, 216 aa, Artificial Sequence
  Synthetic 1449-G09.2 LC2d, LC
MEIVLTQSPGTLSLSPGERATLSCRASQSVSSSYLAWYQEKPGQAPRLLIYGASSRATGIPDRFSGSGSGTDFTLTISRLEPEDFAVYYCQQYGRSPFSFGPGTKVDIKRTVAAPSVFIFPPSDEELKSGTASVVCLLNNFYPREAKVQWKVDNALQSGNSEESVTEQDSKDSTYSLSSTLELSKADYEKHKVYACEVTHQGLSSPVTKSFNRGEC

SEQ ID NO: 397 - protein, 450 aa, Artificial Sequence
  Synthetic 1353-G10 HC1d zwB, HC
MEVQLVQSGAEVKKPGASVKVSCKASGYRFPHYGISWVREAPGQGLEWMGWISAYNGNTNYAQKLQGRVTMTTDTSTNTAYMELRSLRSDDTAVYYCARDVDYGTGSGYWGQGTLVTVSSASTKGPSVFPLAPSSKSTSGGTAALGCEVTDYFPEPVTVSWNSGALTSGVHTFPAVLESSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPGK

SEQ ID NO: 398 - protein, 453 aa, Artificial Sequence
  Synthetic 1449-G09.2 HC2d zwA, HC
MQVQLVESGGGVVQPGRSLRLSCAASGFTFSSYGMHWVRRAPGKGLEWVAVIWYDGSYKYYADSVKGRFTISRDNSKNTLYLQMNSLRAEDTAVYYCAREEAPENWDYALDVWGQGTTVTVSSASTKGPSVFPLAPSSKSTSGGTAALGCLVKDYFPEPVTVSWNSGALTSGVRTFPAVLKSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPGK

SEQ ID NO: 399 - protein, 30 aa, Artificial Sequence; identical to SEQ ID NO: 311
  Synthetic Linker
  misc_feature (1)..(30): At least 3 repeats of “GGGGS” are always present; and one group and up to three groups of “GGGGS” may be present or absent

(19) United States
(12) Patent Application Publication
(10) Pub. No.: US 2020/0079850 A1
(43) Pub. Date: Mar. 12, 2020

(54) PD-1/LAG3 BI-SPECIFIC ANTIBODIES, COMPOSITIONS THEREOF, AND METHODS OF MAKING AND USING THE SAME

(71) Applicant: SUTRO BIOPHARMA, INC., South San Francisco, CA (US)

(72) Inventors: Ryan STAFFORD, Emeryville, CA (US); Alice YAM, Tiburon, CA (US); Stephanie ARMSTRONG, South San Francisco, CA (US); John LEE, San Francisco, CA (US); Alexander STEINER, San Francisco, CA (US); Junhao YANG, Palo Alto, CA (US); Christine CHENG, South San Francisco, CA (US)

(21) Appl. No.: 16/616,149

(22) PCT Filed: May 23, 2018
(86) PCT No.: PCT/US2018/034213

§ 371 Date: Nov. 22, 2019

Related U.S. Application Data
(60) Provisional application No. 62/510,691, filed on May 24, 2017.

(51) Int. Cl. C07K 16/28

(52) U.S. Cl. CPC C07K 16/2803; C07K 16/2818; C07K 2317/565; C07K 2317/31; A61K 2039/505; C07K 2317/76; C07K 2317/92; C07K 2317/94; C07K 2317/622

(57) ABSTRACT

Provided herein are antibodies that selectively bind to LAG3 and its isoforms and homologs, and compositions comprising the antibodies. Also provided herein are antibodies that selectively bind to PD-1 and its isoforms and homologs, and compositions comprising the antibodies. In addition, provided herein are bi-specific antibodies and antigen binding constructs that selectively bind to LAG3 and/or PD-1, their isoforms and homologs, and compositions comprising the antibodies and antigen binding constructs. Also provided are methods of using the antibodies, such as therapeutic and diagnostic methods.

### FIELD

Provided herein are antibodies with dual binding specificity for lymphocyte-activation gene 3 (LAG3) and for programmed cell death protein (PD-1), also referred to as PD-1/LAG3 bi-specific antibodies. Also provided herein are antibodies with binding specificity for PD-1 or LAG3. In addition, provided herein are compositions comprising the antibodies, including pharmaceutical compositions, diagnostic compositions, and kits. Also provided are methods of making the bi-specific antibodies, and methods of using the bi-specific antibodies, for example, for therapeutic purposes, diagnostic purposes, and research purposes.

### BACKGROUND

The lymphocyte activation gene 3 (LAG3) was discovered in 1990. Triebel et al., 1990, J. Exp. Med. 171:1393-4053. It was identified as selectively transcribed in activated natural killer (NK) cells and T lymphocytes. See id. The LAG3 protein was originally described as a type I membrane protein of 498 amino acids including a signal peptide, an extracellular region, a transmembrane region, and a cytoplasmic region. See id. The extracellular region has four Ig domains, and the whole protein has sequence similarity to CD4. See id.

LAG3 is selectively expressed in regulatory T cells, and its natural ligand is MHC class II. Huang et al., 2004, Immunity 21:503-513. Regulatory T cells are important for maintaining immune tolerance to limit autoimmunity and in regulating lymphocyte expansion. See id. They also suppress natural immune responses to parasites and viruses, and they have suppressed antitumor immunity induced by therapeutic vaccines. See id. Antibodies to LAG3 were shown to inhibit suppression by induced regulatory T cells. See id. Antibody targeting of LAG3 has been shown to enhance antitumor immunity in animal models of cancer. Pardoll, 2012, Nature Rev. Cancer 12:252-264; Jing et al., 2015, J. Immunother. Cancer 3:2-29. LAG3 is an immune checkpoint protein target for active drug development, and clinical trials have been proposed for antibodies to LAG3 for the treatment of solid tumors.

Programmed cell death protein 1 (PD-1, also known as CD279) is a cell surface protein molecule that belongs to the immunoglobulin superfamily. It is expressed on T and B lymphocytes and macrophages, and plays a role in cell fate and differentiation. See Ishida et al., EMBO J., 1992, 11:3887-3895, incorporated by reference in its entirety. Activation of PD-1 is thought to negatively regulate the immune response. See Blank et al., Cancer Immunol. Immunother., 2007, 56:739-745; and Freeman et al., J. Exp. Med., 2000, 192:1027-1034, each of which is incorporated by reference in its entirety.

PD-1 has two known ligands, PD-L1 and PD-L2, which are both members of the B7 family. See Freeman et al., supra; and Latchman et al., Nat. Immunol., 2001, 2:261-268, each of which is incorporated by reference in its entirety. The interaction between PD-1 and these ligands is thought to play a role in a variety of diseases, including cancer (see Ribas and Tumeh, Clin. Cancer Res., 2014, Jun. 26, PMID: 24970841), autoimmune disease (see Dai et al., Cell Immunol., 2014, 290:72-79), and infection (see Day et al., Nature, 2006, 443:350-354). Each of the references cited in the preceding sentence is incorporated by reference in its entirety. In particular, the engagement of PD-1 by one of its ligands is thought to inhibit T-cell effector functions in an antigen-specific manner.

In view of the role of PD-1 and LAG3 in multiple disease processes, there is a need for methods of modulating the immune regulation and downstream signaling processes activated by both LAG3 and PD-1. There is also a need for therapeutics that can specifically target cells and tissues that express LAG3 and/or PD-1.

### SUMMARY

Provided herein are antibodies that selectively bind LAG3. In some embodiments, the antibodies bind human LAG3. In some embodiments, the antibodies also bind homologs of human LAG3. In some aspects, the homologs include a cynomolgus monkey homolog.

Also provided herein are antibodies that selectively bind PD-1. In some embodiments, the antibodies bind human PD-1. In some embodiments, the antibodies also bind homologs of human PD-1. In some aspects, the homologs include a cynomolgus monkey homolog.

Also provided herein are bi-specific antibodies or bi-specific antibody constructs that comprise a first binding domain that selectively binds LAG3, including human LAG3 or a homolog thereof, and a second binding domain that selectively binds PD-1, including human PD-1 or a homolog thereof.

In some embodiments, the antibodies comprise at least one CDR sequence defined by a consensus sequence provided in this disclosure. In some embodiments, the antibodies comprise an illustrative CDR, V_{H}, or V_{L} sequence provided in this disclosure, or a variant thereof. In some aspects, the variant is a variant with one or more conservative amino acid substitutions.

Also provided are compositions and kits comprising the antibodies. In some embodiments, the compositions are pharmaceutical compositions. Any suitable pharmaceutical composition may be used. In some embodiments, the pharmaceutical composition is a composition for parenteral administration.

This disclosure also provides methods of using the anti-LAG3 antibodies provided herein. In some embodiments, the method is a method of treatment. In some embodiments, the method is a diagnostic method. In some embodiments, the method is an analytical method. In some embodiments, the method is a method of purifying and/or quantifying LAG3.

In some embodiments, the antibodies are used to treat a disease or condition. In some aspects, the disease or condition is selected from a cancer, autoimmune disease, and infection.

### BRIEF DESCRIPTION OF THE DRAWINGS

FIG. 1 provides a comparison of the Kabat and Chothia numbering systems for CDR-H1. Adapted from Martin A.C.R. (2010). Protein Sequence and Structure Analysis of Antibody Variable Domains. In R. Kontermann & S. Dübel (Eds.), Antibody Engineering vol. 2 (pp. 33-51). Springer-Verlag, Berlin Heidelberg.

FIG. 2(A, B) provides an alignment of the V_{H} sequences provided herein. CDRs according to Chothia are outlined, and CDRs according to Kabat are underlined.

FIG. 3 provides an alignment of the V_{L} sequences provided herein. CDRs according to Chothia are outlined, and CDRs according to Kabat are underlined.

FIG. 4 is a graph illustrating the mean tumor volume for different antibody combinations tested in the MC38 murine tumor model to compare tumor growth control.

FIG. 5 includes two semi-logarithmic graphs that illustrate individual and mean serum concentration-time profiles in a cynomolgous PK study of a PD-1/LAG3 bi-specific antibody.

FIG. 6 includes two semi-logarithmic graphs that illustrate serum concentration-time profiles in a cynomolgous PK study of additional PD-1/LAG3 bi-specific antibodies.

FIG. 7 includes two graphs that illustrate immunogenicity assessments for the additional PD-1/LAG3 bi-specific antibodies in individual cynomolgous monkeys.

FIG. 8 includes two graphs and a table that provide a comparison of the cell binding affinities of three separate PD-1/LAG3 bi-specific antibodies for human PD-1 and cynomolgous PD-1.

FIG. 9 includes two graphs and a table that provide a comparison of the cell binding affinities of three separate PD-1/LAG3 bi-specific antibodies for human LAG3 and cynomolgous LAG3.

FIG. 10 includes two graphs and a table that provide a comparison of the ability of three separate PD-1/LAG3 bi-specific antibodies to inhibit binding between human PD-1 and PD-L1 or PD-L2.

FIG. 11 is a graph and table that provide a comparison of the ability of three separate PD-1/LAG3 bi-specific antibodies to inhibit binding between human LAG3 and MHC-class II.

FIG. 12 is a graph and table that provide a comparison of the ability of three separate PD-1/LAG3 bi-specific antibodies to inhibit binding to both human PD-1 and human LAG3 co-expressed on U2OS engineered cells.

FIG. 13 is a graph that provides a comparison between a mono-specific (“PD-1 stump”) control and three separate PD-1/LAG3 bi-specific antibodies in a PBMC CMV-antigen recall assay.

### DETAILED DESCRIPTION

### 1. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a difference over what is generally understood in the art. The techniques and procedures described or referenced herein are generally well understood and commonly employed using conventional methodologies by those skilled in the art, such as, for example, the widely utilized molecular cloning methodologies described in Sambrook et al., Molecular Cloning: A Laboratory Manual 2nd ed. (1989) Cold Spring Harbor Laboratory Press, Cold Spring Harbor, N.Y. As appropriate, procedures involving the use of commercially available kits and reagents are generally carried out in accordance with manufacturer-defined protocols and conditions unless otherwise noted.

As used herein, the singular forms “a,” “an,” and “the” include the plural referents unless the context clearly indicates otherwise.

The term “about” indicates and encompasses an indicated value and a range above and below that value. In certain embodiments, the term “about” indicates the designated value±10%, ±5%, or ±1%. In certain embodiments, the term “about” indicates the designated value±one standard deviation of that value.

The terms “first” and “second” are intended to indicate two separate entities, but does not mean that one is before the other in time or space, unless otherwise noted.

The term “combinations thereof” includes every possible combination of elements to which the term refers to. For example, a sentence stating that “if α₂ is A, then α₃ is not D; α₅ is not S; or α₆ is not S; or combinations thereof” includes the following combinations when α₂ is A: (1) α₃ is not D; (2) α₅ is not S; (3) α₆ is not S; (4) α₃ is not D; α₅ is not S; and α₆ is not S; (5) α₃ is not D and as is not S; (6) α₃ is not D and α₆ is not S; and (7) α₅ is not S and α6 is not S.

The terms “LAG3” and “LAG3 antigen” are used interchangeably herein. LAG3 is also known by a variety of synonyms, including lymphocyte-activation gene 3, CD223, cluster of differenetiation 223, and FDC, among others. Unless specified otherwise, the terms include any variants, isoforms and species homologs of human LAG3 that are naturally expressed by cells, or that are expressed by cells transfected with an LAG3 gene. LAG3 proteins include, for example, human LAG3 (GI: 15928632; SEQ ID NO: 1). In some embodiments, LAG3 proteins include cynomolgus monkey LAG3 (GI: 544483249; SEQ ID NO: 2). In some embodiments, LAG3 proteins include murine LAG3 (GI: 112293275; SEQ ID NO: 3). However, as discussed in detail elsewhere in this disclosure, in some embodiments the antibodies provided herein do not bind murine LAG3 proteins. The antibodies provided herein bind to an extracellular domain of LAG3.

The terms “PD-1” and “PD-1 antigen” are used interchangeably herein. Unless specified otherwise, the terms include any variants, isoforms and species homologs of human PD-1 that are naturally expressed by cells, or that are expressed by cells transfected with a PD-1 gene. PD-1 proteins include full-length PD-1 (e.g., human PD-1; GI: 167857792; SEQ ID NO: 318; extracellular domain: Pro21-Gln167), as well as alternative splice variants of PD-1, such as PD-1Δex2, PD-1Δex3, PD-1Δex2,3, and PD-1Δex2,3,4. See Nielsen et al., Cellular Immunology, 2005, 235:109-116, incorporated by reference in its entirety. In some embodiments, PD-1 proteins include murine PD-1 (e.g., SEQ ID NO: 319; extracellular domain: Leu25-Gln167). In some embodiments, PD-1 proteins include cynomolgus PD-1 (e.g., SEQ ID NO: 320; extracellular domain: Pro21-Gln167).

The term “immunoglobulin” refers to a class of structurally related proteins generally comprising two pairs of polypeptide chains: one pair of light (L) chains and one pair of heavy (H) chains. In an “intact immunoglobulin,” all four of these chains are interconnected by disulfide bonds. The structure of immunoglobulins has been well characterized. See, e.g., Paul, Fundamental Immunology 7th ed., Ch. 5 (2013) Lippincott Williams & Wilkins, Philadelphia, Pa. Briefly, each heavy chain typically comprises a heavy chain variable region (V_{H}) and a heavy chain constant region (C_{H}). The heavy chain constant region typically comprises three domains, abbreviated C_{H1}, C_{H2}, and C_{H3}. Each light chain typically comprises a light chain variable region (V_{L}) and a light chain constant region. The light chain constant region typically comprises one domain, abbreviated C_{L}.

The term “antibody” describes a type of immunoglobulin molecule and is used herein in its broadest sense. An antibody specifically includes intact antibodies (e.g., intact immunoglobulins), and antibody fragments. Antibodies comprise at least one antigen-binding domain. One example of an antigen-binding domain is an antigen binding domain formed by a V_{H}-V_{L} dimer.

A “LAG3 antibody,” “anti-LAG3 antibody,” “LAG3 Ab,” “LAG3-specific antibody” or “anti-LAG3 Ab” is an antibody, as described herein, which binds specifically to the antigen LAG3. In some embodiments, the antibody binds the extracellular domain of LAG3.

A “PD-1 antibody,” “anti-PD-1 antibody,” “PD-1 Ab,” “PD-1-specific antibody” or “anti-PD-1 Ab” is an antibody, as described herein, which binds specifically to the antigen PD-1. In some embodiments, the antibody binds the extracellular domain of PD-1.

The V_{H} and V_{L} regions may be further subdivided into regions of hypervariability (“hypervariable regions (HVRs);” also called “complementarity determining regions” (CDRs)) interspersed with regions that are more conserved. The more conserved regions are called framework regions (FRs). Each V_{H} and V_{L} generally comprises three CDRs and four FRs, arranged in the following order (from N-terminus to C-terminus): FR1-CDR1-FR2-CDR2-FR3-CDR3-FR4. The CDRs are involved in antigen binding, and influence antigen specificity and binding affinity of the antibody. See Kabat et al., Sequences of Proteins of Immunological Interest 5th ed. (1991) Public Health Service, National Institutes of Health, Bethesda, Md., incorporated by reference in its entirety.

The light chain from any vertebrate species can be assigned to one of two types, called kappa and lambda, based on the sequence of the constant domain.

The heavy chain from any vertebrate species can be assigned to one of five different classes (or isotypes): IgA, IgD, IgE, IgG, and IgM. These classes are also designated α, δ, ε, γ, and μ, respectively. The IgG and IgA classes are further divided into subclasses on the basis of differences in sequence and function. Humans express the following subclasses: IgG1, IgG2, IgG3, IgG4, IgA1, and IgA2.

The amino acid sequence boundaries of a CDR can be determined by one of skill in the art using any of a number of known numbering schemes, including those described by Kabat et al., supra (“Kabat” numbering scheme); Al-Lazikani et al., 1997, J. Mol. Biol., 273:927-948 (“Chothia” numbering scheme); MacCallum et al., 1996, J. Mol. Biol. 262:732-745 (“Contact” numbering scheme); Lefranc et al., Dev. Comp. Immunol., 2003, 27:55-77 (“IMGT” numbering scheme); and Honegge and Plückthun, J. Mol. Biol., 2001, 309:657-70 (“AHo” numbering scheme), each of which is incorporated by reference in its entirety.

Table 1 provides the positions of CDR-L1, CDR-L2, CDR-L3, CDR-H1, CDR-H2, and CDR-H3 as identified by the Kabat and Chothia schemes. For CDR-H1, residue numbering is provided using both the Kabat and Chothia numbering schemes.

Unless otherwise specified, the numbering scheme used for identification of a particular CDR herein is the Kabat/Chothia numbering scheme. Where the residues encompassed by these two numbering schemes diverge (e.g., CDR-H1 and/or CDR-H2), the numbering scheme is specified as either Kabat or Chothia. For convenience, CDR-H3 is sometimes referred to herein as either Kabat or Chothia. However, this is not intended to imply differences in sequence where they do not exist, and one of skill in the art can readily confirm whether the sequences are the same or different by examining the sequences.

CDRs may be assigned, for example, using antibody numbering software, such as Abnum, available at http://www.bioinf.org.uk/abs/anum/, and described in Abhinandan and Martin, Immunology, 2008, 45:3832-3839, incorporated by reference in its entirety.

TABLE 1
Residues in CDRs according to Kabat
and Chothia numbering schemes.

| CDR | Kabat | Chothia |
|---|---|---|
| L1 | L24-L34 | L24-L34 |
| L2 | L50-L56 | L50-L56 |
| L3 | L89-L97 | L89-L97 |
| H1 (Kabat Numbering) | H31-H35B | H26-H32 or H34* |
| H1 (Chothia Numbering) | H31-H35 | H26-H32 |
| H2 | H50-H65 | H52-H56 |
| H3 | H95-H102 | H95-H102 |

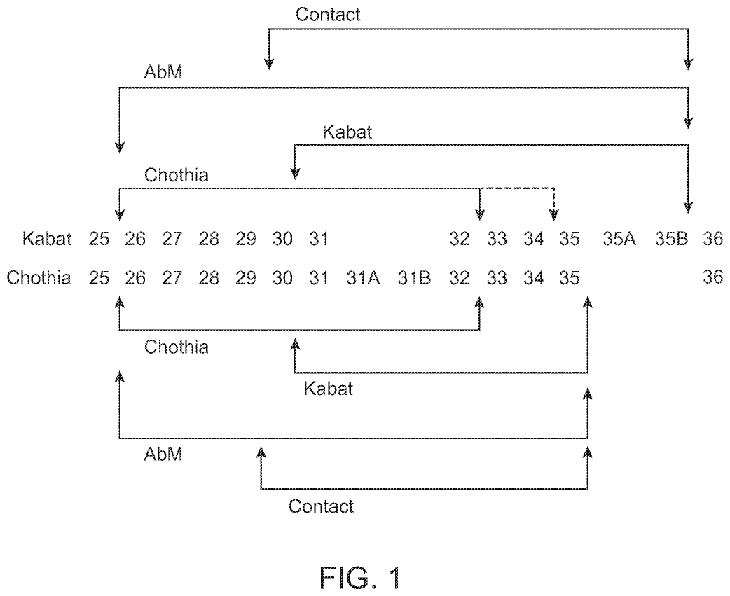

*The C-terminus of CDR-H1, when numbered using the Kabat numbering convention, varies between H32 and H34, depending on the length of the CDR, as illustrated in FIG. 1.

The “EU numbering scheme” is generally used when referring to a residue in an antibody heavy chain constant region (e.g., as reported in Kabat et al., supra). Unless stated otherwise, the EU numbering scheme is used to refer to residues in antibody heavy chain constant regions described herein.

An “antibody fragment” comprises a portion of an intact antibody, such as the antigen binding or variable region of an intact antibody. Antibody fragments include, for example, Fv fragments, Fab fragments, F(ab′)2 fragments, Fab′ fragments, scFv (sFv) fragments, and scFv-Fc fragments.

“Fv” fragments comprise a non-covalently-linked dimer of one heavy chain variable domain and one light chain variable domain.

“Fab” fragments comprise, in addition to the heavy and light chain variable domains, the constant domain of the light chain and the first constant domain (C_{H1}) of the heavy chain. Fab fragments may be generated, for example, by recombinant methods or by papain digestion of a full-length antibody.

“F(ab′)₂” fragments contain two Fab′ fragments joined, near the hinge region, by disulfide bonds. F(ab′)₂ fragments may be generated, for example, by recombinant methods or by pepsin digestion of an intact antibody. The F(ab′) fragments can be dissociated, for example, by treatment with ß-mercaptoethanol.

“Single-chain Fv” or “sFv” or “scFv” antibody fragments comprise a V_{H} domain and a V_{L} domain in a single polypeptide chain. The V_{H} and V_{L} are generally linked by a peptide linker. See Plückthun A. (1994). In some embodiments, the linker is SEQ ID NO: 168. Antibodies from Escherichia coli. In Rosenberg M. & Moore G. P. (Eds.), The Pharmacology of Monoclonal Antibodies vol. 113 (pp. 269-315). Springer-Verlag, New York, incorporated by reference in its entirety.

“scFv-Fc” fragments comprise an scFv attached to an Fc domain. For example, an Fc domain may be attached to the C-terminus of the scFv. The Fc domain may follow the V_{H} or V_{L}, depending on the orientation of the variable domains in the scFv (i.e., V_{H}-V_{L} or V_{L}-V_{H}). Any suitable Fc domain known in the art or described herein may be used. In some cases, the Fc domain comprises an IgG1 Fc domain. In some embodiments, the IgG1 Fc domain comprises SEQ ID NO: 300, or a portion thereof, or SEQ ID NO: 306. SEQ ID NO: 300 provides the sequence of C_{H1}, C_{H2}, and C_{H3} of the human IgG1 constant region. SEQ ID NO: 306 provides the sequence of the constant region used in the illustrative scFv-Fc antibodies provided herein.

The term “monoclonal antibody” refers to an antibody from a population of substantially homogeneous antibodies. A population of substantially homogeneous antibodies comprises antibodies that are substantially similar and that bind the same epitope(s), except for variants that may normally arise during production of the monoclonal antibody. Such variants are generally present in only minor amounts. A monoclonal antibody is typically obtained by a process that includes the selection of a single antibody from a plurality of antibodies. For example, the selection process can be the selection of a unique clone from a plurality of clones, such as a pool of hybridoma clones, phage clones, yeast clones, bacterial clones, or other recombinant DNA clones. The selected antibody can be further altered, for example, to improve affinity for the target (“affinity maturation”), to humanize the antibody, to improve its production in cell culture, and/or to reduce its immunogenicity in a subject.

The term “chimeric antibody” refers to an antibody in which a portion of the heavy and/or light chain is derived from a particular source or species, while the remainder of the heavy and/or light chain is derived from a different source or species.

“Humanized” forms of non-human antibodies are chimeric antibodies that contain minimal sequence derived from the non-human antibody. A humanized antibody is generally a human immunoglobulin (recipient antibody) in which residues from one or more CDRs are replaced by residues from one or more CDRs of a non-human antibody (donor antibody). The donor antibody can be any suitable non-human antibody, such as a mouse, rat, rabbit, chicken, or non-human primate antibody having a desired specificity, affinity, or biological effect. In some instances, selected framework region residues of the recipient antibody are replaced by the corresponding framework region residues from the donor antibody. Humanized antibodies may also comprise residues that are not found in either the recipient antibody or the donor antibody. Such modifications may be made to further refine antibody function. For further details, see Jones et al., Nature, 1986, 321:522-525; Riechmann et al., Nature, 1988, 332:323-329; and Presta, Curr. Op. Struct. Biol., 1992, 2:593-596, each of which is incorporated by reference in its entirety.

A “human antibody” is one which possesses an amino acid sequence corresponding to that of an antibody produced by a human or a human cell, or derived from a non-human source that utilizes a human antibody repertoire or human antibody-encoding sequences (e.g., obtained from human sources or designed de novo). Human antibodies specifically exclude humanized antibodies.

An “isolated antibody” is one that has been separated and/or recovered from a component of its natural environment. Components of the natural environment may include enzymes, hormones, and other proteinaceous or nonproteinaceous materials. In some embodiments, an isolated antibody is purified to a degree sufficient to obtain at least 15 residues of N-terminal or internal amino acid sequence, for example by use of a spinning cup sequenator. In some embodiments, an isolated antibody is purified to homogeneity by gel electrophoresis (e.g., SDS-PAGE) under reducing or nonreducing conditions, with detection by Coomassie blue or silver stain. An isolated antibody includes an antibody in situ within recombinant cells, since at least one component of the antibody's natural environment is not present. In some aspects, an isolated antibody is prepared by at least one purification step.

In some embodiments, an isolated antibody is purified to at least 80%, 85%, 90%, 95%, or 99% by weight. In some embodiments, an isolated antibody is purified to at least 80%, 85%, 90%, 95%, or 99% by volume. In some embodiments, an isolated antibody is provided as a solution comprising at least 85%, 90%, 95%, 98%, 99% to 100% by weight. In some embodiments, an isolated antibody is provided as a solution comprising at least 85%, 90%, 95%, 98%, 99% to 100% by volume.

“Affinity” refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity, which reflects a 1:1 interaction between members of a binding pair (e.g., antibody and antigen). The affinity of a molecule X for its partner Y can be represented by the dissociation constant (K_{D}). Affinity can be measured by common methods known in the art, including those described herein. Affinity can be determined, for example, using surface plasmon resonance (SPR) technology, such as a Biacore® instrument. In some embodiments, the affinity is determined at 25° C.

With regard to the binding of an antibody to a target molecule, the terms “specific binding,” “specifically binds to,” “specific for,” “selectively binds,” and “selective for” a particular antigen (e.g., a polypeptide target) or an epitope on a particular antigen mean binding that is measurably different from a non-specific or non-selective interaction. Specific binding can be measured, for example, by determining binding of a molecule compared to binding of a control molecule. Specific binding can also be determined by competition with a control molecule that mimics the antibody binding site on the target. In that case, specific binding is indicated if the binding of the antibody to the target is competitively inhibited by the control molecule.

The term “k_{d}” (sec⁻¹), as used herein, refers to the dissociation rate constant of a particular antibody-antigen interaction. This value is also referred to as the k_{off} value.

The term “kₐ” (M⁻¹×sec⁻¹), as used herein, refers to the association rate constant of a particular antibody-antigen interaction. This value is also referred to as the kₒₙ value.

The term “K_{D}” (M), as used herein, refers to the dissociation equilibrium constant of a particular antibody-antigen interaction. K_{D}=k_{d}/kₐ.

The term “K_{A}” (M⁻¹), as used herein, refers to the association equilibrium constant of a particular antibody-antigen interaction. K_{A}=kₐ/k_{d}.

An “affinity matured” antibody is one with one or more alterations in one or more CDRs or FRs that result in an improvement in the affinity, or other properties (e.g. biophysical), of the antibody for its antigen, compared to a parent antibody which does not possess the alteration(s). In one embodiment, an affinity matured antibody has nanomolar or picomolar affinity for the target antigen. Affinity matured antibodies may be produced using a variety of methods known in the art. For example, Marks et al. (Bio/Technology, 1992, 10:779-783, incorporated by reference in its entirety) describes affinity maturation by V_{H} and V_{L} domain shuffling. Random mutagenesis of CDR and/or framework residues is described by, for example, Barbas et al. (Proc. Nat. Acad. Sci. U.S.A., 1994, 91:3809-3813); Schier et al., Gene, 1995, 169:147-155; Yelton et al., J. Immunol., 1995, 155:1994-2004; Jackson et al., J. Immunol., 1995, 154:3310-33199; and Hawkins et al, J. Mol. Biol., 1992, 226:889-896, each of which is incorporated by reference in its entirety.

When used herein in the context of two or more antibodies, the term “competes with” or “cross-competes with” indicates that the two or more antibodies compete for binding to an antigen. In one exemplary assay, an antigen is coated on a plate and allowed to bind a first antibody, after which a second, labeled antibody is added. If the presence of the first antibody reduces binding of the second antibody, then the antibodies compete. In another exemplary assay, a first antibody is coated on a plate and allowed to bind an antigen, and then the second antibody is added. The term “competes with” also includes combinations of antibodies where one antibody reduces binding of another antibody, but where no competition is observed when the antibodies are added in the reverse order. However, in some embodiments, the first and second antibodies inhibit binding of each other, regardless of the order in which they are added. In some embodiments, one antibody reduces binding of another antibody to its antigen by at least 50%, at least 60%, at least 70%, at least 80%, or at least 90%.

The term “epitope” means a portion of an antigen capable of specific binding to an antibody. Epitopes frequently consist of surface-accessible amino acid residues and/or sugar side chains and may have specific three dimensional structural characteristics, as well as specific charge characteristics. Conformational and non-conformational epitopes are distinguished in that the binding to the former but not the latter is lost in the presence of denaturing solvents. An epitope may comprise amino acid residues that are directly involved in the binding, and other amino acid residues, which are not directly involved in the binding. The epitope to which an antibody binds can be determined using known techniques for epitope determination such as, for example, testing for antibody binding to LAG3 and/or PD-1 variants with different point-mutations, or to chimeric LAG3 and/or PD-1 variants as described further in the Examples provided herein.

Percent “identity” between a polypeptide sequence and a reference sequence, is defined as the percentage of amino acid residues in the polypeptide sequence that are identical to the amino acid residues in the reference sequence, after aligning the sequences and introducing gaps, if necessary, to achieve the maximum percent sequence identity. Alignment for purposes of determining percent amino acid sequence identity can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

A “conservative substitution” or a “conservative amino acid substitution,” refers to the substitution an amino acid with a chemically or functionally similar amino acid. Conservative substitution tables providing similar amino acids are well known in the art. Polypeptide sequences having such substitutions are known as “conservatively modified variants.” By way of example, the groups of amino acids provided in Tables 2-4 are, in some embodiments, considered conservative substitutions for one another.

TABLE 2
Selected groups of amino acids that are considered conservative
substitutions for one another, in certain embodiments.

| | |
|---|---|
| Acidic Residues | D and E |
| Basic Residues | K, R, and H |
| Hydrophilic Uncharged Residues | S, T, N, and Q |
| Aliphatic Uncharged Residues | G, A, V, L, and I |
| Non-polar Uncharged Residues | C, M, and P |
| Aromatic Residues | F, Y, and W |

TABLE 3
Additional selected groups of amino acids that
are considered conservative substitutions for
one another, in certain embodiments.

| | |
|---|---|
| Group 1 | A, S, and T |
| Group 2 | D and E |
| Group 3 | N and Q |
| Group 4 | R and K |
| Group 5 | I, L, and M |
| Group 6 | F, Y, and W |

TABLE 4
Further selected groups of amino acids that are considered conservative
substitutions for one another, in certain embodiments.

| | |
|---|---|
| Group A | A and G |
| Group B | D and E |
| Group C | N and Q |
| Group D | R, K, and H |
| Group E | I, L, M, V |
| Group F | F, Y, and W |
| Group G | S and T |
| Group H | C and M |

Additional conservative substitutions may be found, for example, in Creighton, Proteins: Structures and Molecular Properties 2nd ed. (1993) W. H. Freeman & Co., New York, N.Y. An antibody generated by making one or more conservative substitutions of amino acid residues in a parent antibody is referred to as a “conservatively modified variant.”

The term “amino acid” refers to the twenty common naturally occurring amino acids. Naturally occurring amino acids include alanine (Ala; A), arginine (Arg; R), asparagine (Asn; N), aspartic acid (Asp; D), cysteine (Cys; C); glutamic acid (Glu; E), glutamine (Gln; Q), Glycine (Gly; G); histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying or preventing the onset of the disease or disorder.

As used herein, the term “therapeutically effective amount” or “effective amount” refers to an amount of an antibody or composition that when administered to a subject is effective to treat a disease or disorder.

As used herein, the term “subject” means a mammalian subject. Exemplary subjects include, but are not limited to humans, monkeys, dogs, cats, mice, rats, cows, horses, camels, avians, goats, and sheep. In certain embodiments, the subject is a human. In some embodiments, the subject has a cancer that can be treated or diagnosed with an antibody provided herein. In some embodiments, the cancer is a cancer of epithelial origin.

### 2. LAG3 Antibodies

Provided herein are antibodies that selectively bind human LAG3. In some aspects, the antibody selectively binds to the extracellular domain of human LAG3.

In some embodiments, the antibody binds to a homolog of human LAG3. In some aspects, the antibody binds to a homolog of human LAG3 from a species selected from monkeys, mice, dogs, cats, rats, cows, horses, goats and sheep. In some aspects, the homolog is a cynomolgus monkey homolog.

In some embodiments, the LAG3 antibody has one or more CDRs having particular lengths, in terms of the number of amino acid residues. In some embodiments, the Chothia CDR-H1 of the antibody is 6, 7, or 8 residues in length. In some embodiments, the Kabat CDR-H1 of the antibody is 4, 5, or 6 residues in length. In some embodiments, the Chothia CDR-H2 of the antibody is 5, 6, or 7 residues in length. In some embodiments, the Kabat CDR-H2 of the antibody is 16, 17, or 18 residues in length. In some embodiments, the Kabat/Chothia CDR-H3 of the antibody is 9, 10, 11, 12, or 13 residues in length.

In some aspects, the Kabat/Chothia CDR-L1 of the antibody is 11, 12, 13, 14, 15, 16, 17, or 18 residues in length. In some aspects, the Kabat/Chothia CDR-L2 of the antibody is 6, 7, or 8 residues in length. In some aspects, the Kabat/Chothia CDR-L3 of the antibody is 8, 9, or 10 residues in length.

In some embodiments, the LAG3 antibody comprises a light chain. In some aspects, the light chain is a kappa light chain. In some aspects, the light chain is a lambda light chain.

In some embodiments, the LAG3 antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the LAG3 antibody is an antibody fragment. In some aspects, the antibody fragment is an Fv fragment. In some aspects, the antibody fragment is a Fab fragment. In some aspects, the antibody fragment is a F(ab′)2 fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the scFv-Fc fragment comprises a constant region wherein the constant region comprises SEQ ID NO: 306. The constant region in SEQ ID NO: 306 differs from the human IgG1 constant region of SEQ ID NO: 300 in several respects. First, the sequence in SEQ ID NO: 306 comprises the linker AAGSDQEPKSS (SEQ ID NO: 312). SEQ ID NO: 306 also does not comprise the CH1 domain of the IgG1 constant region. SEQ ID NO: 306 further comprises a C220S (EU numbering system) mutation, which removes an unpaired cysteine reside that is not needed when the light chain constant region is not present (e.g., in an scFv-Fc format). SEQ ID NO: 306 further comprises two, optional, P to S mutations (P230S and P238S by the EU numbering system). Either or both of these serine residues can be reverted to the naturally occurring proline residues. Finally, SEQ ID NO: 306 comprises an aspartic acid (D) residue at EU position 356 and a leucine (L) residue at EU position 358. In contrast, SEQ ID NO: 300 comprises glutamic acid (E) in EU position 356 and methionine (M) in EU position 358. In some embodiments, the antibodies provided herein comprise constant regions comprising D356/L358, E356/M358, D356/M358, or E356/L358 (EU numbering). However, a skilled person will recognize that the antibodies provide herein may comprise any suitable constant region and that the constant region sequences provided herein are for illustrative purposes.

In some embodiments, the LAG3 antibody is a monoclonal antibody. In some embodiments, the LAG3 antibody is a polyclonal antibody.

In some embodiments, the LAG3 antibody is a chimeric antibody. In some embodiments, the LAG3 antibody is a humanized antibody. In some embodiments, the LAG3 antibody is a human antibody.

In some embodiments, the LAG3 antibody is an affinity matured antibody. In some aspects, the LAG3 antibody is an affinity matured antibody derived from an illustrative sequence provided in this disclosure.

In some embodiments, the LAG3 antibody inhibits the binding of LAG3 to one or more of its ligands. In some aspects, the LAG3 antibody inhibits the binding of LAG3 to a ligand such as MHC class II.

In some embodiments, the LAG3 antibody is provided as a single arm binder. For example, the LAG3 antibody can be provided as part of a bi-specific antibody or bi-specific antibody construct as disclosed here.

The LAG3 antibodies provided herein may be useful for the treatment of a variety of diseases and conditions including cancers. In particular, the LAG3 antibodies provided herein may be useful for the treatment of cancers of epithelial origin.

2.1. LAG3 CDR-H3 Sequences

In some embodiments, the LAG3 antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of an scFv-Fc sequence provided in SEQ ID NO: 379. In some aspects, the CDR-H3 sequence is a CDR-H3 sequence of a V_{H} sequence provided in SEQ ID NOs.: 191-210.

In some embodiments, the LAG3 antibody comprises a CDR-H3 sequence comprising, consisting of, or consisting essentially of a sequence selected from SEQ ID NOs: 110-129. In some aspects, the antibody comprises a CDR-H3 sequence comprising, consisting of, or consisting essentially of SEQ ID NO: 110. In some aspects, the antibody comprises a CDR-H3 sequence comprising, consisting of, or consisting essentially of SEQ ID NO: 111. In some aspects, the antibody comprises a CDR-H3 sequence comprising, consisting of, or consisting essentially of SEQ ID NO: 112. In some aspects, the antibody comprises a CDR-H3 sequence comprising, consisting of, or consisting essentially of SEQ ID NO: 113. In some aspects, the antibody comprises a CDR-H3 sequence comprising, consisting of, or consisting essentially of SEQ ID NO: 114. In some aspects, the antibody comprises a CDR-H3 sequence comprising, consisting of, or consisting essentially of SEQ ID NO: 115. In some aspects, the antibody comprises a CDR-H3 sequence comprising, consisting of, or consisting essentially of SEQ ID NO: 116. In some aspects, the antibody comprises a CDR-H3 sequence comprising, consisting of, or consisting essentially of SEQ ID NO: 117. In some aspects, the antibody comprises a CDR-H3 sequence comprising, consisting of, or consisting essentially of SEQ ID NO: 118. In some aspects, the antibody comprises a CDR-H3 sequence comprising, consisting of, or consisting essentially of SEQ ID NO: 119. In some aspects, the antibody comprises a CDR-H3 sequence comprising, consisting of, or consisting essentially of SEQ ID NO: 120. In some aspects, the antibody comprises a CDR-H3 sequence comprising, consisting of, or consisting essentially of SEQ ID NO: 121. In some aspects, the antibody comprises a CDR-H3 sequence comprising, consisting of, or consisting essentially of SEQ ID NO: 122. In some aspects, the antibody comprises a CDR-H3 sequence comprising, consisting of, or consisting essentially of SEQ ID NO: 123. In some aspects, the antibody comprises a CDR-H3 sequence comprising, consisting of, or consisting essentially of SEQ ID NO: 124. In some aspects, the antibody comprises a CDR-H3 sequence comprising, consisting of, or consisting essentially of SEQ ID NO: 125. In some aspects, the antibody comprises a CDR-H3 sequence comprising, consisting of, or consisting essentially of SEQ ID NO: 126. In some aspects, the antibody comprises a CDR-H3 sequence comprising, consisting of, or consisting essentially of SEQ ID NO: 127. In some aspects, the antibody comprises a CDR-H3 sequence comprising, consisting of, or consisting essentially of SEQ ID NO: 128. In some aspects, the antibody comprises a CDR-H3 sequence comprising, consisting of, or consisting essentially of SEQ ID NO: 129.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

2.2. LAG3 V_{H} Sequences Comprising Illustrative CDRs

In some embodiments, the LAG3 antibody comprises a V_{H} sequence comprising one or more CDR-H sequences comprising, consisting of, or consisting essentially of one or more illustrative CDR-H sequences provided in this disclosure, and variants thereof. In some embodiments, the CDR-H sequences comprise, consist of, or consist essentially of one or more CDR-H sequences provided in a V_{H} sequence selected from SEQ ID NOs: 191-210.

2.2.1. V_{H} Sequences Comprising Illustrative Kabat CDRs

In some embodiments, the LAG3 antibody comprises a V_{H} sequence comprising one or more Kabat CDR-H sequences comprising, consisting of, or consisting essentially of one or more illustrative Kabat CDR-H sequences provided in this disclosure, and variants thereof.

2.2.1.1. Kabat CDR-H3

In some embodiments, the LAG3 antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of an scFv-Fc sequence provided in SEQ ID NO: 379. In some aspects, the Kabat CDR-H3 sequence is a Kabat CDR-H3 sequence of a V_{H} sequence provided in SEQ ID NOs.: 191-210.

In some embodiments, the LAG3 antibody comprises a V_{H} sequence comprising a Kabat CDR-H3 sequence comprising, consisting of, or consisting essentially of a sequence selected from SEQ ID NOs: 110-129. In some aspects, the antibody comprises a V_{H} sequence comprising a Kabat CDR-H3 sequence comprising, consisting of, or consisting essentially of SEQ ID NO: 110. In some aspects, the antibody comprises a V_{H} sequence comprising a Kabat CDR-H3 sequence comprising, consisting of, or consisting essentially of SEQ ID NO: 111. In some aspects, the antibody comprises a V_{H} sequence comprising a Kabat CDR-H3 sequence comprising, consisting of, or consisting essentially of SEQ ID NO: 112. In some aspects, the antibody comprises a V_{H} sequence comprising a Kabat CDR-H3 sequence comprising, consisting of, or consisting essentially of SEQ ID NO: 113. In some aspects, the antibody comprises a V_{H} sequence comprising a Kabat CDR-H3 sequence comprising, consisting of, or consisting essentially of SEQ ID NO: 114. In some aspects, the antibody comprises a V_{H} sequence comprising a Kabat CDR-H3 sequence comprising, consisting of, or consisting essentially of SEQ ID NO: 115. In some aspects, the antibody comprises a V_{H} sequence comprising a Kabat CDR-H3 sequence comprising, consisting of, or consisting essentially of SEQ ID NO: 116. In some aspects, the antibody comprises a V_{H} sequence comprising a Kabat CDR-H3 sequence comprising, consisting of, or consisting essentially of SEQ ID NO: 117. In some aspects, the antibody comprises a V_{H} sequence comprising a Kabat CDR-H3 sequence comprising, consisting of, or consisting essentially of SEQ ID NO: 118. In some aspects, the antibody comprises a V_{H} sequence comprising a Kabat CDR-H3 sequence comprising, consisting of, or consisting essentially of SEQ ID NO: 119. In some aspects, the antibody comprises a V_{H} sequence comprising a Kabat CDR-H3 sequence comprising, consisting of, or consisting essentially of SEQ ID NO: 120. In some aspects, the antibody comprises a V_{H} sequence comprising a Kabat CDR-H3 sequence comprising, consisting of, or consisting essentially of SEQ ID NO: 121. In some aspects, the antibody comprises a V_{H} sequence comprising a Kabat CDR-H3 sequence comprising, consisting of, or consisting essentially of SEQ ID NO: 122. In some aspects, the antibody comprises a V_{H} sequence comprising a Kabat CDR-H3 sequence comprising, consisting of, or consisting essentially of SEQ ID NO: 123. In some aspects, the antibody comprises a V_{H} sequence comprising a Kabat CDR-H3 sequence comprising, consisting of, or consisting essentially of SEQ ID NO: 124. In some aspects, the antibody comprises a V_{H} sequence comprising a Kabat CDR-H3 sequence comprising, consisting of, or consisting essentially of SEQ ID NO: 125. In some aspects, the antibody comprises a V_{H} sequence comprising a Kabat CDR-H3 sequence comprising, consisting of, or consisting essentially of SEQ ID NO: 126. In some aspects, the antibody comprises a V_{H} sequence comprising a Kabat CDR-H3 sequence comprising, consisting of, or consisting essentially of SEQ ID NO: 127. In some aspects, the antibody comprises a V_{H} sequence comprising a Kabat CDR-H3 sequence comprising, consisting of, or consisting essentially of SEQ ID NO: 128. In some aspects, the antibody comprises a V_{H} sequence comprising a Kabat CDR-H3 sequence comprising, consisting of, or consisting essentially of SEQ ID NO: 129.

2.2.1.2. Kabat CDR-H2

In some embodiments, the LAG3 antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of an scFv-Fc sequence provided in SEQ ID NO: 379. In some aspects, the Kabat CDR-H2 sequence is a Kabat CDR-H2 sequence of a V_{H} sequence provided in SEQ ID NOs.: 191-210.

In some embodiments, the LAG3 antibody comprises a V_{H} sequence comprising a Kabat CDR-H2 sequence comprising, consisting of, or consisting essentially of a sequence selected from SEQ ID NOs: 88-107. In some aspects, the antibody comprises a V_{H} sequence comprising a Kabat CDR-H2 sequence comprising, consisting of, or consisting essentially of SEQ ID NO: 88. In some aspects, the antibody comprises a V_{H} sequence comprising a Kabat CDR-H2 sequence comprising, consisting of, or consisting essentially of SEQ ID NO: 89. In some aspects, the antibody comprises a V_{H} sequence comprising a Kabat CDR-H2 sequence comprising, consisting of, or consisting essentially of SEQ ID NO: 90. In some aspects, the antibody comprises a V_{H} sequence comprising a Kabat CDR-H2 sequence comprising, consisting of, or consisting essentially of SEQ ID NO: 91. In some aspects, the antibody comprises a V_{H} sequence comprising a Kabat CDR-H2 sequence comprising, consisting of, or consisting essentially of SEQ ID NO: 92. In some aspects, the antibody comprises a V_{H} sequence comprising a Kabat CDR-H2 sequence comprising, consisting of, or consisting essentially of SEQ ID NO: 93. In some aspects, the antibody comprises a V_{H} sequence comprising a Kabat CDR-H2 sequence comprising, consisting of, or consisting essentially of SEQ ID NO: 94. In some aspects, the antibody comprises a V_{H} sequence comprising a Kabat CDR-H2 sequence comprising, consisting of, or consisting essentially of SEQ ID NO: 95. In some aspects, the antibody comprises a V_{H} sequence comprising a Kabat CDR-H2 sequence comprising, consisting of, or consisting essentially of SEQ ID NO: 96. In some aspects, the antibody comprises a V_{H} sequence comprising a Kabat CDR-H2 sequence comprising, consisting of, or consisting essentially of SEQ ID NO: 97. In some aspects, the antibody comprises a V_{H} sequence comprising a Kabat CDR-H2 sequence comprising, consisting of, or consisting essentially of SEQ ID NO: 98. In some aspects, the antibody comprises a V_{H} sequence comprising a Kabat CDR-H2 sequence comprising, consisting of, or consisting essentially of SEQ ID NO: 99. In some aspects, the antibody comprises a V_{H} sequence comprising a Kabat CDR-H2 sequence comprising, consisting of, or consisting essentially of SEQ ID NO: 100. In some aspects, the antibody comprises a V_{H} sequence comprising a Kabat CDR-H2 sequence comprising, consisting of, or consisting essentially of SEQ ID NO: 101. In some aspects, the antibody comprises a V_{H} sequence comprising a Kabat CDR-H2 sequence comprising, consisting of, or consisting essentially of SEQ ID NO: 102. In some aspects, the antibody comprises a V_{H} sequence comprising a Kabat CDR-H2 sequence comprising, consisting of, or consisting essentially of SEQ ID NO: 103. In some aspects, the antibody comprises a V_{H} sequence comprising a Kabat CDR-H2 sequence comprising, consisting of, or consisting essentially of SEQ ID NO: 104. In some aspects, the antibody comprises a V_{H} sequence comprising a Kabat CDR-H2 sequence comprising, consisting of, or consisting essentially of SEQ ID NO: 105. In some aspects, the antibody comprises a V_{H} sequence comprising a Kabat CDR-H2 sequence comprising, consisting of, or consisting essentially of SEQ ID NO: 106. In some aspects, the antibody comprises a V_{H} sequence comprising a Kabat CDR-H2 sequence comprising, consisting of, or consisting essentially of SEQ ID NO: 107.

2.2.1.3. Kabat CDR-H1

In some embodiments, the LAG3 antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of an scFv-Fc sequence provided in SEQ ID NO: 379. In some aspects, the Kabat CDR-H1 sequence is a Kabat CDR-H1 sequence of a V_{H} sequence provided in SEQ ID NOs.: 191-210.

In some embodiments, the LAG3 antibody comprises a V_{H} sequence comprising a Kabat CDR-H1 sequence comprising, consisting of, or consisting essentially of a sequence selected from SEQ ID NOs: 39-58. In some aspects, the antibody comprises a V_{H} sequence comprising a Kabat CDR-H1 sequence comprising, consisting of, or consisting essentially of SEQ ID NO: 39. In some aspects, the antibody comprises a V_{H} sequence comprising a Kabat CDR-H1 sequence comprising, consisting of, or consisting essentially of SEQ ID NO: 40. In some aspects, the antibody comprises a V_{H} sequence comprising a Kabat CDR-H1 sequence comprising, consisting of, or consisting essentially of SEQ ID NO: 41. In some aspects, the antibody comprises a V_{H} sequence comprising a Kabat CDR-H1 sequence comprising, consisting of, or consisting essentially of SEQ ID NO: 42. In some aspects, the antibody comprises a V_{H} sequence comprising a Kabat CDR-H1 sequence comprising, consisting of, or consisting essentially of SEQ ID NO: 43. In some aspects, the antibody comprises a V_{H} sequence comprising a Kabat CDR-H1 sequence comprising, consisting of, or consisting essentially of SEQ ID NO: 44. In some aspects, the antibody comprises a V_{H} sequence comprising a Kabat CDR-H1 sequence comprising, consisting of, or consisting essentially of SEQ ID NO: 45. In some aspects, the antibody comprises a V_{H} sequence comprising a Kabat CDR-H1 sequence comprising, consisting of, or consisting essentially of SEQ ID NO: 46. In some aspects, the antibody comprises a V_{H} sequence comprising a Kabat CDR-H1 sequence comprising, consisting of, or consisting essentially of SEQ ID NO: 47. In some aspects, the antibody comprises a V_{H} sequence comprising a Kabat CDR-H1 sequence comprising, consisting of, or consisting essentially of SEQ ID NO: 48. In some aspects, the antibody comprises a V_{H} sequence comprising a Kabat CDR-H1 sequence comprising, consisting of, or consisting essentially of SEQ ID NO: 49. In some aspects, the antibody comprises a V_{H} sequence comprising a Kabat CDR-H1 sequence comprising, consisting of, or consisting essentially of SEQ ID NO: 50. In some aspects, the antibody comprises a V_{H} sequence comprising a Kabat CDR-H1 sequence comprising, consisting of, or consisting essentially of SEQ ID NO: 51. In some aspects, the antibody comprises a V_{H} sequence comprising a Kabat CDR-H1 sequence comprising, consisting of, or consisting essentially of SEQ ID NO: 52. In some aspects, the antibody comprises a V_{H} sequence comprising a Kabat CDR-H1 sequence comprising, consisting of, or consisting essentially of SEQ ID NO: 53. In some aspects, the antibody comprises a V_{H} sequence comprising a Kabat CDR-H1 sequence comprising, consisting of, or consisting essentially of SEQ ID NO: 54. In some aspects, the antibody comprises a V_{H} sequence comprising a Kabat CDR-H1 sequence comprising, consisting of, or consisting essentially of SEQ ID NO: 55. In some aspects, the antibody comprises a V_{H} sequence comprising a Kabat CDR-H1 sequence comprising, consisting of, or consisting essentially of SEQ ID NO: 56. In some aspects, the antibody comprises a V_{H} sequence comprising a Kabat CDR-H1 sequence comprising, consisting of, or consisting essentially of SEQ ID NO: 57. In some aspects, the antibody comprises a V_{H} sequence comprising a Kabat CDR-H1 sequence comprising, consisting of, or consisting essentially of SEQ ID NO: 58.

2.2.1.4. Kabat CDR-H3+Kabat CDR-H2

In some embodiments, the LAG3 antibody comprises a V_{H} sequence comprising a Kabat CDR-H3 sequence comprising, consisting of, or consisting essentially of a sequence selected from SEQ ID NOs: 110-129, and a Kabat CDR-H2 sequence comprising, consisting of, or consisting essentially of a sequence selected from SEQ ID NOs: 88-107. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 191-210.

2.2.1.5. Kabat CDR-H3+Kabat CDR-H1

In some embodiments, the LAG3 antibody comprises a V_{H} sequence comprising a Kabat CDR-H3 sequence comprising, consisting of, or consisting essentially of a sequence selected from SEQ ID NOs: 110-129, and a Kabat CDR-H1 sequence comprising, consisting of, or consisting essentially of a sequence selected from SEQ ID NOs: 39-58. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 191-210.

2.2.1.6. Kabat CDR-H1+Kabat CDR-H2

In some embodiments, the LAG3 antibody comprises a V_{H} sequence comprising a Kabat CDR-H1 sequence comprising, consisting of, or consisting essentially of a sequence selected from SEQ ID NOs: 39-58 and a Kabat CDR-H2 sequence comprising, consisting of, or consisting essentially of a sequence selected from SEQ ID NOs: 88-107. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 191-210.

2.2.1.7. Kabat CDR-H1+Kabat CDR-H2+Kabat CDR-H3

In some embodiments, the LAG3 antibody comprises a V_{H} sequence comprising a Kabat CDR-H1 sequence comprising, consisting of, or consisting essentially of a sequence selected from SEQ ID NOs: 39-58, a Kabat CDR-H2 sequence comprising, consisting of, or consisting essentially of a sequence selected from SEQ ID NOs: 88-107, and a Kabat CDR-H3 sequence comprising, consisting of, or consisting essentially of a sequence selected from SEQ ID NOs: 110-129. In some aspects, the Kabat CDR-H1 sequence, Kabat CDR-H2 sequence, and Kabat CDR-H3 sequence are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 191-210.

2.2.1.8. Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

2.2.2. V_{H} Sequences Comprising Illustrative Chothia CDRs

In some embodiments, the LAG3 antibody comprises a V_{H} sequence comprising one or more Chothia CDR-H sequences comprising, consisting of, or consisting essentially of one or more illustrative Chothia CDR-H sequences provided in this disclosure, and variants thereof.

2.2.2.1. Chothia CDR-H3

In some embodiments, the LAG3 antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of an scFv-Fc sequence provided in SEQ ID NO: 379. In some aspects, the Chothia CDR-H3 sequence is a Chothia CDR-H3 sequence of a V_{H} sequence provided in SEQ ID NOs.: 191-210.

In some embodiments, the LAG3 antibody comprises a V_{H} sequence comprising a Chothia CDR-H3 sequence comprising, consisting of, or consisting essentially of a sequence selected from SEQ ID NOs: 110-129. In some aspects, the antibody comprises a V_{H} sequence comprising a Chothia CDR-H3 sequence comprising, consisting of, or consisting essentially of SEQ ID NO: 110. In some aspects, the antibody comprises a V_{H} sequence comprising a Chothia CDR-H3 sequence comprising, consisting of, or consisting essentially of SEQ ID NO: 111. In some aspects, the antibody comprises a V_{H} sequence comprising a Chothia CDR-H3 sequence comprising, consisting of, or consisting essentially of SEQ ID NO: 112. In some aspects, the antibody comprises a V_{H} sequence comprising a Chothia CDR-H3 sequence comprising, consisting of, or consisting essentially of SEQ ID NO: 113. In some aspects, the antibody comprises a V_{H} sequence comprising a Chothia CDR-H3 sequence comprising, consisting of, or consisting essentially of SEQ ID NO: 114. In some aspects, the antibody comprises a V_{H} sequence comprising a Chothia CDR-H3 sequence comprising, consisting of, or consisting essentially of SEQ ID NO: 115. In some aspects, the antibody comprises a V_{H} sequence comprising a Chothia CDR-H3 sequence comprising, consisting of, or consisting essentially of SEQ ID NO: 116. In some aspects, the antibody comprises a V_{H} sequence comprising a Chothia CDR-H3 sequence comprising, consisting of, or consisting essentially of SEQ ID NO: 117. In some aspects, the antibody comprises a V_{H} sequence comprising a Chothia CDR-H3 sequence comprising, consisting of, or consisting essentially of SEQ ID NO: 118. In some aspects, the antibody comprises a V_{H} sequence comprising a Chothia CDR-H3 sequence comprising, consisting of, or consisting essentially of SEQ ID NO: 119. In some aspects, the antibody comprises a V_{H} sequence comprising a Chothia CDR-H3 sequence comprising, consisting of, or consisting essentially of SEQ ID NO: 120. In some aspects, the antibody comprises a V_{H} sequence comprising a Chothia CDR-H3 sequence comprising, consisting of, or consisting essentially of SEQ ID NO: 121. In some aspects, the antibody comprises a V_{H} sequence comprising a Chothia CDR-H3 sequence comprising, consisting of, or consisting essentially of SEQ ID NO: 122. In some aspects, the antibody comprises a V_{H} sequence comprising a Chothia CDR-H3 sequence comprising, consisting of, or consisting essentially of SEQ ID NO: 123. In some aspects, the antibody comprises a V_{H} sequence comprising a Chothia CDR-H3 sequence comprising, consisting of, or consisting essentially of SEQ ID NO: 124. In some aspects, the antibody comprises a V_{H} sequence comprising a Chothia CDR-H3 sequence comprising, consisting of, or consisting essentially of SEQ ID NO: 125. In some aspects, the antibody comprises a V_{H} sequence comprising a Chothia CDR-H3 sequence comprising, consisting of, or consisting essentially of SEQ ID NO: 126. In some aspects, the antibody comprises a V_{H} sequence comprising a Chothia CDR-H3 sequence comprising, consisting of, or consisting essentially of SEQ ID NO: 127. In some aspects, the antibody comprises a V_{H} sequence comprising a Chothia CDR-H3 sequence comprising, consisting of, or consisting essentially of SEQ ID NO: 128. In some aspects, the antibody comprises a V_{H} sequence comprising a Chothia CDR-H3 sequence comprising, consisting of, or consisting essentially of SEQ ID NO: 129.

2.2.2.2. Chothia CDR-H2

In some embodiments, the LAG3 antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of an scFv-Fc sequence provided in SEQ ID NO: 379. In some aspects, the Chothia CDR-H2 sequence is a Chothia CDR-H2 sequence of a V_{H} sequence provided in SEQ ID NOs.: 191-210.

In some embodiments, the LAG3 antibody comprises a V_{H} sequence comprising a Chothia CDR-H2 sequence comprising, consisting of, or consisting essentially of a sequence selected from SEQ ID NOs: 66-85. In some aspects, the antibody comprises a V_{H} sequence comprising a Chothia CDR-H2 sequence comprising, consisting of, or consisting essentially of SEQ ID NO: 66. In some aspects, the antibody comprises a V_{H} sequence comprising a Chothia CDR-H2 sequence comprising, consisting of, or consisting essentially of SEQ ID NO: 67. In some aspects, the antibody comprises a V_{H} sequence comprising a Chothia CDR-H2 sequence comprising, consisting of, or consisting essentially of SEQ ID NO: 68. In some aspects, the antibody comprises a V_{H} sequence comprising a Chothia CDR-H2 sequence comprising, consisting of, or consisting essentially of SEQ ID NO: 69. In some aspects, the antibody comprises a V_{H} sequence comprising a Chothia CDR-H2 sequence comprising, consisting of, or consisting essentially of SEQ ID NO: 70. In some aspects, the antibody comprises a V_{H} sequence comprising a Chothia CDR-H2 sequence comprising, consisting of, or consisting essentially of SEQ ID NO: 71. In some aspects, the antibody comprises a V_{H} sequence comprising a Chothia CDR-H2 sequence comprising, consisting of, or consisting essentially of SEQ ID NO: 72. In some aspects, the antibody comprises a V_{H} sequence comprising a Chothia CDR-H2 sequence comprising, consisting of, or consisting essentially of SEQ ID NO: 73. In some aspects, the antibody comprises a V_{H} sequence comprising a Chothia CDR-H2 sequence comprising, consisting of, or consisting essentially of SEQ ID NO: 74. In some aspects, the antibody comprises a V_{H} sequence comprising a Chothia CDR-H2 sequence comprising, consisting of, or consisting essentially of SEQ ID NO: 75. In some aspects, the antibody comprises a V_{H} sequence comprising a Chothia CDR-H2 sequence comprising, consisting of, or consisting essentially of SEQ ID NO: 76. In some aspects, the antibody comprises a V_{H} sequence comprising a Chothia CDR-H2 sequence comprising, consisting of, or consisting essentially of SEQ ID NO: 77. In some aspects, the antibody comprises a V_{H} sequence comprising a Chothia CDR-H2 sequence comprising, consisting of, or consisting essentially of SEQ ID NO: 78. In some aspects, the antibody comprises a V_{H} sequence comprising a Chothia CDR-H2 sequence comprising, consisting of, or consisting essentially of SEQ ID NO: 79. In some aspects, the antibody comprises a V_{H} sequence comprising a Chothia CDR-H2 sequence comprising, consisting of, or consisting essentially of SEQ ID NO: 80. In some aspects, the antibody comprises a V_{H} sequence comprising a Chothia CDR-H2 sequence comprising, consisting of, or consisting essentially of SEQ ID NO: 81. In some aspects, the antibody comprises a V_{H} sequence comprising a Chothia CDR-H2 sequence comprising, consisting of, or consisting essentially of SEQ ID NO: 82. In some aspects, the antibody comprises a V_{H} sequence comprising a Chothia CDR-H2 sequence comprising, consisting of, or consisting essentially of SEQ ID NO: 83. In some aspects, the antibody comprises a V_{H} sequence comprising a Chothia CDR-H2 sequence comprising, consisting of, or consisting essentially of SEQ ID NO: 84. In some aspects, the antibody comprises a V_{H} sequence comprising a Chothia CDR-H2 sequence comprising, consisting of, or consisting essentially of SEQ ID NO: 85.

2.2.2.3. Chothia CDR-H1

In some embodiments, the LAG3 antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of an scFv-Fc sequence provided in SEQ ID NO: 379. In some aspects, the Chothia CDR-H1 sequence is a Chothia CDR-H1 sequence of a V_{H} sequence provided in SEQ ID NOs.: 191-210.

In some embodiments, the LAG3 antibody comprises a V_{H} sequence comprising a Chothia CDR-H1 sequence comprising, consisting of, or consisting essentially of a sequence selected from SEQ ID NOs: 4-23. In some aspects, the antibody comprises a V_{H} sequence comprising a Chothia CDR-H1 sequence comprising, consisting of, or consisting essentially of SEQ ID NO: 4. In some aspects, the antibody comprises a V_{H} sequence comprising a Chothia CDR-H1 sequence comprising, consisting of, or consisting essentially of SEQ ID NO: 5. In some aspects, the antibody comprises a V_{H} sequence comprising a Chothia CDR-H1 sequence comprising, consisting of, or consisting essentially of SEQ ID NO: 6. In some aspects, the antibody comprises a V_{H} sequence comprising a Chothia CDR-H1 sequence comprising, consisting of, or consisting essentially of SEQ ID NO: 7. In some aspects, the antibody comprises a V_{H} sequence comprising a Chothia CDR-H1 sequence comprising, consisting of, or consisting essentially of SEQ ID NO: 8. In some aspects, the antibody comprises a V_{H} sequence comprising a Chothia CDR-H1 sequence comprising, consisting of, or consisting essentially of SEQ ID NO: 9. In some aspects, the antibody comprises a V_{H} sequence comprising a Chothia CDR-H1 sequence comprising, consisting of, or consisting essentially of SEQ ID NO: 10. In some aspects, the antibody comprises a V_{H} sequence comprising a Chothia CDR-H1 sequence comprising, consisting of, or consisting essentially of SEQ ID NO: 11. In some aspects, the antibody comprises a V_{H} sequence comprising a Chothia CDR-H1 sequence comprising, consisting of, or consisting essentially of SEQ ID NO: 12. In some aspects, the antibody comprises a V_{H} sequence comprising a Chothia CDR-H1 sequence comprising, consisting of, or consisting essentially of SEQ ID NO: 13. In some aspects, the antibody comprises a V_{H} sequence comprising a Chothia CDR-H1 sequence comprising, consisting of, or consisting essentially of SEQ ID NO: 14. In some aspects, the antibody comprises a V_{H} sequence comprising a Chothia CDR-H1 sequence comprising, consisting of, or consisting essentially of SEQ ID NO: 15. In some aspects, the antibody comprises a V_{H} sequence comprising a Chothia CDR-H1 sequence comprising, consisting of, or consisting essentially of SEQ ID NO: 16. In some aspects, the antibody comprises a V_{H} sequence comprising a Chothia CDR-H1 sequence comprising, consisting of, or consisting essentially of SEQ ID NO: 17. In some aspects, the antibody comprises a V_{H} sequence comprising a Chothia CDR-H1 sequence comprising, consisting of, or consisting essentially of SEQ ID NO: 18. In some aspects, the antibody comprises a V_{H} sequence comprising a Chothia CDR-H1 sequence comprising, consisting of, or consisting essentially of SEQ ID NO: 19. In some aspects, the antibody comprises a V_{H} sequence comprising a Chothia CDR-H1 sequence comprising, consisting of, or consisting essentially of SEQ ID NO: 20. In some aspects, the antibody comprises a V_{H} sequence comprising a Chothia CDR-H1 sequence comprising, consisting of, or consisting essentially of SEQ ID NO: 21. In some aspects, the antibody comprises a V_{H} sequence comprising a Chothia CDR-H1 sequence comprising, consisting of, or consisting essentially of SEQ ID NO: 22. In some aspects, the antibody comprises a V_{H} sequence comprising a Chothia CDR-H1 sequence comprising, consisting of, or consisting essentially of SEQ ID NO: 23.

2.2.2.4. Chothia CDR-H3+Chothia CDR-H2

In some embodiments, the LAG3 antibody comprises a V_{H} sequence comprising a Chothia CDR-H3 sequence comprising, consisting of, or consisting essentially of a sequence selected from SEQ ID NOs: 110-129, and a Chothia CDR-H2 sequence comprising, consisting of, or consisting essentially of a sequence selected from SEQ ID NOs: 66-85. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs: 191-210.

2.2.2.5. Chothia CDR-H3+Chothia CDR-H1

In some embodiments, the LAG3 antibody comprises a V_{H} sequence comprising a Chothia CDR-H3 sequence comprising, consisting of, or consisting essentially of a sequence selected from SEQ ID NOs: 110-129, and a Chothia CDR-H1 sequence comprising, consisting of, or consisting essentially of a sequence selected from SEQ ID NOs: 4-23. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 191-210.

2.2.2.6. Chothia CDR-H1+Chothia CDR-H2

In some embodiments, the LAG3 antibody comprises a V_{H} sequence comprising a Chothia CDR-H1 sequence comprising, consisting of, or consisting essentially of a sequence selected from SEQ ID NOs: 4-23 and a Chothia CDR-H2 sequence comprising, consisting of, or consisting essentially of a sequence selected from SEQ ID NOs: 66-85. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 191-210.

2.2.2.7. Chothia CDR-H1+Chothia CDR-H2+Chothia CDR-H3

In some embodiments, the LAG3 antibody comprises a V_{H} sequence comprising a Chothia CDR-H1 sequence comprising, consisting of, or consisting essentially of a sequence selected from SEQ ID NOs: 4-23, a Chothia CDR-H2 sequence comprising, consisting of, or consisting essentially of a sequence selected from SEQ ID NOs: 66-85, and a Chothia CDR-H3 sequence comprising, consisting of, or consisting essentially of a sequence selected from SEQ ID NOs: 110-129. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 191-210.

2.2.2.8. Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

2.3. LAG3 V_{H} Sequences

In some embodiments, the LAG3 antibody comprises, consists of, or consists essentially of a V_{H} sequence of an scFv-Fc sequence provided in SEQ ID NO: 379. In some embodiments, the antibody comprises, consists of, or consists essentially of a V_{H} sequence provided in SEQ ID NOs.: 191-210.

In some embodiments, the LAG3 antibody comprises a V_{H} sequence comprising, consisting of, or consisting essentially of a sequence selected from SEQ ID NOs: 191-210. In some aspects, the antibody comprises a V_{H} sequence comprising, consisting of, or consisting essentially of SEQ ID NO: 191. In some aspects, the antibody comprises a V_{H} sequence comprising, consisting of, or consisting essentially of SEQ ID NO: 192. In some aspects, the antibody comprises a V_{H} sequence comprising, consisting of, or consisting essentially of SEQ ID NO: 193. In some aspects, the antibody comprises a V_{H} sequence comprising, consisting of, or consisting essentially of SEQ ID NO: 194. In some aspects, the antibody comprises a V_{H} sequence comprising, consisting of, or consisting essentially of SEQ ID NO: 195. In some aspects, the antibody comprises a V_{H} sequence comprising, consisting of, or consisting essentially of SEQ ID NO: 196. In some aspects, the antibody comprises a V_{H} sequence comprising, consisting of, or consisting essentially of SEQ ID NO: 197. In some aspects, the antibody comprises a V_{H} sequence comprising, consisting of, or consisting essentially of SEQ ID NO: 198. In some aspects, the antibody comprises a V_{H} sequence comprising, consisting of, or consisting essentially of SEQ ID NO: 199. In some aspects, the antibody comprises a V_{H} sequence comprising, consisting of, or consisting essentially of SEQ ID NO: 200. In some aspects, the antibody comprises a V_{H} sequence comprising, consisting of, or consisting essentially of SEQ ID NO: 201. In some aspects, the antibody comprises a V_{H} sequence comprising, consisting of, or consisting essentially of SEQ ID NO: 202. In some aspects, the antibody comprises a V_{H} sequence comprising, consisting of, or consisting essentially of SEQ ID NO: 203. In some aspects, the antibody comprises a V_{H} sequence comprising, consisting of, or consisting essentially of SEQ ID NO: 204. In some aspects, the antibody comprises a V_{H} sequence comprising, consisting of, or consisting essentially of SEQ ID NO: 205. In some aspects, the antibody comprises a V_{H} sequence comprising, consisting of, or consisting essentially of SEQ ID NO: 206. In some aspects, the antibody comprises a V_{H} sequence comprising, consisting of, or consisting essentially of SEQ ID NO: 207. In some aspects, the antibody comprises a V_{H} sequence comprising, consisting of, or consisting essentially of SEQ ID NO: 208. In some aspects, the antibody comprises a V_{H} sequence comprising, consisting of, or consisting essentially of SEQ ID NO: 209. In some aspects, the antibody comprises a V_{H} sequence comprising, consisting of, or consisting essentially of SEQ ID NO: 210.

2.3.1. Variants of V_{H} Sequences

In some embodiments, the V_{H} sequences provided herein comprise, consist of, or consist essentially of a variant of an illustrative V_{H} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.4. LAG3 CDR-L3 Sequences

In some embodiments, the LAG3 antibody comprises a CDR-L3 sequence comprising, consisting of, or consisting essentially of a CDR-L3 sequence of an illustrative antibody or V_{L} sequence provided herein. In some aspects, the CDR-L3 sequence is a CDR-L3 sequence of an scFv-Fc sequence provided in SEQ ID NO: 379. In some aspects, the CDR-L3 sequence is a CDR-L3 sequence of a V_{L} sequence provided in SEQ ID NOs.: 251-266.

In some embodiments, the LAG3 antibody comprises a CDR-L3 sequence comprising, consisting of, or consisting essentially of a sequence selected from SEQ ID NOs: 173-188. In some aspects, the antibody comprises a CDR-L3 sequence comprising, consisting of, or consisting essentially of SEQ ID NO: 173. In some aspects, the antibody comprises a CDR-L3 sequence comprising, consisting of, or consisting essentially of SEQ ID NO: 174. In some aspects, the antibody comprises a CDR-L3 sequence comprising, consisting of, or consisting essentially of SEQ ID NO: 175. In some aspects, the antibody comprises a CDR-L3 sequence comprising, consisting of, or consisting essentially of SEQ ID NO: 176. In some aspects, the antibody comprises a CDR-L3 sequence comprising, consisting of, or consisting essentially of SEQ ID NO: 177. In some aspects, the antibody comprises a CDR-L3 sequence comprising, consisting of, or consisting essentially of SEQ ID NO: 178. In some aspects, the antibody comprises a CDR-L3 sequence comprising, consisting of, or consisting essentially of SEQ ID NO: 179. In some aspects, the antibody comprises a CDR-L3 sequence comprising, consisting of, or consisting essentially of SEQ ID NO: 180. In some aspects, the antibody comprises a CDR-L3 sequence comprising, consisting of, or consisting essentially of SEQ ID NO: 181. In some aspects, the antibody comprises a CDR-L3 sequence comprising, consisting of, or consisting essentially of SEQ ID NO: 182. In some aspects, the antibody comprises a CDR-L3 sequence comprising, consisting of, or consisting essentially of SEQ ID NO: 183. In some aspects, the antibody comprises a CDR-L3 sequence comprising, consisting of, or consisting essentially of SEQ ID NO: 184. In some aspects, the antibody comprises a CDR-L3 sequence comprising, consisting of, or consisting essentially of SEQ ID NO: 185. In some aspects, the antibody comprises a CDR-L3 sequence comprising, consisting of, or consisting essentially of SEQ ID NO: 186. In some aspects, the antibody comprises a CDR-L3 sequence comprising, consisting of, or consisting essentially of SEQ ID NO: 187. In some aspects, the antibody comprises a CDR-L3 sequence comprising, consisting of, or consisting essentially of SEQ ID NO: 188.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.5. LAG3 V_{L} Sequences Comprising Illustrative CDRs

In some embodiments, the LAG3 antibody comprises a V_{L} sequence comprising one or more CDR-L sequences comprising, consisting of, or consisting essentially of one or more illustrative CDR-L sequences provided in this disclosure, and variants thereof.

2.5.1. CDR-L3

In some embodiments, the LAG3 antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of an scFv-Fc sequence provided in SEQ ID NO: 379. In some aspects, the CDR-L3 sequence is a CDR-L3 sequence of a V_{L} sequence provided in SEQ ID NOs.: 251-266.

In some embodiments, the LAG3 antibody comprises a V_{L} sequence comprising a CDR-L3 sequence comprising, consisting of, or consisting essentially of a sequence selected from SEQ ID NOs: 173-188. In some aspects, the antibody comprises a V_{L} sequence comprising a CDR-L3 sequence comprising, consisting of, or consisting essentially of SEQ ID NO: 173. In some aspects, the antibody comprises a V_{L} sequence comprising a CDR-L3 sequence comprising, consisting of, or consisting essentially of SEQ ID NO: 174. In some aspects, the antibody comprises a V_{L} sequence comprising a CDR-L3 sequence comprising, consisting of, or consisting essentially of SEQ ID NO: 175. In some aspects, the antibody comprises a V_{L} sequence comprising a CDR-L3 sequence comprising, consisting of, or consisting essentially of SEQ ID NO: 176. In some aspects, the antibody comprises a V_{L} sequence comprising a CDR-L3 sequence comprising, consisting of, or consisting essentially of SEQ ID NO: 177. In some aspects, the antibody comprises a V_{L} sequence comprising a CDR-L3 sequence comprising, consisting of, or consisting essentially of SEQ ID NO: 178. In some aspects, the antibody comprises a V_{L} sequence comprising a CDR-L3 sequence comprising, consisting of, or consisting essentially of SEQ ID NO: 179. In some aspects, the antibody comprises a V_{L} sequence comprising a CDR-L3 sequence comprising, consisting of, or consisting essentially of SEQ ID NO: 180. In some aspects, the antibody comprises a V_{L} sequence comprising a CDR-L3 sequence comprising, consisting of, or consisting essentially of SEQ ID NO: 181. In some aspects, the antibody comprises a V_{L} sequence comprising a CDR-L3 sequence comprising, consisting of, or consisting essentially of SEQ ID NO: 182. In some aspects, the antibody comprises a V_{L} sequence comprising a CDR-L3 sequence comprising, consisting of, or consisting essentially of SEQ ID NO: 183. In some aspects, the antibody comprises a V_{L} sequence comprising a CDR-L3 sequence comprising, consisting of, or consisting essentially of SEQ ID NO: 184. In some aspects, the antibody comprises a V_{L} sequence comprising a CDR-L3 sequence comprising, consisting of, or consisting essentially of SEQ ID NO: 185. In some aspects, the antibody comprises a V_{L} sequence comprising a CDR-L3 sequence comprising, consisting of, or consisting essentially of SEQ ID NO: 186. In some aspects, the antibody comprises a V_{L} sequence comprising a CDR-L3 sequence comprising, consisting of, or consisting essentially of SEQ ID NO: 187. In some aspects, the antibody comprises a V_{L} sequence comprising a CDR-L3 sequence comprising, consisting of, or consisting essentially of SEQ ID NO: 188.

2.5.2. CDR-L2

In some embodiments, the LAG3 antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of an scFv-Fc sequence provided in SEQ ID NO: 379. In some aspects, the CDR-L2 sequence is a CDR-L2 sequence of a V_{L} sequence provided in SEQ ID NOs.: 251-266.

In some embodiments, the LAG3 antibody comprises a V_{L} sequence comprising a CDR-L2 sequence comprising, consisting of, or consisting essentially of a sequence selected from SEQ ID NOs: 150-165. In some aspects, the antibody comprises a V_{L} sequence comprising a CDR-L2 sequence comprising, consisting of, or consisting essentially of SEQ ID NO: 150. In some aspects, the antibody comprises a V_{L} sequence comprising a CDR-L2 sequence comprising, consisting of, or consisting essentially of SEQ ID NO: 151. In some aspects, the antibody comprises a V_{L} sequence comprising a CDR-L2 sequence comprising, consisting of, or consisting essentially of SEQ ID NO: 152. In some aspects, the antibody comprises a V_{L} sequence comprising a CDR-L2 sequence comprising, consisting of, or consisting essentially of SEQ ID NO: 153. In some aspects, the antibody comprises a V_{L} sequence comprising a CDR-L2 sequence comprising, consisting of, or consisting essentially of SEQ ID NO: 154. In some aspects, the antibody comprises a V_{L} sequence comprising a CDR-L2 sequence comprising, consisting of, or consisting essentially of SEQ ID NO: 155. In some aspects, the antibody comprises a V_{L} sequence comprising a CDR-L2 sequence comprising, consisting of, or consisting essentially of SEQ ID NO: 156. In some aspects, the antibody comprises a V_{L} sequence comprising a CDR-L2 sequence comprising, consisting of, or consisting essentially of SEQ ID NO: 157. In some aspects, the antibody comprises a V_{L} sequence comprising a CDR-L2 sequence comprising, consisting of, or consisting essentially of SEQ ID NO: 158. In some aspects, the antibody comprises a V_{L} sequence comprising a CDR-L2 sequence comprising, consisting of, or consisting essentially of SEQ ID NO: 159. In some aspects, the antibody comprises a V_{L} sequence comprising a CDR-L2 sequence comprising, consisting of, or consisting essentially of SEQ ID NO: 160. In some aspects, the antibody comprises a V_{L} sequence comprising a CDR-L2 sequence comprising, consisting of, or consisting essentially of SEQ ID NO: 161. In some aspects, the antibody comprises a V_{L} sequence comprising a CDR-L2 sequence comprising, consisting of, or consisting essentially of SEQ ID NO: 162. In some aspects, the antibody comprises a V_{L} sequence comprising a CDR-L2 sequence comprising, consisting of, or consisting essentially of SEQ ID NO: 163. In some aspects, the antibody comprises a V_{L} sequence comprising a CDR-L2 sequence comprising, consisting of, or consisting essentially of SEQ ID NO: 164. In some aspects, the antibody comprises a V_{L} sequence comprising a CDR-L2 sequence comprising, consisting of, or consisting essentially of SEQ ID NO: 165.

2.5.3. CDR-L1

In some embodiments, the LAG3 antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of an scFv-Fc sequence provided in SEQ ID NO: 379. In some aspects, the CDR-L1 sequence is a CDR-L1 sequence of a V_{L} sequence provided in SEQ ID NOs.: 251-266.

In some embodiments, the LAG3 antibody comprises a V_{L} sequence comprising a CDR-L1 sequence comprising, consisting of, or consisting essentially of a sequence selected from SEQ ID NOs: 132-147. In some aspects, the antibody comprises a V_{L} sequence comprising a CDR-L1 sequence comprising, consisting of, or consisting essentially of SEQ ID NO: 132. In some aspects, the antibody comprises a V_{L} sequence comprising a CDR-L1 sequence comprising, consisting of, or consisting essentially of SEQ ID NO: 133. In some aspects, the antibody comprises a V_{L} sequence comprising a CDR-L1 sequence comprising, consisting of, or consisting essentially of SEQ ID NO: 134. In some aspects, the antibody comprises a V_{L} sequence comprising a CDR-L1 sequence comprising, consisting of, or consisting essentially of SEQ ID NO: 135. In some aspects, the antibody comprises a V_{L} sequence comprising a CDR-L1 sequence comprising, consisting of, or consisting essentially of SEQ ID NO: 136. In some aspects, the antibody comprises a V_{L} sequence comprising a CDR-L1 sequence comprising, consisting of, or consisting essentially of SEQ ID NO: 137. In some aspects, the antibody comprises a V_{L} sequence comprising a CDR-L1 sequence comprising, consisting of, or consisting essentially of SEQ ID NO: 138. In some aspects, the antibody comprises a V_{L} sequence comprising a CDR-L1 sequence comprising, consisting of, or consisting essentially of SEQ ID NO: 139. In some aspects, the antibody comprises a V_{L} sequence comprising a CDR-L1 sequence comprising, consisting of, or consisting essentially of SEQ ID NO: 140. In some aspects, the antibody comprises a V_{L} sequence comprising a CDR-L1 sequence comprising, consisting of, or consisting essentially of SEQ ID NO: 141. In some aspects, the antibody comprises a V_{L} sequence comprising a CDR-L1 sequence comprising, consisting of, or consisting essentially of SEQ ID NO: 142. In some aspects, the antibody comprises a V_{L} sequence comprising a CDR-L1 sequence comprising, consisting of, or consisting essentially of SEQ ID NO: 143. In some aspects, the antibody comprises a V_{L} sequence comprising a CDR-L1 sequence comprising, consisting of, or consisting essentially of SEQ ID NO: 144. In some aspects, the antibody comprises a V_{L} sequence comprising a CDR-L1 sequence comprising, consisting of, or consisting essentially of SEQ ID NO: 145. In some aspects, the antibody comprises a V_{L} sequence comprising a CDR-L1 sequence comprising, consisting of, or consisting essentially of SEQ ID NO: 146. In some aspects, the antibody comprises a V_{L} sequence comprising a CDR-L1 sequence comprising, consisting of, or consisting essentially of SEQ ID NO: 147.

2.5.4. CDR-L3+CDR-L2

In some embodiments, the LAG3 antibody comprises a V_{L} sequence comprising a CDR-L3 sequence comprising, consisting of, or consisting essentially of a sequence selected from SEQ ID NOs: 173-188 and a CDR-L2 sequence comprising, consisting of, or consisting essentially of a sequence selected from SEQ ID NOs: 150-165. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs: 251-266.

2.5.5. CDR-L3+CDR-L1

In some embodiments, the LAG3 antibody comprises a V_{L} sequence comprising a CDR-L3 sequence comprising, consisting of, or consisting essentially of a sequence selected from SEQ ID NOs: 173-188 and a CDR-L1 sequence comprising, consisting of, or consisting essentially of a sequence selected from SEQ ID NOs: 132-147. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs: 251-266.

2.5.6. CDR-L1+CDR-L2

In some embodiments, the LAG3 antibody comprises a V_{L} sequence comprising a CDR-L1 sequence comprising, consisting of, or consisting essentially of a sequence selected from SEQ ID NOs: 132-147 and a CDR-L2 sequence comprising, consisting of, or consisting essentially of a sequence selected from SEQ ID NOs: 150-165. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs: 251-266.

2.5.7. CDR-L1+CDR-L2+CDR-L3

In some embodiments, the LAG3 antibody comprises a V_{L} sequence comprising a CDR-L1 sequence comprising, consisting of, or consisting essentially of a sequence selected from SEQ ID NOs: 132-147, a CDR-L2 sequence comprising, consisting of, or consisting essentially of a sequence selected from SEQ ID NOs: 150-165, and a CDR-L3 sequence comprising, consisting of, or consisting essentially of a sequence selected from SEQ ID NOs: 173-188. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs: 251-266.

2.5.8. Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

2.6. LAG3 V_{L} Sequences

In some embodiments, the LAG3 antibody comprises, consists of, or consists essentially of a V_{L} sequence of an scFv-Fc sequence provided in SEQ ID NO: 379. In some embodiments, the antibody comprises, consists of, or consists essentially of a V_{L} sequence provided in SEQ ID NOs.: 251-266.

In some embodiments, the LAG3 antibody comprises a V_{L} sequence comprising, consisting of, or consisting essentially of a sequence selected from SEQ ID NOs: 251-266. In some aspects, the antibody comprises a V_{L} sequence comprising, consisting of, or consisting essentially of SEQ ID NO: 251. In some aspects, the antibody comprises a V_{L} sequence comprising, consisting of, or consisting essentially of SEQ ID NO: 252. In some aspects, the antibody comprises a V_{L} sequence comprising, consisting of, or consisting essentially of SEQ ID NO: 253. In some aspects, the antibody comprises a V_{L} sequence comprising, consisting of, or consisting essentially of SEQ ID NO: 254. In some aspects, the antibody comprises a V_{L} sequence comprising, consisting of, or consisting essentially of SEQ ID NO: 255. In some aspects, the antibody comprises a V_{L} sequence comprising, consisting of, or consisting essentially of SEQ ID NO: 256. In some aspects, the antibody comprises a V_{L} sequence comprising, consisting of, or consisting essentially of SEQ ID NO: 257. In some aspects, the antibody comprises a V_{L} sequence comprising, consisting of, or consisting essentially of SEQ ID NO: 258. In some aspects, the antibody comprises a V_{L} sequence comprising, consisting of, or consisting essentially of SEQ ID NO: 259. In some aspects, the antibody comprises a V_{L} sequence comprising, consisting of, or consisting essentially of SEQ ID NO: 260. In some aspects, the antibody comprises a V_{L} sequence comprising, consisting of, or consisting essentially of SEQ ID NO: 261. In some aspects, the antibody comprises a V_{L} sequence comprising, consisting of, or consisting essentially of SEQ ID NO: 262. In some aspects, the antibody comprises a V_{L} sequence comprising, consisting of, or consisting essentially of SEQ ID NO: 263. In some aspects, the antibody comprises a V_{L} sequence comprising, consisting of, or consisting essentially of SEQ ID NO: 264. In some aspects, the antibody comprises a V_{L} sequence comprising, consisting of, or consisting essentially of SEQ ID NO: 265. In some aspects, the antibody comprises a V_{L} sequence comprising, consisting of, or consisting essentially of SEQ ID NO: 266.

2.6.1. Variants of V_{L} Sequences

In some embodiments, the V_{L} sequences provided herein comprise, consist of, or consist essentially of a variant of an illustrative V_{L} sequence provided in this disclosure.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.7. LAG3 Pairs

2.7.1. CDR-H3-CDR-L3 Pairs

In some embodiments, the LAG3 antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of SEQ ID NOs: 110-129, and the CDR-L3 sequence is a CDR-L3 sequence comprising, consisting of, or consisting essentially of SEQ ID NOs: 173-188.

2.7.1.1. Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.7.2. CDR-H1-CDR-L1 Pairs

In some embodiments, the LAG3 antibody comprises a CDR-H1 sequence and a CDR-L1 sequence. In some aspects, the CDR-H1 sequence is part of a V_{H} and the CDR-L1 sequence is part of a V_{L}.

In some aspects, the CDR-H1 sequence is a Chothia CDR-H1 sequence comprising, consisting of, or consisting essentially of SEQ ID NOs: 4-23, and the CDR-L1 sequence is a CDR-L1 sequence comprising, consisting of, or consisting essentially of SEQ ID NOs: 132-147.

In some aspects, the CDR-H1 sequence is a Kabat CDR-H1 sequence comprising, consisting of, or consisting essentially of SEQ ID NOs: 39-58, and the CDR-L1 sequence is a CDR-L1 sequence comprising, consisting of, or consisting essentially of SEQ ID NOs: 132-147.

2.7.2.1. Variants of CDR-H1-CDR-L1 Pairs

In some embodiments, the CDR-H1-CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

2.7.3. CDR-H2-CDR-L2 Pairs

In some embodiments, the LAG3 antibody comprises a CDR-H2 sequence and a CDR-L2 sequence. In some aspects, the CDR-H2 sequence is part of a V_{H} and the CDR-L2 sequence is part of a V_{L}.

In some aspects, the CDR-H2 sequence is a Chothia CDR-H2 sequence comprising, consisting of, or consisting essentially of SEQ ID NOs: 66-85, and the CDR-L2 sequence is a CDR-L2 sequence comprising, consisting of, or consisting essentially of SEQ ID NOs: 150-165.

In some aspects, the CDR-H1 sequence is a Kabat CDR-H2 sequence comprising, consisting of, or consisting essentially of SEQ ID NOs: 88-107, and the CDR-L2 sequence is a CDR-L2 sequence comprising, consisting of, or consisting essentially of SEQ ID NOs: 150-165.

2.7.3.1. Variants of CDR-H2-CDR-L2 Pairs

In some embodiments, the CDR-H2-CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

2.7.4. V_{H}-V_{L} Pairs

In some embodiments, the LAG3 antibody comprises a V_{H} sequence and a V_{L} sequence.

In some aspects, the V_{H} sequence is a V_{H} sequence comprising, consisting of, or consisting essentially of SEQ ID NOs: 191-210, and the V_{L} sequence is a V_{L} sequence comprising, consisting of, or consisting essentially of SEQ ID NOs: 251-266.

In some aspects, the V_{H}-V_{L} pairs are selected from SEQ ID NO: 191 and SEQ ID NO: 251; SEQ ID NO: 191 and SEQ ID NO: 252; SEQ ID NO: 191 and SEQ ID NO: 253; SEQ ID NO: 191 and SEQ ID NO: 254; SEQ ID NO: 191 and SEQ ID NO: 255; SEQ ID NO: 191 and SEQ ID NO: 256; SEQ ID NO: 191 and SEQ ID NO: 257; SEQ ID NO: 191 and SEQ ID NO: 258; SEQ ID NO: 191 and SEQ ID NO: 259; SEQ ID NO: 191 and SEQ ID NO: 260; SEQ ID NO: 191 and SEQ ID NO: 261; SEQ ID NO: 191 and SEQ ID NO: 262; SEQ ID NO: 191 and SEQ ID NO: 263; SEQ ID NO: 191 and SEQ ID NO: 264; SEQ ID NO: 191 and SEQ ID NO: 265; and SEQ ID NO: 191 and SEQ ID NO: 266.

In some aspects, the V_{H}-V_{L} pairs are selected from SEQ ID NO: 192 and SEQ ID NO: 251; SEQ ID NO: 192 and SEQ ID NO: 252; SEQ ID NO: 192 and SEQ ID NO: 253; SEQ ID NO: 192 and SEQ ID NO: 254; SEQ ID NO: 192 and SEQ ID NO: 255; SEQ ID NO: 192 and SEQ ID NO: 256; SEQ ID NO: 192 and SEQ ID NO: 257; SEQ ID NO: 192 and SEQ ID NO: 258; SEQ ID NO: 192 and SEQ ID NO: 259; SEQ ID NO: 192 and SEQ ID NO: 260; SEQ ID NO: 192 and SEQ ID NO: 261; SEQ ID NO: 192 and SEQ ID NO: 262; SEQ ID NO: 192 and SEQ ID NO: 263; SEQ ID NO: 192 and SEQ ID NO: 264; SEQ ID NO: 192 and SEQ ID NO: 265; and SEQ ID NO: 192 and SEQ ID NO: 266.

In some aspects, the V_{H}-V_{L} pairs are selected from SEQ ID NO: 193 and SEQ ID NO: 251; SEQ ID NO: 193 and SEQ ID NO: 252; SEQ ID NO: 193 and SEQ ID NO: 253; SEQ ID NO: 193 and SEQ ID NO: 254; SEQ ID NO: 193 and SEQ ID NO: 255; SEQ ID NO: 193 and SEQ ID NO: 256; SEQ ID NO: 193 and SEQ ID NO: 257; SEQ ID NO: 193 and SEQ ID NO: 258; SEQ ID NO: 193 and SEQ ID NO: 259; SEQ ID NO: 193 and SEQ ID NO: 260; SEQ ID NO: 193 and SEQ ID NO: 261; SEQ ID NO: 193 and SEQ ID NO: 262; SEQ ID NO: 193 and SEQ ID NO: 263; SEQ ID NO: 193 and SEQ ID NO: 264; SEQ ID NO: 193 and SEQ ID NO: 265; and SEQ ID NO: 193 and SEQ ID NO: 266.

In some aspects, the V_{H}-V_{L} pairs are selected from SEQ ID NO: 194 and SEQ ID NO: 251; SEQ ID NO: 194 and SEQ ID NO: 252; SEQ ID NO: 194 and SEQ ID NO: 253; SEQ ID NO: 194 and SEQ ID NO: 254; SEQ ID NO: 194 and SEQ ID NO: 255; SEQ ID NO: 194 and SEQ ID NO: 256; SEQ ID NO: 194 and SEQ ID NO: 257; SEQ ID NO: 194 and SEQ ID NO: 258; SEQ ID NO: 194 and SEQ ID NO: 259; SEQ ID NO: 194 and SEQ ID NO: 260; SEQ ID NO: 194 and SEQ ID NO: 261; SEQ ID NO: 194 and SEQ ID NO: 262; SEQ ID NO: 194 and SEQ ID NO: 263; SEQ ID NO: 194 and SEQ ID NO: 264; SEQ ID NO: 194 and SEQ ID NO: 265; and SEQ ID NO: 194 and SEQ ID NO: 266.

In some aspects, the V_{H}-V_{L} pairs are selected from SEQ ID NO: 195 and SEQ ID NO: 251; SEQ ID NO: 195 and SEQ ID NO: 252; SEQ ID NO: 195 and SEQ ID NO: 253; SEQ ID NO: 195 and SEQ ID NO: 254; SEQ ID NO: 195 and SEQ ID NO: 255; SEQ ID NO: 195 and SEQ ID NO: 256; SEQ ID NO: 195 and SEQ ID NO: 257; SEQ ID NO: 195 and SEQ ID NO: 258; SEQ ID NO: 195 and SEQ ID NO: 259; SEQ ID NO: 195 and SEQ ID NO: 260; SEQ ID NO: 195 and SEQ ID NO: 261; SEQ ID NO: 195 and SEQ ID NO: 262; SEQ ID NO: 195 and SEQ ID NO: 263; SEQ ID NO: 195 and SEQ ID NO: 264; SEQ ID NO: 195 and SEQ ID NO: 265; and SEQ ID NO: 195 and SEQ ID NO: 266.

In some aspects, the V_{H}-V_{L} pairs are selected from SEQ ID NO: 196 and SEQ ID NO: 251; SEQ ID NO: 196 and SEQ ID NO: 252; SEQ ID NO: 196 and SEQ ID NO: 253; SEQ ID NO: 196 and SEQ ID NO: 254; SEQ ID NO: 196 and SEQ ID NO: 255; SEQ ID NO: 196 and SEQ ID NO: 256; SEQ ID NO: 196 and SEQ ID NO: 257; SEQ ID NO: 196 and SEQ ID NO: 258; SEQ ID NO: 196 and SEQ ID NO: 259; SEQ ID NO: 196 and SEQ ID NO: 260; SEQ ID NO: 196 and SEQ ID NO: 261; SEQ ID NO: 196 and SEQ ID NO: 262; SEQ ID NO: 196 and SEQ ID NO: 263; SEQ ID NO: 196 and SEQ ID NO: 264; SEQ ID NO: 196 and SEQ ID NO: 265; and SEQ ID NO: 196 and SEQ ID NO: 266.

In some aspects, the V_{H}-V_{L} pairs are selected from SEQ ID NO: 197 and SEQ ID NO: 251; SEQ ID NO: 197 and SEQ ID NO: 252; SEQ ID NO: 197 and SEQ ID NO: 253; SEQ ID NO: 197 and SEQ ID NO: 254; SEQ ID NO: 197 and SEQ ID NO: 255; SEQ ID NO: 197 and SEQ ID NO: 256; SEQ ID NO: 197 and SEQ ID NO: 257; SEQ ID NO: 197 and SEQ ID NO: 258; SEQ ID NO: 197 and SEQ ID NO: 259; SEQ ID NO: 197 and SEQ ID NO: 260; SEQ ID NO: 197 and SEQ ID NO: 261; SEQ ID NO: 197 and SEQ ID NO: 262; SEQ ID NO: 197 and SEQ ID NO: 263; SEQ ID NO: 197 and SEQ ID NO: 264; SEQ ID NO: 197 and SEQ ID NO: 265; and SEQ ID NO: 197 and SEQ ID NO: 266.

In some aspects, the V_{H}-V_{L} pairs are selected from SEQ ID NO: 198 and SEQ ID NO: 251; SEQ ID NO: 198 and SEQ ID NO: 252; SEQ ID NO: 198 and SEQ ID NO: 253; SEQ ID NO: 198 and SEQ ID NO: 254; SEQ ID NO: 198 and SEQ ID NO: 255; SEQ ID NO: 198 and SEQ ID NO: 256; SEQ ID NO: 198 and SEQ ID NO: 257; SEQ ID NO: 198 and SEQ ID NO: 258; SEQ ID NO: 198 and SEQ ID NO: 259; SEQ ID NO: 198 and SEQ ID NO: 260; SEQ ID NO: 198 and SEQ ID NO: 261; SEQ ID NO: 198 and SEQ ID NO: 262; SEQ ID NO: 198 and SEQ ID NO: 263; SEQ ID NO: 198 and SEQ ID NO: 264; SEQ ID NO: 198 and SEQ ID NO: 265; and SEQ ID NO: 198 and SEQ ID NO: 266.

In some aspects, the V_{H}-V_{L} pairs are selected from SEQ ID NO: 199 and SEQ ID NO: 251; SEQ ID NO: 199 and SEQ ID NO: 252; SEQ ID NO: 199 and SEQ ID NO: 253; SEQ ID NO: 199 and SEQ ID NO: 254; SEQ ID NO: 199 and SEQ ID NO: 255; SEQ ID NO: 199 and SEQ ID NO: 256; SEQ ID NO: 199 and SEQ ID NO: 257; SEQ ID NO: 199 and SEQ ID NO: 258; SEQ ID NO: 199 and SEQ ID NO: 259; SEQ ID NO: 199 and SEQ ID NO: 260; SEQ ID NO: 199 and SEQ ID NO: 261; SEQ ID NO: 199 and SEQ ID NO: 262; SEQ ID NO: 199 and SEQ ID NO: 263; SEQ ID NO: 199 and SEQ ID NO: 264; SEQ ID NO: 199 and SEQ ID NO: 265; and SEQ ID NO: 199 and SEQ ID NO: 266.

In some aspects, the V_{H}-V_{L} pairs are selected from SEQ ID NO: 200 and SEQ ID NO: 251; SEQ ID NO: 200 and SEQ ID NO: 252; SEQ ID NO: 200 and SEQ ID NO: 253; SEQ ID NO: 200 and SEQ ID NO: 254; SEQ ID NO: 200 and SEQ ID NO: 255; SEQ ID NO: 200 and SEQ ID NO: 256; SEQ ID NO: 200 and SEQ ID NO: 257; SEQ ID NO: 200 and SEQ ID NO: 258; SEQ ID NO: 200 and SEQ ID NO: 259; SEQ ID NO: 200 and SEQ ID NO: 260; SEQ ID NO: 200 and SEQ ID NO: 261; SEQ ID NO: 200 and SEQ ID NO: 262; SEQ ID NO: 200 and SEQ ID NO: 263; SEQ ID NO: 200 and SEQ ID NO: 264; SEQ ID NO: 200 and SEQ ID NO: 265; and SEQ ID NO: 200 and SEQ ID NO: 266.

In some aspects, the V_{H}-V_{L} pairs are selected from SEQ ID NO: 201 and SEQ ID NO: 251; SEQ ID NO: 201 and SEQ ID NO: 252; SEQ ID NO: 201 and SEQ ID NO: 253; SEQ ID NO: 201 and SEQ ID NO: 254; SEQ ID NO: 201 and SEQ ID NO: 255; SEQ ID NO: 201 and SEQ ID NO: 256; SEQ ID NO: 201 and SEQ ID NO: 257; SEQ ID NO: 201 and SEQ ID NO: 258; SEQ ID NO: 201 and SEQ ID NO: 259; SEQ ID NO: 201 and SEQ ID NO: 260; SEQ ID NO: 201 and SEQ ID NO: 261; SEQ ID NO: 201 and SEQ ID NO: 262; SEQ ID NO: 201 and SEQ ID NO: 263; SEQ ID NO: 201 and SEQ ID NO: 264; SEQ ID NO: 201 and SEQ ID NO: 265; and SEQ ID NO: 201 and SEQ ID NO: 266.

In some aspects, the V_{H}-V_{L} pairs are selected from SEQ ID NO: 202 and SEQ ID NO: 251; SEQ ID NO: 202 and SEQ ID NO: 252; SEQ ID NO: 202 and SEQ ID NO: 253; SEQ ID NO: 202 and SEQ ID NO: 254; SEQ ID NO: 202 and SEQ ID NO: 255; SEQ ID NO: 202 and SEQ ID NO: 256; SEQ ID NO: 202 and SEQ ID NO: 257; SEQ ID NO: 202 and SEQ ID NO: 258; SEQ ID NO: 202 and SEQ ID NO: 259; SEQ ID NO: 202 and SEQ ID NO: 260; SEQ ID NO: 202 and SEQ ID NO: 261; SEQ ID NO: 202 and SEQ ID NO: 262; SEQ ID NO: 202 and SEQ ID NO: 263; SEQ ID NO: 202 and SEQ ID NO: 264; SEQ ID NO: 202 and SEQ ID NO: 265; and SEQ ID NO: 202 and SEQ ID NO: 266.

In some aspects, the V_{H}-V_{L} pairs are selected from SEQ ID NO: 203 and SEQ ID NO: 251; SEQ ID NO: 203 and SEQ ID NO: 252; SEQ ID NO: 203 and SEQ ID NO: 253; SEQ ID NO: 203 and SEQ ID NO: 254; SEQ ID NO: 203 and SEQ ID NO: 255; SEQ ID NO: 203 and SEQ ID NO: 256; SEQ ID NO: 203 and SEQ ID NO: 257; SEQ ID NO: 203 and SEQ ID NO: 258; SEQ ID NO: 203 and SEQ ID NO: 259; SEQ ID NO: 203 and SEQ ID NO: 260; SEQ ID NO: 203 and SEQ ID NO: 261; SEQ ID NO: 203 and SEQ ID NO: 262; SEQ ID NO: 203 and SEQ ID NO: 263; SEQ ID NO: 203 and SEQ ID NO: 264; SEQ ID NO: 203 and SEQ ID NO: 265; and SEQ ID NO: 203 and SEQ ID NO: 266.

In some aspects, the V_{H}-V_{L} pairs are selected from SEQ ID NO: 204 and SEQ ID NO: 251; SEQ ID NO: 204 and SEQ ID NO: 252; SEQ ID NO: 204 and SEQ ID NO: 253; SEQ ID NO: 204 and SEQ ID NO: 254; SEQ ID NO: 204 and SEQ ID NO: 255; SEQ ID NO: 204 and SEQ ID NO: 256; SEQ ID NO: 204 and SEQ ID NO: 257; SEQ ID NO: 204 and SEQ ID NO: 258; SEQ ID NO: 204 and SEQ ID NO: 259; SEQ ID NO: 204 and SEQ ID NO: 260; SEQ ID NO: 204 and SEQ ID NO: 261; SEQ ID NO: 204 and SEQ ID NO: 262; SEQ ID NO: 204 and SEQ ID NO: 263; SEQ ID NO: 204 and SEQ ID NO: 264; SEQ ID NO: 204 and SEQ ID NO: 265; and SEQ ID NO: 204 and SEQ ID NO: 266.

In some aspects, the V_{H}-V_{L} pairs are selected from SEQ ID NO: 205 and SEQ ID NO: 251; SEQ ID NO: 205 and SEQ ID NO: 252; SEQ ID NO: 205 and SEQ ID NO: 253; SEQ ID NO: 205 and SEQ ID NO: 254; SEQ ID NO: 205 and SEQ ID NO: 255; SEQ ID NO: 205 and SEQ ID NO: 256; SEQ ID NO: 205 and SEQ ID NO: 257; SEQ ID NO: 205 and SEQ ID NO: 258; SEQ ID NO: 205 and SEQ ID NO: 259; SEQ ID NO: 205 and SEQ ID NO: 260; SEQ ID NO: 205 and SEQ ID NO: 261; SEQ ID NO: 205 and SEQ ID NO: 262; SEQ ID NO: 205 and SEQ ID NO: 263; SEQ ID NO: 205 and SEQ ID NO: 264; SEQ ID NO: 205 and SEQ ID NO: 265; and SEQ ID NO: 205 and SEQ ID NO: 266.

In some aspects, the V_{H}-V_{L} pairs are selected from SEQ ID NO: 206 and SEQ ID NO: 251; SEQ ID NO: 206 and SEQ ID NO: 252; SEQ ID NO: 206 and SEQ ID NO: 253; SEQ ID NO: 206 and SEQ ID NO: 254; SEQ ID NO: 206 and SEQ ID NO: 255; SEQ ID NO: 206 and SEQ ID NO: 256; SEQ ID NO: 206 and SEQ ID NO: 257; SEQ ID NO: 206 and SEQ ID NO: 258; SEQ ID NO: 206 and SEQ ID NO: 259; SEQ ID NO: 206 and SEQ ID NO: 260; SEQ ID NO: 206 and SEQ ID NO: 261; SEQ ID NO: 206 and SEQ ID NO: 262; SEQ ID NO: 206 and SEQ ID NO: 263; SEQ ID NO: 206 and SEQ ID NO: 264; SEQ ID NO: 206 and SEQ ID NO: 265; and SEQ ID NO: 206 and SEQ ID NO: 266.

In some aspects, the V_{H}-V_{L} pairs are selected from SEQ ID NO: 207 and SEQ ID NO: 251; SEQ ID NO: 207 and SEQ ID NO: 252; SEQ ID NO: 207 and SEQ ID NO: 253; SEQ ID NO: 207 and SEQ ID NO: 254; SEQ ID NO: 207 and SEQ ID NO: 255; SEQ ID NO: 207 and SEQ ID NO: 256; SEQ ID NO: 207 and SEQ ID NO: 257; SEQ ID NO: 207 and SEQ ID NO: 258; SEQ ID NO: 207 and SEQ ID NO: 259; SEQ ID NO: 207 and SEQ ID NO: 260; SEQ ID NO: 207 and SEQ ID NO: 261; SEQ ID NO: 207 and SEQ ID NO: 262; SEQ ID NO: 207 and SEQ ID NO: 263; SEQ ID NO: 207 and SEQ ID NO: 264; SEQ ID NO: 207 and SEQ ID NO: 265; and SEQ ID NO: 207 and SEQ ID NO: 266.

In some aspects, the V_{H}-V_{L} pairs are selected from SEQ ID NO: 208 and SEQ ID NO: 251; SEQ ID NO: 208 and SEQ ID NO: 252; SEQ ID NO: 208 and SEQ ID NO: 253; SEQ ID NO: 208 and SEQ ID NO: 254; SEQ ID NO: 208 and SEQ ID NO: 255; SEQ ID NO: 208 and SEQ ID NO: 256; SEQ ID NO: 208 and SEQ ID NO: 257; SEQ ID NO: 208 and SEQ ID NO: 258; SEQ ID NO: 208 and SEQ ID NO: 259; SEQ ID NO: 208 and SEQ ID NO: 260; SEQ ID NO: 208 and SEQ ID NO: 261; SEQ ID NO: 208 and SEQ ID NO: 262; SEQ ID NO: 208 and SEQ ID NO: 263; SEQ ID NO: 208 and SEQ ID NO: 264; SEQ ID NO: 208 and SEQ ID NO: 265; and SEQ ID NO: 208 and SEQ ID NO: 266.

In some aspects, the V_{H}-V_{L} pairs are selected from SEQ ID NO: 209 and SEQ ID NO: 251; SEQ ID NO: 209 and SEQ ID NO: 252; SEQ ID NO: 209 and SEQ ID NO: 253; SEQ ID NO: 209 and SEQ ID NO: 254; SEQ ID NO: 209 and SEQ ID NO: 255; SEQ ID NO: 209 and SEQ ID NO: 256; SEQ ID NO: 209 and SEQ ID NO: 257; SEQ ID NO: 209 and SEQ ID NO: 258; SEQ ID NO: 209 and SEQ ID NO: 259; SEQ ID NO: 209 and SEQ ID NO: 260; SEQ ID NO: 209 and SEQ ID NO: 261; SEQ ID NO: 209 and SEQ ID NO: 262; SEQ ID NO: 209 and SEQ ID NO: 263; SEQ ID NO: 209 and SEQ ID NO: 264; SEQ ID NO: 209 and SEQ ID NO: 265; and SEQ ID NO: 209 and SEQ ID NO: 266.

In some aspects, the V_{H}-V_{L} pairs are selected from SEQ ID NO: 210 and SEQ ID NO: 251; SEQ ID NO: 210 and SEQ ID NO: 252; SEQ ID NO: 210 and SEQ ID NO: 253; SEQ ID NO: 210 and SEQ ID NO: 254; SEQ ID NO: 210 and SEQ ID NO: 255; SEQ ID NO: 210 and SEQ ID NO: 256; SEQ ID NO: 210 and SEQ ID NO: 257; SEQ ID NO: 210 and SEQ ID NO: 258; SEQ ID NO: 210 and SEQ ID NO: 259; SEQ ID NO: 210 and SEQ ID NO: 260; SEQ ID NO: 210 and SEQ ID NO: 261; SEQ ID NO: 210 and SEQ ID NO: 262; SEQ ID NO: 210 and SEQ ID NO: 263; SEQ ID NO: 210 and SEQ ID NO: 264; SEQ ID NO: 210 and SEQ ID NO: 265; and SEQ ID NO: 210 and SEQ ID NO: 266.

2.7.4.1. Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

2.8. LAG3 Antibodies Comprising all Six CDRs

In some embodiments, the LAG3 antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of SEQ ID NOs: 4-23; the CDR-H2 sequence is a Chothia CDR-H2 sequence comprising, consisting of, or consisting essentially of SEQ ID NOs: 66-85; the CDR-H3 sequence is a CDR-H3 sequence comprising, consisting of, or consisting essentially of SEQ ID NOs: 110-129; the CDR-L1 sequence is a CDR-L1 sequence comprising, consisting of, or consisting essentially of SEQ ID NOs: 132-147; the CDR-L2 sequence is a CDR-L2 sequence comprising, consisting of, or consisting essentially of SEQ ID NOs: 150-165; and the CDR-L3 sequence is a CDR-L3 sequence comprising, consisting of, or consisting essentially of SEQ ID NOs: 173-188.

In some aspects, the CDR-H1 sequence is a Kabat CDR-H1 sequence comprising, consisting of, or consisting essentially of SEQ ID NOs: 39-58; the CDR-H2 sequence is a Kabat CDR-H2 sequence comprising, consisting of, or consisting essentially of SEQ ID NOs: 88-107; the CDR-H3 sequence is a CDR-H3 sequence comprising, consisting of, or consisting essentially of SEQ ID NOs: 110-129; the CDR-L1 sequence is a CDR-L1 sequence comprising, consisting of, or consisting essentially of SEQ ID NOs: 132-147; the CDR-L2 sequence is a CDR-L2 sequence comprising, consisting of, or consisting essentially of SEQ ID NOs: 150-165; and the CDR-L3 sequence is a CDR-L3 sequence comprising, consisting of, or consisting essentially of SEQ ID NOs: 173-188.

2.8.1. Variants of Antibodies Comprising All Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

2.9. LAG3 Consensus Sequences

In some embodiments, provided herein are anti-LAG3 antibodies comprising one or more sequences defined by consensus sequences. Each consensus sequence is based, at least in part, on one or more alignments of two or more useful anti-LAG3 CDR sequences provided in this disclosure. Based on such alignments, a person of skill in the art would recognize that different amino acid residues may useful in certain positions of the CDRs. Accordingly, each consensus sequence encompasses two or more useful anti-LAG3 CDR sequences.

In some embodiments, the LAG3 antibodies comprise one to six of the consensus CDR sequences provided herein. In some embodiments, the antibodies comprise two to six of the consensus CDR sequences provided herein. In some embodiments, the antibodies comprise three to six of the consensus CDR sequences provided herein. In some embodiments, the antibodies comprise four to six of the consensus CDR sequences provided herein. In some embodiments, the antibodies comprise five to six of the consensus CDR sequences provided herein. In some embodiments, the antibodies comprise six of the consensus CDR sequences provided herein. In some embodiments, the antibodies comprise a V_{L} comprising the CDR-L consensus sequence(s). In some embodiments, the antibodies comprise a V_{H} comprising the CDR-H consensus sequence(s). In some embodiments, the antibodies comprise a V_{H} comprising the CDR-H consensus sequence(s) and a V_{L} comprising the CDR-L consensus sequence(s).

2.9.1. CDR-H3 Consensus Sequences

In some embodiments, the LAG3 antibody comprises a CDR-H3 sequence defined by the consensus sequence α₁-α₂-α₃-α₄-α₅-α₆-α₇-α₈-α₉-α₁₀-α₁₁-D-α₁₃, where α₁ is absent, E, or V; α₂ is absent I, S, W, E, Y, D, or F; α₃ is absent, F, L, I, E A, A, or N; α₄ is absent, G, V, P, or D; α₅ is absent, A, S, E, V, or G; α₆ is F, S, N, or V; α₇ is absent Y, W, or R; α₈ is W, L, D, P, or S; α₉ is N, Y, A, D, or F; α₁₀ is P, A, G, S, or M; an is absent, F, L, M, or V; and α₁₃ is Y or V. In certain embodiments, each of α₁, α₂, α₃, α₄, α₅, α₆, and α₇ is absent. In certain embodiments, none of α₁, α₂, α₃, α₄, α₅, α₆, and α₇ is absent. In certain embodiments, only α₅ of α₁, α₂, α₃, α₄, as, α₆, and α₇ is absent. In certain embodiments, only as is absent. In certain embodiments, only all is absent. In certain embodiments, when α₂ is W, α₄ is V, α₅ is A, α₆ is S, and α₁₀ is G, then α₁₁ is F, L, or V. In certain embodiments, α₂ is E or D, α₄ is P, α₅ is E, α₆ is N, α₁₀ is A or G, and α₁₁ is F, L, or V. In certain embodiments, the antibody comprises a CDR-H3 sequence defined by the consensus sequence E-α₂-α₃-α₄-α₅-α₆-W-D-α₉-α₁₀-α₁₁-D-V where α₂ is S, W, or E; α₃ is A or E; α₄ is V, P, or D; α₅ is A, S, E, or V; α₆ is S or N; α₉ is Y or A; α₁₀ is A or G; and α₁₁ is L or M wherein when α₂ is W, α₄ is V, α₅ is A, α₆ is S, and α₁₀ is G, then α₁₁ is L.

In some embodiments, the LAG3 antibody comprises a CDR-H3 sequence defined by the consensus sequence V-β₂-β₃-G-G-V-R-P-β₉-S-β₁₁-D-Y, where β₂ is F, Y, or D; β₃ is E or N; β₉ is Y or F; and β₁₁ is absent.

2.9.2. Chothia CDR-H1 Consensus Sequences

In some embodiments, the LAG3 antibody comprises a Chothia CDR-H1 sequence defined by the consensus sequence G-F-γ₃-γ₄-γ₅-γ₆-γ₇, where γ₃ is N or T; γ₄ is I or F; γ₅ is K, N, A, S, R, P, or T; γ₆ is D, S, or E; and γ₇ is T, N, Y, F, S, or L.

In some embodiments, the LAG3 antibody comprises a Chothia CDR-H1 sequence defined by the consensus sequence G-F-T-F-δ₅-δ₆-δ₇, where δ₅ is S, R, P, T, or N; δ₆ is S, D, or E; and δ₇ is F, S, or Y.

2.9.3. Chothia CDR-H2 Consensus Sequences

In some embodiments, the LAG3 antibody comprises a Chothia CDR-H2 sequence defined by the consensus sequence ε₁-ε₂-ε₃-ε₄-ε₅-ε₆, where ε₁ is D, W, or T; ε₂ is P, Y, D, G, or S; ε₃ is Y, D, N, W, or, E; ε₄ is D, A, G, S, T, or N; ε₅ is G or S; and ε₆ is A, D, F, Y, V, N, T, or S. In certain embodiments, ε₁ is W; ε₂ is Y; ε₃ is D; ε₄ is A or G; ε₅ is S; and ε₆ is Y, N, or V. In certain embodiments, ε₁ is T or S; ε₂ is D or S; ε₃ is N or D; ε₄ is S or T; ε₅ is G; and ε₆ is N, T, or S.

2.9.4. Kabat CDR-H1 Consensus Sequences

In some embodiments, the LAG3 antibody comprises a Kabat CDR-H1 sequence defined by the consensus sequence ξ₁-ξ₂-ξ₃-ξ₄-ξ₅, where ξ₁ is D, S, or E; ξ₂ is T, N, Y, F, S, or L; ξ₃ is Y, F, G, S, or T; ξ₄ is I or M; and ξ₅ is H or S.

In some embodiments, the LAG3 antibody comprises a Kabat CDR-H1 sequence defined by the consensus sequence S-η₂-G-M-H, where η₂ is Y or F. In some embodiments, the antibody comprises a Kabat CDR-H1 sequence defined by the consensus sequence η₁-S-η₃-M-H, where η₁ is D, E, or S; and η₃ is S or T.

2.9.5. Kabat CDR-H2 Consensus Sequences

In some embodiments, the LAG3 antibody comprises a Kabat CDR-H2 sequence defined by the consensus sequence θ₁-I-θ₃-θ₄-θ₅-θ₆-θ₇-θ₈-θ₉-θ₁₀-Y-A-θ₁₃-θ₁₄-θ₁₅-θ₁₆-G, where θ₁ is I, A, V, R, or W; θ₃ is D, W, T, or S; θ₄ is P, Y, D, G, or S; θ₅ is Y, D, N, W, or E; θ₆ is D, A, G, S, T, or N; θ₇ is G or S; θ₈ is A, D, F, Y, N, V, T, or S; θ₉ is T or K; θ₁₀ is D, A, Y or E; θ₁₃ is D, or P; θ₁₄ is S or K; θ₁₅ is V or F; and θ₁₆ is K or Q. In some embodiments, the antibody comprises a Kabat CDR-H2 sequence defined by the consensus sequence θ₁-I-θ₃-Y-D-G-S-θ₈-K-Y-Y-A-D-S-V-K-G, where θ₁ is V or A; θ₃ is W or T; and θ₈ is Y, N, or V. In some embodiments, the antibody comprises a Kabat CDR-H2 sequence defined by the consensus sequence θ₁-I-θ₃-θ₄-θ₅-θ₆-G-θ₈-T-D-Y-A-D-S—V-K-G, where θ₁ is F or V; θ3 is T or S; θ₄ is S, D, or G; θ₅ is D or N; θ6 is S or T; and θ₈ is T, S, or N.

2.9.6. CDR-L3 Consensus Sequences

In some embodiments, the LAG3 antibody comprises a CDR-L3 sequence defined by the consensus sequence Q-Q-ι₃-ι₄-ι₅-ι₆-P-ι₈-ι₉, where ι₃ is Y or D; ι₄ is G, D, S, M, or T; ι₅ is R, S, A, or L; ι₆ is S, T, A, or G; ι₈ is F, L or P; and ι₉ is S, T, or K. In certain embodiments, when ι₅ is S, then ι₅ is S.

In some embodiments, the LAG3 antibody comprises a CDR-L3 sequence defined by the consensus sequence ι₁-ι₂-ι₃-ι₄-ι₅-ι₆-P-Q-T where ι₁ is S or W; ι₂ is H, T, or Q; ι₃ is G or Y; ι₄ is N, I, or S; and ι₅ is V or F.

2.9.7. CDR-L2 Consensus Sequences

In some embodiments, the LAG3 antibody comprises a CDR-L2 sequence defined by the consensus sequence GASSRAT (SEQ ID NO:150) and LVSKLDS (SEQ ID NO:161)

2.9.8. CDR-L1 Consensus Sequences

In some embodiments, the LAG3 antibody comprises a CDR-L1 sequence defined by the consensus sequence R-A-S-Q-μ₅-μ₆-μ₇-μ₈-S—V-S-S-μ₁₃-μ₁₄-μ₁₅-A, where μ₅ is absent; μ₆ is absent; μ₇ is absent; μ₈ is absent; μ₁₃ is S, N, or G; μ₁₄ is Y, P or N; and μ₁₅ is L or P. In some embodiments, the antibody comprises a CDR-L1 sequence defined by the consensus sequence KSSQSLLDSDGKTYLN (SEQ ID NO:143).

### 3. PD-1 Antibodies

Provided herein are PD-1 antibodies that selectively bind human PD-1. In some aspects, the antibody selectively binds to the extracellular domain of human PD-1. In some aspects, the antibody selectively binds to one or more of full-length human PD-1, PD-1Δex2, PD-1Δex3, PD-1Δex2,3, and PD-1Δex2,3,4. See Nielsen et al., Cellular Immunology, 2005, 235:109-116, incorporated by reference in its entirety.

In some embodiments, the PD-1 antibody binds to a homolog of human PD-1. In some aspects, the antibody binds to a homolog of human PD-1 from a species selected from monkeys, mice, dogs, cats, rats, cows, horses, goats and sheep. In some aspects, the homolog is a cynomolgus monkey homolog. In some aspects, the homolog is a murine homolog.

In some embodiments, the PD-1 antibody comprises a light chain. In some aspects, the light chain is a kappa light chain. In some aspects, the light chain is a lambda light chain.

In some embodiments, the PD-1 antibody comprises a heavy chain. In some aspects, the heavy chain is an IgA. In some aspects, the heavy chain is an IgD. In some aspects, the heavy chain is an IgE. In some aspects, the heavy chain is an IgG. In some aspects, the heavy chain is an IgM. In some aspects, the heavy chain is an IgG1. In some aspects, the heavy chain is an IgG2. In some aspects, the heavy chain is an IgG3. In some aspects, the heavy chain is an IgG4. In some aspects, the heavy chain is an IgA1. In some aspects, the heavy chain is an IgA2.

In some embodiments, the PD-1 antibody is an antibody fragment. In some aspects, the antibody fragment is an Fv fragment. In some aspects, the antibody fragment is a Fab fragment. In some aspects, the antibody fragment is a F(ab′)₂ fragment. In some aspects, the antibody fragment is a Fab′ fragment. In some aspects, the antibody fragment is an scFv (sFv) fragment. In some aspects, the antibody fragment is an scFv-Fc fragment.

In some embodiments, the PD-1 antibody is a monoclonal antibody. In some embodiments, the antibody is a polyclonal antibody.

In some embodiments, the PD-1 antibody is a chimeric antibody. In some embodiments, the antibody is a humanized antibody. In some embodiments, the antibody is a human antibody.

In some embodiments, the PD-1 antibody is an affinity matured antibody. In some aspects, the antibody is an affinity matured antibody derived from an illustrative sequence provided in this disclosure or in, e.g., WO 2016/077397, which is incorporated herein by reference in its entirety.

In some embodiments, the PD-1 antibody inhibits the binding of PD-1 to its ligands. In some aspects, the antibody inhibits the binding of PD-1 to PD-L1. In some aspects, the antibody inhibits the binding of PD-1 to PD-L2. In some aspects, the antibody inhibits the binding of PD-1 to PD-L1 and PD-L2.

In some embodiments, the PD-1 antibody is provided as a single arm binder. For example, the PD-1 antibody can be provided as part of a bi-specific antibody or bi-specific antibody construct as disclosed here.

The PD-1 antibodies provided herein may be useful for the treatment of a variety of diseases and conditions, including cancers, autoimmune diseases, and infections.

3.1 PD-1 CDR-H3 Sequences

In some embodiments, the PD-1 antibody comprises a CDR-H3 sequence comprising, consisting of, or consisting essentially of a CDR-H3 sequence of an illustrative antibody or V_{H} sequence provided herein. In some aspects, the CDR-H3 sequence is a CDR-H3 sequence of a V_{H} sequence provided in SEQ ID NOs.: 211-250. In some embodiments, the PD-1 antibody comprises a CDR-H3 sequence comprising, consisting of, or consisting essentially of a sequence selected from SEQ ID NOs: 130-131. In some aspects, the antibody comprises a CDR-H3 sequence comprising, consisting of, or consisting essentially of SEQ ID NO: 130. In some aspects, the antibody comprises a CDR-H3 sequence comprising, consisting of, or consisting essentially of SEQ ID NO: 131. In some embodiments, the PD-1 antibody comprises a CDR-H3 sequence comprising, consisting of, or consisting essentially of a CDR-H3 sequence of a V_{H} sequence selected from SEQ ID NOs.: 228-250. In some aspects, the antibody comprises a CDR-H3 sequence comprising, consisting of, or consisting essentially of a CDR-H3 sequence of a V_{H} sequence of SEQ ID NO: 228. In some aspects, the antibody comprises a CDR-H3 sequence comprising, consisting of, or consisting essentially of a CDR-H3 sequence of a V_{H} sequence of SEQ ID NO: 229. In some aspects, the antibody comprises a CDR-H3 sequence comprising, consisting of, or consisting essentially of a CDR-H3 sequence of a V_{H} sequence of SEQ ID NO: 230. In some aspects, the antibody comprises a CDR-H3 sequence comprising, consisting of, or consisting essentially of a CDR-H3 sequence of a V_{H} sequence of SEQ ID NO: 231. In some aspects, the antibody comprises a CDR-H3 sequence comprising, consisting of, or consisting essentially of a CDR-H3 sequence of a V_{H} sequence of SEQ ID NO: 232. In some aspects, the antibody comprises a CDR-H3 sequence comprising, consisting of, or consisting essentially of a CDR-H3 sequence of a V_{H} sequence of SEQ ID NO: 233. In some aspects, the antibody comprises a CDR-H3 sequence comprising, consisting of, or consisting essentially of a CDR-H3 sequence of a V_{H} sequence of SEQ ID NO: 234. In some aspects, the antibody comprises a CDR-H3 sequence comprising, consisting of, or consisting essentially of a CDR-H3 sequence of a V_{H} sequence of SEQ ID NO: 235. In some aspects, the antibody comprises a CDR-H3 sequence comprising, consisting of, or consisting essentially of a CDR-H3 sequence of a V_{H} sequence of SEQ ID NO: 236. In some aspects, the antibody comprises a CDR-H3 sequence comprising, consisting of, or consisting essentially of a CDR-H3 sequence of a V_{H} sequence of SEQ ID NO: 237. In some aspects, the antibody comprises a CDR-H3 sequence comprising, consisting of, or consisting essentially of a CDR-H3 sequence of a V_{H} sequence of SEQ ID NO: 238. In some aspects, the antibody comprises a CDR-H3 sequence comprising, consisting of, or consisting essentially of a CDR-H3 sequence of a V_{H} sequence of SEQ ID NO: 239. In some aspects, the antibody comprises a CDR-H3 sequence comprising, consisting of, or consisting essentially of a CDR-H3 sequence of a V_{H} sequence of SEQ ID NO: 240. In some aspects, the antibody comprises a CDR-H3 sequence comprising, consisting of, or consisting essentially of a CDR-H3 sequence of a V_{H} sequence of SEQ ID NO: 241. In some aspects, the antibody comprises a CDR-H3 sequence comprising, consisting of, or consisting essentially of a CDR-H3 sequence of a V_{H} sequence of SEQ ID NO: 242. In some aspects, the antibody comprises a CDR-H3 sequence comprising, consisting of, or consisting essentially of a CDR-H3 sequence of a V_{H} sequence of SEQ ID NO: 243. In some aspects, the antibody comprises a CDR-H3 sequence comprising, consisting of, or consisting essentially of a CDR-H3 sequence of a V_{H} sequence of SEQ ID NO: 244. In some aspects, the antibody comprises a CDR-H3 sequence comprising, consisting of, or consisting essentially of a CDR-H3 sequence of a V_{H} sequence of SEQ ID NO: 245. In some aspects, the antibody comprises a CDR-H3 sequence comprising, consisting of, or consisting essentially of a CDR-H3 sequence of a V_{H} sequence of SEQ ID NO: 246. In some aspects, the antibody comprises a CDR-H3 sequence comprising, consisting of, or consisting essentially of a CDR-H3 sequence of a V_{H} sequence of SEQ ID NO: 247. In some aspects, the antibody comprises a CDR-H3 sequence comprising, consisting of, or consisting essentially of a CDR-H3 sequence of a V_{H} sequence of SEQ ID NO: 248. In some aspects, the antibody comprises a CDR-H3 sequence comprising, consisting of, or consisting essentially of a CDR-H3 sequence of a V_{H} sequence of SEQ ID NO: 249. In some aspects, the antibody comprises a CDR-H3 sequence comprising, consisting of, or consisting essentially of a CDR-H3 sequence of a V_{H} sequence of SEQ ID NO: 250. In some embodiments, the PD-1 antibody comprises a CDR-H3 sequence comprising, consisting of, or consisting essentially of a CDR-H3 sequence of an illustrative antibody or V_{H} sequence provided in WO 2016/077397.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

3.2 PD-1 V_{H} Sequences Comprising Illustrative CDRs

In some embodiments, the PD-1 antibody comprises a V_{H} sequence comprising one or more CDR-H sequences comprising, consisting of, or consisting essentially of one or more illustrative CDR-H sequences provided in this disclosure, and variants thereof. In some embodiments, the CDR-H sequences comprise, consist of, or consist essentially of one or more CDR-H sequences provided in a V_{H} sequence selected from SEQ ID NOs: 211-250. In some embodiments, the PD-1 antibody comprises a V_{H} sequence comprising one or more CDR-H sequences comprising, consisting of, or consisting essentially of one or more illustrative CDR-H sequences provided in WO 2016/077397.

3.3.1 V_{H} Sequences Comprising Illustrative Kabat CDRs

In some embodiments, the PD-1 antibody comprises a V_{H} sequence comprising one or more Kabat CDR-H sequences comprising, consisting of, or consisting essentially of one or more illustrative Kabat CDR-H sequences provided in this disclosure, and variants thereof. In some embodiments, the CDR-H sequences comprise, consist of, or consist essentially of one or more CDR-H sequences provided in a V_{H} sequence selected from SEQ ID NOs: 211-250.

3.2.2.1 Kabat CDR-H3

In some embodiments, the PD-1 antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided herein. In some aspects, the Kabat CDR-H3 sequence is a Kabat CDR-H3 sequence of a V_{H} sequence provided in SEQ ID NOs.: 211-250. In some embodiments, the PD-1 antibody comprises a V_{H} sequence comprising a CDR-H3 sequence, wherein the CDR-H3 sequence comprises, consists of, or consists essentially of a Kabat CDR-H3 sequence of an illustrative antibody or V_{H} sequence provided in WO 2016/077397.

In some embodiments, the PD-1 antibody comprises a V_{H} sequence comprising a Kabat CDR-H3 sequence comprising, consisting of, or consisting essentially of a sequence selected from SEQ ID NOs: 130-131. In some aspects, the antibody comprises a V_{H} sequence comprising a Kabat CDR-H3 sequence comprising, consisting of, or consisting essentially of SEQ ID NO: 130. In some aspects, the antibody comprises a V_{H} sequence comprising a Kabat CDR-H3 sequence comprising, consisting of, or consisting essentially of SEQ ID NO: 131.

In some embodiments, the PD-1 antibody comprises a V_{H} sequence comprising a Kabat CDR-H3 sequence comprising, consisting of, or consisting essentially of a Kabat CDR-H3 sequence of a V_{H} sequence selected from SEQ ID NOs.: 228-250. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 228. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 229. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 230. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 231. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 232. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 233. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 234. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 235. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 236. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 237. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 238. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 239. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 240. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 241. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 242. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 243. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 244. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 245. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 246. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 247. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 248. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 249. In some aspects, the antibody comprises a V_{H} sequence comprising a Kabat CDR-H3 sequence comprising, consisting of, or consisting essentially of a Kabat CDR-H3 sequence of a V_{H} sequence of SEQ ID NO: 250.

3.2.2.2 Kabat CDR-H2

In some embodiments, the PD-1 antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided herein. In some aspects, the Kabat CDR-H2 sequence is a Kabat CDR-H2 sequence of a V_{H} sequence provided in SEQ ID NOs.: 211-250. In some embodiments, the PD-1 antibody comprises a V_{H} sequence comprising a CDR-H2 sequence, wherein the CDR-H2 sequence comprises, consists of, or consists essentially of a Kabat CDR-H2 sequence of an illustrative antibody or V_{H} sequence provided in WO 2016/077397.

In some embodiments, the PD-1 antibody comprises a V_{H} sequence comprising a Kabat CDR-H2 sequence comprising, consisting of, or consisting essentially of a sequence selected from SEQ ID NOs: 108-109. In some aspects, the antibody comprises a V_{H} sequence comprising a Kabat CDR-H2 sequence comprising, consisting of, or consisting essentially of SEQ ID NO: 108. In some aspects, the antibody comprises a V_{H} sequence comprising a Kabat CDR-H2 sequence comprising, consisting of, or consisting essentially of SEQ ID NO: 109.

In some embodiments, the PD-1 antibody comprises a V_{H} sequence comprising a Kabat CDR-H2 sequence comprising, consisting of, or consisting essentially of a Kabat CDR-H2 sequence of a V_{H} sequence selected from SEQ ID NOs.: 228-250. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 228. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 229. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 230. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 231. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 232. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 233. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 234. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 235. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 236. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 237. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 238. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 239. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 240. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 241. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 242. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 243. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 244. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 245. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 246. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 247. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 248. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 249. In some aspects, the antibody comprises a V_{H} sequence comprising a Kabat CDR-H2 sequence comprising, consisting of, or consisting essentially of a Kabat CDR-H2 sequence of a V_{H} sequence of SEQ ID NO: 250.

3.2.2.3 Kabat CDR-H1

In some embodiments, the PD-1 antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided herein. In some aspects, the Kabat CDR-H1 sequence is a Kabat CDR-H1 sequence of a V_{H} sequence provided in SEQ ID NOs.: 211-250. In some embodiments, the PD-1 antibody comprises a V_{H} sequence comprising a CDR-H1 sequence, wherein the CDR-H1 sequence comprises, consists of, or consists essentially of a Kabat CDR-H1 sequence of an illustrative antibody or V_{H} sequence provided in WO 2016/077397.

In some embodiments, the PD-1 antibody comprises a V_{H} sequence comprising a Kabat CDR-H1 sequence comprising, consisting of, or consisting essentially of a sequence selected from SEQ ID NOs: 59-65. In some aspects, the antibody comprises a V_{H} sequence comprising a Kabat CDR-H1 sequence comprising, consisting of, or consisting essentially of SEQ ID NO: 59. In some aspects, the antibody comprises a V_{H} sequence comprising a Kabat CDR-H1 sequence comprising, consisting of, or consisting essentially of SEQ ID NO: 60. In some aspects, the antibody comprises a V_{H} sequence comprising a Kabat CDR-H1 sequence comprising, consisting of, or consisting essentially of SEQ ID NO: 61. In some aspects, the antibody comprises a V_{H} sequence comprising a Kabat CDR-H1 sequence comprising, consisting of, or consisting essentially of SEQ ID NO: 62. In some aspects, the antibody comprises a V_{H} sequence comprising a Kabat CDR-H1 sequence comprising, consisting of, or consisting essentially of SEQ ID NO: 63. In some aspects, the antibody comprises a V_{H} sequence comprising a Kabat CDR-H1 sequence comprising, consisting of, or consisting essentially of SEQ ID NO: 64. In some aspects, the antibody comprises a V_{H} sequence comprising a Kabat CDR-H1 sequence comprising, consisting of, or consisting essentially of SEQ ID NO: 65.

In some embodiments, the PD-1 antibody comprises a V_{H} sequence comprising a Kabat CDR-H1 sequence comprising, consisting of, or consisting essentially of a Kabat CDR-H1 sequence of a V_{H} sequence selected from SEQ ID NOs.: 228-250. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 228. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 229. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 230. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 231. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 232. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 233. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 234. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 235. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 236. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 237. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 238. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 239. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 240. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 241. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 242. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 243. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 244. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 245. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 246. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 247. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 248. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 249. In some aspects, the antibody comprises a V_{H} sequence comprising a Kabat CDR-H1 sequence comprising, consisting of, or consisting essentially of a Kabat CDR-H1 sequence of a V_{H} sequence of SEQ ID NO: 250.

3.2.2.4 Kabat CDR-H3+Kabat CDR-H2

In some embodiments, the PD-1 antibody comprises a V_{H} sequence comprising a Kabat CDR-H3 sequence comprising, consisting of, or consisting essentially of a sequence selected from SEQ ID NOs: 130-131, and a Kabat CDR-H2 sequence comprising, consisting of, or consisting essentially of a sequence selected from SEQ ID NOs: 108-109. In some embodiments, the PD-1 antibody comprises a V_{H} sequence comprising a Kabat CDR-H3 sequence comprising, consisting of, or consisting essentially of a sequence selected from a Kabat CDR-H3 sequence of a V_{H} sequence selected from SEQ ID NOs.: 211-250, and a Kabat CDR-H2 sequence comprising, consisting of, or consisting essentially of a sequence selected from a Kabat CDR-H2 sequence of a V_{H} sequence selected from SEQ ID NOs.: 211-250. In some aspects, the Kabat CDR-H3 sequence and the Kabat CDR-H2 sequence are both from a single illustrative V_{H} sequence provided in this disclosure. For example, in some aspects, the Kabat CDR-H3 and Kabat CDR-H2 are both from a single illustrative V_{H} sequence selected from SEQ ID NOs: 211-250.

3.2.2.5 Kabat CDR-H3+Kabat CDR-H1

In some embodiments, the PD-1 antibody comprises a V_{H} sequence comprising a Kabat CDR-H3 sequence comprising, consisting of, or consisting essentially of a sequence selected from SEQ ID NOs: 130-131, and a Kabat CDR-H1 sequence comprising, consisting of, or consisting essentially of a sequence selected from SEQ ID NOs: 59-65. In some embodiments, the PD-1 antibody comprises a V_{H} sequence comprising a Kabat CDR-H3 sequence comprising, consisting of, or consisting essentially of a sequence selected from a Kabat CDR-H3 sequence of a V_{H} sequence provided in any one of SEQ ID NOs.: 211-250, and a Kabat CDR-H1 sequence comprising, consisting of, or consisting essentially of a sequence selected from a Kabat CDR-H1 sequence of a V_{H} sequence provided in any one of SEQ ID NOs.: 211-250. In some aspects, the Kabat CDR-H3 sequence and the Kabat CDR-H1 sequence are both from a single illustrative V_{H} sequence provided in this disclosure. For example, in some aspects, the Kabat CDR-H3 and Kabat CDR-H1 are both from a single illustrative V_{H} sequence selected from SEQ ID NOs: 211-250.

3.2.2.6 Kabat CDR-H1+Kabat CDR-H2

In some embodiments, the PD-1 antibody comprises a V_{H} sequence comprising a Kabat CDR-H1 sequence comprising, consisting of, or consisting essentially of a sequence selected from SEQ ID NOs: 59-65, and a Kabat CDR-H2 sequence comprising, consisting of, or consisting essentially of a sequence selected from SEQ ID NOs: 108-109. In some embodiments, the PD-1 antibody comprises a V_{H} sequence comprising a Kabat CDR-H1 sequence comprising, consisting of, or consisting essentially of a sequence selected from a Kabat CDR-H1 sequence of a V_{H} sequence provided in any one of SEQ ID NOs.: 211-250, and a Kabat CDR-H2 sequence comprising, consisting of, or consisting essentially of a sequence selected from a Kabat CDR-H2 sequence of a V_{H} sequence provided in any one of SEQ ID NOs.: 211-250. In some aspects, the Kabat CDR-H1 sequence and the Kabat CDR-H2 sequence are both from a single illustrative V_{H} sequence provided in this disclosure. For example, in some aspects, the Kabat CDR-H1 and Kabat CDR-H2 are both from a single illustrative V_{H} sequence selected from SEQ ID NOs: 211-250.

3.2.2.7 Kabat CDR-H1+Kabat CDR-H2+Kabat CDR-H3

In some embodiments, the PD-1 antibody comprises a V_{H} sequence comprising a Kabat CDR-H1 sequence comprising, consisting of, or consisting essentially of a sequence selected from SEQ ID NOs: 59-65, a Kabat CDR-H2 sequence comprising, consisting of, or consisting essentially of a sequence selected from SEQ ID NOs: 108-109, and a Kabat CDR-H3 sequence comprising, consisting of, or consisting essentially of a sequence selected from SEQ ID NOs: 130-131. In some embodiments, the PD-1 antibody comprises a V_{H} sequence comprising a Kabat CDR-H1 sequence comprising, consisting of, or consisting essentially of a sequence selected from a Kabat CDR-H1 sequence of a V_{H} sequence selected from SEQ ID NOs.: 211-250, a Kabat CDR-H2 sequence comprising, consisting of, or consisting essentially of a sequence selected from a Kabat CDR-H2 sequence of a V_{H} sequence selected from SEQ ID NOs.: 211-250, and a Kabat CDR-H3 sequence comprising, consisting of, or consisting essentially of a sequence selected from a Kabat CDR-H3 sequence of a V_{H} sequence selected from SEQ ID NOs.: 211-250. In some aspects, the Kabat CDR-H1 sequence, Kabat CDR-H2 sequence, and Kabat CDR-H3 are all from a single illustrative V_{H} sequence provided in this disclosure. For example, in some aspects, the Kabat CDR-H1, Kabat CDR-H2, and Kabat CDR-H3 are all from a single illustrative V_{H} sequence selected from SEQ ID NOs: 211-250.

3.2.2.8 Variants of V_{H} Sequences Comprising Illustrative Kabat CDRs

In some embodiments, the V_{H} sequences provided herein comprise a variant of an illustrative Kabat CDR-H3, CDR-H2, and/or CDR-H1 sequence provided in this disclosure. In some embodiments, the V_{H} sequences provided herein comprise a variant of an illustrative Kabat CDR-H3, CDR-H2, and/or CDR-H1 sequence provided in WO 2016/077397.

In some aspects, the Kabat CDR-H3 sequence comprises, consists of, or consists essentially of a variant of an illustrative Kabat CDR-H3 sequence provided in this disclosure. In some aspects, the Kabat CDR-H3 sequence comprises, consists of, or consists essentially of a sequence having at least 70%, 75%, 80%, 85%, 90%, or 95% identity with any of the illustrative Kabat CDR-H3 sequences provided in this disclosure. In some aspects, the Kabat CDR-H3 sequence comprises, consists of, or consists essentially of any of the illustrative Kabat CDR-H3 sequences provided in this disclosure, with 1, 2, or 3 amino acid substitutions. In some aspects, the amino acid substitutions are conservative amino acid substitutions.

In some aspects, the Kabat CDR-H2 sequence comprises, consists of, or consists essentially of a variant of an illustrative Kabat CDR-H2 sequence provided in this disclosure. In some aspects, the Kabat CDR-H2 sequence comprises, consists of, or consists essentially of a sequence having at least 70%, 75%, 80%, 85%, 90%, or 95% identity with any of the illustrative Kabat CDR-H2 sequences provided in this disclosure. In some aspects, the Kabat CDR-H2 sequence comprises, consists of, or consists essentially of any of the illustrative Kabat CDR-H2 sequences provided in this disclosure, with 1, 2, or 3 amino acid substitutions. In some aspects, the amino acid substitutions are conservative amino acid substitutions.

In some aspects, the Kabat CDR-H1 sequence comprises, consists of, or consists essentially of a variant of an illustrative Kabat CDR-H1 sequence provided in this disclosure. In some aspects, the Kabat CDR-H1 sequence comprises, consists of, or consists essentially of a sequence having at least 70%, 75%, 80%, 85%, 90%, or 95% identity with any of the illustrative Kabat CDR-H1 sequences provided in this disclosure. In some aspects, the Kabat CDR-H1 sequence comprises, consists of, or consists essentially of any of the illustrative Kabat CDR-H1 sequences provided in this disclosure, with 1, 2, or 3 amino acid substitutions. In some aspects, the amino acid substitutions are conservative amino acid substitutions.

3.3.2 V_{H} Sequences Comprising Illustrative Chothia CDRs

In some embodiments, the PD-1 antibody comprises a V_{H} sequence comprising one or more Chothia CDR-H sequences comprising, consisting of, or consisting essentially of one or more illustrative Chothia CDR-H sequences provided in this disclosure, and variants thereof. In some embodiments, the PD-1 antibody comprises a V_{H} sequence comprising one or more Chothia CDR-H sequences comprising, consisting of, or consisting essentially of one or more illustrative Chothia CDR-H sequences provided in WO 2016/077397.

3.2.2.1 Chothia CDR-H3

In some embodiments, the PD-1 antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided herein. In some aspects, the Chothia CDR-H3 sequence is a Chothia CDR-H3 sequence of a V_{H} sequence provided in SEQ ID NOs.: 211-250. In some embodiments, the PD-1 antibody comprises a V_{H} sequence comprising a CDR-H3 sequence, wherein the CDR-H3 sequence comprises, consists of, or consists essentially of a Chothia CDR-H3 sequence of an illustrative antibody or V_{H} sequence provided in WO 2016/077397.

In some embodiments, the PD-1 antibody comprises a V_{H} sequence comprising a Chothia CDR-H3 sequence comprising, consisting of, or consisting essentially of a sequence selected from SEQ ID NOs: 130-131. In some aspects, the antibody comprises a V_{H} sequence comprising a Chothia CDR-H3 sequence comprising, consisting of, or consisting essentially of SEQ ID NO: 130. In some aspects, the antibody comprises a V_{H} sequence comprising a Chothia CDR-H3 sequence comprising, consisting of, or consisting essentially of SEQ ID NO: 131.

In some embodiments, the PD-1 antibody comprises a V_{H} sequence comprising a Chothia CDR-H3 sequence comprising, consisting of, or consisting essentially of a Chothia CDR-H3 sequence of a V_{H} sequence selected from SEQ ID NOs.: 228-250. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 228. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 229. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 230. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 231. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 232. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 233. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 234. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 235. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 236. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 237. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 238. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 239. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 240. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 241. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 242. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 243. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 244. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 245. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 246. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 247. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 248. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 249. In some aspects, the antibody comprises a V_{H} sequence comprising a Chothia CDR-H3 sequence comprising, consisting of, or consisting essentially of a Chothia CDR-H3 sequence of a V_{H} sequence of SEQ ID NO: 250.

3.2.2.2 Chothia CDR-H2

In some embodiments, the PD-1 antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided herein. In some aspects, the Chothia CDR-H2 sequence is a Chothia CDR-H2 sequence of a V_{H} sequence provided in SEQ ID NOs.: 211-250. In some embodiments, the PD-1 antibody comprises a V_{H} sequence comprising a CDR-H2 sequence, wherein the CDR-H2 sequence comprises, consists of, or consists essentially of a Chothia CDR-H2 sequence of an illustrative antibody or V_{H} sequence provided in WO 2016/077397.

In some embodiments, the PD-1 antibody comprises a V_{H} sequence comprising a Chothia CDR-H2 sequence comprising, consisting of, or consisting essentially of a sequence selected from SEQ ID NOs: 86-87. In some aspects, the antibody comprises a V_{H} sequence comprising a Chothia CDR-H2 sequence comprising, consisting of, or consisting essentially of SEQ ID NO: 86. In some aspects, the antibody comprises a V_{H} sequence comprising a Chothia CDR-H2 sequence comprising, consisting of, or consisting essentially of SEQ ID NO: 87.

In some embodiments, the PD-1 antibody comprises a V_{H} sequence comprising a Chothia CDR-H2 sequence comprising, consisting of, or consisting essentially of a Chothia CDR-H2 sequence of a V_{H} sequence selected from SEQ ID NOs.: 228-250. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 228. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 229. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 230. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 231. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 232. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 233. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 234. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 235. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 236. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 237. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 238. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 239. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 240. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 241. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 242. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 243. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 244. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 245.

In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 246. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 247. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 248. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 249. In some aspects, the antibody comprises a V_{H} sequence comprising a Chothia CDR-H2 sequence comprising, consisting of, or consisting essentially of a Chothia CDR-H2 sequence of a V_{H} sequence of SEQ ID NO: 250.

3.2.2.3 Chothia CDR-H1

In some embodiments, the PD-1 antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided herein. In some aspects, the Chothia CDR-H1 sequence is a Chothia CDR-H1 sequence of a V_{H} sequence provided in SEQ ID NOs.: 211-250. In some embodiments, the PD-1 antibody comprises a V_{H} sequence comprising a CDR-H1 sequence, wherein the CDR-H1 sequence comprises, consists of, or consists essentially of a Chothia CDR-H1 sequence of an illustrative antibody or V_{H} sequence provided in WO 2016/077397.

In some embodiments, the PD-1 antibody comprises a V_{H} sequence comprising a Chothia CDR-H1 sequence comprising, consisting of, or consisting essentially of a sequence selected from SEQ ID NOs: 24-38. In some aspects, the antibody comprises a V_{H} sequence comprising a Chothia CDR-H1 sequence comprising, consisting of, or consisting essentially of SEQ ID NO: 24. In some aspects, the antibody comprises a V_{H} sequence comprising a Chothia CDR-H1 sequence comprising, consisting of, or consisting essentially of SEQ ID NO: 25. In some aspects, the antibody comprises a V_{H} sequence comprising a Chothia CDR-H1 sequence comprising, consisting of, or consisting essentially of SEQ ID NO: 26. In some aspects, the antibody comprises a V_{H} sequence comprising a Chothia CDR-H1 sequence comprising, consisting of, or consisting essentially of SEQ ID NO: 27. In some aspects, the antibody comprises a V_{H} sequence comprising a Chothia CDR-H1 sequence comprising, consisting of, or consisting essentially of SEQ ID NO: 28. In some aspects, the antibody comprises a V_{H} sequence comprising a Chothia CDR-H1 sequence comprising, consisting of, or consisting essentially of SEQ ID NO: 29. In some aspects, the antibody comprises a V_{H} sequence comprising a Chothia CDR-H1 sequence comprising, consisting of, or consisting essentially of SEQ ID NO: 30. In some aspects, the antibody comprises a V_{H} sequence comprising a Chothia CDR-H1 sequence comprising, consisting of, or consisting essentially of SEQ ID NO: 31. In some aspects, the antibody comprises a V_{H} sequence comprising a Chothia CDR-H1 sequence comprising, consisting of, or consisting essentially of SEQ ID NO: 32. In some aspects, the antibody comprises a V_{H} sequence comprising a Chothia CDR-H1 sequence comprising, consisting of, or consisting essentially of SEQ ID NO: 33. In some aspects, the antibody comprises a V_{H} sequence comprising a Chothia CDR-H1 sequence comprising, consisting of, or consisting essentially of SEQ ID NO: 34. In some aspects, the antibody comprises a V_{H} sequence comprising a Chothia CDR-H1 sequence comprising, consisting of, or consisting essentially of SEQ ID NO: 35. In some aspects, the antibody comprises a V_{H} sequence comprising a Chothia CDR-H1 sequence comprising, consisting of, or consisting essentially of SEQ ID NO: 36. In some aspects, the antibody comprises a V_{H} sequence comprising a Chothia CDR-H1 sequence comprising, consisting of, or consisting essentially of SEQ ID NO: 37. In some aspects, the antibody comprises a V_{H} sequence comprising a Chothia CDR-H1 sequence comprising, consisting of, or consisting essentially of SEQ ID NO: 38.

In some embodiments, the PD-1 antibody comprises a V_{H} sequence comprising a Chothia CDR-H1 sequence comprising, consisting of, or consisting essentially of a Chothia CDR-H1 sequence of a V_{H} sequence selected from SEQ ID NOs.: 228-250. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 228. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 229. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 230. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 231. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 232. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 233. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 234. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 235. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 236. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 237. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 238. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 239. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 240. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 241. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 242. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 243. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 244. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 245. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 246. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 247. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 248. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 249. In some aspects, the antibody comprises a V_{H} sequence comprising a Chothia CDR-H1 sequence comprising, consisting of, or consisting essentially of a Chothia CDR-H1 sequence of a V_{H} sequence of SEQ ID NO: 250.

3.2.2.4 Chothia CDR-H3+Chothia CDR-H2

In some embodiments, the PD-1 antibody comprises a V_{H} sequence comprising a Chothia CDR-H3 sequence comprising, consisting of, or consisting essentially of a sequence selected from SEQ ID NOs: 130-131, and a Chothia CDR-H2 sequence comprising, consisting of, or consisting essentially of a sequence selected from SEQ ID NOs: 86-87. In some embodiments, the PD-1 antibody comprises a V_{H} sequence comprising a Chothia CDR-H3 sequence comprising, consisting of, or consisting essentially of a sequence selected from a Chothia CDR-H3 sequence of a V_{H} sequence selected from SEQ ID NOs.: 211-250, and a Chothia CDR-H2 sequence comprising, consisting of, or consisting essentially of a sequence selected from a Chothia CDR-H2 sequence of a V_{H} sequence selected from SEQ ID NOs.: 211-250. In some aspects, the Chothia CDR-H3 sequence and the Chothia CDR-H2 sequence are both from a single illustrative V_{H} sequence provided in this disclosure. For example, in some aspects, the Chothia CDR-H3 and Chothia CDR-H2 are both from a single illustrative V_{H} sequence selected from SEQ ID NOs: 211-250.

3.2.2.5 Chothia CDR-H3+Chothia CDR-H1

In some embodiments, the PD-1 antibody comprises a V_{H} sequence comprising a Chothia CDR-H3 sequence comprising, consisting of, or consisting essentially of a sequence selected from SEQ ID NOs: 130-131, and a Chothia CDR-H1 sequence comprising, consisting of, or consisting essentially of a sequence selected from SEQ ID NOs: 24-38. In some embodiments, the PD-1 antibody comprises a V_{H} sequence comprising a Chothia CDR-H3 sequence comprising, consisting of, or consisting essentially of a sequence selected from a Chothia CDR-H3 sequence of a V_{H} sequence provided in any one of SEQ ID NOs.: 211-250, and a Chothia CDR-H1 sequence comprising, consisting of, or consisting essentially of a sequence selected from a Chothia CDR-H1 sequence of a V_{H} sequence provided in any one of SEQ ID NOs.: 211-250. In some aspects, the Chothia CDR-H3 sequence and the Chothia CDR-H1 sequence are both from a single illustrative V_{H} sequence provided in this disclosure. For example, in some aspects, the Chothia CDR-H3 and Chothia CDR-H1 are both from a single illustrative V_{H} sequence selected from SEQ ID NOs: 211-250.

3.2.2.6 Chothia CDR-H1+Chothia CDR-H2

In some embodiments, the PD-1 antibody comprises a V_{H} sequence comprising a Chothia CDR-H1 sequence comprising, consisting of, or consisting essentially of a sequence selected from SEQ ID NOs: 24-38 and a Chothia CDR-H2 sequence comprising, consisting of, or consisting essentially of a sequence selected from SEQ ID NOs: 86-87. In some embodiments, the PD-1 antibody comprises a V_{H} sequence comprising a Chothia CDR-H1 sequence comprising, consisting of, or consisting essentially of a sequence selected from a Chothia CDR-H1 sequence of a V_{H} sequence provided in any one of SEQ ID NOs.: 211-250, and a Chothia CDR-H2 sequence comprising, consisting of, or consisting essentially of a sequence selected from a Chothia CDR-H2 sequence of a V_{H} sequence provided in any one of SEQ ID NOs.: 211-250. In some aspects, the Chothia CDR-H1 sequence and the Chothia CDR-H2 sequence are both from a single illustrative V_{H} sequence provided in this disclosure. For example, in some aspects, the Chothia CDR-H1 and Chothia CDR-H2 are both from a single illustrative V_{H} sequence selected from SEQ ID NOs: 211-250.

3.2.2.7 Chothia CDR-H1+Chothia CDR-H2+Chothia CDR-H3

In some embodiments, the PD-1 antibody comprises a V_{H} sequence comprising a Chothia CDR-H1 sequence comprising, consisting of, or consisting essentially of a sequence selected from SEQ ID NOs: 24-38, a Chothia CDR-H2 sequence comprising, consisting of, or consisting essentially of a sequence selected from SEQ ID NOs: 86-87, and a Chothia CDR-H3 sequence comprising, consisting of, or consisting essentially of a sequence selected from SEQ ID NOs: 130-131. In some embodiments, the PD-1 antibody comprises a V_{H} sequence comprising a Chothia CDR-H1 sequence comprising, consisting of, or consisting essentially of a sequence selected from a Chothia CDR-H1 sequence of a Vii sequence provided in any one of SEQ ID NOs.: 211-250, a Chothia CDR-H2 sequence comprising, consisting of, or consisting essentially of a sequence selected from a Chothia CDR-H2 sequence of a V_{H} sequence provided in any one of SEQ ID NOs.: 211-250, and a Chothia CDR-H3 sequence comprising, consisting of, or consisting essentially of a sequence selected from a Chothia CDR-H3 sequence of a V_{H} sequence provided in any one of SEQ ID NOs.: 211-250. In some aspects, the Chothia CDR-H1 sequence, Chothia CDR-H2 sequence, and Chothia CDR-H3 sequence are all from a single illustrative V_{H} sequence provided in this disclosure. For example, in some aspects, the Chothia CDR-H1, Chothia CDR-H2, and Chothia CDR-H3 are all from a single illustrative V_{H} sequence selected from SEQ ID NOs: 211-250.

3.2.2.8 Variants of V_{H} Sequences Comprising Illustrative Chothia CDRs

In some embodiments, the V_{H} sequences provided herein comprise a variant of an illustrative Chothia CDR-H3, CDR-H2, and/or CDR-H1 sequence provided in this disclosure.

In some aspects, the Chothia CDR-H3 sequence comprises, consists of, or consists essentially of a variant of an illustrative Chothia CDR-H3 sequence provided in this disclosure. In some aspects, the Chothia CDR-H3 sequence comprises, consists of, or consists essentially of a sequence having at least 70%, 75%, 80%, 85%, 90%, or 95% identity with any of the illustrative Chothia CDR-H3 sequences provided in this disclosure. In some aspects, the Chothia CDR-H3 sequence comprises, consists of, or consists essentially of any of the illustrative Chothia CDR-H3 sequences provided in this disclosure, with 1, 2, or 3 amino acid substitutions. In some aspects, the amino acid substitutions are conservative amino acid substitutions.

In some aspects, the Chothia CDR-H2 sequence comprises, consists of, or consists essentially of a variant of an illustrative Chothia CDR-H2 sequence provided in this disclosure. In some aspects, the Chothia CDR-H2 sequence comprises, consists of, or consists essentially of a sequence having at least 70%, 75%, 80%, 85%, 90%, or 95% identity with any of the illustrative Chothia CDR-H2 sequences provided in this disclosure. In some aspects, the Chothia CDR-H2 sequence comprises, consists of, or consists essentially of any of the illustrative Chothia CDR-H2 sequences provided in this disclosure, with 1, 2, or 3 amino acid substitutions. In some aspects, the amino acid substitutions are conservative amino acid substitutions.

In some aspects, the Chothia CDR-H1 sequence comprises, consists of, or consists essentially of a variant of an illustrative Chothia CDR-H1 sequence provided in this disclosure. In some aspects, the Chothia CDR-H1 sequence comprises, consists of, or consists essentially of a sequence having at least 70%, 75%, 80%, 85%, 90%, or 95% identity with any of the illustrative Chothia CDR-H1 sequences provided in this disclosure. In some aspects, the Chothia CDR-H1 sequence comprises, consists of, or consists essentially of any of the illustrative Chothia CDR-H1 sequences provided in this disclosure, with 1, 2, or 3 amino acid substitutions. In some aspects, the amino acid substitutions are conservative amino acid substitutions.

3.3 PD-1 V_{H} Sequences

In some embodiments, the PD-1 antibody comprises, consists of, or consists essentially of a V_{H} sequence provided in SEQ ID NOs.: 211-250.

In some embodiments, the PD-1 antibody comprises a V_{H} sequence comprising, consisting of, or consisting essentially of a sequence selected from SEQ ID NOs: 211-250. In some aspects, the antibody comprises a V_{H} sequence comprising, consisting of, or consisting essentially of SEQ ID NO: 211. In some aspects, the antibody comprises a V_{H} sequence comprising, consisting of, or consisting essentially of SEQ ID NO: 212. In some aspects, the antibody comprises a V_{H} sequence comprising, consisting of, or consisting essentially of SEQ ID NO: 213. In some aspects, the antibody comprises a V_{H} sequence comprising, consisting of, or consisting essentially of SEQ ID NO: 214. In some aspects, the antibody comprises a V_{H} sequence comprising, consisting of, or consisting essentially of SEQ ID NO: 215. In some aspects, the antibody comprises a V_{H} sequence comprising, consisting of, or consisting essentially of SEQ ID NO: 216. In some aspects, the antibody comprises a V_{H} sequence comprising, consisting of, or consisting essentially of SEQ ID NO: 217. In some aspects, the antibody comprises a V_{H} sequence comprising, consisting of, or consisting essentially of SEQ ID NO: 218. In some aspects, the antibody comprises a V_{H} sequence comprising, consisting of, or consisting essentially of SEQ ID NO: 219. In some aspects, the antibody comprises a V_{H} sequence comprising, consisting of, or consisting essentially of SEQ ID NO: 220. In some aspects, the antibody comprises a V_{H} sequence comprising, consisting of, or consisting essentially of SEQ ID NO: 221. In some aspects, the antibody comprises a V_{H} sequence comprising, consisting of, or consisting essentially of SEQ ID NO: 222. In some aspects, the antibody comprises a V_{H} sequence comprising, consisting of, or consisting essentially of SEQ ID NO: 223. In some aspects, the antibody comprises a V_{H} sequence comprising, consisting of, or consisting essentially of SEQ ID NO: 224. In some aspects, the antibody comprises a V_{H} sequence comprising, consisting of, or consisting essentially of SEQ ID NO: 225.

In some aspects, the antibody comprises a V_{H} sequence comprising, consisting of, or consisting essentially of SEQ ID NO: 226. In some aspects, the antibody comprises a V_{H} sequence comprising, consisting of, or consisting essentially of SEQ ID NO: 227. In some aspects, the antibody comprises a V_{H} sequence comprising, consisting of, or consisting essentially of SEQ ID NO: 228. In some aspects, the antibody comprises a V_{H} sequence comprising, consisting of, or consisting essentially of SEQ ID NO: 229. In some aspects, the antibody comprises a V_{H} sequence comprising, consisting of, or consisting essentially of SEQ ID NO: 230. In some aspects, the antibody comprises a V_{H} sequence comprising, consisting of, or consisting essentially of SEQ ID NO: 231. In some aspects, the antibody comprises a V_{H} sequence comprising, consisting of, or consisting essentially of SEQ ID NO: 232. In some aspects, the antibody comprises a V_{H} sequence comprising, consisting of, or consisting essentially of SEQ ID NO: 233. In some aspects, the antibody comprises a V_{H} sequence comprising, consisting of, or consisting essentially of SEQ ID NO: 234. In some aspects, the antibody comprises a V_{H} sequence comprising, consisting of, or consisting essentially of SEQ ID NO: 235. In some aspects, the antibody comprises a V_{H} sequence comprising, consisting of, or consisting essentially of SEQ ID NO: 236. In some aspects, the antibody comprises a V_{H} sequence comprising, consisting of, or consisting essentially of SEQ ID NO: 237. In some aspects, the antibody comprises a V_{H} sequence comprising, consisting of, or consisting essentially of SEQ ID NO: 238. In some aspects, the antibody comprises a V_{H} sequence comprising, consisting of, or consisting essentially of SEQ ID NO: 239. In some aspects, the antibody comprises a V_{H} sequence comprising, consisting of, or consisting essentially of SEQ ID NO: 240. In some aspects, the antibody comprises a V_{H} sequence comprising, consisting of, or consisting essentially of SEQ ID NO: 241. In some aspects, the antibody comprises a V_{H} sequence comprising, consisting of, or consisting essentially of SEQ ID NO: 242. In some aspects, the antibody comprises a V_{H} sequence comprising, consisting of, or consisting essentially of SEQ ID NO: 243. In some aspects, the antibody comprises a V_{H} sequence comprising, consisting of, or consisting essentially of SEQ ID NO: 244. In some aspects, the antibody comprises a V_{H} sequence comprising, consisting of, or consisting essentially of SEQ ID NO: 245. In some aspects, the antibody comprises a V_{H} sequence comprising, consisting of, or consisting essentially of SEQ ID NO: 246. In some aspects, the antibody comprises a V_{H} sequence comprising, consisting of, or consisting essentially of SEQ ID NO: 247. In some aspects, the antibody comprises a V_{H} sequence comprising, consisting of, or consisting essentially of SEQ ID NO: 248. In some aspects, the antibody comprises a V_{H} sequence comprising, consisting of, or consisting essentially of SEQ ID NO: 249. In some aspects, the antibody comprises a V_{H} sequence comprising, consisting of, or consisting essentially of SEQ ID NO: 250.

3.3.1 Variants of V_{H} Sequences

In some embodiments, the V_{H} sequences provided herein comprise, consist of, or consist essentially of a variant of an illustrative V_{H} sequence provided in this disclosure. In some embodiments, the V_{H} sequences provided herein comprise, consist of, or consist essentially of a variant of an illustrative V_{H} sequence provided in WO 2016/077397.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.5% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

3.4 PD-1 CDR-L3 Sequences

In some embodiments, the PD-1 antibody comprises a CDR-L3 sequence comprising, consisting of, or consisting essentially of a CDR-L3 sequence of an illustrative antibody or V_{L} sequence provided herein. In some aspects, the CDR-L3 sequence is a CDR-L3 sequence of a V_{L} sequence provided in SEQ ID NOs.: 267-299. In some embodiments, the PD-1 antibody comprises a CDR-L3 sequence comprising, consisting of, or consisting essentially of a CDR-L3 sequence of an illustrative antibody or V_{L} sequence provided in WO 2016/077397.

In some embodiments, the PD-1 antibody comprises a CDR-L3 sequence comprising, consisting of, or consisting essentially of a sequence selected from SEQ ID NOs: 189-190. In some aspects, the antibody comprises a CDR-L3 sequence comprising, consisting of, or consisting essentially of SEQ ID NO: 189. In some aspects, the antibody comprises a CDR-L3 sequence comprising, consisting of, or consisting essentially of SEQ ID NO: 190.

In some embodiments, the PD-1 antibody comprises a CDR-L3 sequence comprising, consisting of, or consisting essentially of a CDR-L3 sequence of a V_{L} sequence selected from SEQ ID NOs.: 277-299. In some aspects, the antibody comprises a CDR-L3 sequence comprising, consisting of, or consisting essentially of a CDR-L3 sequence of a V_{L} sequence of SEQ ID NO: 277. In some aspects, the antibody comprises a CDR-L3 sequence comprising, consisting of, or consisting essentially of a CDR-L3 sequence of a V_{L} sequence of SEQ ID NO: 278. In some aspects, the antibody comprises a CDR-L3 sequence comprising, consisting of, or consisting essentially of a CDR-L3 sequence of a V_{L} sequence of SEQ ID NO: 279. In some aspects, the antibody comprises a CDR-L3 sequence comprising, consisting of, or consisting essentially of a CDR-L3 sequence of a V_{L} sequence of SEQ ID NO: 280. In some aspects, the antibody comprises a CDR-L3 sequence comprising, consisting of, or consisting essentially of a CDR-L3 sequence of a V_{L} sequence of SEQ ID NO: 281. In some aspects, the antibody comprises a CDR-L3 sequence comprising, consisting of, or consisting essentially of a CDR-L3 sequence of a V_{L} sequence of SEQ ID NO: 282. In some aspects, the antibody comprises a CDR-L3 sequence comprising, consisting of, or consisting essentially of a CDR-L3 sequence of a V_{L} sequence of SEQ ID NO: 283. In some aspects, the antibody comprises a CDR-L3 sequence comprising, consisting of, or consisting essentially of a CDR-L3 sequence of a V_{L} sequence of SEQ ID NO: 284. In some aspects, the antibody comprises a CDR-L3 sequence comprising, consisting of, or consisting essentially of a CDR-L3 sequence of a V_{L} sequence of SEQ ID NO: 285. In some aspects, the antibody comprises a CDR-L3 sequence comprising, consisting of, or consisting essentially of a CDR-L3 sequence of a V_{L} sequence of SEQ ID NO: 286. In some aspects, the antibody comprises a CDR-L3 sequence comprising, consisting of, or consisting essentially of a CDR-L3 sequence of a V_{L} sequence of SEQ ID NO: 287. In some aspects, the antibody comprises a CDR-L3 sequence comprising, consisting of, or consisting essentially of a CDR-L3 sequence of a V_{L} sequence of SEQ ID NO: 288. In some aspects, the antibody comprises a CDR-L3 sequence comprising, consisting of, or consisting essentially of a CDR-L3 sequence of a V_{L} sequence of SEQ ID NO: 289. In some aspects, the antibody comprises a CDR-L3 sequence comprising, consisting of, or consisting essentially of a CDR-L3 sequence of a V_{L} sequence of SEQ ID NO: 290. In some aspects, the antibody comprises a CDR-L3 sequence comprising, consisting of, or consisting essentially of a CDR-L3 sequence of a V_{L} sequence of SEQ ID NO: 291. In some aspects, the antibody comprises a CDR-L3 sequence comprising, consisting of, or consisting essentially of a CDR-L3 sequence of a V_{L} sequence of SEQ ID NO: 292. In some aspects, the antibody comprises a CDR-L3 sequence comprising, consisting of, or consisting essentially of a CDR-L3 sequence of a V_{L} sequence of SEQ ID NO: 293. In some aspects, the antibody comprises a CDR-L3 sequence comprising, consisting of, or consisting essentially of a CDR-L3 sequence of a V_{L} sequence of SEQ ID NO: 294. In some aspects, the antibody comprises a CDR-L3 sequence comprising, consisting of, or consisting essentially of a CDR-L3 sequence of a V_{L} sequence of SEQ ID NO: 295. In some aspects, the antibody comprises a CDR-L3 sequence comprising, consisting of, or consisting essentially of a CDR-L3 sequence of a V_{L} sequence of SEQ ID NO: 296. In some aspects, the antibody comprises a CDR-L3 sequence comprising, consisting of, or consisting essentially of a CDR-L3 sequence of a V_{L} sequence of SEQ ID NO: 297. In some aspects, the antibody comprises a CDR-L3 sequence comprising, consisting of, or consisting essentially of a CDR-L3 sequence of a V_{L} sequence of SEQ ID NO: 298. In some aspects, the antibody comprises a CDR-L3 sequence comprising, consisting of, or consisting essentially of a CDR-L3 sequence of a V_{L} sequence of SEQ ID NO: 299.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

3.5 PD-1 V_{L} Sequences Comprising Illustrative CDRs

In some embodiments, the PD-1 antibody comprises a V_{L} sequence comprising one or more CDR-L sequences comprising, consisting of, or consisting essentially of one or more illustrative CDR-L sequences provided in this disclosure, and variants thereof.

3.5.1 CDR-L3

In some embodiments, the PD-1 antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided herein. In some aspects, the CDR-L3 sequence is a CDR-L3 sequence of a V_{L} sequence provided in SEQ ID NOs.: 267-299. In some embodiments, the PD-1 antibody comprises a V_{L} sequence comprising a CDR-L3 sequence, wherein the CDR-L3 sequence comprises, consists of, or consists essentially of a CDR-L3 sequence of an illustrative antibody or V_{L} sequence provided in WO 2016/077397.

In some embodiments, the PD-1 antibody comprises a V_{L} sequence comprising a CDR-L3 sequence comprising, consisting of, or consisting essentially of a sequence selected from SEQ ID NOs: 189-190. In some aspects, the antibody comprises a V_{L} sequence comprising a CDR-L3 sequence comprising, consisting of, or consisting essentially of SEQ ID NO: 189. In some aspects, the antibody comprises a V_{L} sequence comprising a CDR-L3 sequence comprising, consisting of, or consisting essentially of SEQ ID NO: 190.

In some embodiments, the PD-1 antibody comprises a CDR-L3 sequence comprising, consisting of, or consisting essentially of a CDR-L3 sequence of a V_{L} sequence selected from SEQ ID NOs.: 277-299. In some aspects, the antibody comprises a CDR-L3 sequence comprising, consisting of, or consisting essentially of a CDR-L3 sequence of a V_{L} sequence of SEQ ID NO: 277. In some aspects, the antibody comprises a CDR-L3 sequence comprising, consisting of, or consisting essentially of a CDR-L3 sequence of a V_{L} sequence of SEQ ID NO: 278. In some aspects, the antibody comprises a CDR-L3 sequence comprising, consisting of, or consisting essentially of a CDR-L3 sequence of a V_{L} sequence of SEQ ID NO: 279. In some aspects, the antibody comprises a CDR-L3 sequence comprising, consisting of, or consisting essentially of a CDR-L3 sequence of a V_{L} sequence of SEQ ID NO: 280. In some aspects, the antibody comprises a CDR-L3 sequence comprising, consisting of, or consisting essentially of a CDR-L3 sequence of a V_{L} sequence of SEQ ID NO: 281. In some aspects, the antibody comprises a CDR-L3 sequence comprising, consisting of, or consisting essentially of a CDR-L3 sequence of a V_{L} sequence of SEQ ID NO: 282. In some aspects, the antibody comprises a CDR-L3 sequence comprising, consisting of, or consisting essentially of a CDR-L3 sequence of a V_{L} sequence of SEQ ID NO: 283. In some aspects, the antibody comprises a CDR-L3 sequence comprising, consisting of, or consisting essentially of a CDR-L3 sequence of a V_{L} sequence of SEQ ID NO: 284. In some aspects, the antibody comprises a CDR-L3 sequence comprising, consisting of, or consisting essentially of a CDR-L3 sequence of a V_{L} sequence of SEQ ID NO: 285. In some aspects, the antibody comprises a CDR-L3 sequence comprising, consisting of, or consisting essentially of a CDR-L3 sequence of a V_{L} sequence of SEQ ID NO: 286. In some aspects, the antibody comprises a CDR-L3 sequence comprising, consisting of, or consisting essentially of a CDR-L3 sequence of a V_{L} sequence of SEQ ID NO: 287. In some aspects, the antibody comprises a CDR-L3 sequence comprising, consisting of, or consisting essentially of a CDR-L3 sequence of a V_{L} sequence of SEQ ID NO: 288. In some aspects, the antibody comprises a CDR-L3 sequence comprising, consisting of, or consisting essentially of a CDR-L3 sequence of a V_{L} sequence of SEQ ID NO: 289. In some aspects, the antibody comprises a CDR-L3 sequence comprising, consisting of, or consisting essentially of a CDR-L3 sequence of a V_{L} sequence of SEQ ID NO: 290. In some aspects, the antibody comprises a CDR-L3 sequence comprising, consisting of, or consisting essentially of a CDR-L3 sequence of a V_{L} sequence of SEQ ID NO: 291. In some aspects, the antibody comprises a CDR-L3 sequence comprising, consisting of, or consisting essentially of a CDR-L3 sequence of a V_{L} sequence of SEQ ID NO: 292. In some aspects, the antibody comprises a CDR-L3 sequence comprising, consisting of, or consisting essentially of a CDR-L3 sequence of a V_{L} sequence of SEQ ID NO: 293. In some aspects, the antibody comprises a CDR-L3 sequence comprising, consisting of, or consisting essentially of a CDR-L3 sequence of a V_{L} sequence of SEQ ID NO: 294. In some aspects, the antibody comprises a CDR-L3 sequence comprising, consisting of, or consisting essentially of a CDR-L3 sequence of a V_{L} sequence of SEQ ID NO: 295. In some aspects, the antibody comprises a CDR-L3 sequence comprising, consisting of, or consisting essentially of a CDR-L3 sequence of a V_{L} sequence of SEQ ID NO: 296. In some aspects, the antibody comprises a CDR-L3 sequence comprising, consisting of, or consisting essentially of a CDR-L3 sequence of a V_{L} sequence of SEQ ID NO: 297. In some aspects, the antibody comprises a CDR-L3 sequence comprising, consisting of, or consisting essentially of a CDR-L3 sequence of a V_{L} sequence of SEQ ID NO: 298. In some aspects, the antibody comprises a CDR-L3 sequence comprising, consisting of, or consisting essentially of a CDR-L3 sequence of a V_{L} sequence of SEQ ID NO: 299.

3.5.2 CDR-L2

In some embodiments, the PD-1 antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided herein. In some aspects, the CDR-L2 sequence is a CDR-L2 sequence of a V_{L} sequence provided in SEQ ID NOs.: 267-299. In some embodiments, the PD-1 antibody comprises a V_{L} sequence comprising a CDR-L2 sequence, wherein the CDR-L2 sequence comprises, consists of, or consists essentially of a CDR-L2 sequence of an illustrative antibody or V_{L} sequence provided in WO 2016/077397.

In some embodiments, the PD-1 antibody comprises a V_{L} sequence comprising a CDR-L2 sequence comprising, consisting of, or consisting essentially of a sequence selected from SEQ ID NOs: 166-172. In some aspects, the antibody comprises a V_{L} sequence comprising a CDR-L2 sequence comprising, consisting of, or consisting essentially of SEQ ID NO: 166. In some aspects, the antibody comprises a V_{L} sequence comprising a CDR-L2 sequence comprising, consisting of, or consisting essentially of SEQ ID NO: 167. In some aspects, the antibody comprises a V_{L} sequence comprising a CDR-L2 sequence comprising, consisting of, or consisting essentially of SEQ ID NO: 168. In some aspects, the antibody comprises a V_{L} sequence comprising a CDR-L2 sequence comprising, consisting of, or consisting essentially of SEQ ID NO: 169. In some aspects, the antibody comprises a V_{L} sequence comprising a CDR-L2 sequence comprising, consisting of, or consisting essentially of SEQ ID NO: 170. In some aspects, the antibody comprises a V_{L} sequence comprising a CDR-L2 sequence comprising, consisting of, or consisting essentially of SEQ ID NO: 171. In some aspects, the antibody comprises a V_{L} sequence comprising a CDR-L2 sequence comprising, consisting of, or consisting essentially of SEQ ID NO: 172.

In some embodiments, the PD-1 antibody comprises a CDR-L2 sequence comprising, consisting of, or consisting essentially of a CDR-L2 sequence of a V_{L} sequence selected from SEQ ID NOs.: 277-299. In some aspects, the antibody comprises a CDR-L2 sequence comprising, consisting of, or consisting essentially of a CDR-L2 sequence of a V_{L} sequence of SEQ ID NO: 277. In some aspects, the antibody comprises a CDR-L2 sequence comprising, consisting of, or consisting essentially of a CDR-L2 sequence of a V_{L} sequence of SEQ ID NO: 278. In some aspects, the antibody comprises a CDR-L2 sequence comprising, consisting of, or consisting essentially of a CDR-L2 sequence of a V_{L} sequence of SEQ ID NO: 279. In some aspects, the antibody comprises a CDR-L2 sequence comprising, consisting of, or consisting essentially of a CDR-L2 sequence of a V_{L} sequence of SEQ ID NO: 280. In some aspects, the antibody comprises a CDR-L2 sequence comprising, consisting of, or consisting essentially of a CDR-L2 sequence of a V_{L} sequence of SEQ ID NO: 281. In some aspects, the antibody comprises a CDR-L2 sequence comprising, consisting of, or consisting essentially of a CDR-L2 sequence of a V_{L} sequence of SEQ ID NO: 282. In some aspects, the antibody comprises a CDR-L2 sequence comprising, consisting of, or consisting essentially of a CDR-L2 sequence of a V_{L} sequence of SEQ ID NO: 283. In some aspects, the antibody comprises a CDR-L2 sequence comprising, consisting of, or consisting essentially of a CDR-L2 sequence of a V_{L} sequence of SEQ ID NO: 284. In some aspects, the antibody comprises a CDR-L2 sequence comprising, consisting of, or consisting essentially of a CDR-L2 sequence of a V_{L} sequence of SEQ ID NO: 285. In some aspects, the antibody comprises a CDR-L2 sequence comprising, consisting of, or consisting essentially of a CDR-L2 sequence of a V_{L} sequence of SEQ ID NO: 286. In some aspects, the antibody comprises a CDR-L2 sequence comprising, consisting of, or consisting essentially of a CDR-L2 sequence of a V_{L} sequence of SEQ ID NO: 287. In some aspects, the antibody comprises a CDR-L2 sequence comprising, consisting of, or consisting essentially of a CDR-L2 sequence of a V_{L} sequence of SEQ ID NO: 288. In some aspects, the antibody comprises a CDR-L2 sequence comprising, consisting of, or consisting essentially of a CDR-L2 sequence of a V_{L} sequence of SEQ ID NO: 289. In some aspects, the antibody comprises a CDR-L2 sequence comprising, consisting of, or consisting essentially of a CDR-L2 sequence of a V_{L} sequence of SEQ ID NO: 290. In some aspects, the antibody comprises a CDR-L2 sequence comprising, consisting of, or consisting essentially of a CDR-L2 sequence of a V_{L} sequence of SEQ ID NO: 291. In some aspects, the antibody comprises a CDR-L2 sequence comprising, consisting of, or consisting essentially of a CDR-L2 sequence of a V_{L} sequence of SEQ ID NO: 292. In some aspects, the antibody comprises a CDR-L2 sequence comprising, consisting of, or consisting essentially of a CDR-L2 sequence of a V_{L} sequence of SEQ ID NO: 293. In some aspects, the antibody comprises a CDR-L2 sequence comprising, consisting of, or consisting essentially of a CDR-L2 sequence of a V_{L} sequence of SEQ ID NO: 294. In some aspects, the antibody comprises a CDR-L2 sequence comprising, consisting of, or consisting essentially of a CDR-L2 sequence of a V_{L} sequence of SEQ ID NO: 295. In some aspects, the antibody comprises a CDR-L2 sequence comprising, consisting of, or consisting essentially of a CDR-L2 sequence of a V_{L} sequence of SEQ ID NO: 296. In some aspects, the antibody comprises a CDR-L2 sequence comprising, consisting of, or consisting essentially of a CDR-L2 sequence of a V_{L} sequence of SEQ ID NO: 297. In some aspects, the antibody comprises a CDR-L2 sequence comprising, consisting of, or consisting essentially of a CDR-L2 sequence of a V_{L} sequence of SEQ ID NO: 298. In some aspects, the antibody comprises a CDR-L2 sequence comprising, consisting of, or consisting essentially of a CDR-L2 sequence of a V_{L} sequence of SEQ ID NO: 299.

3.5.3 CDR-L1

In some embodiments, the PD-1 antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided herein. In some aspects, the CDR-L1 sequence is a CDR-L1 sequence of a V_{L} sequence provided in SEQ ID NOs.: 267-299. In some embodiments, the PD-1 antibody comprises a V_{L} sequence comprising a CDR-L1 sequence, wherein the CDR-L1 sequence comprises, consists of, or consists essentially of a CDR-L1 sequence of an illustrative antibody or V_{L} sequence provided in WO 2016/077397.

In some embodiments, the PD-1 antibody comprises a V_{L} sequence comprising a CDR-L1 sequence comprising, consisting of, or consisting essentially of a sequence selected from SEQ ID NOs: 148-149. In some aspects, the antibody comprises a V_{L} sequence comprising a CDR-L1 sequence comprising, consisting of, or consisting essentially of SEQ ID NO: 148. In some aspects, the antibody comprises a V_{L} sequence comprising a CDR-L1 sequence comprising, consisting of, or consisting essentially of SEQ ID NO: 149.

In some embodiments, the PD-1 antibody comprises a CDR-L1 sequence comprising, consisting of, or consisting essentially of a CDR-L1 sequence of a V_{L} sequence selected from SEQ ID NOs.: 277-299. In some aspects, the antibody comprises a CDR-L1 sequence comprising, consisting of, or consisting essentially of a CDR-L1 sequence of a V_{L} sequence of SEQ ID NO: 277. In some aspects, the antibody comprises a CDR-L1 sequence comprising, consisting of, or consisting essentially of a CDR-L1 sequence of a V_{L} sequence of SEQ ID NO: 278. In some aspects, the antibody comprises a CDR-L1 sequence comprising, consisting of, or consisting essentially of a CDR-L1 sequence of a V_{L} sequence of SEQ ID NO: 279. In some aspects, the antibody comprises a CDR-L1 sequence comprising, consisting of, or consisting essentially of a CDR-L1 sequence of a V_{L} sequence of SEQ ID NO: 280. In some aspects, the antibody comprises a CDR-L1 sequence comprising, consisting of, or consisting essentially of a CDR-L1 sequence of a V_{L} sequence of SEQ ID NO: 281. In some aspects, the antibody comprises a CDR-L1 sequence comprising, consisting of, or consisting essentially of a CDR-L1 sequence of a V_{L} sequence of SEQ ID NO: 282. In some aspects, the antibody comprises a CDR-L1 sequence comprising, consisting of, or consisting essentially of a CDR-L1 sequence of a V_{L} sequence of SEQ ID NO: 283. In some aspects, the antibody comprises a CDR-L1 sequence comprising, consisting of, or consisting essentially of a CDR-L1 sequence of a V_{L} sequence of SEQ ID NO: 284. In some aspects, the antibody comprises a CDR-L1 sequence comprising, consisting of, or consisting essentially of a CDR-L1 sequence of a V_{L} sequence of SEQ ID NO: 285. In some aspects, the antibody comprises a CDR-L1 sequence comprising, consisting of, or consisting essentially of a CDR-L1 sequence of a V_{L} sequence of SEQ ID NO: 286. In some aspects, the antibody comprises a CDR-L1 sequence comprising, consisting of, or consisting essentially of a CDR-L1 sequence of a V_{L} sequence of SEQ ID NO: 287. In some aspects, the antibody comprises a CDR-L1 sequence comprising, consisting of, or consisting essentially of a CDR-L1 sequence of a V_{L} sequence of SEQ ID NO: 288. In some aspects, the antibody comprises a CDR-L1 sequence comprising, consisting of, or consisting essentially of a CDR-L1 sequence of a V_{L} sequence of SEQ ID NO: 289. In some aspects, the antibody comprises a CDR-L1 sequence comprising, consisting of, or consisting essentially of a CDR-L1 sequence of a V_{L} sequence of SEQ ID NO: 290. In some aspects, the antibody comprises a CDR-L1 sequence comprising, consisting of, or consisting essentially of a CDR-L1 sequence of a V_{L} sequence of SEQ ID NO: 291. In some aspects, the antibody comprises a CDR-L1 sequence comprising, consisting of, or consisting essentially of a CDR-L1 sequence of a V_{L} sequence of SEQ ID NO: 292. In some aspects, the antibody comprises a CDR-L1 sequence comprising, consisting of, or consisting essentially of a CDR-L1 sequence of a V_{L} sequence of SEQ ID NO: 293. In some aspects, the antibody comprises a CDR-L1 sequence comprising, consisting of, or consisting essentially of a CDR-L1 sequence of a V_{L} sequence of SEQ ID NO: 294. In some aspects, the antibody comprises a CDR-L1 sequence comprising, consisting of, or consisting essentially of a CDR-L1 sequence of a V_{L} sequence of SEQ ID NO: 295. In some aspects, the antibody comprises a CDR-L1 sequence comprising, consisting of, or consisting essentially of a CDR-L1 sequence of a V_{L} sequence of SEQ ID NO: 296. In some aspects, the antibody comprises a CDR-L1 sequence comprising, consisting of, or consisting essentially of a CDR-L1 sequence of a V_{L} sequence of SEQ ID NO: 297. In some aspects, the antibody comprises a CDR-L1 sequence comprising, consisting of, or consisting essentially of a CDR-L1 sequence of a V_{L} sequence of SEQ ID NO: 298. In some aspects, the antibody comprises a CDR-L1 sequence comprising, consisting of, or consisting essentially of a CDR-L1 sequence of a V_{L} sequence of SEQ ID NO: 299.

3.5.4 CDR-L3+CDR-L2

In some embodiments, the PD-1 antibody comprises a V_{L} sequence comprising a CDR-L3 sequence comprising, consisting of, or consisting essentially of a sequence selected from SEQ ID NOs: 189-190 and a CDR-L2 sequence comprising, consisting of, or consisting essentially of a sequence selected from SEQ ID NOs: 166-172. In some embodiments, the PD-1 antibody comprises a V_{L} sequence comprising a CDR-L3 sequence comprising, consisting of, or consisting essentially of a sequence selected from a CDR-L3 sequence of a V_{L} sequence selected from SEQ ID NOs.: 267-299, and a CDR-L2 sequence comprising, consisting of, or consisting essentially of a sequence selected from a CDR-L2 sequence of a V_{L} sequence selected from SEQ ID NOs.: 267-299. In some aspects, the CDR-L3 sequence and the CDR-L2 sequence are both from a single illustrative V_{L} sequence provided in this disclosure. For example, in some aspects, the CDR-L3 and CDR-L2 are both from a single illustrative V_{L} sequence selected from SEQ ID NOs: 267-299. In some aspects, the CDR-L3 sequence and the CDR-L2 sequence are both from a single illustrative V_{L} sequence provided in WO 2016/077397.

3.5.5 CDR-L3+CDR-L1

In some embodiments, the PD-1 antibody comprises a V_{L} sequence comprising a CDR-L3 sequence comprising, consisting of, or consisting essentially of a sequence selected from SEQ ID NOs: 189-190 and a CDR-L1 sequence comprising, consisting of, or consisting essentially of a sequence selected from SEQ ID NOs: 148-149. In some embodiments, the PD-1 antibody comprises a V_{L} sequence comprising a CDR-L3 sequence comprising, consisting of, or consisting essentially of a sequence selected from a CDR-L3 sequence of a V_{L} sequence selected from SEQ ID NOs.: 267-299, and a CDR-L1 sequence comprising, consisting of, or consisting essentially of a sequence selected from a CDR-L1 sequence of a V_{L} sequence selected from SEQ ID NOs.: 267-299. In some aspects, the CDR-L3 sequence and the CDR-L1 sequence are both from a single illustrative V_{L} sequence provided in this disclosure. For example, in some aspects, the CDR-L3 and CDR-L1 are both from a single illustrative V_{L} sequence selected from SEQ ID NOs: 267-299. In some aspects, the CDR-L3 sequence and the CDR-L1 sequence are both from a single illustrative V_{L} sequence provided in WO 2016/077397.

3.5.6 CDR-L1+CDR-L2

In some embodiments, the PD-1 antibody comprises a V_{L} sequence comprising a CDR-L1 sequence comprising, consisting of, or consisting essentially of a sequence selected from SEQ ID NOs: 148-149 and a CDR-L2 sequence comprising, consisting of, or consisting essentially of a sequence selected from SEQ ID NOs: 166-172. In some embodiments, the PD-1 antibody comprises a V_{L} sequence comprising a CDR-L1 sequence comprising, consisting of, or consisting essentially of a sequence selected from a CDR-L1 sequence of a V_{L} sequence selected from SEQ ID NOs.: 267-299, and a CDR-L2 sequence comprising, consisting of, or consisting essentially of a sequence selected from a CDR-L2 sequence of a V_{L} sequence selected from SEQ ID NOs.: 267-299. In some aspects, the CDR-L1 sequence and the CDR-L2 sequence are both from a single illustrative V_{L} sequence provided in this disclosure. For example, in some aspects, the CDR-L1 and CDR-L2 are both from a single illustrative V_{L} sequence selected from SEQ ID NOs: 267-299. In some aspects, the CDR-L1 sequence and the CDR-L2 sequence are both from a single illustrative V_{L} sequence provided in WO 2016/077397.

3.5.7 CDR-L1+CDR-L2+CDR-L3

In some embodiments, the PD-1 antibody comprises a V_{L} sequence comprising a CDR-L1 sequence comprising, consisting of, or consisting essentially of a sequence selected from SEQ ID NOs: 148-149, a CDR-L2 sequence comprising, consisting of, or consisting essentially of a sequence selected from SEQ ID NOs: 166-172, and a CDR-L3 sequence comprising, consisting of, or consisting essentially of a sequence selected from SEQ ID NOs: 189-190. In some embodiments, the PD-1 antibody comprises a V_{L} sequence comprising a CDR-L1 sequence comprising, consisting of, or consisting essentially of a sequence selected from a CDR-L1 sequence of a V_{L} sequence selected from SEQ ID NOs.: 267-299, a CDR-L2 sequence comprising, consisting of, or consisting essentially of a sequence selected from a CDR-L2 sequence of a V_{L} sequence selected from SEQ ID NOs.: 267-299, and a CDR-L3 sequence comprising, consisting of, or consisting essentially of a sequence selected from a CDR-L3 sequence of a V_{L} sequence selected from SEQ ID NOs.: 267-299. In some aspects, the CDR-L1 sequence, CDR-L2 sequence, and CDR-L3 sequence are all from a single illustrative V_{L} sequence provided in this disclosure. For example, in some aspects, the CDR-L1, CDR-L2, and CDR-L3 are all from a single illustrative V_{L} sequence selected from SEQ ID NOs: 267-299. In some aspects, the CDR-L1 sequence, CDR-L2 sequence, and CDR-L3 sequence are all from a single illustrative V_{L} sequence provided in WO 2016/077397.

3.5.8 Variants of V_{L} Sequences Comprising Illustrative CDR-Ls

In some embodiments, the V_{L} sequences provided herein comprise a variant of an illustrative CDR-L3, CDR-L2, and/or CDR-L1 sequence provided in this disclosure.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

3.6 PD-1 V_{L} Sequences

In some embodiments, the PD-1 antibody comprises, consists of, or consists essentially of a V_{L} sequence provided in SEQ ID NOs.: 267-299.

In some embodiments, the PD-1 antibody comprises a V_{L} sequence comprising, consisting of, or consisting essentially of a sequence selected from SEQ ID NOs: 267-299. In some aspects, the antibody comprises a V_{L} sequence comprising, consisting of, or consisting essentially of SEQ ID NO: 267. In some aspects, the antibody comprises a V_{L} sequence comprising, consisting of, or consisting essentially of SEQ ID NO: 268. In some aspects, the antibody comprises a V_{L} sequence comprising, consisting of, or consisting essentially of SEQ ID NO: 269. In some aspects, the antibody comprises a V_{L} sequence comprising, consisting of, or consisting essentially of SEQ ID NO: 270. In some aspects, the antibody comprises a V_{L} sequence comprising, consisting of, or consisting essentially of SEQ ID NO: 271. In some aspects, the antibody comprises a V_{L} sequence comprising, consisting of, or consisting essentially of SEQ ID NO: 272. In some aspects, the antibody comprises a V_{L} sequence comprising, consisting of, or consisting essentially of SEQ ID NO: 273. In some aspects, the antibody comprises a V_{L} sequence comprising, consisting of, or consisting essentially of SEQ ID NO: 274.

In some aspects, the antibody comprises a V_{L} sequence comprising, consisting of, or consisting essentially of SEQ ID NO: 275. In some aspects, the antibody comprises a V_{L} sequence comprising, consisting of, or consisting essentially of SEQ ID NO: 276. In some aspects, the antibody comprises a V_{L} sequence comprising, consisting of, or consisting essentially of SEQ ID NO: 277. In some aspects, the antibody comprises a V_{L} sequence comprising, consisting of, or consisting essentially of SEQ ID NO: 278. In some aspects, the antibody comprises a V_{L} sequence comprising, consisting of, or consisting essentially of SEQ ID NO: 279. In some aspects, the antibody comprises a V_{L} sequence comprising, consisting of, or consisting essentially of SEQ ID NO: 280. In some aspects, the antibody comprises a V_{L} sequence comprising, consisting of, or consisting essentially of SEQ ID NO: 281. In some aspects, the antibody comprises a V_{L} sequence comprising, consisting of, or consisting essentially of SEQ ID NO: 282. In some aspects, the antibody comprises a V_{L} sequence comprising, consisting of, or consisting essentially of SEQ ID NO: 283. In some aspects, the antibody comprises a V_{L} sequence comprising, consisting of, or consisting essentially of SEQ ID NO: 284. In some aspects, the antibody comprises a V_{L} sequence comprising, consisting of, or consisting essentially of SEQ ID NO: 285. In some aspects, the antibody comprises a V_{L} sequence comprising, consisting of, or consisting essentially of SEQ ID NO: 286. In some aspects, the antibody comprises a V_{L} sequence comprising, consisting of, or consisting essentially of SEQ ID NO: 287. In some aspects, the antibody comprises a V_{L} sequence comprising, consisting of, or consisting essentially of SEQ ID NO: 288. In some aspects, the antibody comprises a V_{L} sequence comprising, consisting of, or consisting essentially of SEQ ID NO: 289. In some aspects, the antibody comprises a V_{L} sequence comprising, consisting of, or consisting essentially of SEQ ID NO: 290. In some aspects, the antibody comprises a V_{L} sequence comprising, consisting of, or consisting essentially of SEQ ID NO: 291. In some aspects, the antibody comprises a V_{L} sequence comprising, consisting of, or consisting essentially of SEQ ID NO: 292. In some aspects, the antibody comprises a V_{L} sequence comprising, consisting of, or consisting essentially of SEQ ID NO: 293. In some aspects, the antibody comprises a V_{L} sequence comprising, consisting of, or consisting essentially of SEQ ID NO: 294. In some aspects, the antibody comprises a V_{L} sequence comprising, consisting of, or consisting essentially of SEQ ID NO: 295. In some aspects, the antibody comprises a V_{L} sequence comprising, consisting of, or consisting essentially of SEQ ID NO: 296. In some aspects, the antibody comprises a V_{L} sequence comprising, consisting of, or consisting essentially of SEQ ID NO: 297. In some aspects, the antibody comprises a V_{L} sequence comprising, consisting of, or consisting essentially of SEQ ID NO: 298. In some aspects, the antibody comprises a V_{L} sequence comprising, consisting of, or consisting essentially of SEQ ID NO: 299.

3.6.1 Variants of V_{L} Sequences

In some embodiments, the V_{L} sequences provided herein comprise, consist of, or consist essentially of a variant of an illustrative V_{L} sequence provided in this disclosure. In some embodiments, the V_{L} sequences provided herein comprise, consist of, or consist essentially of a variant of an illustrative V_{L} sequence provided in WO 2016/077397.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

3.7 PD-1 Pairs

3.7.1 CDR-H3-CDR-L3 Pairs

In some embodiments, the PD-1 antibody comprises a CDR-H3 sequence and a CDR-L3 sequence. In some aspects, the CDR-H3 sequence is part of a V_{H} and the CDR-L3 sequence is part of a V_{L}.

In some aspects, the CDR-H3 sequence is a CDR-H3 sequence comprising, consisting of, or consisting essentially of a sequence selected from SEQ ID NOs: 130-131 and a CDR-H3 sequence of a a V_{H} sequence selected from SEQ ID NOs.: 228-250, and the CDR-L3 sequence is a CDR-L3 sequence comprising, consisting of, or consisting essentially of a sequence selected from SEQ ID NOs: 189-190 and a CDR-L3 sequence of a a V_{L} sequence selected from SEQ ID NOs.: 277-299.

3.7.1.1 Variants of CDR-H3-CDR-L3 Pairs

In some embodiments, the CDR-H3-CDR-L3 pairs provided herein comprise a variant of an illustrative CDR-H3 and/or CDR-L1 sequence provided in this disclosure.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

3.7.2 CDR-H1-CDR-L1 Pairs

In some embodiments, the PD-1 antibody comprises a CDR-H1 sequence and a CDR-L1 sequence. In some aspects, the CDR-H1 sequence is part of a V_{H} and the CDR-L1 sequence is part of a V_{L}.

In some aspects, the CDR-H1 sequence is a Chothia CDR-H1 sequence comprising, consisting of, or consisting essentially of a sequence selected from SEQ ID NOs: 24-38 and a Chothia CDR-H1 sequence of a a V_{H} sequence selected from SEQ ID NOs.: 228-250, and the CDR-L1 sequence is a CDR-L1 sequence comprising, consisting of, or consisting essentially of a sequence selected from SEQ ID NOs: 148-149 and a CDR-L1 sequence of a a V_{L} sequence selected from SEQ ID NOs.: 277-299.

In some aspects, the CDR-H1 sequence is a Kabat CDR-H1 sequence comprising, consisting of, or consisting essentially of a sequence selected from SEQ ID NOs: 59-65 and a Kabat CDR-H1 sequence of a a V_{H} sequence selected from SEQ ID NOs.: 228-250, and the CDR-L1 sequence is a CDR-L1 sequence comprising, consisting of, or consisting essentially of a sequence selected from SEQ ID NOs: 148-149 and a CDR-L1 sequence of a a V_{L} sequence selected from SEQ ID NOs.: 277-299.

3.7.2.1 Variants of CDR-H1-CDR-L1 Pairs

In some embodiments, the CDR-H1-CDR-L1 pairs provided herein comprise a variant of an illustrative CDR-H1 and/or CDR-L1 sequence provided in this disclosure.

In some aspects, the CDR-H1 sequence comprises, consists of, or consists essentially of a variant of an illustrative CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative CDR-H1 sequences provided in this disclosure. In some aspects, the CDR-H1 sequence comprises, consists of, or consists essentially of any of the illustrative CDR-H1 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

3.7.3 CDR-H2-CDR-L2 Pairs

In some embodiments, the PD-1 antibody comprises a CDR-H2 sequence and a CDR-L2 sequence. In some aspects, the CDR-H2 sequence is part of a V_{H} and the CDR-L2 sequence is part of a V_{L}.

In some aspects, the CDR-H2 sequence is a Chothia CDR-H2 sequence comprising, consisting of, or consisting essentially of a sequence selected from SEQ ID NOs: 86-87 and a Chothia CDR-H2 sequence of a a V_{H} sequence selected from SEQ ID NOs.: 228-250, and the CDR-L2 sequence is a CDR-L2 sequence comprising, consisting of, or consisting essentially of a sequence selected from SEQ ID NOs: 166-172 and a CDR-L2 sequence of a a V_{L} sequence selected from SEQ ID NOs.: 277-299.

In some aspects, the CDR-H2 sequence is a Kabat CDR-H2 sequence comprising, consisting of, or consisting essentially of a sequence selected from SEQ ID NOs: 108-109 and a Kabat CDR-H2 sequence of a a V_{H} sequence selected from SEQ ID NOs.: 228-250, and the CDR-L2 sequence is a CDR-L2 sequence comprising, consisting of, or consisting essentially of a sequence selected from SEQ ID NOs: 166-172 and a CDR-L2 sequence of a a V_{L} sequence selected from SEQ ID NOs.: 277-299.

3.7.3.1 Variants of CDR-H2-CDR-L2 Pairs

In some embodiments, the CDR-H2-CDR-L2 pairs provided herein comprise a variant of an illustrative CDR-H2 and/or CDR-L2 sequence provided in this disclosure.

In some aspects, the CDR-H2 sequence comprises, consists of, or consists essentially of a variant of an illustrative CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative CDR-H2 sequences provided in this disclosure. In some aspects, the CDR-H2 sequence comprises, consists of, or consists essentially of any of the illustrative CDR-H2 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

3.7.4 V_{H}-V_{L} Pairs

In some embodiments, the PD-1 antibody comprises a V_{H} sequence and a V_{L} sequence.

In some aspects, the V_{H} sequence is a V_{H} sequence comprising, consisting of, or consisting essentially of SEQ ID NOs: 211-250, and the V_{L} sequence is a V_{L} sequence comprising, consisting of, or consisting essentially of SEQ ID NOs: 267-299.

In some aspects, the V_{H}-V_{L} pairs are selected from SEQ ID NO: 267 and SEQ ID NO: 211; SEQ ID NO: 267 and SEQ ID NO: 212; SEQ ID NO: 267 and SEQ ID NO: 213; SEQ ID NO: 267 and SEQ ID NO: 214; SEQ ID NO: 267 and SEQ ID NO: 215; SEQ ID NO: 267 and SEQ ID NO: 216; SEQ ID NO: 267 and SEQ ID NO: 217; SEQ ID NO: 267 and SEQ ID NO: 218; SEQ ID NO: 267 and SEQ ID NO: 219; SEQ ID NO: 267 and SEQ ID NO: 220; SEQ ID NO: 267 and SEQ ID NO: 221; SEQ ID NO: 267 and SEQ ID NO: 222; SEQ ID NO: 267 and SEQ ID NO: 223; SEQ ID NO: 267 and SEQ ID NO: 224; SEQ ID NO: 267 and SEQ ID NO: 225; SEQ ID NO: 267 and SEQ ID NO: 226; SEQ ID NO: 267 and SEQ ID NO: 227; SEQ ID NO: 267 and SEQ ID NO: 228; SEQ ID NO: 267 and SEQ ID NO: 229; SEQ ID NO: 267 and SEQ ID NO: 230; SEQ ID NO: 267 and SEQ ID NO: 231; SEQ ID NO: 267 and SEQ ID NO: 232; SEQ ID NO: 267 and SEQ ID NO: 233; SEQ ID NO: 267 and SEQ ID NO: 234; SEQ ID NO: 267 and SEQ ID NO: 235; SEQ ID NO: 267 and SEQ ID NO: 236; SEQ ID NO: 267 and SEQ ID NO: 237; SEQ ID NO: 267 and SEQ ID NO: 238; SEQ ID NO: 267 and SEQ ID NO: 239; SEQ ID NO: 267 and SEQ ID NO: 240; SEQ ID NO: 267 and SEQ ID NO: 241; SEQ ID NO: 267 and SEQ ID NO: 242; SEQ ID NO: 267 and SEQ ID NO: 243; SEQ ID NO: 267 and SEQ ID NO: 244; SEQ ID NO: 267 and SEQ ID NO: 245; SEQ ID NO: 267 and SEQ ID NO: 246; SEQ ID NO: 267 and SEQ ID NO: 247; SEQ ID NO: 267 and SEQ ID NO: 248; SEQ ID NO: 267 and SEQ ID NO: 249; and SEQ ID NO: 267 and SEQ ID NO: 250.

In some aspects, the V_{H}-V_{L} pairs are selected from SEQ ID NO: 268 and SEQ ID NO: 211; SEQ ID NO: 268 and SEQ ID NO: 212; SEQ ID NO: 268 and SEQ ID NO: 213; SEQ ID NO: 268 and SEQ ID NO: 214; SEQ ID NO: 268 and SEQ ID NO: 215; SEQ ID NO: 268 and SEQ ID NO: 216; SEQ ID NO: 268 and SEQ ID NO: 217; SEQ ID NO: 268 and SEQ ID NO: 218; SEQ ID NO: 268 and SEQ ID NO: 219; SEQ ID NO: 268 and SEQ ID NO: 220; SEQ ID NO: 268 and SEQ ID NO: 221; SEQ ID NO: 268 and SEQ ID NO: 222; SEQ ID NO: 268 and SEQ ID NO: 223; SEQ ID NO: 268 and SEQ ID NO: 224; SEQ ID NO: 268 and SEQ ID NO: 225; SEQ ID NO: 268 and SEQ ID NO: 226; SEQ ID NO: 268 and SEQ ID NO: 227; SEQ ID NO: 268 and SEQ ID NO: 228; SEQ ID NO: 268 and SEQ ID NO: 229; SEQ ID NO: 268 and SEQ ID NO: 230; SEQ ID NO: 268 and SEQ ID NO: 231; SEQ ID NO: 268 and SEQ ID NO: 232; SEQ ID NO: 268 and SEQ ID NO: 233; SEQ ID NO: 268 and SEQ ID NO: 234; SEQ ID NO: 268 and SEQ ID NO: 235; SEQ ID NO: 268 and SEQ ID NO: 236; SEQ ID NO: 268 and SEQ ID NO: 237; SEQ ID NO: 268 and SEQ ID NO: 238; SEQ ID NO: 268 and SEQ ID NO: 239; SEQ ID NO: 268 and SEQ ID NO: 240; SEQ ID NO: 268 and SEQ ID NO: 241; SEQ ID NO: 268 and SEQ ID NO: 242; SEQ ID NO: 268 and SEQ ID NO: 243; SEQ ID NO: 268 and SEQ ID NO: 244; SEQ ID NO: 268 and SEQ ID NO: 245; SEQ ID NO: 268 and SEQ ID NO: 246; SEQ ID NO: 268 and SEQ ID NO: 247; SEQ ID NO: 268 and SEQ ID NO: 248; SEQ ID NO: 268 and SEQ ID NO: 249; and SEQ ID NO: 268 and SEQ ID NO: 250.

In some aspects, the V_{H}-V_{L} pairs are selected from SEQ ID NO: 269 and SEQ ID NO: 211; SEQ ID NO: 269 and SEQ ID NO: 212; SEQ ID NO: 269 and SEQ ID NO: 213; SEQ ID NO: 269 and SEQ ID NO: 214; SEQ ID NO: 269 and SEQ ID NO: 215; SEQ ID NO: 269 and SEQ ID NO: 216; SEQ ID NO: 269 and SEQ ID NO: 217; SEQ ID NO: 269 and SEQ ID NO: 218; SEQ ID NO: 269 and SEQ ID NO: 219; SEQ ID NO: 269 and SEQ ID NO: 220; SEQ ID NO: 269 and SEQ ID NO: 221; SEQ ID NO: 269 and SEQ ID NO: 222; SEQ ID NO: 269 and SEQ ID NO: 223; SEQ ID NO: 269 and SEQ ID NO: 224; SEQ ID NO: 269 and SEQ ID NO: 225; SEQ ID NO: 269 and SEQ ID NO: 226; SEQ ID NO: 269 and SEQ ID NO: 227; SEQ ID NO: 269 and SEQ ID NO: 228; SEQ ID NO: 269 and SEQ ID NO: 229; SEQ ID NO: 269 and SEQ ID NO: 230; SEQ ID NO: 269 and SEQ ID NO: 231; SEQ ID NO: 269 and SEQ ID NO: 232; SEQ ID NO: 269 and SEQ ID NO: 233; SEQ ID NO: 269 and SEQ ID NO: 234; SEQ ID NO: 269 and SEQ ID NO: 235; SEQ ID NO: 269 and SEQ ID NO: 236; SEQ ID NO: 269 and SEQ ID NO: 237; SEQ ID NO: 269 and SEQ ID NO: 238; SEQ ID NO: 269 and SEQ ID NO: 239; SEQ ID NO: 269 and SEQ ID NO: 240; SEQ ID NO: 269 and SEQ ID NO: 241; SEQ ID NO: 269 and SEQ ID NO: 242; SEQ ID NO: 269 and SEQ ID NO: 243; SEQ ID NO: 269 and SEQ ID NO: 244; SEQ ID NO: 269 and SEQ ID NO: 245; SEQ ID NO: 269 and SEQ ID NO: 246; SEQ ID NO: 269 and SEQ ID NO: 247; SEQ ID NO: 269 and SEQ ID NO: 248; SEQ ID NO: 269 and SEQ ID NO: 249; and SEQ ID NO: 269 and SEQ ID NO: 250.

In some aspects, the V_{H}-V_{L} pairs are selected from SEQ ID NO: 270 and SEQ ID NO: 211; SEQ ID NO: 270 and SEQ ID NO: 212; SEQ ID NO: 270 and SEQ ID NO: 213; SEQ ID NO: 270 and SEQ ID NO: 214; SEQ ID NO: 270 and SEQ ID NO: 215; SEQ ID NO: 270 and SEQ ID NO: 216; SEQ ID NO: 270 and SEQ ID NO: 217; SEQ ID NO: 270 and SEQ ID NO: 218; SEQ ID NO: 270 and SEQ ID NO: 219; SEQ ID NO: 270 and SEQ ID NO: 220; SEQ ID NO: 270 and SEQ ID NO: 221; SEQ ID NO: 270 and SEQ ID NO: 222; SEQ ID NO: 270 and SEQ ID NO: 223; SEQ ID NO: 270 and SEQ ID NO: 224; SEQ ID NO: 270 and SEQ ID NO: 225; SEQ ID NO: 270 and SEQ ID NO: 226; SEQ ID NO: 270 and SEQ ID NO: 227; SEQ ID NO: 270 and SEQ ID NO: 228; SEQ ID NO: 270 and SEQ ID NO: 229; SEQ ID NO: 270 and SEQ ID NO: 230; SEQ ID NO: 270 and SEQ ID NO: 231; SEQ ID NO: 270 and SEQ ID NO: 232; SEQ ID NO: 270 and SEQ ID NO: 233; SEQ ID NO: 270 and SEQ ID NO: 234; SEQ ID NO: 270 and SEQ ID NO: 235; SEQ ID NO: 270 and SEQ ID NO: 236; SEQ ID NO: 270 and SEQ ID NO: 237; SEQ ID NO: 270 and SEQ ID NO: 238; SEQ ID NO: 270 and SEQ ID NO: 239; SEQ ID NO: 270 and SEQ ID NO: 240; SEQ ID NO: 270 and SEQ ID NO: 241; SEQ ID NO: 270 and SEQ ID NO: 242; SEQ ID NO: 270 and SEQ ID NO: 243; SEQ ID NO: 270 and SEQ ID NO: 244; SEQ ID NO: 270 and SEQ ID NO: 245; SEQ ID NO: 270 and SEQ ID NO: 246; SEQ ID NO: 270 and SEQ ID NO: 247; SEQ ID NO: 270 and SEQ ID NO: 248; SEQ ID NO: 270 and SEQ ID NO: 249; and SEQ ID NO: 270 and SEQ ID NO: 250.

In some aspects, the V_{H}-V_{L} pairs are selected from SEQ ID NO: 271 and SEQ ID NO: 211; SEQ ID NO: 271 and SEQ ID NO: 212; SEQ ID NO: 271 and SEQ ID NO: 213; SEQ ID NO: 271 and SEQ ID NO: 214; SEQ ID NO: 271 and SEQ ID NO: 215; SEQ ID NO: 271 and SEQ ID NO: 216; SEQ ID NO: 271 and SEQ ID NO: 217; SEQ ID NO: 271 and SEQ ID NO: 218; SEQ ID NO: 271 and SEQ ID NO: 219; SEQ ID NO: 271 and SEQ ID NO: 220; SEQ ID NO: 271 and SEQ ID NO: 221; SEQ ID NO: 271 and SEQ ID NO: 222; SEQ ID NO: 271 and SEQ ID NO: 223; SEQ ID NO: 271 and SEQ ID NO: 224; SEQ ID NO: 271 and SEQ ID NO: 225; SEQ ID NO: 271 and SEQ ID NO: 226; SEQ ID NO: 271 and SEQ ID NO: 227; SEQ ID NO: 271 and SEQ ID NO: 228; SEQ ID NO: 271 and SEQ ID NO: 229; SEQ ID NO: 271 and SEQ ID NO: 230; SEQ ID NO: 271 and SEQ ID NO: 231; SEQ ID NO: 271 and SEQ ID NO: 232; SEQ ID NO: 271 and SEQ ID NO: 233; SEQ ID NO: 271 and SEQ ID NO: 234; SEQ ID NO: 271 and SEQ ID NO: 235; SEQ ID NO: 271 and SEQ ID NO: 236; SEQ ID NO: 271 and SEQ ID NO: 237; SEQ ID NO: 271 and SEQ ID NO: 238; SEQ ID NO: 271 and SEQ ID NO: 239; SEQ ID NO: 271 and SEQ ID NO: 240; SEQ ID NO: 271 and SEQ ID NO: 241; SEQ ID NO: 271 and SEQ ID NO: 242; SEQ ID NO: 271 and SEQ ID NO: 243; SEQ ID NO: 271 and SEQ ID NO: 244; SEQ ID NO: 271 and SEQ ID NO: 245; SEQ ID NO: 271 and SEQ ID NO: 246; SEQ ID NO: 271 and SEQ ID NO: 247; SEQ ID NO: 271 and SEQ ID NO: 248; SEQ ID NO: 271 and SEQ ID NO: 249; and SEQ ID NO: 271 and SEQ ID NO: 250.

In some aspects, the V_{H}-V_{L} pairs are selected from SEQ ID NO: 272 and SEQ ID NO: 211; SEQ ID NO: 272 and SEQ ID NO: 212; SEQ ID NO: 272 and SEQ ID NO: 213; SEQ ID NO: 272 and SEQ ID NO: 214; SEQ ID NO: 272 and SEQ ID NO: 215; SEQ ID NO: 272 and SEQ ID NO: 216; SEQ ID NO: 272 and SEQ ID NO: 217; SEQ ID NO: 272 and SEQ ID NO: 218; SEQ ID NO: 272 and SEQ ID NO: 219; SEQ ID NO: 272 and SEQ ID NO: 220; SEQ ID NO: 272 and SEQ ID NO: 221; SEQ ID NO: 272 and SEQ ID NO: 222; SEQ ID NO: 272 and SEQ ID NO: 223; SEQ ID NO: 272 and SEQ ID NO: 224; SEQ ID NO: 272 and SEQ ID NO: 225; SEQ ID NO: 272 and SEQ ID NO: 226; SEQ ID NO: 272 and SEQ ID NO: 227; SEQ ID NO: 272 and SEQ ID NO: 228; SEQ ID NO: 272 and SEQ ID NO: 229; SEQ ID NO: 272 and SEQ ID NO: 230; SEQ ID NO: 272 and SEQ ID NO: 231; SEQ ID NO: 272 and SEQ ID NO: 232; SEQ ID NO: 272 and SEQ ID NO: 233; SEQ ID NO: 272 and SEQ ID NO: 234; SEQ ID NO: 272 and SEQ ID NO: 235; SEQ ID NO: 272 and SEQ ID NO: 236; SEQ ID NO: 272 and SEQ ID NO: 237; SEQ ID NO: 272 and SEQ ID NO: 238; SEQ ID NO: 272 and SEQ ID NO: 239; SEQ ID NO: 272 and SEQ ID NO: 240; SEQ ID NO: 272 and SEQ ID NO: 241; SEQ ID NO: 272 and SEQ ID NO: 242; SEQ ID NO: 272 and SEQ ID NO: 243; SEQ ID NO: 272 and SEQ ID NO: 244; SEQ ID NO: 272 and SEQ ID NO: 245; SEQ ID NO: 272 and SEQ ID NO: 246; SEQ ID NO: 272 and SEQ ID NO: 247; SEQ ID NO: 272 and SEQ ID NO: 248; SEQ ID NO: 272 and SEQ ID NO: 249; and SEQ ID NO: 272 and SEQ ID NO: 250.

In some aspects, the V_{H}-V_{L} pairs are selected from SEQ ID NO: 273 and SEQ ID NO: 211; SEQ ID NO: 273 and SEQ ID NO: 212; SEQ ID NO: 273 and SEQ ID NO: 213; SEQ ID NO: 273 and SEQ ID NO: 214; SEQ ID NO: 273 and SEQ ID NO: 215; SEQ ID NO: 273 and SEQ ID NO: 216; SEQ ID NO: 273 and SEQ ID NO: 217; SEQ ID NO: 273 and SEQ ID NO: 218; SEQ ID NO: 273 and SEQ ID NO: 219; SEQ ID NO: 273 and SEQ ID NO: 220; SEQ ID NO: 273 and SEQ ID NO: 221; SEQ ID NO: 273 and SEQ ID NO: 222; SEQ ID NO: 273 and SEQ ID NO: 223; SEQ ID NO: 273 and SEQ ID NO: 224; SEQ ID NO: 273 and SEQ ID NO: 225; SEQ ID NO: 273 and SEQ ID NO: 226; SEQ ID NO: 273 and SEQ ID NO: 227; SEQ ID NO: 273 and SEQ ID NO: 228; SEQ ID NO: 273 and SEQ ID NO: 229; SEQ ID NO: 273 and SEQ ID NO: 230; SEQ ID NO: 273 and SEQ ID NO: 231; SEQ ID NO: 273 and SEQ ID NO: 232; SEQ ID NO: 273 and SEQ ID NO: 233; SEQ ID NO: 273 and SEQ ID NO: 234; SEQ ID NO: 273 and SEQ ID NO: 235; SEQ ID NO: 273 and SEQ ID NO: 236; SEQ ID NO: 273 and SEQ ID NO: 237; SEQ ID NO: 273 and SEQ ID NO: 238; SEQ ID NO: 273 and SEQ ID NO: 239; SEQ ID NO: 273 and SEQ ID NO: 240; SEQ ID NO: 273 and SEQ ID NO: 241; SEQ ID NO: 273 and SEQ ID NO: 242; SEQ ID NO: 273 and SEQ ID NO: 243; SEQ ID NO: 273 and SEQ ID NO: 244; SEQ ID NO: 273 and SEQ ID NO: 245; SEQ ID NO: 273 and SEQ ID NO: 246; SEQ ID NO: 273 and SEQ ID NO: 247; SEQ ID NO: 273 and SEQ ID NO: 248; SEQ ID NO: 273 and SEQ ID NO: 249; and SEQ ID NO: 273 and SEQ ID NO: 250.

In some aspects, the V_{H}-V_{L} pairs are selected from SEQ ID NO: 274 and SEQ ID NO: 211; SEQ ID NO: 274 and SEQ ID NO: 212; SEQ ID NO: 274 and SEQ ID NO: 213; SEQ ID NO: 274 and SEQ ID NO: 214; SEQ ID NO: 274 and SEQ ID NO: 215; SEQ ID NO: 274 and SEQ ID NO: 216; SEQ ID NO: 274 and SEQ ID NO: 217; SEQ ID NO: 274 and SEQ ID NO: 218; SEQ ID NO: 274 and SEQ ID NO: 219; SEQ ID NO: 274 and SEQ ID NO: 220; SEQ ID NO: 274 and SEQ ID NO: 221; SEQ ID NO: 274 and SEQ ID NO: 222; SEQ ID NO: 274 and SEQ ID NO: 223; SEQ ID NO: 274 and SEQ ID NO: 224; SEQ ID NO: 274 and SEQ ID NO: 225; SEQ ID NO: 274 and SEQ ID NO: 226; SEQ ID NO: 274 and SEQ ID NO: 227; SEQ ID NO: 274 and SEQ ID NO: 228; SEQ ID NO: 274 and SEQ ID NO: 229; SEQ ID NO: 274 and SEQ ID NO: 230; SEQ ID NO: 274 and SEQ ID NO: 231; SEQ ID NO: 274 and SEQ ID NO: 232; SEQ ID NO: 274 and SEQ ID NO: 233; SEQ ID NO: 274 and SEQ ID NO: 234; SEQ ID NO: 274 and SEQ ID NO: 235; SEQ ID NO: 274 and SEQ ID NO: 236; SEQ ID NO: 274 and SEQ ID NO: 237; SEQ ID NO: 274 and SEQ ID NO: 238; SEQ ID NO: 274 and SEQ ID NO: 239; SEQ ID NO: 274 and SEQ ID NO: 240; SEQ ID NO: 274 and SEQ ID NO: 241; SEQ ID NO: 274 and SEQ ID NO: 242; SEQ ID NO: 274 and SEQ ID NO: 243; SEQ ID NO: 274 and SEQ ID NO: 244; SEQ ID NO: 274 and SEQ ID NO: 245; SEQ ID NO: 274 and SEQ ID NO: 246; SEQ ID NO: 274 and SEQ ID NO: 247; SEQ ID NO: 274 and SEQ ID NO: 248; SEQ ID NO: 274 and SEQ ID NO: 249; and SEQ ID NO: 274 and SEQ ID NO: 250.

In some aspects, the V_{H}-V_{L} pairs are selected from SEQ ID NO: 275 and SEQ ID NO: 211; SEQ ID NO: 275 and SEQ ID NO: 212; SEQ ID NO: 275 and SEQ ID NO: 213; SEQ ID NO: 275 and SEQ ID NO: 214; SEQ ID NO: 275 and SEQ ID NO: 215; SEQ ID NO: 275 and SEQ ID NO: 216; SEQ ID NO: 275 and SEQ ID NO: 217; SEQ ID NO: 275 and SEQ ID NO: 218; SEQ ID NO: 275 and SEQ ID NO: 219; SEQ ID NO: 275 and SEQ ID NO: 220; SEQ ID NO: 275 and SEQ ID NO: 221; SEQ ID NO: 275 and SEQ ID NO: 222; SEQ ID NO: 275 and SEQ ID NO: 223; SEQ ID NO: 275 and SEQ ID NO: 224; SEQ ID NO: 275 and SEQ ID NO: 225; SEQ ID NO: 275 and SEQ ID NO: 226; SEQ ID NO: 275 and SEQ ID NO: 227; SEQ ID NO: 275 and SEQ ID NO: 228; SEQ ID NO: 275 and SEQ ID NO: 229; SEQ ID NO: 275 and SEQ ID NO: 230; SEQ ID NO: 275 and SEQ ID NO: 231; SEQ ID NO: 275 and SEQ ID NO: 232; SEQ ID NO: 275 and SEQ ID NO: 233; SEQ ID NO: 275 and SEQ ID NO: 234; SEQ ID NO: 275 and SEQ ID NO: 235; SEQ ID NO: 275 and SEQ ID NO: 236; SEQ ID NO: 275 and SEQ ID NO: 237; SEQ ID NO: 275 and SEQ ID NO: 238; SEQ ID NO: 275 and SEQ ID NO: 239; SEQ ID NO: 275 and SEQ ID NO: 240; SEQ ID NO: 275 and SEQ ID NO: 241; SEQ ID NO: 275 and SEQ ID NO: 242; SEQ ID NO: 275 and SEQ ID NO: 243; SEQ ID NO: 275 and SEQ ID NO: 244; SEQ ID NO: 275 and SEQ ID NO: 245; SEQ ID NO: 275 and SEQ ID NO: 246; SEQ ID NO: 275 and SEQ ID NO: 247; SEQ ID NO: 275 and SEQ ID NO: 248; SEQ ID NO: 275 and SEQ ID NO: 249; and SEQ ID NO: 275 and SEQ ID NO: 250.

In some aspects, the V_{H}-V_{L} pairs are selected from SEQ ID NO: 276 and SEQ ID NO: 211; SEQ ID NO: 276 and SEQ ID NO: 212; SEQ ID NO: 276 and SEQ ID NO: 213; SEQ ID NO: 276 and SEQ ID NO: 214; SEQ ID NO: 276 and SEQ ID NO: 215; SEQ ID NO: 276 and SEQ ID NO: 216; SEQ ID NO: 276 and SEQ ID NO: 217; SEQ ID NO: 276 and SEQ ID NO: 218; SEQ ID NO: 276 and SEQ ID NO: 219; SEQ ID NO: 276 and SEQ ID NO: 220; SEQ ID NO: 276 and SEQ ID NO: 221; SEQ ID NO: 276 and SEQ ID NO: 222; SEQ ID NO: 276 and SEQ ID NO: 223; SEQ ID NO: 276 and SEQ ID NO: 224; SEQ ID NO: 276 and SEQ ID NO: 225; SEQ ID NO: 276 and SEQ ID NO: 226; SEQ ID NO: 276 and SEQ ID NO: 227; SEQ ID NO: 276 and SEQ ID NO: 228; SEQ ID NO: 276 and SEQ ID NO: 229; SEQ ID NO: 276 and SEQ ID NO: 230; SEQ ID NO: 276 and SEQ ID NO: 231; SEQ ID NO: 276 and SEQ ID NO: 232; SEQ ID NO: 276 and SEQ ID NO: 233; SEQ ID NO: 276 and SEQ ID NO: 234; SEQ ID NO: 276 and SEQ ID NO: 235; SEQ ID NO: 276 and SEQ ID NO: 236; SEQ ID NO: 276 and SEQ ID NO: 237; SEQ ID NO: 276 and SEQ ID NO: 238; SEQ ID NO: 276 and SEQ ID NO: 239; SEQ ID NO: 276 and SEQ ID NO: 240; SEQ ID NO: 276 and SEQ ID NO: 241; SEQ ID NO: 276 and SEQ ID NO: 242; SEQ ID NO: 276 and SEQ ID NO: 243; SEQ ID NO: 276 and SEQ ID NO: 244; SEQ ID NO: 276 and SEQ ID NO: 245; SEQ ID NO: 276 and SEQ ID NO: 246; SEQ ID NO: 276 and SEQ ID NO: 247; SEQ ID NO: 276 and SEQ ID NO: 248; SEQ ID NO: 276 and SEQ ID NO: 249; and SEQ ID NO: 276 and SEQ ID NO: 250.

In some aspects, the V_{H}-V_{L} pairs are selected from SEQ ID NO: 277 and SEQ ID NO: 211; SEQ ID NO: 277 and SEQ ID NO: 212; SEQ ID NO: 277 and SEQ ID NO: 213; SEQ ID NO: 277 and SEQ ID NO: 214; SEQ ID NO: 277 and SEQ ID NO: 215; SEQ ID NO: 277 and SEQ ID NO: 216; SEQ ID NO: 277 and SEQ ID NO: 217; SEQ ID NO: 277 and SEQ ID NO: 218; SEQ ID NO: 277 and SEQ ID NO: 219; SEQ ID NO: 277 and SEQ ID NO: 220; SEQ ID NO: 277 and SEQ ID NO: 221; SEQ ID NO: 277 and SEQ ID NO: 222; SEQ ID NO: 277 and SEQ ID NO: 223; SEQ ID NO: 277 and SEQ ID NO: 224; SEQ ID NO: 277 and SEQ ID NO: 225; SEQ ID NO: 277 and SEQ ID NO: 226; SEQ ID NO: 277 and SEQ ID NO: 227; SEQ ID NO: 277 and SEQ ID NO: 228; SEQ ID NO: 277 and SEQ ID NO: 229; SEQ ID NO: 277 and SEQ ID NO: 230; SEQ ID NO: 277 and SEQ ID NO: 231; SEQ ID NO: 277 and SEQ ID NO: 232; SEQ ID NO: 277 and SEQ ID NO: 233; SEQ ID NO: 277 and SEQ ID NO: 234; SEQ ID NO: 277 and SEQ ID NO: 235; SEQ ID NO: 277 and SEQ ID NO: 236; SEQ ID NO: 277 and SEQ ID NO: 237; SEQ ID NO: 277 and SEQ ID NO: 238; SEQ ID NO: 277 and SEQ ID NO: 239; SEQ ID NO: 277 and SEQ ID NO: 240; SEQ ID NO: 277 and SEQ ID NO: 241; SEQ ID NO: 277 and SEQ ID NO: 242; SEQ ID NO: 277 and SEQ ID NO: 243; SEQ ID NO: 277 and SEQ ID NO: 244; SEQ ID NO: 277 and SEQ ID NO: 245; SEQ ID NO: 277 and SEQ ID NO: 246; SEQ ID NO: 277 and SEQ ID NO: 247; SEQ ID NO: 277 and SEQ ID NO: 248; SEQ ID NO: 277 and SEQ ID NO: 249; and SEQ ID NO: 277 and SEQ ID NO: 250.

In some aspects, the V_{H}-V_{L} pairs are selected from SEQ ID NO: 278 and SEQ ID NO: 211; SEQ ID NO: 278 and SEQ ID NO: 212; SEQ ID NO: 278 and SEQ ID NO: 213; SEQ ID NO: 278 and SEQ ID NO: 214; SEQ ID NO: 278 and SEQ ID NO: 215; SEQ ID NO: 278 and SEQ ID NO: 216; SEQ ID NO: 278 and SEQ ID NO: 217; SEQ ID NO: 278 and SEQ ID NO: 218; SEQ ID NO: 278 and SEQ ID NO: 219; SEQ ID NO: 278 and SEQ ID NO: 220; SEQ ID NO: 278 and SEQ ID NO: 221; SEQ ID NO: 278 and SEQ ID NO: 222; SEQ ID NO: 278 and SEQ ID NO: 223; SEQ ID NO: 278 and SEQ ID NO: 224; SEQ ID NO: 278 and SEQ ID NO: 225; SEQ ID NO: 278 and SEQ ID NO: 226; SEQ ID NO: 278 and SEQ ID NO: 227; SEQ ID NO: 278 and SEQ ID NO: 228; SEQ ID NO: 278 and SEQ ID NO: 229; SEQ ID NO: 278 and SEQ ID NO: 230; SEQ ID NO: 278 and SEQ ID NO: 231; SEQ ID NO: 278 and SEQ ID NO: 232; SEQ ID NO: 278 and SEQ ID NO: 233; SEQ ID NO: 278 and SEQ ID NO: 234; SEQ ID NO: 278 and SEQ ID NO: 235; SEQ ID NO: 278 and SEQ ID NO: 236; SEQ ID NO: 278 and SEQ ID NO: 237; SEQ ID NO: 278 and SEQ ID NO: 238; SEQ ID NO: 278 and SEQ ID NO: 239; SEQ ID NO: 278 and SEQ ID NO: 240; SEQ ID NO: 278 and SEQ ID NO: 241; SEQ ID NO: 278 and SEQ ID NO: 242; SEQ ID NO: 278 and SEQ ID NO: 243; SEQ ID NO: 278 and SEQ ID NO: 244; SEQ ID NO: 278 and SEQ ID NO: 245; SEQ ID NO: 278 and SEQ ID NO: 246; SEQ ID NO: 278 and SEQ ID NO: 247; SEQ ID NO: 278 and SEQ ID NO: 248; SEQ ID NO: 278 and SEQ ID NO: 249; and SEQ ID NO: 278 and SEQ ID NO: 250.

In some aspects, the V_{H}-V_{L} pairs are selected from SEQ ID NO: 279 and SEQ ID NO: 211; SEQ ID NO: 279 and SEQ ID NO: 212; SEQ ID NO: 279 and SEQ ID NO: 213; SEQ ID NO: 279 and SEQ ID NO: 214; SEQ ID NO: 279 and SEQ ID NO: 215; SEQ ID NO: 279 and SEQ ID NO: 216; SEQ ID NO: 279 and SEQ ID NO: 217; SEQ ID NO: 279 and SEQ ID NO: 218; SEQ ID NO: 279 and SEQ ID NO: 219; SEQ ID NO: 279 and SEQ ID NO: 220; SEQ ID NO: 279 and SEQ ID NO: 221; SEQ ID NO: 279 and SEQ ID NO: 222; SEQ ID NO: 279 and SEQ ID NO: 223; SEQ ID NO: 279 and SEQ ID NO: 224; SEQ ID NO: 279 and SEQ ID NO: 225; SEQ ID NO: 279 and SEQ ID NO: 226; SEQ ID NO: 279 and SEQ ID NO: 227; SEQ ID NO: 279 and SEQ ID NO: 228; SEQ ID NO: 279 and SEQ ID NO: 229; SEQ ID NO: 279 and SEQ ID NO: 230; SEQ ID NO: 279 and SEQ ID NO: 231; SEQ ID NO: 279 and SEQ ID NO: 232; SEQ ID NO: 279 and SEQ ID NO: 233; SEQ ID NO: 279 and SEQ ID NO: 234; SEQ ID NO: 279 and SEQ ID NO: 235; SEQ ID NO: 279 and SEQ ID NO: 236; SEQ ID NO: 279 and SEQ ID NO: 237; SEQ ID NO: 279 and SEQ ID NO: 238; SEQ ID NO: 279 and SEQ ID NO: 239; SEQ ID NO: 279 and SEQ ID NO: 240; SEQ ID NO: 279 and SEQ ID NO: 241; SEQ ID NO: 279 and SEQ ID NO: 242; SEQ ID NO: 279 and SEQ ID NO: 243; SEQ ID NO: 279 and SEQ ID NO: 244; SEQ ID NO: 279 and SEQ ID NO: 245; SEQ ID NO: 279 and SEQ ID NO: 246; SEQ ID NO: 279 and SEQ ID NO: 247; SEQ ID NO: 279 and SEQ ID NO: 248; SEQ ID NO: 279 and SEQ ID NO: 249; and SEQ ID NO: 279 and SEQ ID NO: 250.

In some aspects, the V_{H}-V_{L} pairs are selected from SEQ ID NO: 280 and SEQ ID NO: 211; SEQ ID NO: 280 and SEQ ID NO: 212; SEQ ID NO: 280 and SEQ ID NO: 213; SEQ ID NO: 280 and SEQ ID NO: 214; SEQ ID NO: 280 and SEQ ID NO: 215; SEQ ID NO: 280 and SEQ ID NO: 216; SEQ ID NO: 280 and SEQ ID NO: 217; SEQ ID NO: 280 and SEQ ID NO: 218; SEQ ID NO: 280 and SEQ ID NO: 219; SEQ ID NO: 280 and SEQ ID NO: 220; SEQ ID NO: 280 and SEQ ID NO: 221; SEQ ID NO: 280 and SEQ ID NO: 222; SEQ ID NO: 280 and SEQ ID NO: 223; SEQ ID NO: 280 and SEQ ID NO: 224; SEQ ID NO: 280 and SEQ ID NO: 225; SEQ ID NO: 280 and SEQ ID NO: 226; SEQ ID NO: 280 and SEQ ID NO: 227; SEQ ID NO: 280 and SEQ ID NO: 228; SEQ ID NO: 280 and SEQ ID NO: 229; SEQ ID NO: 280 and SEQ ID NO: 230; SEQ ID NO: 280 and SEQ ID NO: 231; SEQ ID NO: 280 and SEQ ID NO: 232; SEQ ID NO: 280 and SEQ ID NO: 233; SEQ ID NO: 280 and SEQ ID NO: 234; SEQ ID NO: 280 and SEQ ID NO: 235; SEQ ID NO: 280 and SEQ ID NO: 236; SEQ ID NO: 280 and SEQ ID NO: 237; SEQ ID NO: 280 and SEQ ID NO: 238; SEQ ID NO: 280 and SEQ ID NO: 239; SEQ ID NO: 280 and SEQ ID NO: 240; SEQ ID NO: 280 and SEQ ID NO: 241; SEQ ID NO: 280 and SEQ ID NO: 242; SEQ ID NO: 280 and SEQ ID NO: 243; SEQ ID NO: 280 and SEQ ID NO: 244; SEQ ID NO: 280 and SEQ ID NO: 245; SEQ ID NO: 280 and SEQ ID NO: 246; SEQ ID NO: 280 and SEQ ID NO: 247; SEQ ID NO: 280 and SEQ ID NO: 248; SEQ ID NO: 280 and SEQ ID NO: 249; and SEQ ID NO: 280 and SEQ ID NO: 250.

In some aspects, the V_{H}-V_{L} pairs are selected from SEQ ID NO: 281 and SEQ ID NO: 211; SEQ ID NO: 281 and SEQ ID NO: 212; SEQ ID NO: 281 and SEQ ID NO: 213; SEQ ID NO: 281 and SEQ ID NO: 214; SEQ ID NO: 281 and SEQ ID NO: 215; SEQ ID NO: 281 and SEQ ID NO: 216; SEQ ID NO: 281 and SEQ ID NO: 217; SEQ ID NO: 281 and SEQ ID NO: 218; SEQ ID NO: 281 and SEQ ID NO: 219; SEQ ID NO: 281 and SEQ ID NO: 220; SEQ ID NO: 281 and SEQ ID NO: 221; SEQ ID NO: 281 and SEQ ID NO: 222; SEQ ID NO: 281 and SEQ ID NO: 223; SEQ ID NO: 281 and SEQ ID NO: 224; SEQ ID NO: 281 and SEQ ID NO: 225; SEQ ID NO: 281 and SEQ ID NO: 226; SEQ ID NO: 281 and SEQ ID NO: 227; SEQ ID NO: 281 and SEQ ID NO: 228; SEQ ID NO: 281 and SEQ ID NO: 229; SEQ ID NO: 281 and SEQ ID NO: 230; SEQ ID NO: 281 and SEQ ID NO: 231; SEQ ID NO: 281 and SEQ ID NO: 232; SEQ ID NO: 281 and SEQ ID NO: 233; SEQ ID NO: 281 and SEQ ID NO: 234; SEQ ID NO: 281 and SEQ ID NO: 235; SEQ ID NO: 281 and SEQ ID NO: 236; SEQ ID NO: 281 and SEQ ID NO: 237; SEQ ID NO: 281 and SEQ ID NO: 238; SEQ ID NO: 281 and SEQ ID NO: 239; SEQ ID NO: 281 and SEQ ID NO: 240; SEQ ID NO: 281 and SEQ ID NO: 241; SEQ ID NO: 281 and SEQ ID NO: 242; SEQ ID NO: 281 and SEQ ID NO: 243; SEQ ID NO: 281 and SEQ ID NO: 244; SEQ ID NO: 281 and SEQ ID NO: 245; SEQ ID NO: 281 and SEQ ID NO: 246; SEQ ID NO: 281 and SEQ ID NO: 247; SEQ ID NO: 281 and SEQ ID NO: 248; SEQ ID NO: 281 and SEQ ID NO: 249; and SEQ ID NO: 281 and SEQ ID NO: 250.

In some aspects, the V_{H}-V_{L} pairs are selected from SEQ ID NO: 282 and SEQ ID NO: 211; SEQ ID NO: 282 and SEQ ID NO: 212; SEQ ID NO: 282 and SEQ ID NO: 213; SEQ ID NO: 282 and SEQ ID NO: 214; SEQ ID NO: 282 and SEQ ID NO: 215; SEQ ID NO: 282 and SEQ ID NO: 216; SEQ ID NO: 282 and SEQ ID NO: 217; SEQ ID NO: 282 and SEQ ID NO: 218; SEQ ID NO: 282 and SEQ ID NO: 219; SEQ ID NO: 282 and SEQ ID NO: 220; SEQ ID NO: 282 and SEQ ID NO: 221; SEQ ID NO: 282 and SEQ ID NO: 222; SEQ ID NO: 282 and SEQ ID NO: 223; SEQ ID NO: 282 and SEQ ID NO: 224; SEQ ID NO: 282 and SEQ ID NO: 225; SEQ ID NO: 282 and SEQ ID NO: 226; SEQ ID NO: 282 and SEQ ID NO: 227; SEQ ID NO: 282 and SEQ ID NO: 228; SEQ ID NO: 282 and SEQ ID NO: 229; SEQ ID NO: 282 and SEQ ID NO: 230; SEQ ID NO: 282 and SEQ ID NO: 231; SEQ ID NO: 282 and SEQ ID NO: 232; SEQ ID NO: 282 and SEQ ID NO: 233; SEQ ID NO: 282 and SEQ ID NO: 234; SEQ ID NO: 282 and SEQ ID NO: 235; SEQ ID NO: 282 and SEQ ID NO: 236; SEQ ID NO: 282 and SEQ ID NO: 237; SEQ ID NO: 282 and SEQ ID NO: 238; SEQ ID NO: 282 and SEQ ID NO: 239; SEQ ID NO: 282 and SEQ ID NO: 240; SEQ ID NO: 282 and SEQ ID NO: 241; SEQ ID NO: 282 and SEQ ID NO: 242; SEQ ID NO: 282 and SEQ ID NO: 243; SEQ ID NO: 282 and SEQ ID NO: 244; SEQ ID NO: 282 and SEQ ID NO: 245; SEQ ID NO: 282 and SEQ ID NO: 246; SEQ ID NO: 282 and SEQ ID NO: 247; SEQ ID NO: 282 and SEQ ID NO: 248; SEQ ID NO: 282 and SEQ ID NO: 249; and SEQ ID NO: 282 and SEQ ID NO: 250.

In some aspects, the V_{H}-V_{L} pairs are selected from SEQ ID NO: 283 and SEQ ID NO: 211; SEQ ID NO: 283 and SEQ ID NO: 212; SEQ ID NO: 283 and SEQ ID NO: 213; SEQ ID NO: 283 and SEQ ID NO: 214; SEQ ID NO: 283 and SEQ ID NO: 215; SEQ ID NO: 283 and SEQ ID NO: 216; SEQ ID NO: 283 and SEQ ID NO: 217; SEQ ID NO: 283 and SEQ ID NO: 218; SEQ ID NO: 283 and SEQ ID NO: 219; SEQ ID NO: 283 and SEQ ID NO: 220; SEQ ID NO: 283 and SEQ ID NO: 221; SEQ ID NO: 283 and SEQ ID NO: 222; SEQ ID NO: 283 and SEQ ID NO: 223; SEQ ID NO: 283 and SEQ ID NO: 224; SEQ ID NO: 283 and SEQ ID NO: 225; SEQ ID NO: 283 and SEQ ID NO: 226; SEQ ID NO: 283 and SEQ ID NO: 227; SEQ ID NO: 283 and SEQ ID NO: 228; SEQ ID NO: 283 and SEQ ID NO: 229; SEQ ID NO: 283 and SEQ ID NO: 230; SEQ ID NO: 283 and SEQ ID NO: 231; SEQ ID NO: 283 and SEQ ID NO: 232; SEQ ID NO: 283 and SEQ ID NO: 233; SEQ ID NO: 283 and SEQ ID NO: 234; SEQ ID NO: 283 and SEQ ID NO: 235; SEQ ID NO: 283 and SEQ ID NO: 236; SEQ ID NO: 283 and SEQ ID NO: 237; SEQ ID NO: 283 and SEQ ID NO: 238; SEQ ID NO: 283 and SEQ ID NO: 239; SEQ ID NO: 283 and SEQ ID NO: 240; SEQ ID NO: 283 and SEQ ID NO: 241; SEQ ID NO: 283 and SEQ ID NO: 242; SEQ ID NO: 283 and SEQ ID NO: 243; SEQ ID NO: 283 and SEQ ID NO: 244; SEQ ID NO: 283 and SEQ ID NO: 245; SEQ ID NO: 283 and SEQ ID NO: 246; SEQ ID NO: 283 and SEQ ID NO: 247; SEQ ID NO: 283 and SEQ ID NO: 248; SEQ ID NO: 283 and SEQ ID NO: 249; and SEQ ID NO: 283 and SEQ ID NO: 250.

In some aspects, the V_{H}-V_{L} pairs are selected from SEQ ID NO: 284 and SEQ ID NO: 211; SEQ ID NO: 284 and SEQ ID NO: 212; SEQ ID NO: 284 and SEQ ID NO: 213; SEQ ID NO: 284 and SEQ ID NO: 214; SEQ ID NO: 284 and SEQ ID NO: 215; SEQ ID NO: 284 and SEQ ID NO: 216; SEQ ID NO: 284 and SEQ ID NO: 217; SEQ ID NO: 284 and SEQ ID NO: 218; SEQ ID NO: 284 and SEQ ID NO: 219; SEQ ID NO: 284 and SEQ ID NO: 220; SEQ ID NO: 284 and SEQ ID NO: 221; SEQ ID NO: 284 and SEQ ID NO: 222; SEQ ID NO: 284 and SEQ ID NO: 223; SEQ ID NO: 284 and SEQ ID NO: 224; SEQ ID NO: 284 and SEQ ID NO: 225; SEQ ID NO: 284 and SEQ ID NO: 226; SEQ ID NO: 284 and SEQ ID NO: 227; SEQ ID NO: 284 and SEQ ID NO: 228; SEQ ID NO: 284 and SEQ ID NO: 229; SEQ ID NO: 284 and SEQ ID NO: 230; SEQ ID NO: 284 and SEQ ID NO: 231; SEQ ID NO: 284 and SEQ ID NO: 232; SEQ ID NO: 284 and SEQ ID NO: 233; SEQ ID NO: 284 and SEQ ID NO: 234; SEQ ID NO: 284 and SEQ ID NO: 235; SEQ ID NO: 284 and SEQ ID NO: 236; SEQ ID NO: 284 and SEQ ID NO: 237; SEQ ID NO: 284 and SEQ ID NO: 238; SEQ ID NO: 284 and SEQ ID NO: 239; SEQ ID NO: 284 and SEQ ID NO: 240; SEQ ID NO: 284 and SEQ ID NO: 241; SEQ ID NO: 284 and SEQ ID NO: 242; SEQ ID NO: 284 and SEQ ID NO: 243; SEQ ID NO: 284 and SEQ ID NO: 244; SEQ ID NO: 284 and SEQ ID NO: 245; SEQ ID NO: 284 and SEQ ID NO: 246; SEQ ID NO: 284 and SEQ ID NO: 247; SEQ ID NO: 284 and SEQ ID NO: 248; SEQ ID NO: 284 and SEQ ID NO: 249; and SEQ ID NO: 284 and SEQ ID NO: 250.

In some aspects, the V_{H}-V_{L} pairs are selected from SEQ ID NO: 285 and SEQ ID NO: 211; SEQ ID NO: 285 and SEQ ID NO: 212; SEQ ID NO: 285 and SEQ ID NO: 213; SEQ ID NO: 285 and SEQ ID NO: 214; SEQ ID NO: 285 and SEQ ID NO: 215; SEQ ID NO: 285 and SEQ ID NO: 216; SEQ ID NO: 285 and SEQ ID NO: 217; SEQ ID NO: 285 and SEQ ID NO: 218; SEQ ID NO: 285 and SEQ ID NO: 219; SEQ ID NO: 285 and SEQ ID NO: 220; SEQ ID NO: 285 and SEQ ID NO: 221; SEQ ID NO: 285 and SEQ ID NO: 222; SEQ ID NO: 285 and SEQ ID NO: 223; SEQ ID NO: 285 and SEQ ID NO: 224; SEQ ID NO: 285 and SEQ ID NO: 225; SEQ ID NO: 285 and SEQ ID NO: 226; SEQ ID NO: 285 and SEQ ID NO: 227; SEQ ID NO: 285 and SEQ ID NO: 228; SEQ ID NO: 285 and SEQ ID NO: 229; SEQ ID NO: 285 and SEQ ID NO: 230; SEQ ID NO: 285 and SEQ ID NO: 231; SEQ ID NO: 285 and SEQ ID NO: 232; SEQ ID NO: 285 and SEQ ID NO: 233; SEQ ID NO: 285 and SEQ ID NO: 234; SEQ ID NO: 285 and SEQ ID NO: 235; SEQ ID NO: 285 and SEQ ID NO: 236; SEQ ID NO: 285 and SEQ ID NO: 237; SEQ ID NO: 285 and SEQ ID NO: 238; SEQ ID NO: 285 and SEQ ID NO: 239; SEQ ID NO: 285 and SEQ ID NO: 240; SEQ ID NO: 285 and SEQ ID NO: 241; SEQ ID NO: 285 and SEQ ID NO: 242; SEQ ID NO: 285 and SEQ ID NO: 243; SEQ ID NO: 285 and SEQ ID NO: 244; SEQ ID NO: 285 and SEQ ID NO: 245; SEQ ID NO: 285 and SEQ ID NO: 246; SEQ ID NO: 285 and SEQ ID NO: 247; SEQ ID NO: 285 and SEQ ID NO: 248; SEQ ID NO: 285 and SEQ ID NO: 249; and SEQ ID NO: 285 and SEQ ID NO: 250.

In some aspects, the V_{H}-V_{L} pairs are selected from SEQ ID NO: 286 and SEQ ID NO: 211; SEQ ID NO: 286 and SEQ ID NO: 212; SEQ ID NO: 286 and SEQ ID NO: 213; SEQ ID NO: 286 and SEQ ID NO: 214; SEQ ID NO: 286 and SEQ ID NO: 215; SEQ ID NO: 286 and SEQ ID NO: 216; SEQ ID NO: 286 and SEQ ID NO: 217; SEQ ID NO: 286 and SEQ ID NO: 218; SEQ ID NO: 286 and SEQ ID NO: 219; SEQ ID NO: 286 and SEQ ID NO: 220; SEQ ID NO: 286 and SEQ ID NO: 221; SEQ ID NO: 286 and SEQ ID NO: 222; SEQ ID NO: 286 and SEQ ID NO: 223; SEQ ID NO: 286 and SEQ ID NO: 224; SEQ ID NO: 286 and SEQ ID NO: 225; SEQ ID NO: 286 and SEQ ID NO: 226; SEQ ID NO: 286 and SEQ ID NO: 227; SEQ ID NO: 286 and SEQ ID NO: 228; SEQ ID NO: 286 and SEQ ID NO: 229; SEQ ID NO: 286 and SEQ ID NO: 230; SEQ ID NO: 286 and SEQ ID NO: 231; SEQ ID NO: 286 and SEQ ID NO: 232; SEQ ID NO: 286 and SEQ ID NO: 233; SEQ ID NO: 286 and SEQ ID NO: 234; SEQ ID NO: 286 and SEQ ID NO: 235; SEQ ID NO: 286 and SEQ ID NO: 236; SEQ ID NO: 286 and SEQ ID NO: 237; SEQ ID NO: 286 and SEQ ID NO: 238; SEQ ID NO: 286 and SEQ ID NO: 239; SEQ ID NO: 286 and SEQ ID NO: 240; SEQ ID NO: 286 and SEQ ID NO: 241; SEQ ID NO: 286 and SEQ ID NO: 242; SEQ ID NO: 286 and SEQ ID NO: 243; SEQ ID NO: 286 and SEQ ID NO: 244; SEQ ID NO: 286 and SEQ ID NO: 245; SEQ ID NO: 286 and SEQ ID NO: 246; SEQ ID NO: 286 and SEQ ID NO: 247; SEQ ID NO: 286 and SEQ ID NO: 248; SEQ ID NO: 286 and SEQ ID NO: 249; and SEQ ID NO: 286 and SEQ ID NO: 250.

In some aspects, the V_{H}-V_{L} pairs are selected from SEQ ID NO: 287 and SEQ ID NO: 211; SEQ ID NO: 287 and SEQ ID NO: 212; SEQ ID NO: 287 and SEQ ID NO: 213; SEQ ID NO: 287 and SEQ ID NO: 214; SEQ ID NO: 287 and SEQ ID NO: 215; SEQ ID NO: 287 and SEQ ID NO: 216; SEQ ID NO: 287 and SEQ ID NO: 217; SEQ ID NO: 287 and SEQ ID NO: 218; SEQ ID NO: 287 and SEQ ID NO: 219; SEQ ID NO: 287 and SEQ ID NO: 220; SEQ ID NO: 287 and SEQ ID NO: 221; SEQ ID NO: 287 and SEQ ID NO: 222; SEQ ID NO: 287 and SEQ ID NO: 223; SEQ ID NO: 287 and SEQ ID NO: 224; SEQ ID NO: 287 and SEQ ID NO: 225; SEQ ID NO: 287 and SEQ ID NO: 226; SEQ ID NO: 287 and SEQ ID NO: 227; SEQ ID NO: 287 and SEQ ID NO: 228; SEQ ID NO: 287 and SEQ ID NO: 229; SEQ ID NO: 287 and SEQ ID NO: 230; SEQ ID NO: 287 and SEQ ID NO: 231; SEQ ID NO: 287 and SEQ ID NO: 232; SEQ ID NO: 287 and SEQ ID NO: 233; SEQ ID NO: 287 and SEQ ID NO: 234; SEQ ID NO: 287 and SEQ ID NO: 235; SEQ ID NO: 287 and SEQ ID NO: 236; SEQ ID NO: 287 and SEQ ID NO: 237; SEQ ID NO: 287 and SEQ ID NO: 238; SEQ ID NO: 287 and SEQ ID NO: 239; SEQ ID NO: 287 and SEQ ID NO: 240; SEQ ID NO: 287 and SEQ ID NO: 241; SEQ ID NO: 287 and SEQ ID NO: 242; SEQ ID NO: 287 and SEQ ID NO: 243; SEQ ID NO: 287 and SEQ ID NO: 244; SEQ ID NO: 287 and SEQ ID NO: 245; SEQ ID NO: 287 and SEQ ID NO: 246; SEQ ID NO: 287 and SEQ ID NO: 247; SEQ ID NO: 287 and SEQ ID NO: 248; SEQ ID NO: 287 and SEQ ID NO: 249; and SEQ ID NO: 287 and SEQ ID NO: 250.

In some aspects, the V_{H}-V_{L} pairs are selected from SEQ ID NO: 288 and SEQ ID NO: 211; SEQ ID NO: 288 and SEQ ID NO: 212; SEQ ID NO: 288 and SEQ ID NO: 213; SEQ ID NO: 288 and SEQ ID NO: 214; SEQ ID NO: 288 and SEQ ID NO: 215; SEQ ID NO: 288 and SEQ ID NO: 216; SEQ ID NO: 288 and SEQ ID NO: 217; SEQ ID NO: 288 and SEQ ID NO: 218; SEQ ID NO: 288 and SEQ ID NO: 219; SEQ ID NO: 288 and SEQ ID NO: 220; SEQ ID NO: 288 and SEQ ID NO: 221; SEQ ID NO: 288 and SEQ ID NO: 222; SEQ ID NO: 288 and SEQ ID NO: 223; SEQ ID NO: 288 and SEQ ID NO: 224; SEQ ID NO: 288 and SEQ ID NO: 225; SEQ ID NO: 288 and SEQ ID NO: 226; SEQ ID NO: 288 and SEQ ID NO: 227; SEQ ID NO: 288 and SEQ ID NO: 228; SEQ ID NO: 288 and SEQ ID NO: 229; SEQ ID NO: 288 and SEQ ID NO: 230; SEQ ID NO: 288 and SEQ ID NO: 231; SEQ ID NO: 288 and SEQ ID NO: 232; SEQ ID NO: 288 and SEQ ID NO: 233; SEQ ID NO: 288 and SEQ ID NO: 234; SEQ ID NO: 288 and SEQ ID NO: 235; SEQ ID NO: 288 and SEQ ID NO: 236; SEQ ID NO: 288 and SEQ ID NO: 237; SEQ ID NO: 288 and SEQ ID NO: 238; SEQ ID NO: 288 and SEQ ID NO: 239; SEQ ID NO: 288 and SEQ ID NO: 240; SEQ ID NO: 288 and SEQ ID NO: 241; SEQ ID NO: 288 and SEQ ID NO: 242; SEQ ID NO: 288 and SEQ ID NO: 243; SEQ ID NO: 288 and SEQ ID NO: 244; SEQ ID NO: 288 and SEQ ID NO: 245; SEQ ID NO: 288 and SEQ ID NO: 246; SEQ ID NO: 288 and SEQ ID NO: 247; SEQ ID NO: 288 and SEQ ID NO: 248; SEQ ID NO: 288 and SEQ ID NO: 249; and SEQ ID NO: 288 and SEQ ID NO: 250.

In some aspects, the V_{H}-V_{L} pairs are selected from SEQ ID NO: 289 and SEQ ID NO: 211; SEQ ID NO: 289 and SEQ ID NO: 212; SEQ ID NO: 289 and SEQ ID NO: 213; SEQ ID NO: 289 and SEQ ID NO: 214; SEQ ID NO: 289 and SEQ ID NO: 215; SEQ ID NO: 289 and SEQ ID NO: 216; SEQ ID NO: 289 and SEQ ID NO: 217; SEQ ID NO: 289 and SEQ ID NO: 218; SEQ ID NO: 289 and SEQ ID NO: 219; SEQ ID NO: 289 and SEQ ID NO: 220; SEQ ID NO: 289 and SEQ ID NO: 221; SEQ ID NO: 289 and SEQ ID NO: 222; SEQ ID NO: 289 and SEQ ID NO: 223; SEQ ID NO: 289 and SEQ ID NO: 224; SEQ ID NO: 289 and SEQ ID NO: 225; SEQ ID NO: 289 and SEQ ID NO: 226; SEQ ID NO: 289 and SEQ ID NO: 227; SEQ ID NO: 289 and SEQ ID NO: 228; SEQ ID NO: 289 and SEQ ID NO: 229; SEQ ID NO: 289 and SEQ ID NO: 230; SEQ ID NO: 289 and SEQ ID NO: 231; SEQ ID NO: 289 and SEQ ID NO: 232; SEQ ID NO: 289 and SEQ ID NO: 233; SEQ ID NO: 289 and SEQ ID NO: 234; SEQ ID NO: 289 and SEQ ID NO: 235; SEQ ID NO: 289 and SEQ ID NO: 236; SEQ ID NO: 289 and SEQ ID NO: 237; SEQ ID NO: 289 and SEQ ID NO: 238; SEQ ID NO: 289 and SEQ ID NO: 239; SEQ ID NO: 289 and SEQ ID NO: 240; SEQ ID NO: 289 and SEQ ID NO: 241; SEQ ID NO: 289 and SEQ ID NO: 242; SEQ ID NO: 289 and SEQ ID NO: 243; SEQ ID NO: 289 and SEQ ID NO: 244; SEQ ID NO: 289 and SEQ ID NO: 245; SEQ ID NO: 289 and SEQ ID NO: 246; SEQ ID NO: 289 and SEQ ID NO: 247; SEQ ID NO: 289 and SEQ ID NO: 248; SEQ ID NO: 289 and SEQ ID NO: 249; and SEQ ID NO: 289 and SEQ ID NO: 250.

In some aspects, the V_{H}-V_{L} pairs are selected from SEQ ID NO: 290 and SEQ ID NO: 211; SEQ ID NO: 290 and SEQ ID NO: 212; SEQ ID NO: 290 and SEQ ID NO: 213; SEQ ID NO: 290 and SEQ ID NO: 214; SEQ ID NO: 290 and SEQ ID NO: 215; SEQ ID NO: 290 and SEQ ID NO: 216; SEQ ID NO: 290 and SEQ ID NO: 217; SEQ ID NO: 290 and SEQ ID NO: 218; SEQ ID NO: 290 and SEQ ID NO: 219; SEQ ID NO: 290 and SEQ ID NO: 220; SEQ ID NO: 290 and SEQ ID NO: 221; SEQ ID NO: 290 and SEQ ID NO: 222; SEQ ID NO: 290 and SEQ ID NO: 223; SEQ ID NO: 290 and SEQ ID NO: 224; SEQ ID NO: 290 and SEQ ID NO: 225; SEQ ID NO: 290 and SEQ ID NO: 226; SEQ ID NO: 290 and SEQ ID NO: 227; SEQ ID NO: 290 and SEQ ID NO: 228; SEQ ID NO: 290 and SEQ ID NO: 229; SEQ ID NO: 290 and SEQ ID NO: 230; SEQ ID NO: 290 and SEQ ID NO: 231; SEQ ID NO: 290 and SEQ ID NO: 232; SEQ ID NO: 290 and SEQ ID NO: 233; SEQ ID NO: 290 and SEQ ID NO: 234; SEQ ID NO: 290 and SEQ ID NO: 235; SEQ ID NO: 290 and SEQ ID NO: 236; SEQ ID NO: 290 and SEQ ID NO: 237; SEQ ID NO: 290 and SEQ ID NO: 238; SEQ ID NO: 290 and SEQ ID NO: 239; SEQ ID NO: 290 and SEQ ID NO: 240; SEQ ID NO: 290 and SEQ ID NO: 241; SEQ ID NO: 290 and SEQ ID NO: 242; SEQ ID NO: 290 and SEQ ID NO: 243; SEQ ID NO: 290 and SEQ ID NO: 244; SEQ ID NO: 290 and SEQ ID NO: 245; SEQ ID NO: 290 and SEQ ID NO: 246; SEQ ID NO: 290 and SEQ ID NO: 247; SEQ ID NO: 290 and SEQ ID NO: 248; SEQ ID NO: 290 and SEQ ID NO: 249; and SEQ ID NO: 290 and SEQ ID NO: 250.

In some aspects, the V_{H}-V_{L} pairs are selected from SEQ ID NO: 291 and SEQ ID NO: 211; SEQ ID NO: 291 and SEQ ID NO: 212; SEQ ID NO: 291 and SEQ ID NO: 213; SEQ ID NO: 291 and SEQ ID NO: 214; SEQ ID NO: 291 and SEQ ID NO: 215; SEQ ID NO: 291 and SEQ ID NO: 216; SEQ ID NO: 291 and SEQ ID NO: 217; SEQ ID NO: 291 and SEQ ID NO: 218; SEQ ID NO: 291 and SEQ ID NO: 219; SEQ ID NO: 291 and SEQ ID NO: 220; SEQ ID NO: 291 and SEQ ID NO: 221; SEQ ID NO: 291 and SEQ ID NO: 222; SEQ ID NO: 291 and SEQ ID NO: 223; SEQ ID NO: 291 and SEQ ID NO: 224; SEQ ID NO: 291 and SEQ ID NO: 225; SEQ ID NO: 291 and SEQ ID NO: 226; SEQ ID NO: 291 and SEQ ID NO: 227; SEQ ID NO: 291 and SEQ ID NO: 228; SEQ ID NO: 291 and SEQ ID NO: 229; SEQ ID NO: 291 and SEQ ID NO: 230; SEQ ID NO: 291 and SEQ ID NO: 231; SEQ ID NO: 291 and SEQ ID NO: 232; SEQ ID NO: 291 and SEQ ID NO: 233; SEQ ID NO: 291 and SEQ ID NO: 234; SEQ ID NO: 291 and SEQ ID NO: 235; SEQ ID NO: 291 and SEQ ID NO: 236; SEQ ID NO: 291 and SEQ ID NO: 237; SEQ ID NO: 291 and SEQ ID NO: 238; SEQ ID NO: 291 and SEQ ID NO: 239; SEQ ID NO: 291 and SEQ ID NO: 240; SEQ ID NO: 291 and SEQ ID NO: 241; SEQ ID NO: 291 and SEQ ID NO: 242; SEQ ID NO: 291 and SEQ ID NO: 243; SEQ ID NO: 291 and SEQ ID NO: 244; SEQ ID NO: 291 and SEQ ID NO: 245; SEQ ID NO: 291 and SEQ ID NO: 246; SEQ ID NO: 291 and SEQ ID NO: 247; SEQ ID NO: 291 and SEQ ID NO: 248; SEQ ID NO: 291 and SEQ ID NO: 249; and SEQ ID NO: 291 and SEQ ID NO: 250.

In some aspects, the V_{H}-V_{L} pairs are selected from SEQ ID NO: 292 and SEQ ID NO: 211; SEQ ID NO: 292 and SEQ ID NO: 212; SEQ ID NO: 292 and SEQ ID NO: 213; SEQ ID NO: 292 and SEQ ID NO: 214; SEQ ID NO: 292 and SEQ ID NO: 215; SEQ ID NO: 292 and SEQ ID NO: 216; SEQ ID NO: 292 and SEQ ID NO: 217; SEQ ID NO: 292 and SEQ ID NO: 218; SEQ ID NO: 292 and SEQ ID NO: 219; SEQ ID NO: 292 and SEQ ID NO: 220; SEQ ID NO: 292 and SEQ ID NO: 221; SEQ ID NO: 292 and SEQ ID NO: 222; SEQ ID NO: 292 and SEQ ID NO: 223; SEQ ID NO: 292 and SEQ ID NO: 224; SEQ ID NO: 292 and SEQ ID NO: 225; SEQ ID NO: 292 and SEQ ID NO: 226; SEQ ID NO: 292 and SEQ ID NO: 227; SEQ ID NO: 292 and SEQ ID NO: 228; SEQ ID NO: 292 and SEQ ID NO: 229; SEQ ID NO: 292 and SEQ ID NO: 230; SEQ ID NO: 292 and SEQ ID NO: 231; SEQ ID NO: 292 and SEQ ID NO: 232; SEQ ID NO: 292 and SEQ ID NO: 233; SEQ ID NO: 292 and SEQ ID NO: 234; SEQ ID NO: 292 and SEQ ID NO: 235; SEQ ID NO: 292 and SEQ ID NO: 236; SEQ ID NO: 292 and SEQ ID NO: 237; SEQ ID NO: 292 and SEQ ID NO: 238; SEQ ID NO: 292 and SEQ ID NO: 239; SEQ ID NO: 292 and SEQ ID NO: 240; SEQ ID NO: 292 and SEQ ID NO: 241; SEQ ID NO: 292 and SEQ ID NO: 242; SEQ ID NO: 292 and SEQ ID NO: 243; SEQ ID NO: 292 and SEQ ID NO: 244; SEQ ID NO: 292 and SEQ ID NO: 245; SEQ ID NO: 292 and SEQ ID NO: 246; SEQ ID NO: 292 and SEQ ID NO: 247; SEQ ID NO: 292 and SEQ ID NO: 248; SEQ ID NO: 292 and SEQ ID NO: 249; and SEQ ID NO: 292 and SEQ ID NO: 250.

In some aspects, the V_{H}-V_{L} pairs are selected from SEQ ID NO: 293 and SEQ ID NO: 211; SEQ ID NO: 293 and SEQ ID NO: 212; SEQ ID NO: 293 and SEQ ID NO: 213; SEQ ID NO: 293 and SEQ ID NO: 214; SEQ ID NO: 293 and SEQ ID NO: 215; SEQ ID NO: 293 and SEQ ID NO: 216; SEQ ID NO: 293 and SEQ ID NO: 217; SEQ ID NO: 293 and SEQ ID NO: 218; SEQ ID NO: 293 and SEQ ID NO: 219; SEQ ID NO: 293 and SEQ ID NO: 220; SEQ ID NO: 293 and SEQ ID NO: 221; SEQ ID NO: 293 and SEQ ID NO: 222; SEQ ID NO: 293 and SEQ ID NO: 223; SEQ ID NO: 293 and SEQ ID NO: 224; SEQ ID NO: 293 and SEQ ID NO: 225; SEQ ID NO: 293 and SEQ ID NO: 226; SEQ ID NO: 293 and SEQ ID NO: 227; SEQ ID NO: 293 and SEQ ID NO: 228; SEQ ID NO: 293 and SEQ ID NO: 229; SEQ ID NO: 293 and SEQ ID NO: 230; SEQ ID NO: 293 and SEQ ID NO: 231; SEQ ID NO: 293 and SEQ ID NO: 232; SEQ ID NO: 293 and SEQ ID NO: 233; SEQ ID NO: 293 and SEQ ID NO: 234; SEQ ID NO: 293 and SEQ ID NO: 235; SEQ ID NO: 293 and SEQ ID NO: 236; SEQ ID NO: 293 and SEQ ID NO: 237; SEQ ID NO: 293 and SEQ ID NO: 238; SEQ ID NO: 293 and SEQ ID NO: 239; SEQ ID NO: 293 and SEQ ID NO: 240; SEQ ID NO: 293 and SEQ ID NO: 241; SEQ ID NO: 293 and SEQ ID NO: 242; SEQ ID NO: 293 and SEQ ID NO: 243; SEQ ID NO: 293 and SEQ ID NO: 244; SEQ ID NO: 293 and SEQ ID NO: 245; SEQ ID NO: 293 and SEQ ID NO: 246; SEQ ID NO: 293 and SEQ ID NO: 247; SEQ ID NO: 293 and SEQ ID NO: 248; SEQ ID NO: 293 and SEQ ID NO: 249; and SEQ ID NO: 293 and SEQ ID NO: 250.

In some aspects, the V_{H}-V_{L} pairs are selected from SEQ ID NO: 294 and SEQ ID NO: 211; SEQ ID NO: 294 and SEQ ID NO: 212; SEQ ID NO: 294 and SEQ ID NO: 213; SEQ ID NO: 294 and SEQ ID NO: 214; SEQ ID NO: 294 and SEQ ID NO: 215; SEQ ID NO: 294 and SEQ ID NO: 216; SEQ ID NO: 294 and SEQ ID NO: 217; SEQ ID NO: 294 and SEQ ID NO: 218; SEQ ID NO: 294 and SEQ ID NO: 219; SEQ ID NO: 294 and SEQ ID NO: 220; SEQ ID NO: 294 and SEQ ID NO: 221; SEQ ID NO: 294 and SEQ ID NO: 222; SEQ ID NO: 294 and SEQ ID NO: 223; SEQ ID NO: 294 and SEQ ID NO: 224; SEQ ID NO: 294 and SEQ ID NO: 225; SEQ ID NO: 294 and SEQ ID NO: 226; SEQ ID NO: 294 and SEQ ID NO: 227; SEQ ID NO: 294 and SEQ ID NO: 228; SEQ ID NO: 294 and SEQ ID NO: 229; SEQ ID NO: 294 and SEQ ID NO: 230; SEQ ID NO: 294 and SEQ ID NO: 231; SEQ ID NO: 294 and SEQ ID NO: 232; SEQ ID NO: 294 and SEQ ID NO: 233; SEQ ID NO: 294 and SEQ ID NO: 234; SEQ ID NO: 294 and SEQ ID NO: 235; SEQ ID NO: 294 and SEQ ID NO: 236; SEQ ID NO: 294 and SEQ ID NO: 237; SEQ ID NO: 294 and SEQ ID NO: 238; SEQ ID NO: 294 and SEQ ID NO: 239; SEQ ID NO: 294 and SEQ ID NO: 240; SEQ ID NO: 294 and SEQ ID NO: 241; SEQ ID NO: 294 and SEQ ID NO: 242; SEQ ID NO: 294 and SEQ ID NO: 243; SEQ ID NO: 294 and SEQ ID NO: 244; SEQ ID NO: 294 and SEQ ID NO: 245; SEQ ID NO: 294 and SEQ ID NO: 246; SEQ ID NO: 294 and SEQ ID NO: 247; SEQ ID NO: 294 and SEQ ID NO: 248; SEQ ID NO: 294 and SEQ ID NO: 249; and SEQ ID NO: 294 and SEQ ID NO: 250.

In some aspects, the V_{H}-V_{L} pairs are selected from SEQ ID NO: 295 and SEQ ID NO: 211; SEQ ID NO: 295 and SEQ ID NO: 212; SEQ ID NO: 295 and SEQ ID NO: 213; SEQ ID NO: 295 and SEQ ID NO: 214; SEQ ID NO: 295 and SEQ ID NO: 215; SEQ ID NO: 295 and SEQ ID NO: 216; SEQ ID NO: 295 and SEQ ID NO: 217; SEQ ID NO: 295 and SEQ ID NO: 218; SEQ ID NO: 295 and SEQ ID NO: 219; SEQ ID NO: 295 and SEQ ID NO: 220; SEQ ID NO: 295 and SEQ ID NO: 221; SEQ ID NO: 295 and SEQ ID NO: 222; SEQ ID NO: 295 and SEQ ID NO: 223; SEQ ID NO: 295 and SEQ ID NO: 224; SEQ ID NO: 295 and SEQ ID NO: 225; SEQ ID NO: 295 and SEQ ID NO: 226; SEQ ID NO: 295 and SEQ ID NO: 227; SEQ ID NO: 295 and SEQ ID NO: 228; SEQ ID NO: 295 and SEQ ID NO: 229; SEQ ID NO: 295 and SEQ ID NO: 230; SEQ ID NO: 295 and SEQ ID NO: 231; SEQ ID NO: 295 and SEQ ID NO: 232; SEQ ID NO: 295 and SEQ ID NO: 233; SEQ ID NO: 295 and SEQ ID NO: 234; SEQ ID NO: 295 and SEQ ID NO: 235; SEQ ID NO: 295 and SEQ ID NO: 236; SEQ ID NO: 295 and SEQ ID NO: 237; SEQ ID NO: 295 and SEQ ID NO: 238; SEQ ID NO: 295 and SEQ ID NO: 239; SEQ ID NO: 295 and SEQ ID NO: 240; SEQ ID NO: 295 and SEQ ID NO: 241; SEQ ID NO: 295 and SEQ ID NO: 242; SEQ ID NO: 295 and SEQ ID NO: 243; SEQ ID NO: 295 and SEQ ID NO: 244; SEQ ID NO: 295 and SEQ ID NO: 245; SEQ ID NO: 295 and SEQ ID NO: 246; SEQ ID NO: 295 and SEQ ID NO: 247; SEQ ID NO: 295 and SEQ ID NO: 248; SEQ ID NO: 295 and SEQ ID NO: 249; and SEQ ID NO: 295 and SEQ ID NO: 250.

In some aspects, the V_{H}-V_{L} pairs are selected from SEQ ID NO: 296 and SEQ ID NO: 211; SEQ ID NO: 296 and SEQ ID NO: 212; SEQ ID NO: 296 and SEQ ID NO: 213; SEQ ID NO: 296 and SEQ ID NO: 214; SEQ ID NO: 296 and SEQ ID NO: 215; SEQ ID NO: 296 and SEQ ID NO: 216; SEQ ID NO: 296 and SEQ ID NO: 217; SEQ ID NO: 296 and SEQ ID NO: 218; SEQ ID NO: 296 and SEQ ID NO: 219; SEQ ID NO: 296 and SEQ ID NO: 220; SEQ ID NO: 296 and SEQ ID NO: 221; SEQ ID NO: 296 and SEQ ID NO: 222; SEQ ID NO: 296 and SEQ ID NO: 223; SEQ ID NO: 296 and SEQ ID NO: 224; SEQ ID NO: 296 and SEQ ID NO: 225; SEQ ID NO: 296 and SEQ ID NO: 226; SEQ ID NO: 296 and SEQ ID NO: 227; SEQ ID NO: 296 and SEQ ID NO: 228; SEQ ID NO: 296 and SEQ ID NO: 229; SEQ ID NO: 296 and SEQ ID NO: 230; SEQ ID NO: 296 and SEQ ID NO: 231; SEQ ID NO: 296 and SEQ ID NO: 232; SEQ ID NO: 296 and SEQ ID NO: 233; SEQ ID NO: 296 and SEQ ID NO: 234; SEQ ID NO: 296 and SEQ ID NO: 235; SEQ ID NO: 296 and SEQ ID NO: 236; SEQ ID NO: 296 and SEQ ID NO: 237; SEQ ID NO: 296 and SEQ ID NO: 238; SEQ ID NO: 296 and SEQ ID NO: 239; SEQ ID NO: 296 and SEQ ID NO: 240; SEQ ID NO: 296 and SEQ ID NO: 241; SEQ ID NO: 296 and SEQ ID NO: 242; SEQ ID NO: 296 and SEQ ID NO: 243; SEQ ID NO: 296 and SEQ ID NO: 244; SEQ ID NO: 296 and SEQ ID NO: 245; SEQ ID NO: 296 and SEQ ID NO: 246; SEQ ID NO: 296 and SEQ ID NO: 247; SEQ ID NO: 296 and SEQ ID NO: 248; SEQ ID NO: 296 and SEQ ID NO: 249; and SEQ ID NO: 296 and SEQ ID NO: 250.

In some aspects, the V_{H}-V_{L} pairs are selected from SEQ ID NO: 297 and SEQ ID NO: 211; SEQ ID NO: 297 and SEQ ID NO: 212; SEQ ID NO: 297 and SEQ ID NO: 213; SEQ ID NO: 297 and SEQ ID NO: 214; SEQ ID NO: 297 and SEQ ID NO: 215; SEQ ID NO: 297 and SEQ ID NO: 216; SEQ ID NO: 297 and SEQ ID NO: 217; SEQ ID NO: 297 and SEQ ID NO: 218; SEQ ID NO: 297 and SEQ ID NO: 219; SEQ ID NO: 297 and SEQ ID NO: 220; SEQ ID NO: 297 and SEQ ID NO: 221; SEQ ID NO: 297 and SEQ ID NO: 222; SEQ ID NO: 297 and SEQ ID NO: 223; SEQ ID NO: 297 and SEQ ID NO: 224; SEQ ID NO: 297 and SEQ ID NO: 225; SEQ ID NO: 297 and SEQ ID NO: 226; SEQ ID NO: 297 and SEQ ID NO: 227; SEQ ID NO: 297 and SEQ ID NO: 228; SEQ ID NO: 297 and SEQ ID NO: 229; SEQ ID NO: 297 and SEQ ID NO: 230; SEQ ID NO: 297 and SEQ ID NO: 231; SEQ ID NO: 297 and SEQ ID NO: 232; SEQ ID NO: 297 and SEQ ID NO: 233; SEQ ID NO: 297 and SEQ ID NO: 234; SEQ ID NO: 297 and SEQ ID NO: 235; SEQ ID NO: 297 and SEQ ID NO: 236; SEQ ID NO: 297 and SEQ ID NO: 237; SEQ ID NO: 297 and SEQ ID NO: 238; SEQ ID NO: 297 and SEQ ID NO: 239; SEQ ID NO: 297 and SEQ ID NO: 240; SEQ ID NO: 297 and SEQ ID NO: 241; SEQ ID NO: 297 and SEQ ID NO: 242; SEQ ID NO: 297 and SEQ ID NO: 243; SEQ ID NO: 297 and SEQ ID NO: 244; SEQ ID NO: 297 and SEQ ID NO: 245; SEQ ID NO: 297 and SEQ ID NO: 246; SEQ ID NO: 297 and SEQ ID NO: 247; SEQ ID NO: 297 and SEQ ID NO: 248; SEQ ID NO: 297 and SEQ ID NO: 249; and SEQ ID NO: 297 and SEQ ID NO: 250.

In some aspects, the V_{H}-V_{L} pairs are selected from SEQ ID NO: 298 and SEQ ID NO: 211; SEQ ID NO: 298 and SEQ ID NO: 212; SEQ ID NO: 298 and SEQ ID NO: 213; SEQ ID NO: 298 and SEQ ID NO: 214; SEQ ID NO: 298 and SEQ ID NO: 215; SEQ ID NO: 298 and SEQ ID NO: 216; SEQ ID NO: 298 and SEQ ID NO: 217; SEQ ID NO: 298 and SEQ ID NO: 218; SEQ ID NO: 298 and SEQ ID NO: 219; SEQ ID NO: 298 and SEQ ID NO: 220; SEQ ID NO: 298 and SEQ ID NO: 221; SEQ ID NO: 298 and SEQ ID NO: 222; SEQ ID NO: 298 and SEQ ID NO: 223; SEQ ID NO: 298 and SEQ ID NO: 224; SEQ ID NO: 298 and SEQ ID NO: 225; SEQ ID NO: 298 and SEQ ID NO: 226; SEQ ID NO: 298 and SEQ ID NO: 227; SEQ ID NO: 298 and SEQ ID NO: 228; SEQ ID NO: 298 and SEQ ID NO: 229; SEQ ID NO: 298 and SEQ ID NO: 230; SEQ ID NO: 298 and SEQ ID NO: 231; SEQ ID NO: 298 and SEQ ID NO: 232; SEQ ID NO: 298 and SEQ ID NO: 233; SEQ ID NO: 298 and SEQ ID NO: 234; SEQ ID NO: 298 and SEQ ID NO: 235; SEQ ID NO: 298 and SEQ ID NO: 236; SEQ ID NO: 298 and SEQ ID NO: 237; SEQ ID NO: 298 and SEQ ID NO: 238; SEQ ID NO: 298 and SEQ ID NO: 239; SEQ ID NO: 298 and SEQ ID NO: 240; SEQ ID NO: 298 and SEQ ID NO: 241; SEQ ID NO: 298 and SEQ ID NO: 242; SEQ ID NO: 298 and SEQ ID NO: 243; SEQ ID NO: 298 and SEQ ID NO: 244; SEQ ID NO: 298 and SEQ ID NO: 245; SEQ ID NO: 298 and SEQ ID NO: 246; SEQ ID NO: 298 and SEQ ID NO: 247; SEQ ID NO: 298 and SEQ ID NO: 248; SEQ ID NO: 298 and SEQ ID NO: 249; and SEQ ID NO: 298 and SEQ ID NO: 250.

In some aspects, the V_{H}-V_{L} pairs are selected from SEQ ID NO: 299 and SEQ ID NO: 211; SEQ ID NO: 299 and SEQ ID NO: 212; SEQ ID NO: 299 and SEQ ID NO: 213; SEQ ID NO: 299 and SEQ ID NO: 214; SEQ ID NO: 299 and SEQ ID NO: 215; SEQ ID NO: 299 and SEQ ID NO: 216; SEQ ID NO: 299 and SEQ ID NO: 217; SEQ ID NO: 299 and SEQ ID NO: 218; SEQ ID NO: 299 and SEQ ID NO: 219; SEQ ID NO: 299 and SEQ ID NO: 220; SEQ ID NO: 299 and SEQ ID NO: 221; SEQ ID NO: 299 and SEQ ID NO: 222; SEQ ID NO: 299 and SEQ ID NO: 223; SEQ ID NO: 299 and SEQ ID NO: 224; SEQ ID NO: 299 and SEQ ID NO: 225; SEQ ID NO: 299 and SEQ ID NO: 226; SEQ ID NO: 299 and SEQ ID NO: 227; SEQ ID NO: 299 and SEQ ID NO: 228; SEQ ID NO: 299 and SEQ ID NO: 229; SEQ ID NO: 299 and SEQ ID NO: 230; SEQ ID NO: 299 and SEQ ID NO: 231; SEQ ID NO: 299 and SEQ ID NO: 232; SEQ ID NO: 299 and SEQ ID NO: 233; SEQ ID NO: 299 and SEQ ID NO: 234; SEQ ID NO: 299 and SEQ ID NO: 235; SEQ ID NO: 299 and SEQ ID NO: 236; SEQ ID NO: 299 and SEQ ID NO: 237; SEQ ID NO: 299 and SEQ ID NO: 238; SEQ ID NO: 299 and SEQ ID NO: 239; SEQ ID NO: 299 and SEQ ID NO: 240; SEQ ID NO: 299 and SEQ ID NO: 241; SEQ ID NO: 299 and SEQ ID NO: 242; SEQ ID NO: 299 and SEQ ID NO: 243; SEQ ID NO: 299 and SEQ ID NO: 244; SEQ ID NO: 299 and SEQ ID NO: 245; SEQ ID NO: 299 and SEQ ID NO: 246; SEQ ID NO: 299 and SEQ ID NO: 247; SEQ ID NO: 299 and SEQ ID NO: 248; SEQ ID NO: 299 and SEQ ID NO: 249; and SEQ ID NO: 299 and SEQ ID NO: 250.

3.7.4.1 Variants of V_{H}-V_{L} Pairs

In some embodiments, the V_{H}-V_{L} pairs provided herein comprise a variant of an illustrative V_{H} and/or V_{L} sequence provided in this disclosure.

In some aspects, the V_{H} sequence comprises, consists of, or consists essentially of a variant of an illustrative V_{H} sequence provided in this disclosure. In some aspects, the V_{H} sequence comprises, consists of, or consists essentially of a sequence having at least 85%, 90%, 95%, 96%, 97%, 98%, 99%, or 99.1% identity with any of the illustrative V_{H} sequences provided in this disclosure.

In some embodiments, the V_{H} sequence comprises, consists of, or consists essentially of any of the illustrative V_{H} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

In some aspects, the V_{L} sequence comprises, consists of, or consists essentially of a variant of an illustrative V_{L} sequence provided in this disclosure. In some aspects, the V_{L} sequence comprises, consists of, or consists essentially of a sequence having at least 85%, 90%, 95%, 96%, 97%, 98%, 99%, or 99.5% identity with any of the illustrative V_{L} sequences provided in this disclosure.

In some embodiments, the V_{L} sequence comprises, consists of, or consists essentially of any of the illustrative V_{L} sequences provided in this disclosure having 20 or fewer, 19 or fewer, 18 or fewer, 17 or fewer, 16 or fewer, 15 or fewer, 14 or fewer, 13 or fewer, 12 or fewer, 11 or fewer, 10 or fewer, 9 or fewer, 8 or fewer, 7 or fewer, 6 or fewer, 5 or fewer, 4 or fewer, 3 or fewer, 2 or fewer, or 1 or fewer amino acid substitutions. In some aspects, the amino acid substitutions are conservative amino acid substitutions.

3.8 PD-1 Antibodies Comprising All Six CDRs

In some embodiments, the PD-1 antibody comprises a CDR-H1 sequence, a CDR-H2 sequence, a CDR-H3 sequence, a CDR-L1 sequence, and a CDR-L3 sequence. In some aspects, the CDR sequences are part of a V_{H} (for CDR-H) or V_{L} (for CDR-L).

In some aspects, the CDR-H1 sequence is a Chothia CDR-H1 sequence comprising, consisting of, or consisting essentially of SEQ ID NOs: 24-38 or a Chothia CDR-H1 sequence of a V_{H} sequence provided in any one of SEQ ID NOs.: 228-250; the CDR-H2 sequence is a Chothia CDR-H2 sequence comprising, consisting of, or consisting essentially of SEQ ID NOs: 86-87 or a Chothia CDR-H2 sequence of a V_{H} sequence provided in any one of SEQ ID NOs.: 228-250; the CDR-H3 sequence is a CDR-H3 sequence comprising, consisting of, or consisting essentially of SEQ ID NOs: 130-131 or a CDR-H3 sequence of a V_{H} sequence provided in any one of SEQ ID NOs.: 228-250; the CDR-L1 sequence is a CDR-L1 sequence comprising, consisting of, or consisting essentially of SEQ ID NOs: 148-149 or a CDR-L1 sequence of a V_{L} sequence selected from SEQ ID NOs.: 277-299; the CDR-L2 sequence is a CDR-L2 sequence comprising, consisting of, or consisting essentially of SEQ ID NOs: 166-172 or a CDR-L2 sequence of a V_{L} sequence selected from SEQ ID NOs.: 277-299; and the CDR-L3 sequence is a CDR-L3 sequence comprising, consisting of, or consisting essentially of SEQ ID NOs: 189-190 or a CDR-L3 sequence of a V_{L} sequence selected from SEQ ID NOs.: 277-299.

In some aspects, the CDR-H1 sequence is a Kabat CDR-H1 sequence comprising, consisting of, or consisting essentially of SEQ ID NOs: 59-65 or a Kabat CDR-H1 sequence of a V_{H} sequence selected from SEQ ID NOs.: 228-250; the CDR-H2 sequence is a Kabat CDR-H2 sequence comprising, consisting of, or consisting essentially of SEQ ID NOs: 108-109 or a Kabat CDR-H2 sequence of a V_{H} sequence selected from SEQ ID NOs.: 228-250; the CDR-H3 sequence is a CDR-H3 sequence comprising, consisting of, or consisting essentially of SEQ ID NOs: 130-131 or a Kabat CDR-H3 sequence of a V_{H} sequence selected from SEQ ID NOs.: 228-250; the CDR-L1 sequence is a CDR-L1 sequence comprising, consisting of, or consisting essentially of SEQ ID NOs: 148-149 or a CDR-L1 sequence of a V_{L} sequence selected from SEQ ID NOs.: 277-299; the CDR-L2 sequence is a CDR-L2 sequence comprising, consisting of, or consisting essentially of SEQ ID NOs: 166-172 or a CDR-L2 sequence of a V_{L} sequence selected from SEQ ID NOs.: 277-299; and the CDR-L3 sequence is a CDR-L3 sequence comprising, consisting of, or consisting essentially of SEQ ID NOs: 189-190 or or a CDR-L3 sequence of a V_{L} sequence selected from SEQ ID NOs.: 277-299.

In some embodiments, the PD-1 antibody comprises a CDR-H1 sequence of SEQ ID NO: 24, a CDR-H2 sequence of SEQ ID NO: 86, a CDR-H3 sequence of SEQ ID NO: 130, a CDR-L1 sequence of SEQ ID NO: 148, a CDR-L2 sequence of SEQ ID NO: 166, and a CDR-L3 sequence of SEQ ID NO: 189.

In some embodiments, the PD-1 antibody comprises a CDR-H1 sequence of SEQ ID NO: 24, a CDR-H2 sequence of SEQ ID NO: 86, a CDR-H3 sequence of SEQ ID NO: 130, a CDR-L1 sequence of SEQ ID NO: 149, a CDR-L2 sequence of SEQ ID NO: 172, and a CDR-L3 sequence of SEQ ID NO: 190.

In some embodiments, the PD-1 antibody comprises a CDR-H1 sequence of SEQ ID NO: 38, a CDR-H2 sequence of SEQ ID NO: 87, a CDR-H3 sequence of SEQ ID NO: 131, a CDR-L1 sequence of SEQ ID NO: 148, a CDR-L2 sequence of SEQ ID NO: 166, and a CDR-L3 sequence of SEQ ID NO: 189.

In some embodiments, the PD-1 antibody comprises a CDR-H1 sequence of SEQ ID NO: 38, a CDR-H2 sequence of SEQ ID NO: 87, a CDR-H3 sequence of SEQ ID NO: 131, a CDR-L1 sequence of SEQ ID NO: 149, a CDR-L2 sequence of SEQ ID NO: 172, and a CDR-L3 sequence of SEQ ID NO: 190.

In some embodiments, the PD-1 antibody comprises a CDR-H1 sequence of SEQ ID NO: 59, a CDR-H2 sequence of SEQ ID NO: 108, a CDR-H3 sequence of SEQ ID NO: 130, a CDR-L1 sequence of SEQ ID NO: 148, a CDR-L2 sequence of SEQ ID NO: 166, and a CDR-L3 sequence of SEQ ID NO: 189.

In some embodiments, the PD-1 antibody comprises a CDR-H1 sequence of SEQ ID NO: 59, a CDR-H2 sequence of SEQ ID NO: 108, a CDR-H3 sequence of SEQ ID NO: 130, a CDR-L1 sequence of SEQ ID NO: 149, a CDR-L2 sequence of SEQ ID NO: 172, and a CDR-L3 sequence of SEQ ID NO: 190.

In some embodiments, the PD-1 antibody comprises a CDR-H1 sequence of SEQ ID NO: 65, a CDR-H2 sequence of SEQ ID NO: 109, a CDR-H3 sequence of SEQ ID NO: 131, a CDR-L1 sequence of SEQ ID NO: 148, a CDR-L2 sequence of SEQ ID NO: 166, and a CDR-L3 sequence of SEQ ID NO: 189.

In some embodiments, the PD-1 antibody comprises a CDR-H1 sequence of SEQ ID NO: 65, a CDR-H2 sequence of SEQ ID NO: 109, a CDR-H3 sequence of SEQ ID NO: 131, a CDR-L1 sequence of SEQ ID NO: 149, a CDR-L2 sequence of SEQ ID NO: 172, and a CDR-L3 sequence of SEQ ID NO: 190.

3.8.1 Variants of Antibodies Comprising All Six CDRs

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions.

### 4. Bi-Specific Antibodies and Antigen-Binding Constructs

Provided herein are bi-specific antigen-binding constructs, e.g., antibodies, that bind LAG3 and PD-1. The bi-specific antigen-binding construct includes two antigen-binding polypeptide constructs, e.g., antigen binding domains, wherein at least one polypeptide construct specifically binds to LAG3 and at least one polypeptide construct specifically binds to PD-1. In some embodiments, the antigen-binding construct is derived from known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

In some embodiments, in the bi-specific antibodies disclosed herein, the first and second antigen-binding polypeptide constructs independently comprise different light chains. For example, in some embodiments, the first antigen-binding polypeptide construct comprises a V_{L} sequence selected from any one of SEQ ID NOs: 251-266, and the second antigen-binding polypeptide construct comprises a V_{L} sequence selected from any one of SEQ ID NOs: 267-276. In some embodiments, the first antigen-binding polypeptide construct comprises a V_{L} sequence selected from any one of SEQ ID NOs: 267-276, and the second antigen-binding polypeptide construct comprises a V_{L} sequence selected from any one of SEQ ID NOs: 251-266. In some embodiments, the first and second antigen-binding polypeptide constructs comprise the same light chain. For example, in some embodiments, the first and second antigen-binding polypeptide constructs comprise a same V_{L} sequence selected from any one of SEQ ID NOs: 251-276. In some embodiments, the first and second antigen-binding polypeptide constructs further comprise a C_{L} sequence selected from any one of SEQ ID NOs: 353-363 and 386-387. In some embodiments, the first and second antigen-binding polypeptide constructs comprise the same C_{L} sequence. In some embodiments, the first and second antigen-binding polypeptide constructs comprise different C_{L} sequences.

The term “antigen-binding construct” refers to any agent, e.g., polypeptide or polypeptide complex capable of binding to an antigen. In some aspects an antigen-binding construct is a polypeptide that specifically binds to an antigen of interest. An antigen-binding construct can be a monomer, dimer, multimer, a protein, a peptide, or a protein or peptide complex; an antibody, an antibody fragment, or an antigen-binding fragment thereof; an scFv and the like. An antigen-binding construct can be a polypeptide construct that is monospecific, bi-specific, or multispecific. In some aspects, an antigen-binding construct can include, e.g., one or more antigen-binding components (e.g., Fabs or scFvs) linked to one or more Fc. Further examples of antigen-binding constructs are described below and provided in the Examples.

The term “bi-specific” includes any agent, e.g., an antigen-binding construct, which has two antigen-binding moieties (e.g. antigen-binding polypeptide constructs), each with a unique binding specificity. For example, a first antigen-binding moiety binds to an epitope on a first antigen, and a second antigen-binding moiety binds to an epitope on a second antigen, where the first antigen is different from the second antigen.

For example, in some embodiments, a bi-specific agent can bind to, or interact with, (a) a cell surface target molecule and (b) an Fc receptor on the surface of an effector cell. In some embodiments, the agent can bind to, or interact with (a) a first cell surface target molecule and (b) a second cell surface target molecule that is different from the first cell surface target molecule. In some embodiments, the agent can bind to and bridge two cells, i.e. interact with (a) a first cell surface target molecule on a first call and (b) a second cell surface target molecule on a second cell that is different from the first cells surface target molecule on the first cell.

In contrast, a monospecific antigen-binding construct refers to an antigen-binding construct with a single binding specificity. In other words, both antigen-binding moieties bind to the same epitope on the same antigen. Examples of monospecific antigen-binding constructs include the anti-CD19 antibody HD37 and the anti-CD3 antibody OKT3 for example.

An antigen-binding construct can be an antibody or antigen-binding portion thereof as disclosed herein.

Methods of generating bi-specific antibodies and bi-specific antigen-binding constructs are described, for example, in Briukmann, U., and R. E. Kontermann. 2017. mAbs 9(2):182-212; and in Yang, et al. 2017. Int. J. Mol. Sci. 18(1):48 (21 pages), each of which is incorporated herein by reference in its entirety.

4.1 Antigen-Binding Polypeptide Construct—Format

The bi-specific antigen-binding construct comprises at least two antigen-binding polypeptide constructs, e.g., antigen binding domains. The format of the antigen-binding polypeptide construct determines certain functional characteristics of the bi-specific antigen-binding construct. In some embodiments, the bi-specific antigen-binding construct has an scFv-scFv format, i.e. both antigen-binding polypeptide constructs are scFvs. In some embodiments, the bi-specific antigen-binding construct has a Fab-Fab format, i.e. both antigen-binding polypeptide constructs are Fabs. In some embodiments, the bi-specific antigen-binding construct has an scFv-Fab format, i.e. a first antigen-binding polypeptide construct is an scFv, and a second antigen-binding polypeptide construct is an Fab. The bi-specific antibody or antigen-binding construct can have any form suitable for the antibody or antigen-binding construct, so long as it comprises a first antigen binding domain and a second antigen binding domain that bind to distinct targets.

In some embodiments, the bi-specific antibody or antigen-binding construct is provided, comprising a first antigen binding domain that specifically binds LAG3 and a second antigen binding domain that specifically binds PD-1. In some embodiments, a bi-specific antigen construct is provided, comprising a first scFv that specifically binds LAG3 and a second scFv that specifically binds PD-1. In some embodiments, a bi-specific antigen construct is provided, comprising a first Fab that specifically binds LAG3 and a second Fab that specifically binds PD-1. In some embodiments, a bi-specific antigen construct is provided, comprising an scFv that specifically binds LAG3 and a Fab that specifically binds PD-1. In some embodiments, a bi-specific antigen construct is provided, comprising a Fab that specifically binds LAG3 and an scFv that specifically binds PD-1.

In some embodiments, the bi-specific antibody or bi-specific antigen-binding construct can be generated as a dual-variable domain antibody. A “dual-variable domain antibody” (also referred to as a DVD-Ig) refers to fusion of an additional V_{H} domain and V_{L} domain of a second specificity to a given IgG heavy chain and light chain. Generation of dual-variable domain antibody formats are described, for example, in Wu et al. 2007. Nature Biotechnology 25:1290-1297 and U.S. 2007/0071675, each of which is incorporated herein by reference in its entirety.

In some embodiments, the bi-specific antibody or bi-specific antigen-binding construct is generated as a cross-over dual-variable domain antibody. A “cross-over dual-variable domain antibody” (also referred to as a CODV-Ig) refers to a format related to the dual-variable domain antibody format wherein the two V_{H} domains and two V_{L} domains are linked to allow cross-over pairing of the variable V_{H}-V_{L} domains. Generation of cross-over dual-variable domain antibody formats are described, for example, in Steinmetz et al. 2016. mAbs 8:867-878, which is incorporated herein by reference in its entirety.

Other formats for synthesis and use in bi-specific antibodies or bi-specific antigen-binding constructs are contemplated and described below.

The format “Single-chain Fv” or “scFv” includes the V_{H} and V_{L} domains of an antibody, wherein these domains are present in a single polypeptide chain. In some embodiments, the scFv polypeptide further comprises a polypeptide linker between the V_{H} and V_{L} domains. See, e.g., Pluckthun in The Pharmacology of Monoclonal Antibodies, vol. 113, Rosenburg and Moore eds., Springer-Verlag, New York, pp. 269-315 (1994).

The “Fab fragment” (also referred to as fragment antigen-binding) contains the constant domain (C_{L}) of the light chain and the first constant domain (C_{H}1) of the heavy chain along with the variable domains V_{L} and V_{H} on the light and heavy chains respectively. The variable domains comprise the complementarity determining loops (CDR, also referred to as hypervariable region) that are involved in antigen-binding. Fab′ fragments differ from Fab fragments by the addition of a few residues at the carboxy terminus of the heavy chain CH1 domain including one or more cysteines from the antibody hinge region.

The “Single domain antibodies” or “sdAb” format is an individual immunoglobulin domain. SdAbs are fairly stable and easy to express as fusion partner with the Fc chain of an antibody (see Harmsen M M, De Haard H J (2007). “Properties, production, and applications of camelid single-domain antibody fragments.” Appl. Microbiol Biotechnol. 77(1): 13-22).

4.1.1 Format scFv

Embodiments are directed to bi-specific antigen-binding constructs comprising two antigen-binding polypeptide constructs that are each capable of specific binding to a distinct antigen. In some embodiments, each antigen-binding polypeptide construct is in an scFv format. (i.e., antigen-binding domains composed of a heavy chain variable domain and a light chain variable domain, connected with a polypeptide linker). In some embodiments, the scFv molecules are human. In some embodiments, the scFv molecules are humanized. The scFvs can be optimized for protein expression and yield by the modifications disclosed herein.

In some embodiments, the scFv is optimized by changing the order of the variable domains V_{L} and V_{H} in the scFv. In some embodiments, in the scFv, the C-terminus of the light chain variable region can be linked to the N-terminus of the heavy chain variable region. In some embodiments, in the scFv, the C-terminus of the heavy chain variable region can be linked to the N-terminus of the light chain variable region.

The variable regions of the scFv can be connected via a linker peptide, or scFv linker, that allows the formation of a functional antigen-binding moiety. In some embodiments, the scFv can be optimized for protein expression and yield by changing composition and/or length of the scFv linker polypeptide. Typical peptide linkers comprise about 2-20 amino acids, and are described herein or known in the art. Suitable, non-immunogenic linker peptides include, for example, (G₄S)ₙ, (SG₄)ₙ, (G₄S)ₙ, G₄(SG₄)ₙ or G₂(SG₂)ₙ linker peptides, wherein n is generally a number between 1 and 10. In some embodiments, n is a number between 4 and 8. In some embodiments, n is a number between 3 and 6. In some embodiments, n is a number between 2 and 4. Other linkers are described, for example, in Bird et al. 1988. Science 242:423-426; Huston et al. 1988. PNAS 85:5879-5883; and McCafferty et al. 1990. Nature 348:552-554.

The scFv molecule can be optimized for protein expression and yield by including stabilizing disulfide bridges between the heavy and light chain variable domains, for example as described in Reiter et al. (Nat Biotechnol 14, 1239-1245 (1996)). Accordingly, in some embodiments, the bi-specific antigen-binding molecule disclosed herein can comprise an scFv molecule wherein an amino acid in the heavy chain variable domain and an amino acid in the light chain variable domain have been replaced by cysteine so that a disulfide bridge can be formed between the heavy and light chain variable domain.

ScFvs can also be stabilized by mutation of CDR sequences, as described in the art (Miller et al., Protein Eng Des Sel. 2010 July; 23(7):549-57; Igawa et al., MAbs. 2011 May-June; 3(3):243-5; Perchiacca & Tessier, Annu Rev Chem Biomol Eng. 2012; 3:263-286, each of which is incorporated herein by reference in its entirety.) and as disclosed herein in exemplary embodiments.

4.1.2 Fc Domains of Antigen-Binding Constructs

In some embodiments, the antigen-binding constructs described herein comprise an Fc domain, e.g., a dimeric Fc. The Fc domain is a heterodimeric Fc comprising first and second Fc polypeptides each comprising a modified C_{H}3 sequence, wherein each modified C_{H}3 sequence comprises asymmetric amino acid modifications that promote the formation of a heterodimeric Fc and the dimerized C_{H}3 domains have a melting temperature (Tm) of about 68° C. or higher, and wherein the first Fc polypeptide is linked to the first antigen-binding polypeptide construct, with a first hinge linker, and the second Fc polypeptide is linked to the second antigen-binding polypeptide construct with a second hinge linker.

The term “Fc domain” or “Fc region” herein refers to a C-terminal region of an immunoglobulin heavy chain that contains at least a portion of the constant region. The term includes native sequence Fc regions and variant Fc regions and is used interchangeably with “Fc.” Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md., 1991. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG C_{H}2 and an IgG C_{H}3 constant domain sequence.

An Fc domain comprises either a C_{H}3 domain or a C_{H}3 and a C_{H}2 domain. The C_{H}3 domain comprises two C_{H}3 sequences, one from each of the two Fc polypeptides of the dimeric Fc. The C_{H}2 domain comprises two C_{H}2 sequences, one from each of the two Fc polypeptides of the dimeric Fc.

In some embodiments, the Fc comprises at least one or two C_{H}3 sequences. In some aspects, the Fc is coupled, with or without one or more linkers, to a first antigen-binding construct and/or a second antigen-binding construct. In some embodiments, the Fc is a human Fc. In some embodiments, the Fc is a human IgG or IgG1 Fc. In some embodiments, the Fc is a heterodimeric Fc. In some embodiments, the Fc comprises at least one or two C_{H}2 sequences.

In some embodiments, the Fc comprises one or more modifications in at least one of the C_{H}3 sequences. In some embodiments, the Fc comprises one or more modifications in at least one of the C_{H}2 sequences. For example, the Fc can include one or modifications selected from the group consisting of: V262E, V262D, V262K, V262R, V262S, V264S, V303R, and V305R. In some embodiments, an Fc is a single polypeptide. In some embodiments, an Fc is multiple peptides, e.g., two polypeptides.

4.1.3 Modified C_{H}3 Domains

In some embodiments, the antigen-binding construct described herein comprises a heterodimeric Fc comprising a modified C_{H}3 domain that has been asymmetrically modified. The heterodimeric Fc can comprise two heavy chain constant domain polypeptides: a first Fc polypeptide and a second Fc polypeptide, which can be used interchangeably provided that Fc comprises one first Fc polypeptide and one second Fc polypeptide. Generally, the first Fc polypeptide comprises a first C_{H}3 sequence and the second Fc polypeptide comprises a second C_{H}3 sequence.

Two C_{H}3 sequences that comprise one or more amino acid modifications introduced in an asymmetric fashion generally results in a heterodimeric Fc, rather than a homodimer, when the two C_{H}3 sequences dimerize. As used herein, “asymmetric amino acid modifications” refers to any modification where an amino acid at a specific position on a first C_{H}3 sequence is different from the amino acid on a second C_{H}3 sequence at the same position, and the first and second C_{H}3 sequence preferentially pair to form a heterodimer, rather than a homodimer. This heterodimerization can be a result of modification of one of the two amino acids at the same respective amino acid position on each sequence; or modification of both amino acids on each sequence at the same respective position on each of the first and second C_{H}3 sequences. The first and second C_{H}3 sequence of a heterodimeric Fc can comprise one or more than one asymmetric amino acid modification.

Typically an Fc can include two contiguous heavy chain sequences (A and B) that are capable of dimerizing. With respect to the antigen binding constructs described herein, in some embodiments the first scFv is linked to chain A of the heterodimeric Fc and the second scFv is linked to chain B of the heterodimeric Fc. In some embodiments the second scFv is linked to chain A of the heterodimeric Fc and the first scFv is linked to chain B of the heterodimeric Fc.

In some embodiments, one or both sequences of an Fc include one or more mutations or modifications at the following locations: L351, L368, F405, Y407, T366, K392, T394, T350, 5400, and/or N390, using EU numbering. In some embodiments, an Fc includes the mutations as disclosed in the art (see Von Kreudenstein et al. 2013. mAbs 5(5):646-654; Von Kreudenstein et al. 2014. Methods 65:77-94; U.S. 2013/0195849; Ridgway et al. 1996. Protein Eng 9(7):617-621; and Brinkmann, U., and R. E. Kontermann. 2017. mAbs 9(2):182-212, each of which is incorporated herein by reference in its entirety.)

The first and second C_{H}3 sequences can comprise amino acid mutations as described herein. In some embodiments, the heterodimeric Fc comprises a modified C_{H}3 domain with a first C_{H}3 sequence having one or more amino acid modifications selected from L351Y, F405A, and Y407V, and the second C_{H}3 sequence having one or more amino acid modifications selected from T366L, T366I, K392L, K392M, and T394W. In some embodiments, the heterodimeric Fc comprises a modified C_{H}3 domain with a first C_{H}3 sequence having amino acid modifications T350V/T366L/K392L/T394W, and a second C_{H}3 sequence having amino acid modifications T350V/L351Y/F405A/Y407V.

Additional modifications within the first and second C_{H}3, or within the amino acid sequence of human IgG1 Fc, can be found, for example, at U.S. 2016/0326249, which is incorporated herein by reference in its entirety.

4.1.4 Modified V_{H}/V_{L} and/or C_{H}1/C_{L}1 Interactions

In some embodiments, the bi-specific antibodies or bi-specific antigen-binding constructs disclosed herein comprise one or more light chains. In some embodiments, the bi-specific antibody or bi-specific antigen-binding construct comprises two light chains. In some embodiments, the two light chains are different. In some embodiments, the two light chains are the same.

In embodiments wherein the two light chains are different, one or more modifications can be introduced into one or both light chains to allow for pairing of a cognate heavy chain and light chain. Accordingly, in some embodiments, the interaction between the variable domain a first light chain and the variable domain of a corresponding first heavy chain (i.e., V_{H}-V_{L} interaction) is modified. In some embodiments, the interaction the constant domain of a first light chain and the first constant domain of a corresponding first heavy chain (i.e., C_{H}1-C_{L} interaction) is modified. In some embodiments, the modification comprises genetically engineering or genetically modifying residues that are involved in the V_{H}-V_{L} and/or the Cal-C_{L} interaction. In some embodiments, the modification involves mutating residues to modify electrostatic interactions between the V_{H}-V_{L} pairs and/or the C_{H}1-C_{L} pairs. The result of modification to the V_{H}-V_{L} and/or the C_{H}1-C_{L} interactions can result in improved accuracy (or improved “steering”) in pairing of cognate heavy and light chains. Exemplary modifications in these domains are described, for example, in Lewis et al. 2014. Nature Biotechnology 32:191-198 and WO 2014/082179, each of which is incorporated herein by reference in its entirety.

4.1.5 Hinge Linkers

In some embodiments, in the bi-specific antigen-binding constructs disclosed herein, the first Fc polypeptide is linked to the first antigen-binding polypeptide construct with a first hinge linker, and the second Fc polypeptide is linked to the second antigen-binding polypeptide construct with a second hinge linker. Examples of hinge linker sequences are well-known to one of skill in the art and can be used in the antigen-binding constructs described herein. Alternatively, modified versions of known hinge linkers can be used.

The hinge linker polypeptides are selected such that they maintain or optimize the functional activity of the antigen-binding construct. Suitable linker polypeptides include IgG hinge regions such as, for example those from IgG₁, IgG₂, or IgG₄, including the upper hinge sequences and core hinge sequences. The amino acid residues corresponding to the upper and core hinge sequences vary depending on the IgG type, as is known in the art and one of skill in the art would readily be able to identify such sequences for a given IgG type. Modified versions of these exemplary linkers can also be used. For example, modifications to improve the stability of the IgG4 hinge are known in the art (see for example, Labrijn et al. (2009) Nature Biotechnology 27, 767-771). Examples of hinge linker sequences are found, for example, in U.S. 2016/0326249.

4.1.6. Bispecific Scaffold Arrangements

The bi-specific antigen-binding construct can have a variety of different arrangements. For example, the bi-specific antigen-binding construct can comprise a 2-chain scFvFc, a 3-chain Fab×scFvFc, or a 4-chain IgG-like bispecific construct as described below.

4.1.6.1. Generation of a PD1/LAG3 Bi-Specific Antigen-Binding Construct (Two-chain scFvFc)

A general scheme for generating embodiments of scFvs for use in a PD1/LAG3 bi-specific antigen-binding construct is provided below in Tables 5 and 6. In some embodiments, the bi-specific antigen-binding construct comprises a 2-chain scFvFc HC/LC pairing maintained by genetically fusing the V_{H} to the V_{L} of both antibodies to form an scFv. In some embodiments, the order of the HC/LC pairing is V_{H}/V_{L}. In some embodiments, the order of the HC/LC pairing is V_{L}/V_{H}. In any of the foregoing embodiments, the HC/LC pairing can comprise a linker sequence. In some embodiments, the linker sequence comprises a Gly/Ser-rich linker of the sequence (GGGGS)n where n=3, 4, 5, or 6 to provide linkers with lengths of 15, 20, 25, or 30 residues, respectively.

In some embodiments, the scFv is arranged in a V_{H}-V_{L} arrangement. In some embodiments, the scFv comprises a V_{H} sequence comprising, consisting of, or consisting essentially of any one of SEQ ID NOs: 211-227; a linker selected from SEQ ID NOs: 308-314; and a V_{L} sequence comprising, consisting of, or consisting essentially of any one of SEQ ID NOs: 267-276. In some embodiments, the scFv comprises a V_{H} sequence comprising, consisting of, or consisting essentially of any one of SEQ ID NOs: 191-210; a linker selected from SEQ ID NOs: 308-314; and a V_{L} sequence comprising, consisting of, or consisting essentially of any one of SEQ ID NOs: 251-266.

TABLE 5
Exemplary V_{H} - V_{L} scFv Arrangements

| Design | Name | V_{H} | Linker | V_{L} |
|---|---|---|---|---|
| (a) | 1353-G10 | 1353-G10 V_{H} | (GGGGS)3, | 1353-G10 V_{L} |
| | V_{H} - | | (GGGGS)4, | (λ), or |
| | V_{L} scFv | | (GGGGS)5, or | 1353-G10 |
| | | | (GGGGS)6 | V_{L} (κ) |
| (b) | 1449-G09.2 | 1449-G09.2 V_{H} | (GGGGS)3, | 1449-G09.2 V_{L} |
| | V_{H} - | | (GGGGS)4, | |
| | V_{L} scFv | | (GGGGS)5, or | |
| | | | (GGGGS)6 | |

In some embodiments, the scFv is arranged in a V_{L}-V_{H} arrangement. In some embodiments, the scFv comprises a V_{L} sequence comprising, consisting of, or consisting essentially of any one of SEQ ID NOs: 267-276; a linker selected from SEQ ID NOs: 308-314; and a V_{H} sequence comprising, consisting of, or consisting essentially of any one of SEQ ID NOs: 211-227. In some embodiments, the scFv comprises a V_{L} sequence comprising, consisting of, or consisting essentially of any one of SEQ ID NOs: 251-266; a linker selected from SEQ ID NOs: 308-314; and a V_{H} sequence comprising, consisting of, or consisting essentially of any one of SEQ ID NOs: 191-210.

TABLE 6
Exemplary V_{L} - V_{H} scFv Arrangements

| Design | Name | V_{L} | Linker | V_{H} |
|---|---|---|---|---|
| (a) | 1353-G10 | 1353-G10 V_{L} | (GGGGS)3, | 1353-G10 |
| | V_{L} - | (λ), or | (GGGGS)4, | V_{H} |
| | V_{H} scFv | 1353-G10 V_{L} (κ) | (GGGGS)5, or | |
| | | | (GGGGS)6 | |
| (b) | 1449-G09.2 | 1449-G09.2 V_{L} | (GGGGS)3, | 1449-G09.2 |
| | V_{L}- | | (GGGGS)4, | V_{H} |
| | V_{H} scFv | | (GGGGS)5, or | |
| | | | (GGGGS)6 | |

4.1.6.2. Generation of a PD1/LAG3 Bi-Specific Antigen-Binding Construct (Two-Chain scFvFc with Knob-in-Hole Mutations)

In some embodiments, a PD1/LAG3 bi-specific antigen-binding construct comprising a two-chain scFvFc arrangement, wherein the scFvFc pairing comprises knob-in-hole mutations, is provided. In some embodiments, a PD1/LAG3 bi-specific antigen-binding construct is provided, comprising an anti-PD1 scFvFc knob paired with an anti-LAG3 scFvFc hole. In some embodiments, a PD1/LAG3 bi-specific antigen-binding construct is provided, comprising an anti-PD1 scFvFc hole paired with an anti-LAG3 scFvFc knob. The scFvFcs include scFvs generated in accordance with Section 4.1.6.1.

A general scheme for scFvFcs useful in a PD1/LAG3 bi-specific antigen-binding construct comprising a two-chain scFv arrangement with knob-in-hole mutations is provided below in Table 7.

TABLE 7
Exemplary scFvFcs (with Knob-in-hole Mutations)
for Two-chain scFvFc Arrangements

| Design | Name | scFv | Linker | C_{H}2-C_{H}3 |
|---|---|---|---|---|
| (a) | aPD1 scFvFc | 1353-G10 V_{H}- | Linker or | Fc-Knob, Fc- |
| | knob | V_{L} scFv, or | Linker- | Knob V262E, |
| | | 1353-G10 V_{L}- | hinge | Fc-Knob- |
| | | V_{H} scFv | | V264S, Fc- |
| | | | | knob-D399C, |
| | | | | Fc-knob-S354C |
| (b) | aLAG3 scFvFc | 1449-G09.2 V_{H}- | Linker or | Fc-Knob, Fc- |
| | knob | V_{L} scFv, or | Linker- | Knob V262E, |
| | | 1449-G09.2 V_{L}- | hinge | Fc-Knob- |
| | | V_{H} scFv | | V264S, Fc- |
| | | | | knob-D399C, |
| | | | | Fc-knob-S354C |
| (c) | aPD1 scFvFc | 1353-G10 V_{H}- | Linker or | Fc-Hole, Fc- |
| | hole | V_{L} scFv, or | Linker- | hole V262E, |
| | | 1353-G10 V_{L}- | hinge | Fc-hole |
| | | V_{H} scFv | | V264S, Fc- |
| | | | | hole-Y349C, |
| | | | | or Fc-hole- |
| | | | | K392C |
| (d) | aLAG3 scFvFc | 1449-G09.2 V_{H}- | Linker or | Fc-Hole, Fc- |
| | hole | V_{L} scFv, or | Linker- | hole V262E, |
| | | 1449-G09.2 V_{L}- | hinge | Fc-hole |
| | | V_{H} scFv | | V264S, or |
| | | | | Fc-hole- |
| | | | | Y349C, or |
| | | | | Fc-hole-K392C |

In some embodiments, a PD1/LAG3 bi-specific antigen-binding construct comprising a two-chain scFvFc arrangement is prepared using the following arrangement: an anti-PD1 scFvFc knob (Table 12, design (a)) paired with an anti-LAG3 scFvFc hole (Table 12, design (d)). In some embodiments, a PD1/LAG3 bi-specific antigen-binding construct comprising a two-chain scFvFc arrangement is prepared using the following arrangement: an anti-PD1 scFvFc hole (Table 12, design (c)) paired with an anti-LAG3 scFvFc knob (Table 12, design (b)).

In some embodiments, the anti-PD1 scFvFc knob is constructed from: (1) an anti-PD1 scFv; (2) a linker or linker-hinge; and (3) a CH₂-CH₃ region. In some embodiments, the anti-PD1 scFv comprises a V_{H}-V_{L} arrangement and comprises a V_{H} sequence comprising, consisting of, or consisting essentially of any one of SEQ ID NOs: 211-227; a linker selected from SEQ ID NOs: 308-314; and a V_{L} sequence comprising, consisting of, or consisting essentially of any one of SEQ ID NOs: 267-276. In some embodiments, the anti-PD1 scFv comprises a V_{L}-V_{H} arrangement and comprises a V_{L} sequence comprising, consisting of, or consisting essentially of SEQ ID NOs: 267-276; a linker selected from SEQ ID NOs: 308-314; and a V_{H} sequence comprising, consisting of, or consisting essentially of SEQ ID NOs: 211-227. In some embodiments, the linker-hinge comprises, consists of, or consists essentially of SEQ ID NO: 315. In some embodiments, the CH2-CH3 region comprises, consists of, or consists essentially of any one of SEQ ID NOs.: 321-325.

In some embodiments, the anti-PD1 scFvFc hole is constructed from: (1) an anti-PD1 scFv; (2) a linker or linker-hinge; and (3) a CH₂-CH₃ region. In some embodiments, the anti-PD1 scFv comprises a V_{H}-V_{L} arrangement and comprises a V_{H} sequence comprising, consisting of, or consisting essentially of any one of SEQ ID NOs: 211-227; a linker selected from SEQ ID NOs: 308-314; and a V_{L} sequence comprising, consisting of, or consisting essentially of any one of SEQ ID NOs: 267-276. In some embodiments, the anti-PD1 scFv comprises a V_{L}-V_{H} arrangement and comprises a V_{L} sequence comprising, consisting of, or consisting essentially of SEQ ID NOs: 267-276; a linker selected from SEQ ID NOs: 308-314; and a V_{H} sequence comprising, consisting of, or consisting essentially of SEQ ID NOs: 211-227. In some embodiments, the linker-hinge comprises, consists of, or consists essentially of SEQ ID NO: 315. In some embodiments, the CH2-CH3 region comprises, consists of, or consists essentially of any one of SEQ ID NOs.: 326-330.

In some embodiments, the anti-LAG3 scFvFc knob is constructed from: (1) an anti-LAG3 scFv; (2) a linker or linker-hinge; and (3) a CH2-CH3 region. In some embodiments, the anti-LAG3 scFv comprises a V_{H}-V_{L} arrangement and comprises a V_{H} sequence comprising, consisting of, or consisting essentially of any one of SEQ ID NOs: 191-210; a linker selected from SEQ ID NOs: 308-314; and a V_{L} sequence comprising, consisting of, or consisting essentially of any one of SEQ ID NOs: 251-266. In some embodiments, the anti-LAG3 scFv comprises a V_{L}-V_{H} arrangement and comprises a V_{L} sequence comprising, consisting of, or consisting essentially of SEQ ID NOs: 251-266; a linker selected from SEQ ID NOs: 308-314; and a V_{H} sequence comprising, consisting of, or consisting essentially of SEQ ID NOs: 191-210. In some embodiments, the linker-hinge comprises, consists of, or consists essentially of SEQ ID NO: 315. In some embodiments, the CH2-CH3 region comprises, consists of, or consists essentially of any one of SEQ ID NOs.: 321-325.

In some embodiments, the anti-LAG3 scFvFc hole is constructed from: (1) an anti-LAG3 scFv; (2) a linker or linker-hinge; and (3) a CH₂-CH₃ region. In some embodiments, the anti-LAG3 scFv comprises a V_{H}-V_{L} arrangement and comprises a V_{H} sequence comprising, consisting of, or consisting essentially of any one of SEQ ID NOs: 191-210; a linker selected from SEQ ID NOs: 308-314; and a V_{L} sequence comprising, consisting of, or consisting essentially of any one of SEQ ID NOs: 251-266. In some embodiments, the anti-LAG3 scFv comprises a V_{L}-V_{H} arrangement and comprises a V_{L} sequence comprising, consisting of, or consisting essentially of SEQ ID NOs: 251-266; a linker selected from SEQ ID NOs: 308-314; and a V_{H} sequence comprising, consisting of, or consisting essentially of SEQ ID NOs: 191-210. In some embodiments, the linker-hinge comprises, consists of, or consists essentially of SEQ ID NO: 315. In some embodiments, the CH₂-CH₃ region comprises, consists of, or consists essentially of any one of SEQ ID NOs.: 326-330.

4.1.6.3. Generation of a PD1/LAG3 Bi-Specific Antigen-Binding Construct (Three-chain Fab×scFvFc with Knob-in-Hole Mutations)

In some embodiments, the bi-specific antigen-binding construct comprises a 3-chain Fab×scFvFc scaffolds in which an scFv is replaced with a Fab domain. In these embodiments, the asymmetry of the scaffold facilitates correct HC/LC pairing as there is only one HC/LC pairing that can correctly form the Fab domain; the other arm is an scFv.

In some embodiments, a PD1/LAG3 bi-specific antigen-binding construct comprising a three-chain Fab×scFvFc structure can be prepared using the following arrangements: (1) an anti-PD1 scFvFc knob paired with an anti-LAG3 half IgG (HC+LC) hole, (2) an anti-PD1 scFvFc hole paired with an anti-LAG3 half IgG (HC+LC) knob, (3) an anti-PD1 half IgG (HC+LC) knob paired with an anti-LAG3 scFvFc hole, and (4) an anti-PD1 half IgG (HC+LC) hole paired with an anti-LAG3 scFvFc knob. The scFvs included within such scFvFc arrangements can be generated in accordance with Section 4.1.6.1.

A general scheme for anti-PD1 half IgGs (HC+LC, knob or hole) and anti-LAG3 half IgGs (HC+LC, knob or hole) for use in a PD1/LAG3 bi-specific antigen-binding construct comprising a three-chain Fab×scFv arrangement with knob-in-hole mutations is provided below in Tables 8 and 9.

TABLE 8
Exemplary HCs of anti-PD1 and anti-LAG3 Half IgGs

| Design | Name | V_{H} | C_{H}1 | Linker | C_{H}2-C_{H}3 |
|---|---|---|---|---|---|
| (a) | aPD1 | 1353-G10 V_{H} | C_{H}1-wt | Hinge-wt | Fc-Knob, Fc- |
| | HC | | | | Knob V262E, |
| | knob | | | | Fc-Knob- |
| | | | | | V264S, Fc- |
| | | | | | knob-D399C, |
| | | | | | Fc-knob- |
| | | | | | S354C |
| (b) | aLAG3 | 1449-G09.2 V_{H} | C_{H}1-wt | Hinge-wt | Fc-Knob, Fc- |
| | HC | | | | Knob V262E, |
| | knob | | | | Fc-Knob- |
| | | | | | V264S, Fc- |
| | | | | | knob-D399C, |
| | | | | | Fc-knob- |
| | | | | | S354C |
| (c) | aPD1 | 1353-G10 V_{H} | C_{H}1-wt | Hinge-wt | Fc-Hole, Fc- |
| | HC | | | | hole V262E, |
| | hole | | | | Fc-hole |
| | | | | | V264S, or |
| | | | | | Fc-hole- |
| | | | | | Y349C, or |
| | | | | | Fc-hole- |
| | | | | | K392C |
| (d) | aLAG3 | 1449-G09.2 V_{H} | C_{H}1-wt | Hinge-wt | Fc-Hole, Fc- |
| | HC | | | | hole V262E, |
| | hole | | | | Fc-hole |
| | | | | | V264S, or |
| | | | | | Fc-hole- |
| | | | | | Y349C, or |
| | | | | | Fc-hole- |
| | | | | | K392C |

TABLE 9
Exemplary LCs of anti-PD1 and anti-LAG3 Half IgGs

| Design | Name | V_{L} | C_{L} |
|---|---|---|---|
| (a) | aPD1 LC (λ) | 1353-G10 V_{L} (λ) | Cλ-wt |
| (b) | aPD1 LC (κ) | 1353-G10 V_{L} (κ) | Cκ-wt |
| (c) | aLAG3 LC | 1449-G09.2 V_{L} | Cκ-wt |

In some embodiments, the anti-PD1 half IgG knob comprises a heavy chain and a light chain, wherein the heavy chain comprises: a V_{H} comprising, consisting of, or consisting essentially of any one of SEQ ID NOs: 211-227; a CH₁ region comprising, consisting of, or consisting essentially of SEQ ID NO: 385; a linker comprising, consisting of, or consisting essentially of SEQ ID NO: 316; and a CH₂-CH₃ region comprising, consisting of, or consisting essentially of any one of SEQ ID NOs.: 321-325, and wherein the light chain comprises: a V_{L} comprising, consisting of, or consisting essentially of any one of SEQ ID NOs: 267-276 and a C_{L} comprising, consisting of, or consisting essentially of any one of SEQ ID NOs: 386-387.

In some embodiments, the anti-PD1 half IgG hole comprises a heavy chain and a light chain, wherein the heavy chain comprises: a V_{H} comprising, consisting of, or consisting essentially of any one of SEQ ID NOs: 211-227; a CH₁ region comprising, consisting of, or consisting essentially of SEQ ID NO: 385; a linker comprising, consisting of, or consisting essentially of SEQ ID NO: 316; and a CH₂-CH₃ region comprising, consisting of, or consisting essentially of any one of SEQ ID NOs.: 326-330, and wherein the light chain comprises: a V_{L} comprising, consisting of, or consisting essentially of any one of SEQ ID NOs: 267-276 and a C_{L} comprising, consisting of, or consisting essentially of any one of SEQ ID NOs:386-387.

In some embodiments, the anti-LAG3 half IgG knob comprises a heavy chain and a light chain, wherein the heavy chain comprises: a V_{H} comprising, consisting of, or consisting essentially of any one of SEQ ID NOs: 191-210; a CH₁ region comprising, consisting of, or consisting essentially of SEQ ID NO: 385; a linker comprising, consisting of, or consisting essentially of SEQ ID NO: 316; and a C_{H}2-C_{H}3 region comprising, consisting of, or consisting essentially of any one of SEQ ID NOs.: 321-325, and wherein the light chain comprises: a V_{L} comprising, consisting of, or consisting essentially of any one of SEQ ID NOs: 251-266 and a C_{L} comprising, consisting of, or consisting essentially of SEQ ID NO:386.

In some embodiments, the anti-LAG3 half IgG hole comprises a heavy chain and a light chain, wherein the heavy chain comprises: a V_{H} comprising, consisting of, or consisting essentially of any one of SEQ ID NOs: 191-210; a CH₁ region comprising, consisting of, or consisting essentially of SEQ ID NO: 385; a linker comprising, consisting of, or consisting essentially of SEQ ID NO: 316; and a CH₂-CH₃ region comprising, consisting of, or consisting essentially of any one of SEQ ID NOs.: 326-330, and wherein the light chain comprises: a V_{L} comprising, consisting of, or consisting essentially of any one of SEQ ID NOs: 251-266 and a C_{L} comprising, consisting of, or consisting essentially of SEQ ID NO:386.

In some embodiments, the anti-LAG3 scFvFc hole is constructed from: (1) an anti-LAG3 scFv; (2) a linker or linker-hinge; and (3) a CH₂-CH₃ region. In some embodiments, the anti-LAG3 scFv comprises a V_{H}-V_{L} arrangement and comprises a V_{H} sequence comprising, consisting of, or consisting essentially of any one of SEQ ID NOs: 191-210; a linker selected from SEQ ID NOs: 308-314; and a V_{L} sequence comprising, consisting of, or consisting essentially of any one of SEQ ID NOs: 251-266. In some embodiments, the anti-LAG3 scFv comprises a V_{L}-V_{H} arrangement and comprises a V_{L} sequence comprising, consisting of, or consisting essentially of SEQ ID NOs: 251-266; a linker selected from SEQ ID NOs: 308-314; and a V_{H} sequence comprising, consisting of, or consisting essentially of SEQ ID NOs: 191-210. In some embodiments, the linker-hinge comprises, consists of, or consists essentially of SEQ ID NO: 315. In some embodiments, the CH₂-CH₃ region comprises, consists of, or consists essentially of any one of SEQ ID NOs.: 326-330.

In some embodiments, a PD1/LAG3 bi-specific antigen-binding construct comprising a three-chain Fab×scFvFc arrangement is prepared using the following arrangement: an anti-PD1 scFvFc knob (Table 7, design (a)) paired with an anti-LAG3 half IgG hole (HC=Table 8, design (d), LC=Table 9, design (c)).

In some embodiments, a PD1/LAG3 bi-specific antigen-binding construct comprising a three-chain Fab×scFvFc arrangement is prepared using the following arrangement: an anti-PD1 scFvFc hole (Table 7, design (c)) paired with an anti-LAG3 half IgG knob (HC=Table 8, design (b), LC=Table 9, design (c)).

In some embodiments, a PD1/LAG3 bi-specific antigen-binding construct comprising a three-chain Fab× scFvFc arrangement is prepared using the following arrangement: an anti-PD1 half IgG knob (HC=Table 8, design (a), LC=Table 9, design (a) or (b)) paired with an anti-LAG3 scFvFc hole (Table 7, design (d)).

In some embodiments, a PD1/LAG3 bi-specific antigen-binding construct comprising a three-chain Fab× scFvFc arrangement is prepared using the following arrangement: an anti-PD1 half IgG hole (HC=Table 8, design (c), LC=Table 9, design (a) or (b)) hole paired with an anti-LAG3 scFvFc knob (Table 7, design (b)).

4.1.6.4. Generation of a PD1/LAG3 Bi-Specific Antigen-Binding Construct (Three-Chain Fab× scFvFc with zw Mutations)

In some embodiments, the bi-specific antigen-binding construct comprises a three-chain Fab× scFvFc scaffold, wherein the Fab and scFvFc structures comprise knob-in-hole mutations. In these embodiments, the asymmetry of the scaffold facilitates correct HC/LC pairing as there is only one HC/LC pairing that can correctly form the Fab domain; the other arm is an scFv.

In some embodiments, a PD1/LAG3 bi-specific antigen-binding construct is provided, comprising a three-chain Fab× scFvFc structure comprising an anti-PD1 scFvFc with zwA mutations paired with an anti-LAG3 half IgG (HC+LC) with zwB mutations. In some embodiments, a PD1/LAG3 bi-specific antigen-binding construct is provided, comprising a three-chain Fab× scFvFc structure comprising an anti-PD1 scFvFc with zwB mutations paired with an anti-LAG3 half IgG (HC+LC) with zwA mutations. In some embodiments, a PD1/LAG3 bi-specific antigen-binding construct is provided, comprising a three-chain Fab× scFvFc structure comprising an anti-PD1 half IgG (HC+LC) with zwA mutations paired with an anti-LAG3 scFvFc with zwB mutations. In some embodiments, a PD1/LAG3 bi-specific antigen-binding construct is provided, comprising a three-chain Fab× scFvFc structure comprising an anti-PD1 half IgG (HC+LC) with zwB mutations paired with an anti-LAG3 scFvFc with zwA mutations. The mutations encompassed by “zwA” mutations include T350V/L351Y/F405A/Y407V in the C_{H}3 domain. The mutations encompassed by “zwB” mutations include T350V/T366L/K392L/T394W in the C_{H}3 domain.

A general scheme for scFvFcs with zw mutations and for anti-PD1 half IgGs (HC+LC) and anti-LAG3 half IgGs (HC+LC) with zw mutations for use in a PD1/LAG3 bi-specific antigen-binding construct comprising a three-chain Fab× scFv arrangement is provided below in Tables 10 and 11. Exemplary LC embodiments for the half IgGs correspond to those in Table 9 above.

TABLE 10
Exemplary scFvFcs (with zw Mutations) for
Three-chain Fab × scFvFc Arrangement

| Design | Name | scFv | Linker | CH₂-CH₃ |
|---|---|---|---|---|
| (a) | aPD1 scFvFc | 1353-G10 V_{H}- | Linker or | Fc-zwA, Fc- |
| | zwA | V_{L} scFv, or | Linker- | zwA V262E, or |
| | | 1353-G10 V_{L}- | hinge | Fc-zwA V264S |
| | | V_{H} scFv | | |
| (b) | aLAG3 scFvFc | 1449-G09.2 V_{H}- | Linker or | Fc-zwA, Fc- |
| | zwA | V_{L} scFv, or | Linker- | zwA V262E, or |
| | | 1449-G09.2 V_{L}- | hinge | Fc-zwA V264S |
| | | V_{H} scFv | | |
| (c) | aPD1 scFvFc | 1353-G10 V_{H}- | Linker or | Fc-zwB, Fc- |
| | zwB | V_{L} scFv, or | Linker- | zwB V262E, or |
| | | 1353-G10 V_{L}- | hinge | Fc-zwB V264S |
| | | V_{H} scFv | | |
| (d) | aLAG3 scFvFc | 1449-G09.2 V_{H}- | Linker or | Fc-zwB, Fc- |
| | zwB | V_{L} scFv, or | Linker- | zwB V262E, or |
| | | 1449-G09.2 V_{L}- | hinge | Fc-zwB V264S |
| | | V_{H} scFv | | |

In some embodiments, the anti-PD1 scFvFc zwA is constructed from: (1) an anti-PD1 scFv; (2) a linker or linker-hinge; and (3) a CH₂—CH₃ region. In some embodiments, the anti-PD1 scFv comprises a V_{H}-V_{L} arrangement and comprises a V_{H} sequence comprising, consisting of, or consisting essentially of any one of SEQ ID NOs: 211-227; a linker selected from SEQ ID NOs: 308-314; and a V_{L} sequence comprising, consisting of, or consisting essentially of any one of SEQ ID NOs: 267-276. In some embodiments, the anti-PD1 scFv comprises a V_{L}-V_{H} arrangement and comprises a V_{L} sequence comprising, consisting of, or consisting essentially of SEQ ID NOs: 267-276; a linker selected from SEQ ID NOs: 308-314; and a V_{H} sequence comprising, consisting of, or consisting essentially of SEQ ID NOs: 211-227. In some embodiments, the linker-hinge comprises, consists of, or consists essentially of SEQ ID NO: 315. In some embodiments, the CH₂-CH₃ region comprises, consists of, or consists essentially of any one of SEQ ID NOs.: 331-333.

In some embodiments, the anti-PD1 scFvFc zwB is constructed from: (1) an anti-PD1 scFv; (2) a linker or linker-hinge; and (3) a CH₂-CH₃ region. In some embodiments, the anti-PD1 scFv comprises a V_{H}-V_{L} arrangement and comprises a V_{H} sequence comprising, consisting of, or consisting essentially of any one of SEQ ID NOs: 211-227; a linker selected from SEQ ID NOs: 308-314; and a V_{L} sequence comprising, consisting of, or consisting essentially of any one of SEQ ID NOs: 267-276. In some embodiments, the anti-PD1 scFv comprises a V_{L}-V_{H} arrangement and comprises a V_{L} sequence comprising, consisting of, or consisting essentially of SEQ ID NOs: 267-276; a linker selected from SEQ ID NOs: 308-314; and a V_{H} sequence comprising, consisting of, or consisting essentially of SEQ ID NOs: 211-227. In some embodiments, the linker-hinge comprises, consists of, or consists essentially of SEQ ID NO: 315. In some embodiments, the C_{H}2-C_{H}3 region comprises, consists of, or consists essentially of any one of SEQ ID NOs.: 334-336.

In some embodiments, the anti-LAG3 scFvFc zwA is constructed from: (1) an anti-LAG3 scFv; (2) a linker or linker-hinge; and (3) a CH₂-CH₃ region. In some embodiments, the anti-LAG3 scFv comprises a V_{H}-V_{L} arrangement and comprises a V_{H} sequence comprising, consisting of, or consisting essentially of any one of SEQ ID NOs: 191-210; a linker selected from SEQ ID NOs: 308-314; and a V_{L} sequence comprising, consisting of, or consisting essentially of any one of SEQ ID NOs: 251-266. In some embodiments, the anti-LAG3 scFv comprises a V_{L}-V_{H} arrangement and comprises a V_{L} sequence comprising, consisting of, or consisting essentially of SEQ ID NOs: 251-266; a linker selected from SEQ ID NOs: 308-314; and a V_{H} sequence comprising, consisting of, or consisting essentially of SEQ ID NOs: 191-210. In some embodiments, the linker-hinge comprises, consists of, or consists essentially of SEQ ID NO: 315. In some embodiments, the CH₂-CH₃ region comprises, consists of, or consists essentially of any one of SEQ ID NOs.: 331-333.

In some embodiments, the anti-LAG3 scFvFc zwB is constructed from: (1) an anti-LAG3 scFv; (2) a linker or linker-hinge; and (3) a CH₂—CH₃ region. In some embodiments, the anti-LAG3 scFv comprises a V_{H}-V_{L} arrangement and comprises a V_{H} sequence comprising, consisting of, or consisting essentially of any one of SEQ ID NOs: 191-210; a linker selected from SEQ ID NOs: 308-314; and a V_{L} sequence comprising, consisting of, or consisting essentially of any one of SEQ ID NOs: 251-266. In some embodiments, the anti-LAG3 scFv comprises a V_{L}-V_{H} arrangement and comprises a V_{L} sequence comprising, consisting of, or consisting essentially of SEQ ID NOs: 251-266; a linker selected from SEQ ID NOs: 308-314; and a V_{H} sequence comprising, consisting of, or consisting essentially of SEQ ID NOs: 191-210. In some embodiments, the linker-hinge comprises, consists of, or consists essentially of SEQ ID NO: 315. In some embodiments, the CH₂-CH₃ region comprises, consists of, or consists essentially of any one of SEQ ID NOs.: 334-336.

TABLE 11
Exemplary HCs of anti-PD1 and anti-
LAG3 Half IgGs with zw Mutations

| Design | Name | V_{H} | C_{H}1 | Linker | CH₂-CH₃ |
|---|---|---|---|---|---|
| (a) | aPD1 | 1353-G10 VH | C_{H}1-wt | Hinge-wt | Fc-zwA, |
| | HC | | | | Fc-zwA |
| | zwA | | | | V262E, or |
| | | | | | Fc-zwA |
| | | | | | V264S |
| (b) | aLAG3 | 1449-G09.2 VH | C_{H}1-wt | Hinge-wt | Fc-zwA, |
| | HC | | | | Fc-zwA |
| | zwA | | | | V262E, or |
| | | | | | Fc-zwA |
| | | | | | V264S |
| (c) | aPD1 | 1353-G10 VH | C_{H}1-wt | Hinge-wt | Fc-zwB, |
| | HC | | | | Fc-zwB |
| | zwB | | | | V262E, or |
| | | | | | Fc-zwB |
| | | | | | V264S |
| (d) | aLAG3 | 1449-G09.2 VH | C_{H}1-wt | Hinge-wt | Fc-zwB, |
| | HC | | | | Fc-zwB |
| | zwB | | | | V262E, or |
| | | | | | Fc-zwB |
| | | | | | V264S |

In some embodiments, the anti-PD1 half IgG with zwA mutations comprises a heavy chain and a light chain, wherein the heavy chain comprises: a V_{H} comprising, consisting of, or consisting essentially of any one of SEQ ID NOs: 211-227; a CH₁ region comprising, consisting of, or consisting essentially of SEQ ID NO: 385; a linker comprising, consisting of, or consisting essentially of SEQ ID NO: 316; and a CH₂-CH₃ region comprising, consisting of, or consisting essentially of any one of SEQ ID NOs.: 331-333, and wherein the light chain comprises: a V_{L} comprising, consisting of, or consisting essentially of any one of SEQ ID NOs: 267-276 and a C_{L} comprising, consisting of, or consisting essentially of any one of SEQ ID NOs: 386-387.

In some embodiments, the anti-PD1 half IgG with zwB mutations comprises a heavy chain and a light chain, wherein the heavy chain comprises: a V_{H} comprising, consisting of, or consisting essentially of any one of SEQ ID NOs: 211-227; a CH₁ region comprising, consisting of, or consisting essentially of SEQ ID NO: 385; a linker comprising, consisting of, or consisting essentially of SEQ ID NO: 316; and a CH₂-CH₃ region comprising, consisting of, or consisting essentially of any one of SEQ ID NOs.: 334-336, and wherein the light chain comprises: a V_{L} comprising, consisting of, or consisting essentially of any one of SEQ ID NOs: 267-276 and a C_{L} comprising, consisting of, or consisting essentially of any one of SEQ ID NOs: 386-387.

In some embodiments, the anti-LAG3 half IgG with zwA mutations comprises a heavy chain and a light chain, wherein the heavy chain comprises: a V_{H} comprising, consisting of, or consisting essentially of any one of SEQ ID NOs: 191-210; a CH₁ region comprising, consisting of, or consisting essentially of SEQ ID NO: 385; a linker comprising, consisting of, or consisting essentially of SEQ ID NO: 316; and a CH₂-CH₃ region comprising, consisting of, or consisting essentially of any one of SEQ ID NOs.: 331-333, and wherein the light chain comprises: a V_{L} comprising, consisting of, or consisting essentially of any one of SEQ ID NOs: 251-266 and a C_{L} comprising, consisting of, or consisting essentially of SEQ ID NO: 386.

In some embodiments, the anti-LAG3 half IgG with zwB mutations comprises a heavy chain and a light chain, wherein the heavy chain comprises: a V_{H} comprising, consisting of, or consisting essentially of any one of SEQ ID NOs: 191-210; a CH₁ region comprising, consisting of, or consisting essentially of SEQ ID NO: 385; a linker comprising, consisting of, or consisting essentially of SEQ ID NO: 316; and a CH₂-CH₃ region comprising, consisting of, or consisting essentially of any one of SEQ ID NOs.: 334-336, and wherein the light chain comprises: a V_{L} comprising, consisting of, or consisting essentially of any one of SEQ ID NOs: 251-266 and a C_{L} comprising, consisting of, or consisting essentially of SEQ ID NO: 386.

4.1.6.5. Generation of a PD1/LAG3 Bi-Specific Antigen-Binding Construct (Four-chain Fab-Fc×Fab-Fc (IgG-like) with zw Mutations)

In some embodiments, the bi-specific antigen-binding construct comprises a four-chain IgG-like scaffold comprising a Fab domain fused to the N-termini of a heterodimeric Fc. In such embodiments, this bispecific format comprises four chains: heavy chain 1 (HC1), light chain 1 (LC1), heavy chain 2 (HC2), and light chain 2 (LC2). In some embodiments, the HC and LC sequences are mutated as described herein in sections 4.1.2-4.1.4 to facilitate correct pairing between HCs and LCs. For example, the HC/LC pairing designs listed in Tables 12-14 can be incorporated into the construct to facilitate correct HC/LC pairing. HC1 is designed such that it pairs specifically with LC1 rather than LC2. HC2 is designed such this it pairs specifically with LC2 rather than LC1. Six designs are shown below in Tables 12 and 13 that enforce correct light-chain pairing: (a), (b) (c), (d), (e), and (f). Designs (a) and (b) in Table 12 can be incorporated for an embodiment in which one light chain is a kappa chain (LC1) and the second light chain is a lambda chain (LC2). Designs (c), (d), (e) and (f) in Table 13 can be incorporated for embodiments in which the first and second light chains (LC1, LC2) are kappa chains.

TABLE 12
Exemplary embodiments of a four-chain bi-specific
antibody arrangement (kappa × lambda)

| Bispecific | LAG3 HC1 | LAG3 LC1 | PD-1 HC2 | PD-1 LC2 |
|---|---|---|---|---|
| (a) | HC1(a) | LC1(a) | HC2(a) | LC2(a) |
| (b) | HC1(b) | LC1(b) | HC2(b) | LC2(b) |

TABLE 13
Exemplary embodiments of a four-chain bi-specific
antibody arrangement (kappa × kappa)

| Bispecific | LAG3 HC1 | LAG3 LC1 | PD-1 HC2 | PD-1 LC2 |
|---|---|---|---|---|
| (c) | HC1(c) | LC1(c) | HC2(c) | LC2(c) |
| (d) | HC1(d) | LC1(d) | HC2(d) | LC2(d) |
| (e) | HC1(e)(f) | LC1(e)(f) | HC2(e)(f) | LC2(e) |
| (f) | HC1(e)(f) | LC1(e)(f) | HC2(e)(f) | LC2(f) |

In some embodiments, the HCs and LCs for a kappa×kappa bi-specific construct can be switched around. For example, in some embodiments, the (c), (d), (e), and (f) designs of Table 13 can be swapped such that the LAG3 HC1+LC1 and PD-1 HC2+LC2 use the opposite light-chain pairing mutations, as illustrated in Table 14.

TABLE 14
Exemplary embodiments of a four-chain bi-specific
antibody arrangement (kappa × kappa)

| Bispecific | PD-1 HC1 | PD-1 LC1 | LAG3 HC2 | LAG3 LC2 |
|---|---|---|---|---|
| (c) | HC1(c) | LC1(c) | HC2(c) | LC2(c) |
| (d) | HC1(d) | LC1(d) | HC2(d) | LC2(d) |
| (e) | HC1(e)(f) | LC1(e)(f) | HC2(e)(f) | LC2(e) |
| (f) | HC1(e)(f) | LC1(e)(f) | HC2(e)(f) | LC2(f) |

In some embodiments, HC1 and HC2 incorporate complementary Zymeworks mutations A (T350V/L351Y/F405A/Y407V; “zwA”) and B (T350V/T366L/K392L/T394W; “zwB”) to enforce heterodimerization of HC1 with HC2 (see, e.g., Von Kreudenstein et al. 2013. mAbs 5(5):646-654; Von Kreudenstein et al. 2014. Methods 65:77-94; U.S. 2013/0195849, each of which is incorporated herein by reference in its entirety). Accordingly, if HC1 has zwA then HC2 must have zwB; if HC1 has zwB then HC2 must have zwA. Many alternatives to these C_{H}3 pairing mutations, e.g., knobs-in-holes mutations, can be used instead. Some of these alternatives are described, for example, in Brinkmann, U., and R. E. Kontermann. 2017. mAbs 9(2):182-212; and Yang, et al. 2017. Int. J. Mol. Sci. 18(1):48 (21 pages), each of which is incorporated herein by reference in its entirety. The C_{H}2 domains can either be wild-type or have stability/solubility/assembly/yield enhancing mutations V262E or V264S.

4.1.6.5.1 LAG3 Heavy Chains (HC1 or HC2)

A full-length antibody LAG3 heavy chain typically includes a V_{H} domain, a C_{H}1 domain, a linker, and a C_{H}2-C_{H}3 region. In some embodiments, the V_{H} domain comprises, consists, or consists essentially of any one of SEQ ID NOs: 191-210. In some embodiments, the C_{H}1 domain is selected from SEQ ID NOs: 343-352. In some embodiments, the linker comprises, consists of, or consists essentially of SEQ ID NO: 316. In some embodiments, the C_{H}2-C_{H}3 region comprises, consists of, or consists essentially of any one of SEQ ID NOs: 331-336. For example, in an embodiment where the anti-LAG3 arm is antibody “1” in the bi-specific antibody, an anti-LAG3 heavy chain with design (a) (“HC1(a)”) can be constructed from: (1) a V_{H} sequence of 1449.G09.2 (SEQ ID NO: 200); (2) C_{H}1-(a)1; (3) a linker comprising, consisting of, or consisting essentially of SEQ ID NO: 316, and (4) a C_{H}2-C_{H}3 region comprising, consisting of, or consisting essentially of Fc-zwA. Table 15 provides various exemplary components for the V_{H} domain, C_{H}1 domain, linker, and C_{H}2-C_{H}3 region that are contemplated for generation of a LAG-3 heavy chain sequence.

TABLE 15
Exemplary Combinations of Components
for LAG-3 Heavy Chain (HC1 or HC2)

| Design | V_{H} | C_{H}1 | Linker | C_{H}2-C_{H}3 |
|---|---|---|---|---|
| (a) | 1449-G09.2 | C_{H}1-(a)1 | Hinge-wt | Fc-zwA; |
| | | C_{H}1-(a)2 | | Fc-zwA V262E; |
| | | | | Fc-zwA V264S; |
| | | | | Fc-zwB; |
| | | | | Fc-zwB V262E; |
| | | | | Fc-zwB V264S |
| (b) | 1449-G09.2 | C_{H}1-(b)1 | Hinge-wt | Fc-zwA; |
| | | C_{H}1-(b)2 | | Fc-zwA V262E; |
| | | | | Fc-zwA V264S; |
| | | | | Fc-zwB; |
| | | | | Fc-zwB V262E; |
| | | | | Fc-zwB V264S |
| (c) | 1449-G09.2 | C_{H}1-(c)1 | Hinge-wt | Fc-zwA; |
| | | C_{H}1-(c)2 | | Fc-zwA V262E; |
| | | | | Fc-zwA V264S; |
| | | | | Fc-zwB; |
| | | | | Fc-zwB V262E; |
| | | | | Fc-zwB V264S |
| (d) | 1449-G09.2 | C_{H}1-(d)1 | Hinge-wt | Fc-zwA; |
| | | C_{H}1-(d)2 | | Fc-zwA V262E; |
| | | | | Fc-zwA V264S; |
| | | | | Fc-zwB; |
| | | | | Fc-zwB V262E; |
| | | | | Fc-zwB V264S |
| (e)(f) | 1449-G09.2 | C_{H}1-(e)(f)1 | Hinge-wt | Fc-zwA; |
| | | C_{H}1-(e)(f)2 | | Fc-zwA V262E; |
| | | | | Fc-zwA V264S; |
| | | | | Fc-zwB; |
| | | | | Fc-zwB V262E; |
| | | | | Fc-zwB V264S |

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449.G09.2 (SEQ ID NO: 200), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(d)1 (SEQ ID NO: 346, a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)1 (SEQ ID NO: 346, a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)1 (SEQ ID NO: 346, a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)1 (SEQ ID NO: 346, a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)1 (SEQ ID NO: 346, a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(d)2 (SEQ ID NO: 351, a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)2 (SEQ ID NO: 351, a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)2 (SEQ ID NO: 351, a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)2 (SEQ ID NO: 351, a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)2 (SEQ ID NO: 351, a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(e)(f)1 (SEQ ID NO: 347, a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(f)1 (SEQ ID NO: 347, a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(f)1 (SEQ ID NO: 347, a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(f)1 (SEQ ID NO: 347, a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(01 (SEQ ID NO: 347, a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwB V264S (SEQ ID NO: 336).

In some embodiments, the anti-LAG-3 heavy chain comprises a V_{H} sequence comprising the V_{H} sequence of 1449-G09.2 (SEQ ID NO: 200), a C_{H}1 sequence comprising the sequence of C_{H}1-(e)(02 (SEQ ID NO: 352, a linker of hinge-wt (SEQ ID NO: 316), and a C_{H}2-C_{H}3 region comprising the sequence of Fc-zwA (SEQ ID NO: 331). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(02 (SEQ ID NO: 352, a linker of hinge-wt (SEQ ID NO: 316), and a C_{H}2-C_{H}3 region comprising the sequence of Fc-zwA V262E (SEQ ID NO: 332). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(02 (SEQ ID NO: 352, a linker of hinge-wt (SEQ ID NO: 316), and a C_{H}2-C_{H}3 region comprising the sequence of Fc-zwA V264S (SEQ ID NO: 333). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(02 (SEQ ID NO: 352, a linker of hinge-wt (SEQ ID NO: 316), and a C_{H}2-C_{H}3 region comprising the sequence of Fc-zwB (SEQ ID NO: 334). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(02 (SEQ ID NO: 352, a linker of hinge-wt (SEQ ID NO: 316), and a C_{H}2-C_{H}3 region comprising the sequence of Fc-zwB V262E (SEQ ID NO: 335). In some embodiments, the anti-LAG3 heavy chain comprises a V_{H} sequence comprising the V_{H} sequence of 1449-G09.2 (SEQ ID NO: 200), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwB V264S (SEQ ID NO: 336).

4.1.6.5.2 LAG3 Light Chains (LC1 or LC2)

A full-length anti-LAG3 light chain typically includes a V_{L} domain and a C_{L} domain. In some embodiments, the V_{L} domain comprises, consists of, or consists essentially of any one of SEQ ID NOs: 251-266. In some embodiments, the C_{L} domain comprises, consists of, or consists essentially of any one of SEQ ID NOs: 353-364. For example, in an embodiment where the anti-LAG3 arm is antibody “1” in the bi-specific antibody, an anti-LAG3 light chain with design (a) (“LC1(a)”) can be constructed from: (1) V_{L} sequence 1449-G09.2 (SEQ ID NO: 254); and (2) Ck-(a)1. Table 16 provides various exemplary components for the V_{L} domain and C_{L} domain for generation of a LAG3 light chain sequence.

TABLE 16
Exemplary Combinations of Components
for LAG-3 Light Chain (LC1 or LC2)

| Design | V_{L} | C_{L} |
|---|---|---|
| (a) | 1449-G09.2 | Ck-(a)1; |
| | | Ck-(a)2 |
| (b) | 1449-G09.2 | Ck-(b)1; |
| | | Ck-(b)2 |
| (c) | 1449-G09.2 | Ck-(c)1; |
| | | Ck-(c)2 |
| (d) | 1449-G09.2 | Ck-(d)1; |
| | | Ck-(d)2 |
| (e) | 1449-G09.2 | Ck-(e)(f)1; |
| | | Ck-(e)2 |
| (f) | 1449-G09.2 | Ck-(e)(f)1; |
| | | Ck-(f)2 |

In some embodiments, the anti-LAG3 light chain comprises a V_{L} sequence comprising the V_{L} sequence of 1449-G09.2 (SEQ ID NO: 254) and a C_{L} sequence comprising the sequence of Ck-(a)1 (SEQ ID NO: 353).

In some embodiments, the anti-LAG3 light chain comprises a V_{L} sequence comprising the V_{L} sequence of 1449-G09.2 (SEQ ID NO: 254) and a C_{L} sequence comprising the sequence of Ck-(b)1 (SEQ ID NO: 354).

In some embodiments, the anti-LAG3 light chain comprises a V_{L} sequence comprising the V_{L} sequence of 1449-G09.2 (SEQ ID NO: 254) and a C_{L} sequence comprising the sequence of Ck-(c)1 (SEQ ID NO: 355). In some embodiments, the anti-LAG3 light chain comprises a V_{L} sequence comprising the V_{L} sequence of 1449-G09.2 (SEQ ID NO: 254) and a C_{L} sequence comprising the sequence of Ck-(c)2 (SEQ ID NO: 360).

In some embodiments, the anti-LAG3 light chain comprises a V_{L} sequence comprising the V_{L} sequence of 1449-G09.2 (SEQ ID NO: 254) and a C_{L} sequence comprising the sequence of Ck-(d)1 (SEQ ID NO: 356). In some embodiments, the anti-LAG3 light chain comprises a V_{L} sequence comprising the V_{L} sequence of 1449-G09.2 (SEQ ID NO: 254) and a C_{L} sequence comprising the sequence of Ck-(d)2 (SEQ ID NO: 361).

In some embodiments, the anti-LAG3 light chain comprises a V_{L} sequence comprising the V_{L} sequence of 1449-G09.2 (SEQ ID NO: 254) and a C_{L} sequence comprising the sequence of Ck-(e)(f)1 (SEQ ID NO: 357). In some embodiments, the anti-LAG3 light chain comprises a V_{L} sequence comprising the V_{L} sequence of 1449-G09.2 (SEQ ID NO: 254) and a C_{L} sequence comprising the sequence of Ck-(e)2 (SEQ ID NO: 362). In some embodiments, the anti-LAG3 light chain comprises a V_{L} sequence comprising the V_{L} sequence of 1449-G09.2 (SEQ ID NO: 254) and a C_{L} sequence comprising the sequence of Ck-(f)2 (SEQ ID NO: 363).

4.1.6.5.3 PD-1 Heavy Chains (HC1 or HC2)

A full-length anti-PD-1 heavy chain typically includes a V_{H} domain, a C_{H}1 domain, a linker, and a C_{H}2-C_{H}3 region. In some embodiments, the V_{H} domain comprises, consists, or consists essentially of any one of SEQ ID NOs: 211-227. In some embodiments, the C_{H}1 domain is selected from SEQ ID NOs: 343-352. In some embodiments, the linker comprises, consists of, or consists essentially of SEQ ID NO: 316. In some embodiments, the C_{H}2-C_{H}3 region comprises, consists of, or consists essentially of any one of SEQ ID NOs: 331-336. For example, in an embodiment where the anti-PD-1 arm is antibody “2” in the bi-specific antibody, an anti-PD-1 hearby chain with design (a) (“HC2a”) can be constructed from: (1) V_{H} sequence 1353-G10 R28T/P30D/H31S (SEQ ID NO: 227); (2) C_{H}1(a)2; and (3) a linker comprising, consisting of, or consisting essentially of SEQ ID NO: 316, and (4) a C_{H}2-C_{H}3 region comprising, consisting of, or consisting essentially of Fc-zwA. Table 17 provides various exemplary components for the V_{H} domain, C_{H}1 domain, linker, and C_{H}2-C_{H}3 region that are contemplated for generation of a PD-1 heavy chain sequence.

TABLE 17
Exemplary Combinations of Components
for PD-1 Heavy Chain (HC1 or HC2)

| Design | V_{H} | C_{H}1 | Linker | C_{H}2-C_{H}3 |
|---|---|---|---|---|
| (a) | 1353-G10-wt; | C_{H}1-(a)1; | Hinge-wt | Fc-zwA; |
| | 1353-G10-28T/ | C_{H}1-(a)2 | | Fc-zwA V262E; |
| | P30D/H31S | | | Fc-zwA V264S; |
| | | | | Fc-zwB; |
| | | | | Fc-zwB V262E; |
| | | | | Fc-zwB V264S |
| (b) | 1353-G10-wt; | C_{H}1-(b)1; | Hinge-wt | Fc-zwA; |
| | 1353-G10-28T/ | C_{H}1-(b)2 | | Fc-zwA V262E; |
| | P30D/H31S | | | Fc-zwA V264S; |
| | | | | Fc-zwB; |
| | | | | Fc-zwB V262E; |
| | | | | Fc-zwB V264S |
| (c) | 1353-G10-wt; | C_{H}1-(c)1; | Hinge-wt | Fc-zwA; |
| | 1353-G10-28T/ | C_{H}1-(c)2 | | Fc-zwA V262E; |
| | P30D/H31S | | | Fc-zwA V264S; |
| | | | | Fc-zwB; |
| | | | | Fc-zwB V262E; |
| | | | | Fc-zwB V264S |
| (d) | 1353-G10-wt; | C_{H}1-(d)1; | Hinge-wt | Fc-zwA; |
| | 1353-G10-28T/ | C_{H}1-(d)2 | | Fc-zwA V262E; |
| | P30D/H31S | | | Fc-zwA V264S; |
| | | | | Fc-zwB; |
| | | | | Fc-zwB V262E; |
| | | | | Fc-zwB V264S |
| (e)(f) | 1353-G10-wt; | C_{H}1-(e)(f)1; | Hinge-wt | Fc-zwA; |
| | 1353-G10-28T/ | C_{H}1-(e)(f)2 | | Fc-zwA V262E; |
| | P30D/H31S | | | Fc-zwA V264S; |
| | | | | Fc-zwB; |
| | | | | Fc-zwB V262E; |
| | | | | Fc-zwB V264S |

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)1 (SEQ ID NO: 343), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(a)2 (SEQ ID NO: 348), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)1 (SEQ ID NO: 344), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(b)2 (SEQ ID NO: 349), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)1 (SEQ ID NO: 345), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(c)2 (SEQ ID NO: 350), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)1 (SEQ ID NO: 346), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(d)2 (SEQ ID NO: 351), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwA (SEQ ID NO: 331). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwA (SEQ ID NO: 331).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwA V262E (SEQ ID NO: 332). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwA V262E (SEQ ID NO: 332).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwA V264S (SEQ ID NO: 333). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwA V264S (SEQ ID NO: 333).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwB (SEQ ID NO: 334). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwB (SEQ ID NO: 334).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwB V262E (SEQ ID NO: 335). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwB V262E (SEQ ID NO: 335).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)1 (SEQ ID NO: 347), a linker of hinge-wt (SEQ ID NO: 316), and a C_{H}2-C_{H}3 region comprising the sequence of Fc-zwB V264S (SEQ ID NO: 336).

In some embodiments, the anti-PD-1 heavy chain comprises a V_{H} sequence comprising the V_{H} sequence of 1353-G10-wt (SEQ ID NO: 211), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwB V264S (SEQ ID NO: 336). In some embodiments, the anti-PD-1 heavy chain comprises a V_{H} sequence comprising the V_{H} sequence of 1353-G10-R28T/P30D/H31S (SEQ ID NO: 227), a C_{H}1 sequence comprising the sequence of C_{H}1-(e)(f)2 (SEQ ID NO: 352), a linker of hinge-wt (SEQ ID NO: 316), and a C_{H}2-C_{H}3 region comprising the sequence of Fc-zwB V264S (SEQ ID NO: 336).

4.1.6.5.4 PD-1 Light Chains (LC1 or LC2)

A full-length anti-PD-1 light chain typically includes a V_{L} domain and a C_{L} domain. In some embodiments, the V_{L} domain comprises, consists of, or consists essentially of any one of SEQ ID NOs: 267-276. In some embodiments, the C_{L} domain comprises, consists of, or consists essentially of any one of SEQ ID NOs: 353-364. For example, in an embodiment where the anti-PD-1 arm is antibody “2” in the bi-specific antibody, an anti-PD-1 light chain with design (a) (“LC2a”) can be constructed from: (1) V_{L} sequence 1353-G10 wt (SEQ ID NO: 267); and (2) Cl-(a)2. Table 18 provides various exemplary components for the V_{L} domain and C_{L} domain for generation of a PD-1 light chain sequence.

TABLE 18
Exemplary Combinations of Components
for PD-1 Light Chain (LC1 or LC2)

| Design | V_{L} | C_{L} |
|---|---|---|
| (a) | 1353-G10 wt (λ) | Cl-(a)2 |
| (b) | 1353-G10 wt (λ) | Cl-(b)2 |
| (c) | 1353-G10 Vk1-39 (κ) | Ck-(c)1; |
| | | Ck-(c)2 |
| (d) | 1353-G10 Vk1-39 (κ) | Ck-(d)1; |
| | | Ck-(d)2 |
| (e) | 1353-G10 Vk1-39 (κ) | Ck-(e)2; |
| | | Ck-(e)(f)2 |
| (f) | 1353-G10 Vk1-39 (κ) | Ck-(f)2; |
| | | Ck-(e)(f)2 |

In some embodiments, the anti-PD-1 light chain comprises a V_{L} sequence comprising the V_{L} sequence of 1353-G10 wt (λ) (SEQ ID NO: 267) and a C_{L} sequence comprising the sequence of Cl-(a)2 (SEQ ID NO: 358).

In some embodiments, the anti-PD-1 light chain comprises a V_{L} sequence comprising the V_{L} sequence of 1353-G10 wt (λ) (SEQ ID NO: 267) and a C_{L} sequence comprising the sequence of Cl-(b)2 (SEQ ID NO: 359).

In some embodiments, the anti-PD-1 light chain comprises a V_{L} sequence comprising the V_{L} sequence of 1353-G10 Vk1-39 (κ) (SEQ ID NO: 275) and a C_{L} sequence comprising the sequence of Ck-(c)1 (SEQ ID NO: 355). In some embodiments, the anti-PD-1 light chain comprises a V_{L} sequence comprising the V_{L} sequence of 1353-G10 Vk1-39 (K) (SEQ ID NO: 275) and a C_{L} sequence comprising the sequence of Ck-(c)2 (SEQ ID NO: 360).

In some embodiments, the anti-PD-1 light chain comprises a V_{L} sequence comprising the V_{L} sequence of 1353-G10 Vk1-39 (κ) (SEQ ID NO: 275) and a C_{L} sequence comprising the sequence of Ck-(d)1 (SEQ ID NO: 356). In some embodiments, the anti-PD-1 light chain comprises a V_{L} sequence comprising the V_{L} sequence of 1353-G10 Vk1-39 (κ) (SEQ ID NO: 275) and a C_{L} sequence comprising the sequence of Ck-(d)2 (SEQ ID NO: 361).

In some embodiments, the anti-PD-1 light chain comprises a V_{L} sequence comprising the V_{L} sequence of 1353-G10 Vk1-39 (κ) (SEQ ID NO: 275) and a C_{L} sequence comprising the sequence of Ck-(e)2 (SEQ ID NO: 362). In some embodiments, the anti-PD-1 light chain comprises a V_{L} sequence comprising the V_{L} sequence of 1353-G10 Vk1-39 (κ) (SEQ ID NO: 275) and a C_{L} sequence comprising the sequence of Ck-(e)(f)1 (SEQ ID NO: 357).

In some embodiments, the anti-PD-1 light chain comprises a V_{L} sequence comprising the V_{L} sequence of 1353-G10 Vk1-39 (κ) (SEQ ID NO: 275) and a C_{L} sequence comprising the sequence of Ck-(f)2 (SEQ ID NO: 363). In some embodiments, the anti-PD-1 light chain comprises a V_{L} sequence comprising the V_{L} sequence of 1353-G10 Vk1-39 (K) (SEQ ID NO: 275) and a C_{L} sequence comprising the sequence of Ck-(e)(f)1 (SEQ ID NO: 357).

4.1.6.5.5 Hybrid Light Chains (LC1 or LC2=Vλ+Cκ)

In some embodiments, the anti-LAG3 or anti-PD-1 light chain comprises a V_{L} lambda sequence (“Vλ”) and a C_{L} kappa sequence (“Cκ”). In such embodiments, the Vλ, sequence comprises one or more mutations selected from the group consisting of: E38F, D85T, T105E, V1061, and L106K. In some embodiments, the light chain is an anti-PD-1 light chain comprising, consisting of, or consisting essentially of SEQ ID NOs: 394 or 395.

In some embodiments, a PD-1/LAG3 bi-specific antigen-binding construct is provided, comprising a hybrid light chain comprising a Vλ, and a Cκ sequence, wherein HC1, LC1, HC2, and LC2 comprise one or more mutations selected from the Table 19:

TABLE 19
Table of mutations for a bi-specific
construct with a hybrid light chain

| Bispecific | HC1 | LC1 | HC2 | LC2 |
|---|---|---|---|---|
| (c) | A139W; | F116A; | Q179K | Q124E; |
| | L143E; | Q124R; | | L135W; |
| | K145T; | L135V; | | Q160E; |
| | Q179E | T178R | | T180E |
| (d) | Q39E; | Q38R; | Q39R; | Q38E; |
| | L143E; | Q124R; | H172R; | Q124E; |
| | K145T; | Q160K; | Q179K | Q160E; |
| | Q179E | T178R | | T180E |

In some embodiments, HC1 and LC1 indicate PD-1 heavy chain and light chain sequences, respectively, and HC2 and LC2 indicate LAG3 heavy chain and light chain sequences, respectively. In some embodiments, HC1 and LC1 indicate LAG3 heavy chain and light chain sequences, respectively, and HC2 and LC2 indicate PD-1 heavy chain and light chain sequences, respectively.

In some embodiments, the V_{H} and V_{L} sequences of HC1, HC2, LC1 and LC2 comprise the following mutations from Table 20:

TABLE 20
Exemplary mutations for a bi-specific
construct with a hybrid light chain

| V_{H}1 | V_{L}1 | V_{H}2 | V_{L}2 |
|---|---|---|---|
| Q39R | Q38E | Q39E | Q38R |

In some embodiments, HC1 comprises, consists of, or consists essentially of SEQ ID NO: 397; LC1 comprises, consists of, or consists essentially of SEQ ID NO: 395; HC2 comprises, consists of, or consists essentially of SEQ ID NO: 398; and LC2 comprises, consists of, or consists essentially of SEQ ID NO: 396.

The sequences of the various components contemplated for V_{H}, C_{H}1, linker, C_{H}2-C_{H}3, V_{L}, and C_{L} in Section 4.1.6 can be found in Table 35.

4.1.7. Additional Mutations

In some embodiments, an bi-specific antibody or antigen-binding construct as disclosed herein can include additional mutations. In some embodiments, the bi-specific antibody or antigen-binding construct can include a mutation to remove a methionine start residue. In some embodiments, the bi-specific antibody or antigen-binding construct can include a mutation to remove glycosylation (e.g. N297A). In some embodiments, the bi-specific antibody or antigen-binding construct can include a mutation to remove effector function (e.g., AAS mutation, as described in U.S. Patent Publication No. 2016/0075792, which is incorporated herein by reference in its entirety). For example, in some embodiments, one or more of the additional mutations disclosed herein can be used to improve production of bi-specific antibody constructs or bi-specific antibody components in a host cell.

### 5. Germline

In some embodiments, an antibody or bi-specific antibody as disclosed herein that specifically binds LAG3 is an antibody comprising a variable region that is encoded by a particular germline gene, or a variant thereof. The illustrative antibodies provided herein comprise variable regions that are encoded by the heavy chain variable region germline genes VH3-23 and VH5-51, or variants thereof; and the light chain variable region germline genes Vκ3-20 and Vκ4-1, or variants thereof.

One of skill in the art would recognize that the CDR sequences provided herein may also be useful when combined with variable regions encoded by other variable region germline genes, or variants thereof. In particular, the CDR sequences provided herein may be useful when combined with variable regions encoded by variable region germline genes, or variants thereof, that are structurally similar to the variable region germline genes recited above. For example, in some embodiments, a CDR-H sequence provided herein may be combined with a variable region encoded by a variable region germline gene selected from the V_{H} 3 or V_{H} 5 families, or a variant thereof. In some embodiments, a CDR-L sequence provided herein may be combined with a variable region encoded by a variable region germline gene selected from the Vκ3 or Vκ4 families, or a variant thereof.

### 6. Affinity

In some embodiments, the affinity of an antibody or bi-specific antibody as disclosed herein for LAG3 as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody or bi-specific antibody is between about 10⁻⁷ M and 10⁻¹¹ M. In some embodiments, the affinity of the antibody or bi-specific antibody is between about 10⁻⁷ M and 10⁻¹⁰ M. In some embodiments, the affinity of the antibody or bi-specific antibody is between about 10⁻⁷ M and 10⁻⁹ M. In some embodiments, the affinity of the antibody or bi-specific antibody is between about 10⁻⁷ M and 10⁻⁸ M. In some embodiments, the affinity of the antibody or bi-specific antibody is between about 10⁻⁸ M and 10⁻¹¹ M. In some embodiments, the affinity of the antibody or bi-specific antibody is between about 10⁻⁸ M and 10⁻¹⁰ M. In some embodiments, the affinity of the antibody or bi-specific antibody is between about 10⁻⁹ M and 10⁻¹¹ M. In some embodiments, the affinity of the antibody or bi-specific antibody is between about 10⁻¹⁰ M and 10⁻¹¹M.

In some embodiments, the affinity of an antibody or bi-specific antibody as disclosed herein for human LAG3, as determined by surface plasmon resonance at 25° C., and as indicated by K_{D}, is between about 1.3×10⁻⁸ M and about 1.93×10⁻¹⁰ M. In some embodiments, the affinity of the antibody or bi-specific antibody for human LAG3 is about 8.63×10⁻⁷ M, about 4.33×10⁻⁸ M, about 3.90×10⁻⁸ M, about 3.10×10⁻⁸ M, about 2.40×10⁻⁸ M, about 2.13×10⁻⁸ M, about 1.89×10⁻⁸ M, about 1.52×10⁻⁸ M, about 1.47×10⁻⁸M, about 1.35×10⁻⁸ M, about 1.30×10⁻⁸ M, about 1.03×10⁻⁸ M, about 3.10×10⁻⁹ M, about 2.46×10⁻⁹ M, about 2.27×10⁻⁹ M, about 1.36×10⁻⁹ M, about 6.76×10⁻¹⁰ M, about 6.40×10⁻¹⁰ M, or about 4.12×10⁻¹¹ M.

In some embodiments an antibody or bi-specific antibody as disclosed herein has a kₐ of at least about 10⁴ M⁻¹×sec⁻¹. In some embodiments the antibody or bi-specific antibody has a kₐ of at least about 10⁵ M⁻¹×sec⁻¹. In some embodiments the antibody or bi-specific antibody has a kₐ of at least about 10⁶ M⁻¹×sec⁻¹. In some embodiments the antibody or bi-specific antibody has a kₐ of between about 10⁴ M⁻¹×sec⁻¹ and about 10⁵ M⁻¹×sec⁻¹. In some embodiments the antibody or bi-specific antibody has a kₐ of between about 10⁵ M⁻¹×sec⁻¹ and about 10⁶ M⁻¹×sec⁻¹.

In some embodiments an antibody or bi-specific antibody as disclosed herein has a kₐ when associating with human LAG3, as determined by surface plasmon resonance at 25° C., of between about 5.02×10⁴ M⁻¹×sec⁻¹ and about 5.31×10⁷ M⁻¹×sec⁻¹. In some embodiments the antibody or bi-specific antibody has a kₐ when associating with human LAG3 of about 2.67×10³ M⁻¹×sec⁻¹, about 5.02×10⁴ M⁻¹×sec⁻¹, about 1.61×10⁵ M⁻¹×sec⁻¹, about 2.61×10⁵ M⁻¹×sec⁻¹, about 3.12×10⁵ M⁻¹×sec⁻¹, about 4.35×10⁵ M⁻¹×sec⁻¹, about 4.60×10⁵ M⁻¹×sec⁻¹, about 4.72×10⁵ M⁻¹×sec⁻¹, about 5.60×10⁵ M⁻¹×sec⁻¹, about 7.90×10⁵ M⁻¹×sec⁻¹, about 7.94×10⁵ M⁻¹×sec⁻¹, about 1.06×10⁶ M⁻¹×sec⁻¹, about 1.24×10⁶ M⁻¹×sec⁻¹, about 1.29×10⁶ M⁻¹×sec⁻¹, about 1.31×10⁶ M⁻¹×sec⁻¹, about 1.64×10⁶M⁻¹×sec⁻¹, about 1.65×10⁶M⁻¹×sec⁻¹, about 1.12×10⁷ M⁻¹×sec⁻¹, or about 5.35×10⁷M⁻¹×sec⁻¹.

In some embodiments an antibody or bi-specific antibody as disclosed herein has a k_{d} of about 10⁻⁵ sec⁻¹ or less. In some embodiments the antibody or bi-specific antibody has a k_{d} of about 10⁻⁴ sec⁻¹ or less. In some embodiments the antibody or bi-specific antibody has a k_{d} of about 10⁻³ sec⁻¹ or less. In some embodiments the antibody or bi-specific antibody has a k_{d} of between about 10⁻² sec⁻¹ and about 10⁻⁵ sec⁻¹. In some embodiments the antibody or bi-specific antibody has a k_{d} of between about 10⁻² sec⁻¹ and about 10⁻⁴ sec⁻¹. In some embodiments the antibody or bi-specific antibody has a k_{d} of between about 10⁻³ sec⁻¹ and about 10⁻⁵ sec⁻¹.

In some embodiments an antibody or bi-specific antibody as disclosed herein has a k_{d} when dissociating from human LAG3, as determined by surface plasmon resonance at 25° C., of between about 2.79×10⁻² sec⁻¹ and about 6.78×10⁻⁵ sec⁻¹. In some embodiments the antibody or bi-specific antibody has a k_{d} when dissociating from human LAG3 of about 1.22×10⁻¹ sec⁻¹, about 7.10×10⁻² sec⁻¹, about 2.79×10⁻² sec⁻¹, about 2.75×10⁻² sec⁻¹, about 2.34×10⁻² sec⁻¹, about 1.96×10⁻² sec⁻¹, about 1.70×10⁻² sec⁻¹, about 1.52×10⁻² sec⁻¹, about 1.10×10⁻² sec⁻¹, about 9.90×10⁻³ sec⁻¹, about 6.20×10⁻³ sec⁻¹, about 4.22×10⁻³ sec⁻¹, about 2.30×10⁻³ sec⁻¹, about 8.07×10⁻⁴ sec⁻¹, about 6.27×10⁻⁴ sec⁻¹, about 5.36×10⁻⁴ sec⁻¹, about 5.15×10⁻⁴ sec⁻¹, about 3.02×10⁻⁴ sec⁻¹, or about 6.78×10⁻⁵ sec⁻¹.

In some embodiments, the affinity of an antibody or bi-specific antibody as disclosed herein for human LAG3 expressed on the surface of a cell, as indicated by K_{D}, is between about 78.0 and about 0.19 nM. In some embodiments, the affinity of an antibody or bi-specific antibody as disclosed herein for human LAG3 expressed on the surface of a cell is about 78.0 nM, about 40.6 nM, about 39.4 nM, about 35.0 nM, about 3.37 nM, about 1.92 nM, about 1.54 nM, about 1.06 nM, about 0.97 nM, about 0.74 nM, about 0.50 nM, about 0.40 nM, about 0.32 nM, about 0.30 nM, and about 0.19 nM. In some embodiments, the cell is a CHO cell. In some embodiments, the cell is a 293T cell.

In some embodiments, the affinity of an antibody or bi-specific antibody as disclosed herein for cynomolgus LAG3, as determined by surface plasmon resonance at 25° C., and as indicated by K_{D}, is between about 4.5×10⁻⁹ M and about 0.3×10⁻⁹ M. In some embodiments, the affinity of the antibody or bi-specific antibody for cynomolgus LAG3 is about 4.5×10⁻⁹ M, about 1.6×10⁻⁹ M, about 1.0×10⁻⁹ M, about 0.7×10⁻⁹ M, or about 0.3×10⁻⁹ M.

In some embodiments, the affinity of an antibody or bi-specific antibody as disclosed herein for PD-1, as indicated by K_{D}, is less than about 10⁻⁵ M, less than about 10⁻⁶ M, less than about 10⁻⁷ M, less than about 10⁻⁸ M, less than about 10⁻⁹ M, less than about 10⁻¹⁰ M, less than about 10⁻¹¹ M, or less than about 10⁻¹² M. In some embodiments, the affinity of the antibody or bi-specific antibody is between about 10⁻⁷ M and 10⁻¹¹ M. In some embodiments, the affinity of the antibody or bi-specific antibody is between about 10⁻⁷ M and 10⁻¹⁰ M. In some embodiments, the affinity of the antibody or bi-specific antibody is between about 10⁻⁷ M and 10⁻⁹ M. In some embodiments, the affinity of the antibody or bi-specific antibody is between about 10⁻⁷ M and 10⁻⁸ M. In some embodiments, the affinity of the antibody or bi-specific antibody is between about 10⁻⁸ M and 10⁻¹¹ M. In some embodiments, the affinity of the antibody or bi-specific antibody is between about 10⁻⁸ M and 10⁻¹⁰ M. In some embodiments, the affinity of the antibody or bi-specific antibody is between about 10⁻⁹M and 10⁻¹¹ M. In some embodiments, the affinity of the antibody or bi-specific antibody is between about 10⁻¹⁰ M and 10⁻¹¹M.

In some embodiments, the affinity of an antibody or bi-specific antibody as disclosed herein for human PD-1 is between about 3.85×10⁻⁸ M and 2.52×10⁻¹° M. In some embodiment, the affinity of the antibody or bi-specific antibody for human PD-1 is about 2.55×10⁻⁸ M, about 1.52×10⁻⁸ M, about 9.52×10⁻⁹ M, about 1.09×10⁻⁸ M, about 4.50×10⁻⁹ M, about 1.90×10⁻⁹ M, about 4.76×10⁻⁹ M, about 4.5×10⁻⁹ M, about 1.04×10⁻⁸ M, about 9.90×10⁻⁹ M, about 9.13×10⁻¹⁰ M, about 2.52×10⁻¹° M, about 2.58×10⁻⁹ M, about 3.85×10⁻⁸ M, about 3.66×10⁻⁹ M, about 3.15×10⁻⁹ M, about 5.14×10⁻⁹ M, about 2.47×10⁻⁹ M, about 2.79×10⁻⁹ M, about 1.20×10⁻⁹ M, or about 1.28×10⁻⁸ M

In some embodiments, the affinity of an antibody or bi-specific antibody as disclosed herein for human PD-1 expressed on the surface of a cell is between about 3.2 and about 0.2 nM. In some embodiment, the affinity of the antibody or bi-specific antibody for human PD-1 expressed on the surface of a cell is about 0.2 nM, about 0.4 nM, about 0.9 nM, about 1 nM, about 0.3 nM, about 0.7 nM, about 0.2 nM, about 0.8 nM, about 3.2 nM, about 2.9 nM, about 1.39 nM, or about 1.34 nM.

In some embodiments, the affinity of an antibody or bi-specific antibody as disclosed herein for murine PD-1 is between about 6.09×10⁻⁸ M and 9.08×10⁻⁹ M. In some embodiment, the affinity of the antibody or bi-specific antibody for murine PD-1 is about 6.09×10⁻⁸ M, about 6.22×10⁻⁸M, or about 9.08×10⁻⁹ M.

In some embodiments, the affinity of an antibody or bi-specific antibody as disclosed herein for cynomolgus PD-1 is between about 2.43×10⁻⁸ M and 1.95×10⁻¹° M. In some embodiment, the affinity of the antibody or bi-specific antibody for cynomolgus PD-1 is about 2.43×10⁻⁸ M, about 1.55×10⁻⁸ M, about 2.22×10⁻⁸ M, about 2.56×10⁻⁹ M, about 2.54×10⁻⁹M, about 5.61×10⁻¹⁰ M, or about 1.95×10⁻¹⁰ M

In some embodiments an antibody or bi-specific antibody as disclosed herein has a kₐ of at least about 10⁴ M⁻¹×sec⁻¹. In some embodiments the antibody or bi-specific antibody has a kₐ of at least about 10⁵ M⁻¹×sec⁻¹. In some embodiments the antibody or bi-specific antibody has a kₐ of at least about 10⁶ M⁻¹×sec⁻¹. In some embodiments the antibody or bi-specific antibody has a kₐ of between about 10⁴ M⁻¹×sec⁻¹ and about 10⁵ M⁻¹×sec⁻¹. In some embodiments the antibody or bi-specific antibody has a kₐ of between about 10⁵ M⁻¹×sec⁻¹ and about 10⁶ M⁻¹×sec⁻¹.

In some embodiments an antibody or bi-specific antibody as disclosed herein has a kₐ when associating with human PD-1 of between about 4.74×10⁴ M⁻¹×sec⁻¹ and about 1.23×10⁶ M⁻¹×sec⁻¹. In some embodiments the antibody or bi-specific antibody has a kₐ when associating with human PD-1 of about 4.88×10⁵ M⁻¹×sec⁻¹, about 1.23×10⁶ M⁻¹×sec⁻¹, about 7.37×10⁵ M⁻¹×sec⁻¹, about 6.87×10⁵ M⁻¹×sec⁻¹, about 5.63×10⁵ M⁻¹×sec⁻¹, about 5.16×10⁵ M⁻¹×sec⁻¹, about 2.48×10⁵ M⁻¹×sec⁻¹, about 7.98×10⁵ M⁻¹×sec⁻¹, about 1.82×10⁵ M⁻¹×sec⁻¹, about 4.74×10⁴ M⁻¹×sec⁻¹, about 1.85×10⁵ M⁻¹×sec⁻¹, about 2.00×10⁵ M⁻¹×sec⁻¹, about 8.12×10⁴ M⁻¹×sec⁻¹, about 1.21×10⁶ M⁻¹×sec⁻¹, about 1.16×10⁶M⁻¹×sec⁻¹, about 5.13×10⁵ M⁻¹×sec⁻¹, or about 1.86×10⁵ M⁻¹×sec⁻¹.

In some embodiments an antibody or bi-specific antibody as disclosed herein has a k_{d} of about 10⁻⁵ sec⁻¹ or less. In some embodiments the antibody or bi-specific antibody has a k_{d} of about 10⁻⁴ sec⁻¹ or less. In some embodiments the antibody or bi-specific antibody has a k_{d} of about 10⁻³ sec⁻¹ or less. In some embodiments the antibody or bi-specific antibody has a k_{d} of between about 10⁻² sec⁻¹ and about 10⁻⁵ sec⁻¹. In some embodiments the antibody or bi-specific antibody has a k_{d} of between about 10⁻² sec⁻¹ and about 10⁻⁴ sec⁻¹. In some embodiments the antibody or bi-specific antibody has a kₐ of between about 10⁻³ sec⁻¹ and about 10⁻⁵ sec⁻¹.

In some embodiments an antibody or bi-specific antibody as disclosed herein has a k_{d} when dissociating from human PD-1 of between about 1.87×10⁻² sec⁻¹ and about 4.17×10⁻⁴ sec⁻¹. In some embodiments the antibody or bi-specific antibody has a k_{d} when dissociating from human PD-1 of about 1.24×10⁻² sec⁻¹, about 1.87×10⁻² sec⁻¹, about 7.01×10⁻³ sec⁻¹, about 7.74×10⁻³ sec⁻¹, about 2.54×10⁻³ sec⁻¹, about 9.80×10⁻⁴ sec⁻¹, about 1.18×10⁻³ sec⁻¹, about 3.59×10⁻³ sec⁻¹, about 4.68×10⁻⁴ sec⁻¹, about 1.82×10⁻³ sec⁻¹, about 6.79×10⁻⁴ sec⁻¹, about 6.28×10⁻⁴ sec⁻¹, about 4.17×10⁻⁴ sec⁻¹, about 2.99×10⁻³ sec⁻¹, about 3.24×10⁻³ sec⁻¹, about 6.17×10⁻⁴ sec⁻¹, or about 2.39×10⁻³ sec⁻¹.

In some aspects, the K_{D}, kₐ, and k_{d} are determined at 25° C. In some embodiments, the KID, kₐ, and k_{d} are determined by surface plasmon resonance. In some embodiments, the K_{D}, kₐ, and k_{d} are determined according to the methods described in the Examples provided herein.

### 7. Inhibition of PD-L1 and PD-L2 Binding

In some embodiments, an antibody or bi-specific antibody as disclosed herein inhibits binding of one or more of PD-L1 and PD-L2 to PD-1.

In some embodiments, the antibody or bi-specific antibody inhibits binding of PD-L1 to PD-1 with an IC₅₀ of about 1 to about 7 nM. In some aspects, the antibody or bi-specific antibody inhibits binding of PD-L1 to PD-1 with an IC₅₀ of about 1.99, about 2.53, about 5.86, or about 5.96 nM.

In some embodiments, the antibody or bi-specific antibody inhibits binding of PD-L2 to PD-1 with an IC₅₀ of about 0.01 to about 1 nM. In some aspects, the antibody or bi-specific antibody inhibits binding of PD-L2 to PD-1 with an IC₅₀ of about 0.01, about 0.18, about 0.56, or about 0.58 nM.

In some aspects, the antibody or bi-specific antibody inhibits binding of PD-L1 to PD-1 with an IC₅₀ of about 5.96 nM, and inhibits binding of PD-L2 to PD-1 with an IC₅₀ of about 0.56 nM. In some aspects, the antibody or bi-specific antibody inhibits binding of PD-L1 to PD-1 with an IC₅₀ of about 5.86 nM, and inhibits binding of PD-L2 to PD-1 with an IC₅₀ of about 0.58 nM. In some aspects, the antibody or bi-specific antibody inhibits binding of PD-L1 to PD-1 with an IC₅₀ of about 1.99 nM, and inhibits binding of PD-L2 to PD-1 with an IC₅₀ of about 0.01 nM. In some aspects, the antibody or bi-specific antibody inhibits binding of PD-L1 to PD-1 with an IC₅₀ of about 2.53 nM, and inhibits binding of PD-L2 to PD-1 with an IC₅₀ of about 0.18 nM.

### 8. Epitope Bins

In some embodiments, an antibody or bi-specific antibody as disclosed herein binds the same epitope as the scFv antibody provided in SEQ ID NO: 379. In some embodiments, the antibody or bi-specific antibody binds to a different epitope from the scFv antibody provided in SEQ ID NO: 379. In some embodiments, the antibody or bi-specific antibody binds the same epitope as antibody encompassing any of SEQ ID NOs: 191-210. In some embodiments, the antibody or bi-specific antibody binds the same epitope as an antibody comprising any of the V_{H}-V_{L} pairs, above. In some embodiments, the antibody or bi-specific antibody binds to part of the epitope bound by the scFv antibody provided in SEQ ID NO: 379. In some embodiments, the antibody or bi-specific antibody competes for epitope binding with the scFv antibody provided in SEQ ID NO: 379. In some embodiments, the antibody or bi-specific antibody does not compete for epitope binding with scFv antibody provided in SEQ ID NO: 379. In some embodiments, the antibody or bi-specific antibody competes for epitope binding with an antibody encompassing any of SEQ ID NOs: 191-210. In some embodiments, the antibody or bi-specific antibody competes for epitope binding with an antibody comprising any of the V_{H}-V_{L} pairs, above.

In some embodiments, an antibody or bi-specific antibody as disclosed herein binds the same epitope as the scFv antibody provided in SEQ ID NO: 367. In some embodiments, the antibody or bi-specific antibody binds to a different epitope from the scFv antibody provided in SEQ ID NO: 367. In some embodiments, the antibody or bi-specific antibody binds the same epitope as antibody encompassing any of SEQ ID NOs: 211-250. In some embodiments, the antibody or bi-specific antibody binds the same epitope as an antibody comprising any of the V_{H}-V_{L} pairs, above. In some embodiments, the antibody or bi-specific antibody binds to part of the epitope bound by the scFv antibody provided in SEQ ID NO: 367. In some embodiments, the antibody or bi-specific antibody competes for epitope binding with the scFv antibody provided in SEQ ID NO: 367. In some embodiments, the antibody or bi-specific antibody does not compete for epitope binding with scFv antibody provided in SEQ ID NO: 367. In some embodiments, the antibody or bi-specific antibody competes for epitope binding with an antibody encompassing any of SEQ ID NOs: 211-250. In some embodiments, the antibody or bi-specific antibody competes for epitope binding with an antibody comprising any of the V_{H}-V_{L} pairs, above.

### 9. Glycosylation Variants

In certain embodiments, an antibody or bi-specific antibody as disclosed herein may be altered to increase, decrease or eliminate the extent to which it is glycosylated. Glycosylation of polypeptides is typically either “N-linked” or “O-linked.”

“N-linked” glycosylation refers to the attachment of a carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site.

“O-linked” glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition or deletion of N-linked glycosylation sites to the antibody or bi-specific antibody may be accomplished by altering the amino acid sequence such that one or more of the above-described tripeptide sequences is created or removed. Addition or deletion of O-linked glycosylation sites may be accomplished by addition, deletion, or substitution of one or more serine or threonine residues in or to (as the case may be) the sequence of an antibody.

In some embodiments, an antibody or bi-specific antibody as disclosed herein may be aglycosylated.

In some embodiments, an antibody or bi-specific antibody as disclosed herein may be deglycosylated.

### 10. Fc Variants

In certain embodiments, amino acid modifications may be introduced into the Fc region of an antibody or bi-specific antibody provided herein to generate an Fc region variant. In certain embodiments, the Fc region variant possesses some, but not all, effector functions. Such antibodies may be useful, for example, in applications in which the half-life of the antibody or bi-specific antibody in vivo is important, yet certain effector functions are unnecessary or deleterious. Examples of effector functions include complement-dependent cytotoxicity (CDC) and antibody-directed complement-mediated cytotoxicity (ADCC). Numerous substitutions or substitutions or deletions with altered effector function are known in the art.

An alteration in in CDC and/or ADCC activity can be confirmed using in vitro and/or in vivo assays. For example, Fc receptor (FcR) binding assays can be conducted to measure FcγR binding. The primary cells for mediating ADCC, NK cells, express FcγRIII only, whereas monocytes express FcγRI, FcγRII and FcγRIII. FcR expression on hematopoietic cells is summarized in Ravetch and Kinet, Ann. Rev. Immunol., 1991, 9:457-492, incorporated by reference in its entirety.

Non-limiting examples of in vitro assays to assess ADCC activity of a molecule of interest are provided in U.S. Pat. Nos. 5,500,362 and 5,821,337; Hellstrom et al., Proc. Natl. Acad. Sci. U.S.A., 1986, 83:7059-7063; Hellstrom et al., Proc. Natl. Acad. Sci. U.S.A., 1985, 82:1499-1502; and Bruggemann et al., J. Exp. Med., 1987, 166:1351-1361; each of which is incorporated by reference in its entirety. Useful effector cells for such assays include peripheral blood mononuclear cells (PBMC) and Natural Killer (NK) cells. Alternatively, or additionally, ADCC activity of the molecule of interest may be assessed in vivo, using an animal model such as that disclosed in Clynes et al. Proc. Natl. Acad. Sci. U.S.A., 1998, 95:652-656, incorporated by reference in its entirety.

C1q binding assays may also be carried out to confirm that the antibody or bi-specific antibody is unable to bind C1q and hence lacks CDC activity. Examples of C1q binding assays include those described in WO 2006/029879 and WO 2005/100402, each of which is incorporated by reference in its entirety.

Complement activation assays include those described, for example, in Gazzano-Santoro et al., J. Immunol. Methods, 1996, 202:163-171; Cragg et al., Blood, 2003, 101:1045-1052; and Cragg and Glennie, Blood, 2004, 103:2738-2743; each of which is incorporated by reference in its entirety.

FcRn binding and in vivo clearance (half-life determination) can also be measured, for example, using the methods described in Petkova et al., Intl. Immunol., 2006, 18:1759-1769, incorporated by reference in its entirety.

### 11. Preparation of Antibodies and Bi-Specific Antigen Binding Constructs

11.1. Antigen Preparation

The LAG3 antigen to be used for isolation of the antibodies and bi-specific antigen binding constructs disclosed herein may be intact LAG3 or a fragment of LAG3. The intact LAG3, or fragment of LAG3, may be in the form of an isolated protein or protein expressed by a cell. Other forms of LAG3 useful for generating antibodies will be apparent to those skilled in the art.

The PD-1 antigen to be used for production of antibodies and bi-specific antigen binding constructs disclosed herein may be intact PD-1 or a fragment of PD-1. The intact PD-1, or fragment of PD-1, may be in the form of an isolated protein or expressed by a cell. Other forms of PD-1 useful for generating antibodies will be apparent to those skilled in the art.

11.2. Monoclonal Antibodies

Monoclonal antibodies may be obtained, for example, using the hybridoma method first described by Kohler et al., Nature, 1975, 256:495-497 (incorporated by reference in its entirety), and/or by recombinant DNA methods (see e.g., U.S. Pat. No. 4,816,567, incorporated by reference in its entirety). Monoclonal antibodies may also be obtained, for example, using phage or yeast-based libraries. See e.g., U.S. Pat. Nos. 8,258,082 and 8,691,730, each of which is incorporated by reference in its entirety.

In the hybridoma method, a mouse or other appropriate host animal is immunized to elicit lymphocytes that produce or are capable of producing antibodies that will specifically bind to the protein used for immunization. Alternatively, lymphocytes may be immunized in vitro. Lymphocytes are then fused with myeloma cells using a suitable fusing agent, such as polyethylene glycol, to form a hybridoma cell. See Goding J. W., Monoclonal Antibodies: Principles and Practice 3^{rd} ed. (1986) Academic Press, San Diego, Calif., incorporated by reference in its entirety.

The hybridoma cells are seeded and grown in a suitable culture medium that contains one or more substances that inhibit the growth or survival of the unfused, parental myeloma cells. For example, if the parental myeloma cells lack the enzyme hypoxanthine guanine phosphoribosyl transferase (HGPRT or HPRT), the culture medium for the hybridomas typically will include hypoxanthine, aminopterin, and thymidine (HAT medium), which substances prevent the growth of HGPRT-deficient cells.

Useful myeloma cells are those that fuse efficiently, support stable high-level production of antibody by the selected antibody-producing cells, and are sensitive media conditions, such as the presence or absence of HAT medium. Among these, preferred myeloma cell lines are murine myeloma lines, such as those derived from MOP-21 and MC-11 mouse tumors (available from the Salk Institute Cell Distribution Center, San Diego, Calif.), and SP-2 or X63-Ag8-653 cells (available from the American Type Culture Collection, Rockville, Md.). Human myeloma and mouse-human heteromyeloma cell lines also have been described for the production of human monoclonal antibodies. See e.g., Kozbor, J. Immunol., 1984, 133:3001, incorporated by reference in its entirety.

After the identification of hybridoma cells that produce antibodies of the desired specificity, affinity, and/or biological activity, selected clones may be subcloned by limiting dilution procedures and grown by standard methods. See Goding, supra. Suitable culture media for this purpose include, for example, D-MEM or RPMI-1640 medium. In addition, the hybridoma cells may be grown in vivo as ascites tumors in an animal.

DNA encoding the monoclonal antibodies may be readily isolated and sequenced using conventional procedures (e.g., by using oligonucleotide probes that are capable of binding specifically to genes encoding the heavy and light chains of the monoclonal antibodies). Thus, the hybridoma cells can serve as a useful source of DNA encoding antibodies with the desired properties. Once isolated, the DNA may be placed into expression vectors, which are then transfected into host cells such as bacteria (e.g., E. coli), yeast (e.g., Saccharomyces or Pichia sp.), COS cells, Chinese hamster ovary (CHO) cells, or myeloma cells that do not otherwise produce antibody, to produce the monoclonal antibodies.

11.3. Humanized Antibodies

Humanized antibodies may be generated by replacing most, or all, of the structural portions of a non-human monoclonal antibody with corresponding human antibody sequences. Consequently, a hybrid molecule is generated in which only the antigen-specific variable, or CDR, is composed of non-human sequence. Methods to obtain humanized antibodies include those described in, for example, Winter and Milstein, Nature, 1991, 349:293-299; Rader et al., Proc. Nat. Acad. Sci. U.S.A., 1998, 95:8910-8915; Steinberger et al., J. Biol. Chem., 2000, 275:36073-36078; Queen et al., Proc. Natl. Acad. Sci. U.S.A., 1989, 86:10029-10033; and U.S. Pat. Nos. 5,585,089, 5,693,761, 5,693,762, and 6,180,370; each of which is incorporated by reference in its entirety.

11.4. Human Antibodies

Human antibodies can be generated by a variety of techniques known in the art, for example by using transgenic animals (e.g., humanized mice). See, e.g., Jakobovits et al., Proc. Natl. Acad. Sci. U.S.A., 1993, 90:2551; Jakobovits et al., Nature, 1993, 362:255-258; Bruggermann et al., Year in Immuno., 1993, 7:33; and U.S. Pat. Nos. 5,591,669, 5,589,369 and 5,545,807; each of which is incorporated by reference in its entirety. Human antibodies can also be derived from phage-display libraries (see e.g., Hoogenboom et al., J. Mol. Biol., 1991, 227:381-388; Marks et al., J. Mol. Biol., 1991, 222:581-597; and U.S. Pat. Nos. 5,565,332 and 5,573,905; each of which is incorporated by reference in its entirety). Human antibodies may also be generated by in vitro activated B cells (see e.g., U.S. Pat. Nos. 5,567,610 and 5,229,275, each of which is incorporated by reference in its entirety). Human antibodies may also be derived from yeast-based libraries (see e.g., U.S. Pat. No. 8,691,730, incorporated by reference in its entirety). Human antibodies can also be generated and screened by ribosome display (see, e.g., WO 2014/176327 and WO 2014/176439, each of which is incorporated herein by reference in its entirety).

### 12. Vectors, Host Cells, and Recombinant Methods

The invention also provides isolated nucleic acids encoding an antibody or bispecific antigen binding construct disclosed herein, vectors and host cells comprising the nucleic acids, and recombinant techniques for the production of the antibodies.

For recombinant production of the antibody, the nucleic acid(s) encoding it may be isolated and inserted into a replicable vector for further cloning (i.e., amplification of the DNA) or expression. In some aspects, the nucleic acid may be produced by homologous recombination, for example as described in U.S. Pat. No. 5,204,244, incorporated by reference in its entirety.

Many different vectors are known in the art. The vector components generally include, but are not limited to, one or more of the following: a signal sequence, an origin of replication, one or more marker genes, an enhancer element, a promoter, and a transcription termination sequence, for example as described in U.S. Pat. No. 5,534,615, incorporated by reference in its entirety.

Illustrative examples of suitable host cells are provided below. These host cells are not meant to be limiting.

Suitable host cells include any prokaryotic (e.g., bacterial), lower eukaryotic (e.g., yeast), or higher eukaryotic (e.g., mammalian) cells. Suitable prokaryotes include eubacteria, such as Gram-negative or Gram-positive organisms, for example, Enterobacteriaceae such as Escherichia (E. coli), Enterobacter, Erwinia, Klebsiella, Proteus, Salmonella (S. typhimurium), Serratia (S. marcescans), Shigella, Bacilli (B. subtilis and B. licheniformis), Pseudomonas (P. aeruginosa), and Streptomyces. One useful E. coli cloning host is E. coli 294, although other strains such as E. coli B, E. coli X1776, and E. coli W3110 are suitable.

In addition to prokaryotes, eukaryotic microbes such as filamentous fungi or yeast are also suitable cloning or expression hosts for anti-LAG3 antibody-encoding vectors. Saccharomyces cerevisiae, or common baker's yeast, is a commonly used lower eukaryotic host microorganism. However, a number of other genera, species, and strains are available and useful, such as Schizosaccharomyces pombe, Kluyveromyces (K. lactis, K. fragilis, K. bulgaricus K. wickeramii, K. waltii, K. drosophilarum, K thermotolerans, and K. marxianus), Yarrowia, Pichia pastoris, Candida (C. albicans), Trichoderma reesia, Neurospora crassa, Schwanniomyces (S. occidentalis), and filamentous fungi such as, for example Penicillium, Tolypocladium, and Aspergillus (A. nidulans and A. niger).

Suitable host cells can also include insect cells, such as, for example, Drosophila systems S2, SF9, and SF21 cells and High Five™ cells (ThermoFisher Scientific). Drosophila cells can be grown in a suitable medium, such as, for example, Schneider's Drosophila medium or other commercially available media,

Useful mammalian host cells include COS-7 cells, HEK293 cells; baby hamster kidney (BHK) cells; Chinese hamster ovary (CHO); mouse sertoli cells; African green monkey kidney cells (VERO-76), and the like.

The host cells used to produce the anti-LAG3 antibody of this invention may be cultured in a variety of media. Commercially available media such as, for example, Ham's F10, Minimal Essential Medium (MEM), RPMI-1640, and Dulbecco's Modified Eagle's Medium (DMEM) are suitable for culturing the host cells. In addition, any of the media described in Ham et al., Meth. Enz., 1979, 58:44; Barnes et al., Anal. Biochem., 1980, 102:255; and U.S. Pat. Nos. 4,767,704, 4,657,866, 4,927,762, 4,560,655, and 5,122,469, or WO 90/03430 and WO 87/00195 may be used. Each of the foregoing references is incorporated by reference in its entirety.

Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics, trace elements (defined as inorganic compounds usually present at final concentrations in the micromolar range), and glucose or an equivalent energy source. Any other necessary supplements may also be included at appropriate concentrations that would be known to those skilled in the art.

The culture conditions, such as temperature, pH, and the like, are those previously used with the host cell selected for expression, and will be apparent to the ordinarily skilled artisan.

When using recombinant techniques, the antibody can be produced intracellularly, in the periplasmic space, or directly secreted into the medium. If the antibody is produced intracellularly, as a first step, the particulate debris, either host cells or lysed fragments, is removed, for example, by centrifugation or ultrafiltration. For example, Carter et al. (Bio/Technology, 1992, 10:163-167) describes a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min Cell debris can be removed by centrifugation.

In some embodiments, the antibody is produced in a cell-free system. In some aspects, the cell-free system is an in vitro transcription and translation system as described in Yin et al., mAbs, 2012, 4:217-225, incorporated by reference in its entirety. In some aspects, the cell-free system utilizes a cell-free extract from a eukaryotic cell or from a prokaryotic cell. In some aspects, the prokaryotic cell is E. coli. Cell-free expression of the antibody may be useful, for example, where the antibody accumulates in a cell as an insoluble aggregate, or where yields from periplasmic expression are low.

Where the antibody is secreted into the medium, supernatants from such expression systems are generally first concentrated using a commercially available protein concentration filter, for example, an Amicon® or Millipore® Pellcon® ultrafiltration unit. A protease inhibitor such as PMSF may be included in any of the foregoing steps to inhibit proteolysis and antibiotics may be included to prevent the growth of adventitious contaminants.

The antibody composition prepared from the cells can be purified using, for example, hydroxylapatite chromatography, gel electrophoresis, dialysis, and affinity chromatography, with affinity chromatography being a particularly useful purification technique. The suitability of protein A as an affinity ligand depends on the species and isotype of any immunoglobulin Fc domain that is present in the antibody. Protein A can be used to purify antibodies that are based on human γ1, γ2, or γ4 heavy chains (Lindmark et al., J. Immunol. Meth., 1983, 62:1-13, incorporated by reference in its entirety). Protein G is useful for all mouse isotypes and for human γ3 (Guss et al., EMBO J., 1986, 5:1567-1575, incorporated by reference in its entirety).

The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H3} domain, the BakerBond ABX® resin is useful for purification.

Other techniques for protein purification, such as fractionation on an ion-exchange column, ethanol precipitation, Reverse Phase HPLC, chromatography on silica, chromatography on heparin Sepharose®, chromatofocusing, SDS-PAGE, and ammonium sulfate precipitation are also available, and can be applied by one of skill in the art. In embodiments where the antibody is a bi-specific antibody or antigen binding construct, separation techniques suitable for such molecules are described, for example, in Xu et al. 2015. mAbs 7:231-242, which is incorporated herein by reference in its entirety.

Following any preliminary purification step(s), the mixture comprising the antibody of interest and contaminants may be subjected to low pH hydrophobic interaction chromatography using an elution buffer at a pH between about 2.5 to about 4.5, generally performed at low salt concentrations (e.g., from about 0 to about 0.25 M salt).

### 13. Pharmaceutical Compositions and Methods of Administration

Any of the antibodies or bi-specific antigen binding constructs provided herein can be provided in any appropriate pharmaceutical composition and be administered by any suitable route of administration. Suitable routes of administration include, but are not limited to, the inhalation, intraarterial, intradermal, intramuscular, intraperitoneal, intravenous, nasal, parenteral, pulmonary, and subcutaneous routes.

The pharmaceutical composition may comprise one or more pharmaceutical excipients. Any suitable pharmaceutical excipient may be used, and one of ordinary skill in the art is capable of selecting suitable pharmaceutical excipients. Accordingly, the pharmaceutical excipients provided below are intended to be illustrative, and not limiting. Additional pharmaceutical excipients include, for example, those described in the Handbook of Pharmaceutical Excipients, Rowe et al. (Eds.) 6th Ed. (2009), incorporated by reference in its entirety.

In some embodiments, the pharmaceutical composition comprises an anti-foaming agent. Any suitable anti-foaming agent may be used. In some aspects, the anti-foaming agent is selected from an alcohol, an ether, an oil, a wax, a silicone, a surfactant, and combinations thereof. In some aspects, the anti-foaming agent is selected from a mineral oil, a vegetable oil, ethylene bis stearamide, a paraffin wax, an ester wax, a fatty alcohol wax, a long chain fatty alcohol, a fatty acid soap, a fatty acid ester, a silicon glycol, a fluorosilicone, a polyethylene glycol-polypropylene glycol copolymer, polydimethylsiloxane-silicon dioxide, ether, octyl alcohol, capryl alcohol, sorbitan trioleate, ethyl alcohol, 2-ethyl-hexanol, dimethicone, oleyl alcohol, simethicone, and combinations thereof.

In some embodiments, the pharmaceutical composition comprises a cosolvent. Illustrative examples of cosolvents include ethanol, poly(ethylene) glycol, butylene glycol, dimethylacetamide, glycerin, and propylene glycol.

In some embodiments, the pharmaceutical composition comprises a buffer. Illustrative examples of buffers include acetate, borate, carbonate, lactate, malate, phosphate, citrate, hydroxide, diethanolamine, monoethanolamine, glycine, methionine, guar gum, and monosodium glutamate.

In some embodiments, the pharmaceutical composition comprises a carrier or filler. Illustrative examples of carriers or fillers include lactose, maltodextrin, mannitol, sorbitol, chitosan, stearic acid, xanthan gum, and guar gum.

In some embodiments, the pharmaceutical composition comprises a surfactant. Illustrative examples of surfactants include d-alpha tocopherol, benzalkonium chloride, benzethonium chloride, cetrimide, cetylpyridinium chloride, docusate sodium, glyceryl behenate, glyceryl monooleate, lauric acid, macrogol 15 hydroxystearate, myristyl alcohol, phospholipids, polyoxyethylene alkyl ethers, polyoxyethylene sorbitan fatty acid esters, polyoxyethylene stearates, polyoxylglycerides, sodium lauryl sulfate, sorbitan esters, and vitamin E polyethylene(glycol) succinate.

In some embodiments, the pharmaceutical composition comprises an anti-caking agent. Illustrative examples of anti-caking agents include calcium phosphate (tribasic), hydroxymethyl cellulose, hydroxypropyl cellulose, and magnesium oxide.

Other excipients that may be used with the pharmaceutical compositions include, for example, albumin, antioxidants, antibacterial agents, antifungal agents, bioabsorbable polymers, chelating agents, controlled release agents, diluents, dispersing agents, dissolution enhancers, emulsifying agents, gelling agents, ointment bases, penetration enhancers, preservatives, solubilizing agents, solvents, stabilizing agents, and sugars. Specific examples of each of these agents are described, for example, in the Handbook of Pharmaceutical Excipients, Rowe et al. (Eds.) 6th Ed. (2009), The Pharmaceutical Press, incorporated by reference in its entirety.

In some embodiments, the pharmaceutical composition comprises a solvent. In some aspects, the solvent is saline solution, such as a sterile isotonic saline solution or dextrose solution. In some aspects, the solvent is water for injection.

In some embodiments, the pharmaceutical compositions are in a particulate form, such as a microparticle or a nanoparticle. Microparticles and nanoparticles may be formed from any suitable material, such as a polymer or a lipid. In some aspects, the microparticles or nanoparticles are micelles, liposomes, or polymersomes.

Further provided herein are anhydrous pharmaceutical compositions and dosage forms comprising an antibody, since water can facilitate the degradation of some antibodies.

Anhydrous pharmaceutical compositions and dosage forms provided herein can be prepared using anhydrous or low moisture containing ingredients and low moisture or low humidity conditions. Pharmaceutical compositions and dosage forms that comprise lactose and at least one active ingredient that comprises a primary or secondary amine can be anhydrous if substantial contact with moisture and/or humidity during manufacturing, packaging, and/or storage is expected.

An anhydrous pharmaceutical composition should be prepared and stored such that its anhydrous nature is maintained. Accordingly, anhydrous compositions can be packaged using materials known to prevent exposure to water such that they can be included in suitable formulary kits. Examples of suitable packaging include, but are not limited to, hermetically sealed foils, plastics, unit dose containers (e.g., vials), blister packs, and strip packs.

In some embodiments, the CDR-H1, CDR-H2, CDR-H3, CDR-L1, CDR-L2, and CDR-L3 provided herein comprise a variant of an illustrative CDR-H1, CDR-H2, CDR-H3, CDR-L1, CDR-L2, and/or CDR-L3 sequence provided in this disclosure.

In some aspects, the CDR-H1 sequence comprises, consists of, or consists essentially of a variant of an illustrative Chothia or Kabat CDR-H1 sequence provided in this disclosure. In some aspects, the CDR-H1 sequence comprises, consists of, or consists essentially of a sequence having at least 70%, 75%, 80%, 85%, 90%, or 95% identity with any of the illustrative Chothia or Kabat CDR-H1 sequences provided in this disclosure. In some aspects, the CDR-H1 sequence comprises, consists of, or consists essentially of any of the illustrative Chothia or Kabat CDR-H1 sequences provided in this disclosure, with 1, 2, or 3 amino acid substitutions. In some aspects, the amino acid substitutions are conservative amino acid substitutions.

In some aspects, the CDR-H2 sequence comprises, consists of, or consists essentially of a variant of an illustrative Chothia or Kabat CDR-H2 sequence provided in this disclosure. In some aspects, the CDR-H2 sequence comprises, consists of, or consists essentially of a sequence having at least 70%, 75%, 80%, 85%, 90%, or 95% identity with any of the illustrative Chothia or Kabat CDR-H2 sequences provided in this disclosure. In some aspects, the CDR-H2 sequence comprises, consists of, or consists essentially of any of the illustrative Chothia or Kabat CDR-H2 sequences provided in this disclosure, with 1, 2, or 3 amino acid substitutions. In some aspects, the amino acid substitutions are conservative amino acid substitutions.

In some aspects, the CDR-H3 sequence comprises, consists of, or consists essentially of a variant of an illustrative CDR-H3 sequence provided in this disclosure. In some aspects, the CDR-H3 sequence comprises, consists of, or consists essentially of a sequence having at least 70%, 75%, 80%, 85%, 90%, or 95% identity with any of the illustrative CDR-H3 sequences provided in this disclosure. In some aspects, the CDR-H3 sequence comprises, consists of, or consists essentially of any of the illustrative CDR-H3 sequences provided in this disclosure, with 1, 2, or 3 amino acid substitutions. In some aspects, the amino acid substitutions are conservative amino acid substitutions.

In some aspects, the CDR-L1 sequence comprises, consists of, or consists essentially of a variant of an illustrative CDR-L1 sequence provided in this disclosure. In some aspects, the CDR-L1 sequence comprises, consists of, or consists essentially of a sequence having at least 70%, 75%, 80%, 85%, 90%, or 95% identity with any of the illustrative CDR-L1 sequences provided in this disclosure. In some aspects, the CDR-L1 sequence comprises, consists of, or consists essentially of any of the illustrative CDR-L1 sequences provided in this disclosure, with 1, 2, or 3 amino acid substitutions. In some aspects, the amino acid substitutions are conservative amino acid substitutions.

In some aspects, the CDR-L2 sequence comprises, consists of, or consists essentially of a variant of an illustrative CDR-L2 sequence provided in this disclosure. In some aspects, the CDR-L2 sequence comprises, consists of, or consists essentially of a sequence having at least 70%, 75%, 80%, 85%, 90%, or 95% identity with any of the illustrative CDR-L2 sequences provided in this disclosure. In some aspects, the CDR-L2 sequence comprises, consists of, or consists essentially of any of the illustrative CDR-L2 sequences provided in this disclosure, with 1, 2, or 3 amino acid substitutions. In some aspects, the amino acid substitutions are conservative amino acid substitutions.

In some aspects, the CDR-L3 sequence comprises, consists of, or consists essentially of a variant of an illustrative CDR-L3 sequence provided in this disclosure. In some aspects, the CDR-L3 sequence comprises, consists of, or consists essentially of a sequence having at least 70%, 75%, 80%, 85%, 90%, or 95% identity with any of the illustrative CDR-L3 sequences provided in this disclosure. In some aspects, the CDR-L3 sequence comprises, consists of, or consists essentially of any of the illustrative CDR-L3 sequences provided in this disclosure, with 1, 2, or 3 amino acid substitutions. In some aspects, the amino acid substitutions are conservative amino acid substitutions. In certain embodiments, provided are parenteral dosage forms. Parenteral dosage forms can be administered to subjects by various routes including, but not limited to, subcutaneous, intravenous (including bolus injection), intramuscular, and intraarterial. Because their administration typically bypasses subjects' natural defenses against contaminants, parenteral dosage forms are typically, sterile or capable of being sterilized prior to administration to a subject. Examples of parenteral dosage forms include, but are not limited to, solutions ready for injection, dry products ready to be dissolved or suspended in a pharmaceutically acceptable vehicle for injection, suspensions ready for injection, and emulsions.

Suitable vehicles that can be used to provide parenteral dosage forms are well known to those skilled in the art. Examples include, but are not limited to: Water for Injection USP; aqueous vehicles such as, but not limited to, Sodium Chloride Injection, Ringer's Injection, Dextrose Injection, Dextrose and Sodium Chloride Injection, and Lactated Ringer's Injection; water miscible vehicles such as, but not limited to, ethyl alcohol, polyethylene glycol, and polypropylene glycol; and non-aqueous vehicles such as, but not limited to, corn oil, cottonseed oil, peanut oil, sesame oil, ethyl oleate, isopropyl myristate, and benzyl benzoate.

Excipients that increase the solubility of one or more of the antibodies disclosed herein can also be incorporated into the parenteral dosage forms.

13.1. Dosage and Unit Dosage Forms

In human therapeutics, the doctor will determine the posology which he considers most appropriate according to a preventive or curative treatment and according to the age, weight, condition and other factors specific to the subject to be treated.

In certain embodiments, a composition provided herein is a pharmaceutical composition or a single unit dosage form. Pharmaceutical compositions and single unit dosage forms provided herein comprise a prophylactically or therapeutically effective amount of one or more prophylactic or therapeutic antibodies.

The amount of the antibody or composition which will be effective in the prevention or treatment of a disorder or one or more symptoms thereof will vary with the nature and severity of the disease or condition, and the route by which the antibody is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, exemplary doses of a composition include milligram or microgram amounts of the antibody per kilogram of subject or sample weight (e.g., about 10 micrograms per kilogram to about 50 milligrams per kilogram, about 100 micrograms per kilogram to about 25 milligrams per kilogram, or about 100 microgram per kilogram to about 10 milligrams per kilogram). In certain embodiment, the dosage of the antibody provided herein, based on weight of the antibody, administered to prevent, treat, manage, or ameliorate a disorder, or one or more symptoms thereof in a subject is about 0.1 mg/kg, about 1 mg/kg, about 2 mg/kg, about 3 mg/kg, about 4 mg/kg, about 5 mg/kg, about 6 mg/kg, about 10 mg/kg, or about 15 mg/kg or more of a subject's body weight. In another embodiment, the dosage of the composition or a composition provided herein administered to prevent, treat, manage, or ameliorate a disorder, or one or more symptoms thereof in a subject is about 0.1 mg to about 200 mg, about 0.1 mg to about 100 mg, about 0.1 mg to about 50 mg, about 0.1 mg to about 25 mg, about 0.1 mg to about 20 mg, about 0.1 mg to about 15 mg, about 0.1 mg to about 10 mg, about 0.1 mg to about 7.5 mg, about 0.1 mg to about 5 mg, about 0.1 to about 2.5 mg, about 0.25 mg to about 20 mg, about 0.25 to about 15 mg, about 0.25 to about 12 mg, about 0.25 to about 10 mg, about 0.25 mg to about 7.5 mg, about 0.25 mg to about 5 mg, about 0.25 mg to about 2.5 mg, about 0.5 mg to about 20 mg, about 0.5 to about 15 mg, about 0.5 to about 12 mg, about 0.5 to about 10 mg, about 0.5 mg to about 7.5 mg, about 0.5 mg to about 5 mg, about 0.5 mg to about 2.5 mg, about 1 mg to about 20 mg, about 1 mg to about 15 mg, about 1 mg to about 12 mg, about 1 mg to about 10 mg, about 1 mg to about 7.5 mg, about 1 mg to about 5 mg, or about 1 mg to about 2.5 mg.

The dose can be administered according to a suitable schedule, for example, once, two times, three times, or for times weekly. It may be necessary to use dosages of the antibody outside the ranges disclosed herein in some cases, as will be apparent to those of ordinary skill in the art. Furthermore, it is noted that the clinician or treating physician will know how and when to interrupt, adjust, or terminate therapy in conjunction with subject response.

Different therapeutically effective amounts may be applicable for different diseases and conditions, as will be readily known by those of ordinary skill in the art. Similarly, amounts sufficient to prevent, manage, treat or ameliorate such disorders, but insufficient to cause, or sufficient to reduce, adverse effects associated with the antibodies provided herein are also encompassed by the herein described dosage amounts and dose frequency schedules. Further, when a subject is administered multiple dosages of a composition provided herein, not all of the dosages need be the same. For example, the dosage administered to the subject may be increased to improve the prophylactic or therapeutic effect of the composition or it may be decreased to reduce one or more side effects that a particular subject is experiencing.

In certain embodiments, treatment or prevention can be initiated with one or more loading doses of an antibody or composition provided herein followed by one or more maintenance doses.

In certain embodiments, a dose of an antibody or composition provided herein can be administered to achieve a steady-state concentration of the antibody in blood or serum of the subject. The steady-state concentration can be determined by measurement according to techniques available to those of skill or can be based on the physical characteristics of the subject such as height, weight and age.

In certain embodiments, administration of the same composition may be repeated and the administrations may be separated by at least about 1 day, about 2 days, about 3 days, about 5 days, about 10 days, about 15 days, about 30 days, about 45 days, about 2 months, about 75 days, about 3 months, or about 6 months. In other embodiments, administration of the same prophylactic or therapeutic agent may be repeated and the administration may be separated by at least about 1 day, about 2 days, about 3 days, about 5 days, about 10 days, about 15 days, about 30 days, about 45 days, about 2 months, about 75 days, about 3 months, or about 6 months.

### 14. Therapeutic Applications

For therapeutic applications, the antibodies of the invention are administered to a mammal, generally a human, in a pharmaceutically acceptable dosage form such as those known in the art and those discussed above. For example, the antibodies of the invention may be administered to a human intravenously as a bolus or by continuous infusion over a period of time, by intramuscular, intraperitoneal, intra-cerebrospinal, subcutaneous, intra-articular, intrasynovial, intrathecal, or intratumoral routes. The antibodies also are suitably administered by peritumoral, intralesional, or perilesional routes, to exert local as well as systemic therapeutic effects. The intraperitoneal route may be particularly useful, for example, in the treatment of ovarian tumors.

The antibodies provided herein may be useful for the treatment of any disease or condition involving LAG3 and/or PD-1. In some embodiments, the disease or condition is a disease or condition that can be diagnosed by overexpression of LAG3 and/or PD-1. In some embodiments, the disease or condition is a disease or condition that can benefit from treatment with an anti-LAG3 antibody and/or anti-PD-1 antibody. In some embodiments, the disease or condition is a cancer. In some embodiments, the disease or condition is an autoimmune disease. In some embodiments, the disease or condition is an infection.

Any suitable cancer may be treated with the antibodies provided herein. Illustrative suitable cancers include, for example, acute lymphoblastic leukemia (ALL), acute myeloid leukemia (AML), adrenocortical carcinoma, anal cancer, appendix cancer, astrocytoma, basal cell carcinoma, brain tumor, bile duct cancer, bladder cancer, bone cancer, breast cancer, bronchial tumor, carcinoma of unknown primary origin, cardiac tumor, cervical cancer, chordoma, colon cancer, colorectal cancer, craniopharyngioma, ductal carcinoma, embryonal tumor, endometrial cancer, ependymoma, esophageal cancer, esthesioneuroblastoma, fibrous histiocytoma, Ewing sarcoma, eye cancer, germ cell tumor, gallbladder cancer, gastric cancer, gastrointestinal carcinoid tumor, gastrointestinal stromal tumor, gestational trophoblastic disease, glioma, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, lip and oral cavity cancer, liver cancer, lobular carcinoma in situ, lung cancer, macroglobulinemia, malignant fibrous histiocytoma, melanoma, Merkel cell carcinoma, mesothelioma, metastatic squamous neck cancer with occult primary, midline tract carcinoma involving NUT gene, mouth cancer, multiple endocrine neoplasia syndrome, multiple myeloma, mycosis fungoides, myelodysplastic syndrome, myelodysplastic/myeloproliferative neoplasm, nasal cavity and par nasal sinus cancer, nasopharyngeal cancer, neuroblastoma, non-small cell lung cancer, oropharyngeal cancer, osteosarcoma, ovarian cancer, pancreatic cancer, papillomatosis, paraganglioma, parathyroid cancer, penile cancer, pharyngeal cancer, pheochromocytomas, pituitary tumor, pleuropulmonary blastoma, primary central nervous system lymphoma, prostate cancer, rectal cancer, renal cell cancer, renal pelvis and ureter cancer, retinoblastoma, rhabdoid tumor, salivary gland cancer, Sezary syndrome, skin cancer, small cell lung cancer, small intestine cancer, soft tissue sarcoma, spinal cord tumor, stomach cancer, T-cell lymphoma, teratoid tumor, testicular cancer, throat cancer, thymoma and thymic carcinoma, thyroid cancer, urethral cancer, uterine cancer, vaginal cancer, vulvar cancer, and Wilms tumor.

In particular embodiments, the cancer is a cancer of epithelial origin. In some aspects, the cancer is a carcinoma. In some aspects, the cancer is selected from an adenocarcinoma, a squamous cell carcinoma, an adenosquamos carcinoma, an anaplastic carcinoma, a large cell carcinoma, small cell carcinoma, and carcinoma of unknown primary origin.

### 15. Diagnostic Applications

In some embodiments, the antibodies provided herein are used in diagnostic applications.

In some embodiments, an antibody or bi-specific antibody as disclosed herein may be useful in assays for LAG3 protein. In some aspects the antibody or bi-specific antibody can be used to detect the expression of LAG3 in various cells and tissues. These assays may be useful, for example, in making a diagnosis and/or prognosis for a disease, such as a cancer.

In some embodiments, an antibody or bi-specific antibody as disclosed herein may be useful in assays for PD-1 protein. In some aspects the antibody or bi-specific antibody can be used to detect the expression of PD-1 in various cells and tissues. These assays may be useful, for example, in making a diagnosis and/or prognosis for a disease, such as a cancer.

In some diagnostic and prognostic applications, the antibody or bi-specific antibody may be labeled with a detectable moiety. Suitable detectable moieties include, but are not limited to radioisotopes, fluorescent labels, and enzyme-substrate labels. In another embodiment, the antibody or bi-specific antibody need not be labeled, and the presence of the antibody or bi-specific antibody can be detected using a labeled antibody which specifically binds to the anti-LAG3 antibody.

### 16. Affinity Purification Reagents

The antibodies and bi-specific antigen binding constructs disclosed herein may be used as affinity purification agents. In this process, the antibodies or bi-specific antibodies may be immobilized on a solid phase such a resin or filter paper, using methods well known in the art. The immobilized antibody or bi-specific antibody is contacted with a sample containing an antigen protein (or fragment thereof) to be purified, and thereafter the support is washed with a suitable solvent that will remove substantially all the material in the sample except the antigen protein, which is bound to the immobilized antibody. Finally, the support is washed with another suitable solvent, such as glycine buffer, pH 5.0 or glycine buffer, pH 3 to 4, that will release the antigen protein from the antibody. In some embodiments, the antigen protein is LAG3. In some embodiments, the antigen protein is PD-1.

### 17. Kits

In some embodiments, an antibody or bi-specific antigen binding construct provided herein is provided in the form of a kit, i.e., a packaged combination of reagents in predetermined amounts with instructions for performing a procedure. In some embodiments, the procedure is a diagnostic assay. In other embodiments, the procedure is a therapeutic procedure.

In some embodiments, the kit further comprises a solvent for the reconstitution of the antibody or bispecific antibody. In some embodiments, the antibody or bispecific antibody is provided in the form of a pharmaceutical composition.

### EXAMPLES

### Example 1: Generation of Mouse and Humanized LAG3 Antibodies

Balb/C mice were immunized with the extracellular domain of human LAG3 fused with human Fc (R&D Systems) using standard immunization methods. The spleens and/or lymph nodes of the mice were harvested and fused with P3X cells to generate hybridomas (Aragen Biosciences, Morgan Hill, Calif.), similar to what has been previously described (Chronopoulou et al., 2014, Methods Mol Biol. 1131:47-70; Kim, et al., 2014, Methods Mol Biol. 1131:33-45; each incorporated by reference in its entirety).

Total RNA was extracted from hybridoma cells using QIAGEN RNeasy Mini Kit (Cat No. 74104) and converted to cDNA using a Clontech SMARTer RACE cDNA Amplification Kit (Cat. No. 634923; Lake Pharma, Belmont, Calif.). Positive clones were identified by gel electrophoresis, cloned using an Invitrogen TOPO kit, and sequenced using standard Sanger methods. Mouse single-chain antibodies were constructed by using total gene synthesis using optimized E. Coli codons and cloned into a standard cell-free expression vector (Yin et al., 2012, mAbs 4:217-225, incorporated by reference in its entirety). Murine IgG 421.61.4.5G11 (5G11) was selected.

The CDRs for 5G11 were grafted onto human antibody frameworks V_{H}1-69 and Vk2-30 by standard methodology (Kuramochi et al., 2014, Methods Mol. Biol. 1060:123-137, incorporated by reference in its entirety) to yield humanized antibody h5G11-2.

### Example 2: Generation and Primary Screening of Anti-LAG3 Antibodies

Antibody Fab and scFv libraries were constructed using a standard overlap extension PCR protocol with mutagenic primers targeting complementary determining regions (CDRs). See Heckman and Pease, Nat. Protoc., 2007, 2:924-932, incorporated by reference in its entirety. Selections for novel antibodies were performed using standard ribosome display protocols. See Dreier and Plückthun, Methods Mol. Biol., 2003, 687:283-306, Clifton, N.J., incorporated by reference in its entirety. Specifically, scFv and Fab ribosome display selections were performed according to published protocols. See Hanes and Plückthun, Proc. Natl. Acad. Sci. U.S.A., 1997, 94:4937-4942; Stafford et al., 2014, Protein Eng. Des. Sel. 27:97-109; each incorporated by reference in its entirety. After multiple rounds of selection, the DNA from RT-PCR output was cloned into an optimized vector for cell-free expression using standard molecular biology techniques. See Yin et al., mAbs, 2012, 4:217-225, incorporated by reference in its entirety. All constructs were HIS- and FLAG-tagged to streamline purification and testing during screening.

Libraries of antibody variants generated by the selection workflow were transformed into E. coli and grown on agar plates with antibiotic (kanamycin). Individual colonies were grown in liquid broth (TB+kanamycin), and used as a template for DNA amplification via rolling circle amplification (RCA). The variants were then expressed in cell-free protein synthesis reactions as described in Zawada et al., 2011, Biotechnol. Bioeng. 108:1570-1578, incorporated by reference in its entirety.

Briefly, cell-free extracts were treated with 50 μM iodoacetamide for 30 min at room temperature (20° C.) and added to a premix containing cell-free components (see Groff et al., mAbs, 2014, 6:671-678, incorporated by reference in its entirety) and 10% (v/v) RCA DNA template (approximately 10 μg/mL DNA) for variants of interest. For Fab selection, 2.5 μg/mL trastuzumab LC DNA was also added to the reactions. Sixty microliters of cell-free reactions were incubated at 30° C. for 12 hr on a shaker at 650 rpm in 96-well plates. Four hundred to one-thousand-five-hundred colonies were screened, depending on the predicted diversity of different selection campaigns.

Following synthesis, each reaction was diluted 1:50 into PBST (PBS at pH 7.4 with 0.2% Tween-20+0.2% BSA) and expressed variants were tested for functional activity via ELISA-based binding to recombinant human LAG3 extracellular domain (ECD) (Acro Biosystems; R&D Systems). Standard ELISA-based methods were employed. Specifically, 384-well plates were coated with 2 μg/mL recombinant LAG3 diluted in bicarbonate buffer, and then blocked with BSA. Antibody variants of interest were allowed to bind to the LAG3-coated plates, and detected with secondary antibodies (e.g., HRP-conjugated anti-human Fc or anti-FLAG) and then detected with chemiluminescent substrate (Pierce ELISA SuperSignal™ Substrate). Chemiluminescence was quantified on a Molecular Devices SpectraMax® M5 plate reader. Top hits were selected based on ELISA signal or signal/noise ratio and their associated DNA constructs were sequenced. Based on functional activity and sequence analysis, a subset of variants was selected for further scale-up and characterization.

### Example 3: Secondary Screening of LAG3 Antibodies

The top leads from the initial round of screening were cultured and plasmid minipreps were performed using a QIAprep® 96 Turbo miniprep kit (Qiagen) according to the manufacturer's instructions. 10 μg/mL miniprepped DNA was added to 4 mL cell-free reactions and incubated overnight for 12 hr at 30° C., at 650 rpm. For Fab selection, 2.5 ug/mL trastuzumab LC DNA was also added.

Expressed variants from clarified cell-free reactions were purified via immobilized metal ion affinity chromatography (IMAC) purification using a semi-automated high throughput batch purification method. Briefly, purifications were performed in a 96-well plate format where 50 μL/well of IMAC resin (Ni Sepharose High Performance, GE Healthcare) was equilibrated in IMAC binding buffer (50 mM Tris pH 8.0, 300 mM NaCl, 10 mM imidazole), incubated with 1 mL cell-free reaction for 15 minutes followed by two washes in IMAC binding buffer. His-tagged antibody variants were then eluted using 200 μL IMAC elution buffer (50 mM Tris pH 8.0, 300 mM NaCl, 500 mM imidazole) and buffer exchanged into PBS using a 96-well Zeba plate (7 kD MWCO, Thermo Fisher). Purified antibodies were quantified via high throughput capillary electrophoresis using the LabChip GXII (Perkin Elmer) against a Herceptin standard curve, according to the manufacturer's instructions.

### Example 4: LAG3 Antibody Selection and Maturation

Primary and secondary screening with humanized antibody h5G11-2 yielded antibodies designated SRP1627 in the Examples below. Ribosome display was used to affinity mature antibody 26H10 (SEQ ID NOs: 191 and 251) yielding antibodies designated SRP1449 or 1449 in the Examples below. Antibody SRP1448-D09 was identified by screening a naive scFv antibody library against LAG3. Affinity maturation of SRP1448-D09 using ribosome display yielded antibodies designated SRP1558 in the Examples below. Anti-LAG-3 Fabs were identified by selecting from a Fab TRIM library against recombinant LAG3 protein using ribosome display (Stafford et al., Protein Eng Des Sel 2014, 27:97-109, incorporated by reference in its entirety). Primary and secondary screening yielded LAG-3 Fab antibodies designated SRP1496 in the Examples below. Affinity maturation of antibody SRP1496-A04 using ribosome display yielded antibodies designated SRP1648 in the Examples below. All ribosome display selections were screened by cloning the output into a cell free expression vector for small-scale expression followed by characterization by ELISA, biacore, cell binding, and ligand competition.

The mouse antibody 421.61.4.5G11 was constructed from the V_{H} and V_{L} variable domains in the table below and mouse constant domains. The human and humanized antibodies were constructed from the V_{H} and V_{L} variable domains in the table below and human constant domains. Additional human antibodies are constructed in either scFvFc or IgG format. The scFvFc format contains a V_{H} domain, followed by a linker domain (for instance, a GGGGSGGGGSGGGGS linker (SEQ ID NO: 308) or a APGPSAPSHRSLPSRAFG linker (SEQ ID NO: 314) from Tang et al., 1996, J. Biol. Chem. 271:15682-15686, incorporated by reference in its entirety), then the V_{L} domain, and then the human scFvFc constant domains. The mouse and human antibody sequences start with an N-terminal methionine to enable expression in cell-free. Additional variable domains can also be expressed in a mammalian system by fusing an N-terminal signal peptide instead of an N-terminal methionine. Additional antibodies can also be expressed with or without a C-terminal affinity tags (e.g., His or FlagHis, SEQ ID NO: 307).

TABLE 21
Antibody Sequences

| | VH | | VL | |
|---|---|---|---|---|
| | | SEQ | | SEQ |
| Antibody | | ID | | ID |
| Name | Name | NO | Name | NO |
| 421.61.4.5G11 | 5G11-VH | 210 | 5G11-VL | 266 |
| SRP1627-A02 | SRP1627-A02-VH | 206 | SRP1627-A02-VL | 262 |
| SRP1627-A11 | SRP1627-A11-VH | 207 | SRP1627-A11-VL | 263 |
| SRP1627-B01 | SRP1627-B01-VH | 209 | SRP1627-B01-VL | 264 |
| h5G11-2 | h5G11-2-VH | 208 | h5G11-2-VL | 265 |
| SRP1449-B03 | SRP1449-B03-VH | 197 | SRP1449-B03-VL | 256 |
| SRP1449-B07 | SRP1449-B07-VH | 199 | SRP1449-B07-VL | 255 |
| SRP1449-D05 | SRP1449-D05-VH | 201 | SRP1449-D05-VL | 252 |
| SRP1449-F01 | SRP1449-F01-VH | 198 | SRP1449-F01-VL | 253 |
| SRP1449- | SRP1449-G09.2- | 200 | SRP1449-G09.2- | 254 |
| G09.2 | VH | | VL | |
| SRP1558-A06 | SRP1558-A06-VH | 204 | SRP1558-A06-VL | 258 |
| SRP1558-E11 | SRP1558-E11-VH | 205 | SRP1558-E11-VL | 257 |
| SRP1558-F01 | SRP1558-F01-VH | 202 | SRP1558-F01-VL | 259 |
| SRP1448-D09 | SRP1448-D09-VH | 203 | SRP1448-D09-VL | 260 |
| SRP1496-A03 | SRP1496-A03-VH | 192 | trastuzumab-VL | 261 |
| SRP1496-A04 | SRP1496-A04-VH | 193 | trastuzumab-VL | 261 |
| SRP1496-B08 | SRP1496-B08-VH | 194 | trastuzumab-VL | 261 |
| SRP1648-B07 | SRP1648-B07-VH | 195 | trastuzumab-VL | 261 |
| SRP1648-E02 | SRP1648-E02-VH | 196 | trastuzumab-VL | 261 |

### Example 5: Affinity and Kinetic Binding Analyses of LAG3 Antibodies

Anti-Flag M2 IgG (Sigma-Aldrich # F9291) was immobilized onto a CMS chip (GE Life Sciences) using amine coupling chemistry (from Amine Coupling Kit, GE Life Sciences). The immobilization steps were carried out at a flow rate of 25 μL/min in 1×HBS-EP+ buffer (GE Life Sciences; 10×Stock diluted before use). The sensor surfaces were activated for 7 min with a mixture of NHS (0.05 M) and EDC (0.2 M). The Anti-Flag M2 IgG was injected over all 4 flow cells at a concentration of 25 μg/mL in 10 mM sodium acetate, pH 4.5, for 7 min. Ethanolamine (1 M, pH 8.5) was injected for 7 min to block any remaining activated groups. An average of 12,000 response units (RU) of capture antibody was immobilized on each flow cell.

Off-rate and kinetic binding experiments were performed at 25° C. using 1×HBS-EP+buffer. Test and control antibodies were injected over the Anti-Flag surface at concentrations of 5-10 μg/mL for 12 seconds at a flow rate of 10 μL/min on flow cells 2, 3 and 4, followed by a buffer wash for 30 seconds at the same flow rate. Kinetic characterization of antibody samples was carried out with a single concentration of antigen (for off-rate ranking) or a dilution series of antigen (for kinetic characterization) and 1 injection of 0 nM antigen. After capturing ligand (antibody) on the anti-Flag surface, the analyte (human LAG3-Fc, R&D Systems #2319-L3; or cynomolgus LAG3-Fc, accession # NC 022282.1) was bound at 50, 25, 12.5, 6.25, and 0 nM for 180 seconds, followed by a 600 second dissociation phase at a flow rate of 50 μl/min Between each ligand capture and analyte binding cycle, regeneration was carried out using 2 injections of 10 mM glycine pH 2.0 for 30 seconds at 30 μL/min, followed by a 30 second buffer wash step.

The data were fit with the Biacore T200 Evaluation software, using a 1:1 Langmuir binding model. K_{D} (affinity, nM) was determined as a ratio of the kinetic rate constants calculated from the fits of the association and dissociation phases.

### Example 6: ELISA Binding Assay of Anti-LAG3 Antibodies

Standard ELISA methods were used to compare binding to human and cynomolgus recombinant LAG-3. Specifically, 384-well plates were coated with 2 μg/mL recombinant LAG3 (human LAG3-Fc or cynomolgus LAG3-Fc) diluted in bicarbonate buffer, and then blocked with BSA. A dilution series of antibody variants were allowed to bind to the LAG3-coated plates, and detected with secondary antibodies (e.g., HRP-conjugated anti-human Fab or anti-FLAG) and then detected with chemiluminescent substrate (Pierce ELISA SuperSignal™ Substrate). Chemiluminescence was quantified on a Molecular Devices SpectraMax® M5 plate reader. ELISA EC50s were calculated.

### Example 7: Cell Binding Assay of Anti-LAG3 Antibodies

LAG3 antibody variants were tested in a fluorescence-activated cell sorting (FACS) cell-binding assay. Chinese Hamster Ovary (CHO) cells or HEK293T cells stably expressing the human target molecule LAG3 on the cell surface (CHO-LAG3, 293T-LAG3) were used to screen for cell binders by flow cytometry. Parental CHO or 293T cells were used as a negative control to determine background-binding levels. Cells were cultured in RPMI with 10% FCS Penicillin/Streptomycin (or Pen/Strep) and glutamine (or Gln) and split every 3-4 days at 10⁵ cells/ml.

A mix of parental CHO cells and CHO-LAG3 cells (or 293T and 293T-LAG3 cells) was prepared as follows: Parental CHO cells were washed 2× in PBS then incubated in PBS containing 1 nM CellTrace™ Oregon Green488® (Life Technologies) at 37° C. for 30 minutes. Cells were then washed 2× with RPMI w/10% fetal calf serum (or FCS), washed 2× with FACS buffer (PBS w/2% FCS), suspended thoroughly in ice-cold FACS buffer at a final concentration of 2×106 cells/ml and kept on ice. CHO-LAG3 cells were similarly washed with FACS buffer and kept on ice at 2×106 cells/ml. Parental CHO cells and CHO-LAG3 cells were then mixed to obtain a 1:1 cell suspension and seeded at 100 μl per well on 96 well polypropylene plates. Plates were spun at 1500 rpm for 5 minutes and cell pellets were suspended in 50 μl FACS buffer containing 6-12 point dilutions of anti-LAG3 variants starting from concentrations of ˜100-200 nM antibody, dispensed using BioMekFX (Beckman Coulter). Cells were then incubated on ice for 1 hr, washed with FACS buffer and incubated for 1 hr on ice with 50 μl FACS buffer containing 2.5 μg/ml R-Phycoerythrin-conjugated Goat Anti-Human IgG (Jackson ImmunoResearch) or AF647-conjugated Goat Anti-mouse IgG (Life Technologies) dispensed using BioMekFX (Beckman Coulter). Cells were then washed 2× with FACS buffer and fixed for 10 minutes in 200 μl PBS with 2% PFA prior to fluorescence detection. Samples were acquired using a Beckton Dickinson LSRII FACS. Mean Fluorescence Intensity of LAG3 antibody binding was analyzed using Tree Star, Inc. FlowJo® software.

### Example 8: Cell-Based MHCII Competition of LAG3 Antibodies

Top variants that showed cell-binding activity were tested in a fluorescence-activated cell sorting (FACS) cell-based competition assay. DAUDI cells express high levels of Major Histocompatibility Class II (MHCII) molecules, a natural ligand for LAG3, on the cell surface. DAUDI cells were used to screen for antibodies that inhibit binding of HIS-tagged (ACRO) or biotinylated recombinant human LAG3 protein (rhLAG3) to MHCII expressed on the cell surface.

DAUDI cells were cultured in RPMI w/10% FCS Pen/Strep and Gln and split every 3-4 days at 105 cells/ml. Cells were washed 2× with FACS buffer (PBS w/2% FCS), thoroughly in ice-cold FACS buffer at a final concentration of 1×10⁶ cells/ml and seeded at 100 μl per well on 96 well polypropylene plates. Plates were spun at 1500 rpm for 5 minutes and cell pellets were suspended in 50 μl FACS buffer containing 8 point 1:3 dilutions (2× concentrated) of anti-LAG3 antibody variants, starting from high concentration of −600 nM. 50 μl FACS buffer containing 10-20 μg/ml of the HIS-tagged rhLAG3 protein or 40 μg/ml of the biotinylated rhLAG3 protein were then added to the cells. Cell were then incubated in ice for 1 hr, washed with FACS buffer and incubated for 1 hr in ice with 50 μl FACS buffer containing 2 μg/ml R-Phycoerythrin-conjugated Streptavidin (eBiosciences) or 1 μg/ml R-Phycoerythrin-conjugated anti-HIS IgG (Abcam). Cell were washed 2× with FACS buffer and fixed for 10 minutes in 200 μl PBS w/2% PFA prior to acquisition.

### Example 9: Effect of Anti-PD-1 in Combination with Anti-LAG3 Antibodies on IFN γ Production in a CMV Recall Assay and Dendritic Cell (DC)/CD-4+ T Cell Mixed Lymphocyte Reaction (MLR)

### CMV Recall Assay

CD14⁺ monocytes and CD3⁺ T cells were obtained from peripheral blood mononuclear (PBMC) isolated from CMV⁺ human donors (AllCells, Alameda, Calif.) using MACS Cell Separation kits (Miltenyi Biotec). CD14⁺ monocytes were differentiated into immature dendritic cells (DC) by culturing cells at 1e6 cells/ml for 7 days in presence of GM-CSF and IL-4 (Peprotech) in X-Vivo 15 media (Lonza) containing 2% human AB serum (Sigma-Aldrich), penicillin-streptomycin (Corning Mediatech) and GlutaMAX (Life Technologies). Following differentiation, DCs were matured by culturing in X-Vivo 15+2% human AB serum media at 1e6 cells/ml for 2 days in the presence of GM-CSF, IL-4, TNF-a, IL-1b, IL-6 (Peprotech) and prostaglandin E2 (Sigma-Aldrich). To set-up the CMV recall assay, mature DCs were collected, washed and 10,000 DCs and 100,000 pan CD3⁺ T cells were plated per well in a 96-well U-bottom plate in a total volume of 100 ul media containing peptide pools for the CMV IE-1 and CMV pp65 protein (Miltenyi Biotec). Anti-PD-1 and/or anti-LAG-3 IgG antibodies (50 ul) were added starting at a final concentration of 133-400 nM with 5-fold serial dilutions. Cells were co-cultured with peptides and antibodies for 5-6 days. Conditioned media was collected and tested for human IFN-g levels by ELISA (BD Biosciences).

### DC/CD4⁺ T Cell Mixed Lymphocyte Reaction (MLR)

Allogeneic CD14⁺ monocytes and CD4⁺ T cells were obtained from PBMC isolated from human donors using MACS Cell Separation kits. CD14⁺ monocytes were differentiated into immature DC by culturing cells at 1e6 cells/ml cell density for 7 days in presence of GM-CSF and IL-4 in RPMI media containing 10% fetal bovine serum, penicillin-streptomycin and GlutaMAX. Following differentiation, DCs were matured by culturing in RPMI+10% FBS media at 1e6 cells/ml cell density for 2 days in the presence of GM-CSF, IL-4, TNF-α, IL-1b, IL-6 and prostaglandin E2. To set-up the DC/CD4⁺ T cell MLR, mature DCs were collected, washed and 10,000 DCs and 100,000 CD4⁺ T cells were plated per well in a 96-well U-bottom plate in a total volume of 100 ul media. Anti-PD-1 and/or anti-LAG-3 IgG antibodies (50 ul, final volume of 150 ul per well) were added starting at a final concentration of 133-400 nM with 5-fold serial dilutions. Cells were co-cultured with peptides and antibodies for 5-6 days. Conditioned media was collected and tested for human IFN-g levels by ELISA.

### Example 10: Characteristics of Illustrative Anti-LAG3 Antibodies

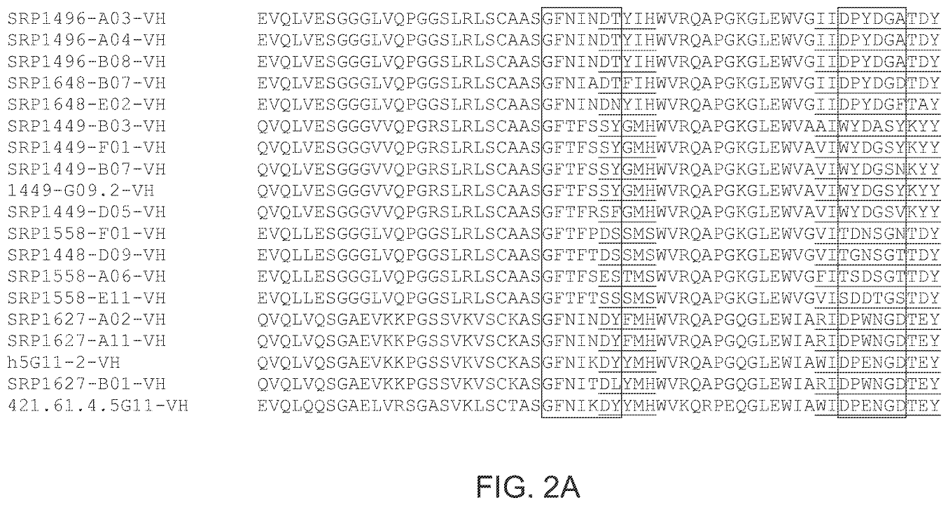

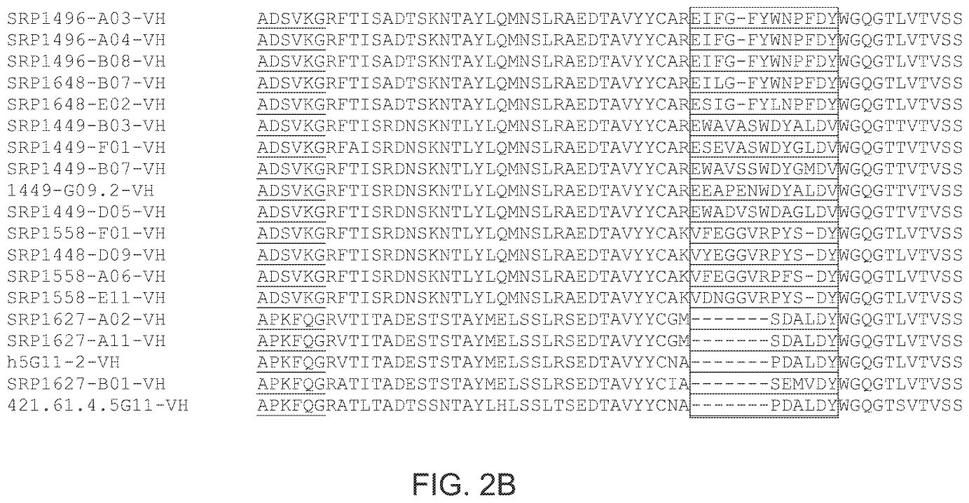

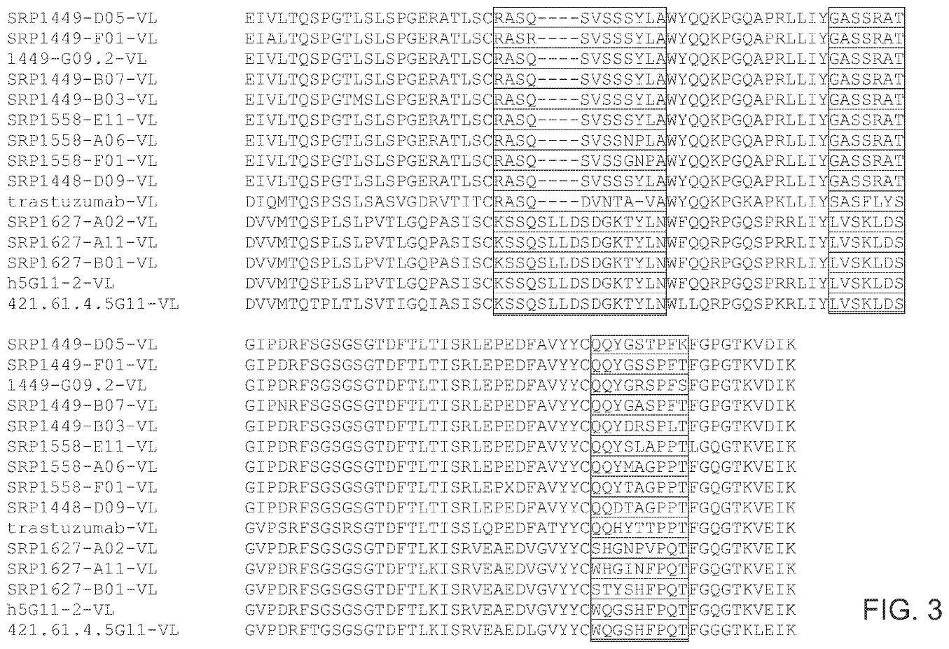

FIG. 2 (A-B) provides an alignment of the V_{H} sequences provided herein. FIG. 3 provides an alignment of the V_{L} sequences provided herein. Chothia CDR sequences are highlighted, and Kabat CDR sequences are underlined.

Tables 22-23 provide results obtained using the illustrative LAG3 antibodies described herein. Table 22 presents the results of binding assays for antibodies provided herein. Table 23 provides the results of functional assays provided herein.

TABLE 22
Binding Assays

| | | | | | | | | Human | Human | Cyno |
|---|---|---|---|---|---|---|---|---|---|---|
| | | | Human LAG3 | | | Cyno LAG3 | | LAG3 | LAG3 | LAG3 |
| Antibody | | | (Biacore) | | | (Biacore) | | (CHO) | (293T) | (293T) |
| | | SEQ ID | kₐ | k_{d} | K_{D} | k_{d} | K_{D} | K_{D} | K_{D} | K_{D} |
| Name | Scaffold | NO(s) | (1/Ms) | (1/s) | (M) | (1/s) | (M) | (nM) | (nM) | (nM) |
| 421.61.4.5G11 | Murine IgG | 210 (V_{H}); | 5.02E+04 | 5.15E−04 | 1.03E−08 | 6.41E−04 | 4.51E−09 | 18.0 | | 4.5 |
| | | 266 (V_{L}) | | | | | | | | |
| SRP1627-A02 | ScFvFc | 216 (V_{H}); | 1.64E+06 | 7.10E−02 | 4.33E−08 | | | | 0.97 | +−− |
| | | 262 (V_{L}) | | | | | | | | |
| SRP1627-A11 | ScFvFc | 207 | 3.12E+05 | 4.22E−03 | 1.35E−08 | | | | 0.74 | +−− |
| | | (V_{H}); 263 | | | | | | | | |
| | | (V_{L}) | | | | | | | | |
| SRP1627-B01 | ScFvFc | 209 (V_{H}); | 1.04E+06 | 1.52E−02 | 1.47E−08 | | | | 0.19 | nd |
| | | 264 (V_{L}) | | | | | | | | |
| h5G11-2 | ScFvFc | 208 (V_{H}); | 2.67E+03 | 2.30E−03 | 8.63E−07 | | | | 78 | nd |
| | | 265 (V_{L}) | | | | | | | | |
| SRP1449-B03 | ScFvFc | 197 (V_{H}); | 4.72E+05 | 3.02E−04 | 6.4E−10 | | | 1.92 | | |
| | | 256 (V_{L}) | | | | | | | | |
| SRP1449-B07 | ScFvFc | 199 (V_{H}); | 7.94E+05 | 5.36E−04 | 6.76E−10 | | | 1.06 | | |
| | | 255 (V_{L}) | | | | | | | | |
| SRP1449-D05 | ScFvFc | 201 (V_{H}); | 4.60E+05 | 6.27E−04 | 1.36E−09 | | | 1.54 | | |
| | | 252 (V_{L}) | | | | | | | | |
| SRP1449-F01 | ScFvFc | 198 (V_{H}); | 2.61E+05 | 8.07E−04 | 3.1E−09 | | | 3.37 | | |
| | | 253 (V_{L}) | | | | | | | | |
| 1449-G09.2 | ScFvFc | 200 (V_{H}); | 1.65E+06 | 6.78E−05 | 4.12E−11 | | | 0.32 | | |
| | | 254 (V_{L}) | | | | | | | | |
| SRP1558-A06 | ScFvFc | 204 (V_{H}); | 7.9E+05 | 9.9E−03 | 1.3E−08 | | | | 0.3 | 0.3 |
| | | 258 (V_{L}) | | | | | | | | |
| SRP1558-E11 | ScFvFc | 205 (V_{H}); | 5.6E+05 | 1.7E−02 | 3.1E−08 | | | | 0.5 | 1.0 |
| | | 257 (V_{L}) | | | | | | | | |
| SRP1558-F01 | ScFvFc | 202 (V_{H}); | 4.35E+05 | 1.1E−02 | 2.4E−08 | | | | 0.5 | 0.7 |
| | | 259 (V_{L}) | | | | | | | | |
| SRP1448-D09 | ScFvFc | 203 (V_{H}); | 1.61E+05 | 6.2E03− | 3.9E−08 | | | | 0.4 | 1.6 |
| | | 260 (V_{L}) | | | | | | | | |
| SRP1496-A03 | IgG | 192 (V_{H}); | 1.24E+06 | 2.34E−02 | 1.89E−08 | | | | 40.6 | |
| | | 261 (V_{L}) | | | | | | | | |
| SRP1496-A04 | IgG | 193 (V_{H}); | 1.29E+06 | 1.96E−02 | 1.52E−08 | | | | 35.0 | |
| | | 261 (V_{L}) | | | | | | | | |
| SRP1496-B08 | IgG | 194 (V_{H}); | 1.31E+06 | 2.79E−02 | 2.13E−08 | | | | 39.4 | |
| | | 261 (V_{L}) | | | | | | | | |
| SRP1648-B07 | IgG | 197 (V_{H}); | 1.12E+07 | 2.75E−02 | 2.46E−09 | | | | positive | |
| | | 261 (V_{L}) | | | | | | | | |
| SRP1648-E02 | IgG | 196 (V_{H}); | 5.35E+07 | 1.22E−01 | 2.27E−09 | | | | positive | |
| | | 261 (V_{L}) | | | | | | | | |

nd = not detected;
+++ = binding observed, K_{D} not calculated

TABLE 23
Functional Assays.

| | | | Cyno |
|---|---|---|---|
| | MHCII | | ELISA |
| | Blockade | | Reactivity |
| | IC₅₀ | Functional | EC₅₀ |
| Antibody | (nM) | Activity | (nM) |
| 421.61.4.5G11 | 64.3 | positive | NT |
| SRP1627-A02 | 1.7 | NT | NT |
| SRP1627-A11 | 1.6 | NT | NT |
| SRP1627-B01 | 0.4 | NT | NT |
| h5G11-2 | nd | NT | NT |
| SRP1449-B03 | 1.0 | NT | NT |
| SRP1449-B07 | 1.2 | NT | NT |
| SRP1449-D05 | 2.3 | NT | NT |
| SRP1449-F01 | 1.7 | NT | NT |
| 1449-G09.2 | 1.1 | Positive | NT |
| SRP1558-A06 | Not tested | Positive | NT |
| SRP1558-E11 | Not tested | NT | NT |
| SRP1558-F01 | Not tested | NT | NT |
| SRP1448-D09 | 1.9 | NT | NT |
| SRP1496-A03 | 41.3 | NT | 4.2 |
| SRP1496-A04 | 55.8 | NT | 1.6 |
| SRP1496-B08 | 28.6 | NT | 2.6 |
| SRP1648-B07 | 4.5 | NT | NT |
| SRP1648-E02 | 4.1 | NT | NT |

NT = not tested

### Example 11: Methods for Assessing Bi-Specific Antigen-Binding Constructs

Bi-specific antigen-binding constructs as described in Examples 12 to 17 were assessed for functional characteristics based on the following methods.

### Cell Lines and Reagents

Stably transfected CHO-k and U2OS cells were cultured in RPMI or DMEM/F12, respectively, supplemented with 10% fetal calf serum, penicillin/streptomycin and glutamine. On day of assay, cells were washed with PBS, detached with Accutase™ (BD Biosciences; San Jose, Calif.), and resuspended in culture media. Daudi cells were cultured in RPMI with 10% fetal calf serum, penicillin/streptomycin and glutamine and maintained at 0.3 to 3×10⁶ cells/mL.

### Cell Binding Assay

CHO-k cells stably expressing PD-1 or LAG3 (human or cynomolgus) on the cell surface were used to assess binding affinity of anti-PD-1/LAG3 bispecific antibody constructs. Cells were seeded at 100,000 cells/well in 100 μL of FACS buffer (1×PBS, 0.5% BSA, 0.1% sodium azide) in 96-well polypropylene plates. The cells were centrifuged at 1.5K rpm and resuspended with test antibodies diluted in FACS buffer and incubated on ice for 1 hour. The cells were washed twice with FACS buffer and incubated on ice for 30 mins with R-Phycoerythrin-AffiniPure F(ab′)2 Fragment Goat Anti-Human IgG (H+L) secondary detection antibody (1:200 dilution, Jackson ImmunoResearch Laboratories, West Grove, Pa.). The cells were washed twice with FACS buffer, fixed in 4% paraformaldehyde in PBS (Santa Cruz Biotechnology; Dallas, Tex.) for 20 mins on ice in the dark, washed twice with FACS buffer, and analyzed using the BD LSR II Flow Cytometer (BD Biosciences; San Jose, Calif.). Data were analyzed using FlowJo (FlowJo, LLC; Ashland, Oreg.) to determine mean fluorescence intensities. Binding constants were calculated using the statistical software, GraphPad Prism (GraphPad Software; La Jolla, Calif.) using the nonlinear regression equation, one site—specific binding with Hill slope. The secondary antibody by itself was used as a control.

### Affinity and Kinetic Binding Analyses

Anti-Fc polyclonal antibodies were immobilized onto a CMS chip (GE Life Sciences) using amine coupling chemistry (from Amine Coupling Kit, GE Life Sciences). The immobilization steps were carried out at a flow rate of 25 μl/min in 1×HBS-EP+buffer (GE Life Sciences; 10× Stock diluted before use). The sensor surfaces were activated for 7 min with a mixture of NHS (0.05 M) and EDC (0.2 M). The Anti-Fc antibodies were injected over all 4 flow cells at a concentration of 25 ug/ml in 10 mM sodium acetate, pH 4.5, for 7 min. Ethanolamine (1 M, pH 8.5) was injected for 7 min to block any remaining activated groups. An average of 12,000 response units (RU) of capture antibody was immobilized on each flow cell.

Kinetic binding experiments were performed at 25° C. using 1×HBS-EP+buffer. Test and control antibodies were injected over the anti-Fc surface at concentrations of 5-10 μg/mL for 12 seconds at a flow rate of 10 μl/min on flow cells 2, 3 and 4, followed by a buffer wash for 30 seconds at the same flow rate. Kinetic characterization of antibody samples was carried out with a dilution series of antigen and 1 injection of 0 nM antigen. After capturing ligand (antibody) on the anti-Fc surface, the analyte (hPD1 or hLAG3, Acro Biosystems, Newark, Del.) was bound at 100, 25, 6.25 and 0 nM for 180 seconds, followed by a 450 second dissociation phase at a flow rate of 50 μl/min. Between each ligand capture and analyte binding cycle, regeneration was carried out using 2 injections of 10 mM Glycine pH 2.0 for 30 seconds at 30 μL/min, followed by a 30 second buffer wash step.

The data was fit with the Biacore T200 Evaluation software, using a 1:1 Langmuir binding model. K_{D} (affinity, nM) was determined as a ratio of the kinetic rate constants calculated from the fits of the association and dissociation phases.

### PD1 Competition Assay

CHO-k cells stably expressing human PD-L1 or PD-L2 were used to measure the ability of anti-PD1/LAG3 bi-specific antibody constructs to block rabbit Fc-tagged recombinant human PD1 (rhPD1-rabbit Fc) protein binding. Cells were seeded at 100,000 cells/well in 100 μL of FACS buffer in 96-well polypropylene plates. The cells were centrifuged and resuspended with test antibodies (2× concentration) diluted in FACS buffer, immediately followed by equal volume of rhPD1-rabbit Fc (added at 2× concentration; 0.2 ng/mL and 0.1 ng/mL final for PD-L1 and PD-L2, respectively). Cells were incubated on ice for 1 hour and washed twice. rhPD1-rabbit Fc binding on CHO-human PD-L1 or PD-L2 cells was detected with R-Phycoerythrin AffiniPure F(ab′)₂ Fragment Goat Anti-Rabbit IgG (H+L) (1:200 dilution, Jackson ImmunoResearch) for 30 minutes. The cells were washed twice with FACS buffer, fixed in 4% paraformaldehyde in PBS for 20 mins on ice in the dark, washed twice with FACS buffer, and analyzed using the BD LSR II Flow Cytometer. Data were analyzed using FlowJo (FlowJo, LLC; Ashland, Oreg.) to determine mean fluorescence intensities. Prism 6 software was used to create one site, specific binding with Hill slope curves (Log transform) to determine IC₅₀ values.

### LAG3 Competition Assay

Daudi cells expressing Major Histocompatibility Class II (MHCII) molecules, the natural ligand for LAG3, were used to measure the ability of anti-PD1/LAG3 bi-specific antibody constructs to block biotinylated (ThermoFisher, Cat #21329) recombinant human LAG3 protein (rhLAG3, R&D Cat #2319-C3) binding. Cells were seeded at 100,000 cells/well in 100 μL of FACS buffer in 96-well polypropylene plates. The cells were centrifuged and resuspended with test antibodies (2× concentration) diluted in FACS buffer, immediately followed by equal volume of biotinylated rhLAG3 (added at 2× concentration; 7.5 μg/mL final). Cells were incubated on ice for 1 hour and washed twice. Biotinylated rhLAG3 binding was detected with 2 μg/mL R-Phycoerythrin-conjugated Streptavidin (eBioscience) for 30 minutes. Cells were washed twice with FACS buffer, fixed in 4% paraformaldehyde in PBS for 20 mins on ice in the dark, washed twice with FACS buffer, and analyzed using the BD LSR II Flow Cytometer. Data were analyzed using FlowJo (FlowJo, LLC; Ashland, Oreg.) to determine mean fluorescence intensities. Prism 6 software was used to create one site, specific binding with Hill slope curves (Log transform) to determine IC₅₀ values.

### PD1×LAG3 Cell-Based Bridging Assay

A cell-based bridging assay was developed using the PathHunter® platform (DiscoverX, Fremont, Calif.) to detect simultaneous binding of PD1 and LAG3 on the cell surface. Briefly, a split beta-galactosidase reporter enzyme was utilized such that enzyme activity could only be detected when the enzyme acceptor (EA) and ProLink (PK) fragments can assemble. The EA-fragment was C-terminally fused to PD-1 (residues 1-199) and the PK fragment was C-terminally fused to LAG3 (residues 1-477). Both fusion constructs were co-expressed in U2OS cells, and stable cell pools were generated. To assess PD1×LAG3 bispecific bridging, 10,000 cells per well were added to opaque 96-well polystyrene plates. The bi-specific antibodies were serially diluted and incubated with the cells for 16 hours at 37° C. Binding was subsequently detected by measuring beta-galactosidase activity using the Beta-Glo® Assay System (Promega Cat. # TM239) and plates were read on an Envision luminometer (integration time of 0.5 sec/well). Prism 6 software was used to create agonist vs. response curves with variable slope to determine EC50 values.

### CMV Recall Assay for Bi-Specific Antibodies

Peripheral blood mononuclear cells (PBMC) were initially isolated from CMV-positive human donors (Stanford Blood Center) by differential gradient centrifugation using NycoPrep™ 1.077 (Axis-Shield). PBMC were cryopreserved with Recovery™ Cell Culture Freezing Medium (Life Technologies) in liquid nitrogen. For the assay, PBMC (0.2×10⁶ cells/well) were cultured in serum-free media containing 2% human AB serum and CMV peptide pools for the CMV IE-1 and CMV pp65 proteins (Miltenyi Biotec) in a total volume of 100 μL per well. Anti-PD1/LAG3 bi-specific antibody candidates (50 μL/well) were added as 3× stock with 5-fold serial dilution titration. Cells were cultured for 5 days. Conditioned media was collected and tested for human IFN-γ levels by ELISA (BD Biosciences).

### Example 12: Generation of a PD1/LAG3 Bi-Specific Antigen-Binding Construct (Two-Chain scFvFc with Knob-in-Hole Mutations)

Embodiments of a PD1/LAG3 bi-specific antigen-binding construct comprising a two-chain scFvFc arrangement were prepared using the following arrangements: (1) an anti-PD1 scFvFc knob paired with an anti-LAG3 scFvFc hole, and (2) an anti-PD1 scFvFc hole paired with an anti-LAG3 scFvFc knob. The scFvFcs include scFvs generated in accordance with Section 4.1.1.1. Table 24 lists exemplary scFvFc fragments and their corresponding SEQ ID NOs that are found within such constructs.

TABLE 24
Exemplary scFvFc Fragments and Sequences
for Two-chain scFvFc Arrangements

| scFvFc | SEQ ID NO. |
|---|---|
| Anti-PD1 1353-G10 scFvFc hole | 368 |
| Anti-LAG3 1449-G09.2 scFvFc knob | 371 |
| Anti-PD1 1353-G10 scFvFc knob | 369 |
| Anti-LAG3 1449-G09.2 scFvFc hole | 380 |

Functional characteristics for the exemplary two-chain scFvFc arrangements described above are provided in Table 25.

TABLE 25
Functional Characteristics of PD1/LAG3 Bispecific Antibodies (Two-chain scFvFc
Arrangements)

| | | AlphaLISA | PD1 | LAG3 | | |
|---|---|---|---|---|---|---|
| | | bridging | ELISA | ELISA | 293T- | CHO- |
| | | (max | EC50 | EC50 | LAG3 Kd | PD1 Kd |
| Construct | Description | signal) | (nM) | (nM) | (nM) | (nM) |
| E | aLAG3 1449-G09.2_K | 31576 | 1.1 | 0.69 | 8 | 3 |
| | x aPD1 1353-G10_H | | | | | |
| F | aLAG3 1449-G09.2_H | 20892 | 1.2 | 0.44 | 15 | 6 |
| | x aPD1 1353-G10_K | | | | | |

“K” = knob
“H” = hole

### Example 13: Generation of a PD1/LAG3 Bi-Specific Antigen-Binding Construct (Three-Chain Fab× scFvFc with Knob-in-Hole Mutations)

Embodiments of a PD1/LAG3 bi-specific antigen-binding construct comprising a three-chain Fab× scFvFc structure were prepared using the following arrangements: (1) an anti-PD1 scFvFc knob paired with an anti-LAG3 half IgG (HC+LC) hole, (2) an anti-PD1 scFvFc hole paired with an anti-LAG3 half IgG (HC+LC) knob, (3) an anti-PD1 half IgG (HC+LC) knob paired with an anti-LAG3 scFvFc hole, and (4) an anti-PD1 half IgG (HC+LC) hole paired with an anti-LAG3 scFvFc knob. Table 26 lists exemplary scFvFc and half IgG fragments and their corresponding SEQ ID NOs that can be used in these embodiments.

TABLE 26
Exemplary scFvFc and Half IgG Fragments and Sequences for Three-
chain Fab × scFvFc Arrangements with Knob-in-Hole Mutations

| scFvFc | SEQ ID NO. |
|---|---|
| Anti-PD1 1353-G10 scFvFc knob | 369 |
| Anti-PD1 1353-G10 scFvFc hole | 368 |
| Anti-PD1 1353-G10 HC knob | 381 |
| Anti-PD1 1353-G10 HC hole | 382 |
| Anti-PD1 1353-G10 LC | 383 |
| Anti-LAG3 1449-G09.2 scFvFc knob | 371 |
| Anti-LAG3 1449-G09.2 scFvFc hole | 384 |
| Anti-LAG3 1449-G09.2 HC hole | 372 |
| Anti-LAG3 1449-G09.2 LC | 366 |

Exemplary anti-PD1 scFvFcs and anti-LAG3 scFvFcs that can be included in the three-chain arrangement are described in Example 13.

Functional characteristics for the exemplary three-chain Fab× scFvFc arrangements described above are provided in Table 27. The half IgG fragments are labeled as “Fab_K” or “Fab_H” where “Fab” indicates the antigen-binding region and “K” or “H” indicates a knob or hole mutation in the Fc region.

TABLE 27
Functional Characteristics of PD1/LAG3 Bispecific Antibodies (Comparison of Two-
chain scFvFc × scFvFc Arrangements to Three-chain Fab × scFvFc Arrangements)

| | | | | CHO-PD1 | CHO-PD1 |
|---|---|---|---|---|---|
| | | AlphaLISA | AlphaLISA | cell binding, | cell binding, |
| Construct | Description | Bmax | EC50 (nM) | Kd (nM) | Bmax |
| F | aPD1 1353-G10 scFvFc_K × | 50547 | 0.069 | 1.1 | 7201 |
| | aLAG3 1449-G09.2 | | | | |
| | scFvFc_H | | | | |
| E | aPD1 1353-G10 scFvFc_H × | 36902 | 0.011 | 1 | 6296 |
| | aLAG3 1449-G09.2 | | | | |
| | scFvFc_K | | | | |
| G | aPD1 1353-G10 Fab_K × | 210201 | 0.051 | 0.8 | 6375 |
| | aLAG3 1449-G09.2 | | | | |
| | scFvFc_H | | | | |
| H | aPD1 1353-G10 Fab_H × | 86841 | 0.011 | 0.4 | 5455 |
| | aLAG3 1449-G09.2 | | | | |
| | scFvFc_K | | | | |

### Example 14: Generation of a PD1/LAG3 Bi-Specific Antigen-Binding Construct (Three-Chain Fab× scFvFc with zw Mutations)

Embodiments of a PD1/LAG3 bi-specific antigen-binding construct comprising a three-chain Fab× scFvFc structure were prepared using the following arrangements: (1) an anti-PD1 scFvFc knob with zwA mutations paired with an anti-LAG3 half IgG (HC+LC) with zwB mutations, (2) an anti-PD1 scFvFc with zwB mutations paired with an anti-LAG3 half IgG (HC+LC) with zwA mutations, (3) an anti-PD1 half IgG (HC+LC) with zwA mutations paired with an anti-LAG3 scFvFc with zwB mutations, and (4) an anti-PD1 half IgG (HC+LC) with zwB mutations paired with an anti-LAG3 scFvFc with zwA mutations. The mutations encompassed by “zwA” mutations include T350V/L351Y/F405A/Y407V in the CH3 domain. The mutations encompassed by “zwB” mutations include T350V/T366L/K392L/T394W in the CH3 domain. Table 28 lists exemplary scFvFc and half IgG fragments and their corresponding SEQ ID NOs that can be used in these embodiments.

TABLE 28
Exemplary scFvFc and Half IgG Fragments and Sequences for
Three-chain Fab × scFvFc Arrangements with zw Mutations

| scFvFc | SEQ ID NO. |
|---|---|
| Anti-PD1 1353-G10 scFvFc zwA | 370 |
| Anti-PD1 1353-G10 scFvFc zwB | 388 |
| Anti-PD1 1353-G10 HC zwA | 389 |
| Anti-PD1 1353-G10 HC zwB | 390 |
| Anti-PD1 1353-G10 LC | 383 |
| Anti-LAG3 1449-G09.2 scFvFc zwA | 391 |
| Anti-LAG3 1449-G09.2 scFvFc zwB | 392 |
| Anti-LAG3 1449-G09.2 HC zwA | 393 |
| Anti-LAG3 1449-G09.2 HC zwB | 373 |
| Anti-LAG3 1449-G09.2 LC | 376 |

Functional characteristics for the exemplary three-chain Fab× scFvFc arrangements described above are provided in Table 29. The half IgG fragments are labeled as “Fab-zwA” or “Fab-zwB” where “Fab” indicates the antigen-binding region and “zwA” or “zwB” indicates the Fc region with the mutations described above.

TABLE 29
Functional Characteristics of PD1/LAG3 Bispecific Antibodies
(Three-chain Fab × scFvFc Arrangements with zw Mutations)

| | | DSC Analysis | | |
|---|---|---|---|---|
| | | TM Onset | TM1 | TM2 |
| Construct | Sample ID | ° C. | ° C. | ° C. |
| I | 1353-G10-Fab-zwB; | 52.4 | 61.1 | 65.6 |
| | 1449-G09.2-scFv-zwA | | | |
| A | 1353-G10-scFv-zwB; | 51.9 | 62.8 | 78.1 |
| | 1449-G09.2-Fab-zwA | | | |

Functional characteristics for exemplary three-chain Fab× scFvFc arrangements described above are provided in Tables 30 to 32.

TABLE 30
Functional Characteristics of PD1/LAG3 Bispecific Antibodies (Three-chain scFvFc × Fab-Fc Arrangements)

| | | | | | | | | AlphaLISA | | | |
|---|---|---|---|---|---|---|---|---|---|---|---|
| | | | | | | | | (LAG3-Fc | | | |
| | | | | | | | | Acceptor/PD1-Fc | | | |
| | Description | Human PD1 kinetics | | | Human LAG3 kinetics | | | Donor) | | DSF (° C.) | |
| Construct | scFv × Fab | ka (1/Ms) | kd (1/s) | KD (M) | ka (1/Ms) | kd (1/s) | KD (M) | EC50 | Bmax | TM1 | TM2 |
| A | aPD1 1353-G10 scFv_zwB × | 5.59E+05 | 8.49E−04 | 1.52E−09 | 1.21E+06 | 1.02E−04 | 8.45E−11 | 0.01 | 4688 | 51.3 | 66 |
| | aLAG3 Fab_zwA | | | | | | | | | | |
| C | aPD1 1353-G10 Vk1-39LC | 5.03E+05 | 6.49E−04 | 1.29E−09 | 1.34E+06 | 6.94E−04 | 5.19E−10 | ND | ND | 54.5 | 72.8 |
| | scFvFc zwB V262E; aLAG3 | | | | | | | | | | |
| | 1449-G09.2 Fab zwA V262E | | | | | | | | | | |
| B | aPD1 1353-G10 scFvFc zwB | 1.12E+06 | 4.87E−03 | 4.37E−09 | 1.46E+06 | 1.38E−04 | 9.44E−11 | ND | ND | 52.5 | 72.7 |
| | R28T/P30D/H31S V262E; | | | | | | | | | | |
| | aLAG3 1449-G09.2 Fab zwA | | | | | | | | | | |
| | V262E | | | | | | | | | | |
| J | aPD1 1353-G10 R28T/P30D | 9.22E+06 | 1.09E−03 | 1.19E−09 | ND | ND | ND | ND | ND | ND | ND |
| | zwB × aLAG3 1449-G09.2 | | | | | | | | | | |
| | Fab zwA | | | | | | | | | | |
| K | a1353-G10 R28T/P30T/H31S | 1.37E+06 | 7.24E−03 | 5.3E−09 | ND | ND | ND | ND | ND | ND | ND |
| | zwB × 1449-G09.2 Fab zwA | | | | | | | | | | |
| L | 1353-G10 R28D/P30T/H31S | 1.50E+06 | 7.77E−03 | 5.18E−09 | ND | ND | ND | ND | ND | ND | ND |
| | zwB × 1449-G09.2 Fab zwA | | | | | | | | | | |
| M | 1353-G10 R28T/P30T/H31D | 2.07E+06 | 1.02E−02 | 4.91E−09 | ND | ND | ND | ND | ND | ND | ND |
| | zwB × 1449-G09.2 Fab zwA | | | | | | | | | | |
| N | 1353-G10 R28T/P30D/H31S | 1.38E+06 | 55.2E−03 | 4.01E−09 | ND | ND | ND | ND | ND | ND | ND |
| | zwB × 1449-G09.2 Fab zwA | | | | | | | | | | |
| O | 1353-G10 R28T/H31D zwB × | 1.68E+06 | 7.48E−03 | 4.44E−09 | ND | ND | ND | ND | ND | ND | ND |
| | 1449-G09.2 Fab zwA | | | | | | | | | | |
| P | 1353-G10 R28D/P30D zwB × | 9.85E+05 | 1.00E−03 | 1.02E−09 | ND | ND | ND | ND | ND | ND | ND |
| | 1449-G09.2 Fab zwA | | | | | | | | | | |
| Q | 1353-G10 R28D/H31D zwB × | 1.55E+06 | 6.70E−03 | 4.32E−09 | ND | ND | ND | ND | ND | ND | ND |
| | 1449-G09.2 Fab zwA | | | | | | | | | | |

TABLE 31

| | | | | | | | | | AlphaLISA (LAG3-Fc | |
|---|---|---|---|---|---|---|---|---|---|---|
| | | | | | | | | | Acceptor/PD1-Fc | |
| | | | human PD1 kinetics | | | human LAG3 kinetics | | | Donor) | |
| Construct | Ligand | Scaffold | ka (1/Ms) | kd (1/s) | KD (M) | ka (1/Ms) | kd (1/s) | KD (M) | EC50 | Bmax |
| A | PD1 scFv_zwB × | scFv × Fab | 5.59E05 | 8.49E−04 | 1.52E−09 | 1.21E06 | 1.02E−04 | 8.45E−11 | 0.01 | 4688 |
| | LAG3 Fab_zwA | | | | | | | | | |
| R | LAG × PD1 (I) | fab × fab | 6.30E05 | 6.42E−04 | 1.02E−09 | 1.46E06 | 1.29E−04 | 8.85E−11 | 0.015 | 33678 |
| S | LAG × PD1 (II) | fab × fab | 6.65E05 | 7.71E−04 | 1.16E−09 | 1.41E06 | 1.36E−04 | 9.65E−11 | 0.015 | 31794 |
| T | LAG × PD1 (III) | fab × fab | 7.24E05 | 8.06E−04 | 1.11E−09 | 1.34E06 | 1.52E−04 | 1.13E−10 | 0.006 | 25158 |
| U | LAG × PD1 (I) + | fab × fab | 5.20E05 | 1.15E−03 | 2.22E−09 | 1.09E06 | 2.63E−04 | 2.41E−10 | ND | ND |
| | V262E | | | | | | | | | |

ND = not determined

TABLE 32
Functional Characteristics of PD1/LAG3 Bispecific Antibodies (Four-chain Fab × Fab-Fc Arrangements)

| | | | CHO-PD1 cell | | | | | | |
|---|---|---|---|---|---|---|---|---|---|
| | | | binding | | CHO-LAG3 cell | | PD1/PDL1 | LAG3/MHCII | PxL cell |
| | | | Kd | | binding | | competition | competition | bridging |
| Construct | Ligand | Scaffold | (nM) | Bmax | Kd (nM) | Bmax | IC50 (nM) | IC50 (nM) | EC50 (nM) |
| A | PD1 scFv_zwB × | scFv × Fab | 3.7 | 10425 | 3 | 2751 | 118 | 17 | 0.6 |
| | LAG3 Fab_zwA | | | | | | | | |
| R | LAG × PD1 (I) | fab × fab | 3.1 | 13671 | 1.8 | 2947 | 34 | 11 | 0.1 |
| S | LAG × PD1 (II) | fab × fab | 8 | 16739 | 2.4 | 2770 | 69 | 16 | 0.1 |
| T | LAG × PD1 (III) | fab × fab | 4.6 | 11999 | 2.4 | 2689 | 149 | 19 | 0.8 |

### Example 15: Mutation Design in Anti-PD1

To improve solubility and reduce aggregation propensity, an aspartic acid mutation was also made to PD-1 antibody 1353-G10 in CDR H1 (P30D). See, e.g., WO 2016/060033, incorporated herein by reference in its entirety. The mutation was introduced using the general approach described above and in the literature (Dudgeon et al. 2012. PNAS 109(27):10879-10884). IgBLAST (Ye et al. 2013. Nucleic Acids Res 41 (Web Server issue):W34-W40, incorporated herein by reference in its entirety) was used to analyze the 1353-G10 heavy chain. The analysis showed that R28T and H31S mutations would render the sequence closer to the native VH1-18 human germline sequence, which could reduce immunogenicity in humans and improve biophysical behavior, e.g., reduction of aggregation propensity, transition melting temperature, etc. Lastly, to improve the stability of the light chain of 1353-G10, the CDRs were grafted onto a kappa framework Vk1-39 in a similar manner as described previously (Lehmann et al. 2015. mAbs 7(6):1058-1071, incorporated herein by reference in its entirety). The stabilizing mutations and kappa-grafted light chain were also made in the context of an scFv framework, both alone and in combination with each other. The sequences of these scFv are presented as SEQ ID NO: 364 (stabilizing mutations and kappa grafted light chain), SEQ ID NO: 365 (stabilizing mutations), and SEQ ID NO: 366 (kappa grafted light chain), respectively.

### Example 16: Differential Scanning Fluorimetry

A protein thermal shift assay was carried out by mixing the protein to be assayed with an environmentally sensitive dye (SYPRO® Orange, Life Technologies Cat. # S-6650) in a phosphate buffered solution (PBS), and monitoring the fluorescence of the mixture in real time as it underwent controlled thermal denaturation. Protein solutions between 0.2-2 mg/mL were mixed at a 1:1 volumetric ratio with a 1:500 PBS-diluted solution of SYPRO® Orange (SYPRO® Orange stock dye is 5000× in DMSO). Aliquots of 10 μL of the protein-dye mixture were dispensed in quadruplicate in a 384-well microplate (Bio-Rad Cat # MSP-3852), and the plate was sealed with an optically clear sealing film (Bio-Rad Cat # MSB-1001) and placed in a 384-well plate real-time thermocycler (Bio-Rad CFX384 Real Time System). The protein-dye mixture was heated from 25° C. to 95° C., at increments of 0.1° C. per cycle (˜1.5° C. per minute), allowing 3 seconds of equilibration at each temperature before taking a fluorescence measurement. At the end of the experiment, the first and second transition melting temperatures (Tm1 and Tm2, respectively) were determined using the Bio-Rad CFX manager software.

### Example 17: In Vivo Efficacy of Combination Therapy (Anti-PD-1 and Anti-LAG3 Antibodies) on Tumor Establishment and Growth

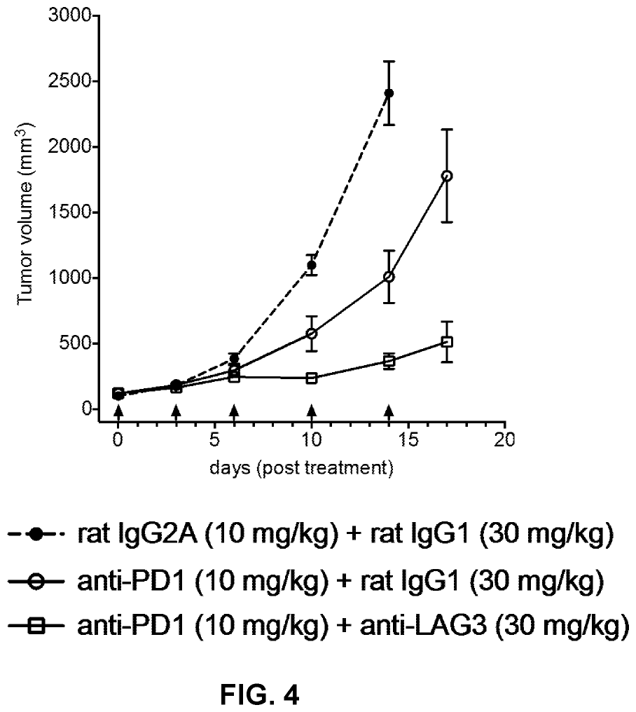

To determine if dual-targeting of PD-1 and LAG3 on mouse T-cells provides additive benefits over treatment with anti-PD-1 alone, MC38 colorectal murine cancer cells (National Cancer Institute, Bethesda, Md.) were subcutaneously implanted in C57BL/6 mice (2e6 cells in 100 μL PBS) in the flank region (10 mice per treatment group). Commercially available mouse surrogate antibodies were dosed in mice intraperitoneally 6 days after tumor cell implantation with a total of 5 doses at 3-4 day intervals (post-initial treatment at days 0, 3, 6, 10 and 14). Commercial antibodies administered (Bio×Cell, West Lebanon, N.H.) were 10 mg/kg anti-PD-1 (clone RMP1-14 rat IgG2A), 10 mg/kg control rat IgG2A (clone 2A3), 30 mg/kg anti-LAG3 (clone C9B7W rat IgG1) and 30 mg/kg for control rat IgG1 (clone HRPN). Treatment groups, dose levels and antibody combinations are shown in Table 33. Results are shown in FIG. 4.

TABLE 33
Treatment Groups
Treatment Groups

control rat IgG2A (10 mg/kg) +
control rat IgG1 (30 mg/kg)
anti-PD-1 RMP1-14 rat IgG2A (10 mg/kg) +
control rat IgG1 (30 mg/kg)
anti-PD-1 RMP1-14 rat IgG2A (10 mg/kg) +
anti-LAG3 C9B7W rat IgG1 (30 mg/kg)

Mean tumor volumes for each treatment groups (mean±standard error of mean (SEM)) are depicted in FIG. 4. Tumor growth is significantly inhibited with anti-PD-1 alone and anti-PD-1/anti-LAG3 combination relative to the control Ab-treated group. Furthermore, addition of anti-LAG3 with anti-PD-1 significantly suppressed tumor growth compared to anti-PD-1 treatment alone suggesting additive effects in targeting both PD-1 and LAG3 in the MC38 tumor model.

### Example 18: Pharmacokinetics and ADA Response of a PD-1/LAG3 Bi-Specific Construct in Cynomolgus Monkeys

PD-1/LAG3 Bi-specific Construct A is a three-chain bi-specific antibody that includes an anti-PD-1 scFvFc with zwB mutation (T350V/T366L/K392L/T394W in C_{H}3 domain) and an anti-LAG3 Fab with zwA mutation (T350V/L351Y/F405A/Y407V in C_{H}3 domain). Bi-specific Construct A was assessed for pharmacokinetic (PK) properties and anti-drug antibody (ADA) response in cynomolgus non-human primates. The bi-specific construct was administered as IV injection to female cynomolgus monkeys (n=5 per group) on day 1 and day 15 at doses of 10, 30 and 100 mg/kg. Serum samples were collected at various timepoints for pharmacokinetic (PK) and ADA assessments. For PK assessments, a sandwich ELISA-based bridging immuno-assay using recombinant PD-1 and LAG3 was used to determine concentrations of the bi-specific construct in serum samples. This assay can detect antibody levels in serum at which the antibody is capable of binding to both PD-1 and LAG3. The assay uses human LAG3 human-Fc recombinant fusion protein as capture protein for the bi-specific construct. Following serum sample binding at appropriate dilutions and wash steps, human PD-1 mouse-Fc recombinant fusion protein was added, and after another series of wash steps, plate-bound bi-specific construct was detected using horse-radish peroxidase (HRP)-conjugated polyclonal anti-mouse-IgG Fc protein. The same dosing material was spiked into cynomolgus monkey serum to generate a standard curve to determine serum concentrations of the bi-specific construct. PK parameters were calculated using the Phoenix WinNonlin (version 6.1) program. The peak serum concentration (Cₘₐₓ) and half-life was determined, and the area under the serum concentration-time curve from 0.25 hour to 168 hours post dose 1 (AUC₁₆₈) was determined by the non-compartmental model using the linear/log trapezoidal rule. Serum concentrations below the limit of quantitation were not used for AUC calculations.

For immunogenicity analysis, pre-dose, day 15 and day 43 samples (last PK timepoint) were tested for ADA levels using an ELISA-based immunogenicity assay. Bi-specific Construct A dosing material was used as the capture reagent and HRP-conjugated mouse anti-non-human primate Fc antibody for detection of ADA complexes. The cut-off point for the assay is defined as the assay response (OD at 450 nm) above which, a sample is identified as ADA-positive, and below which, a sample is identified as ADA-negative. The assay cut-off point was calculated as three times the mean OD of pre-dose monkey serum samples. The cut-point for anti-construct antibody was at an OD of 0.70.

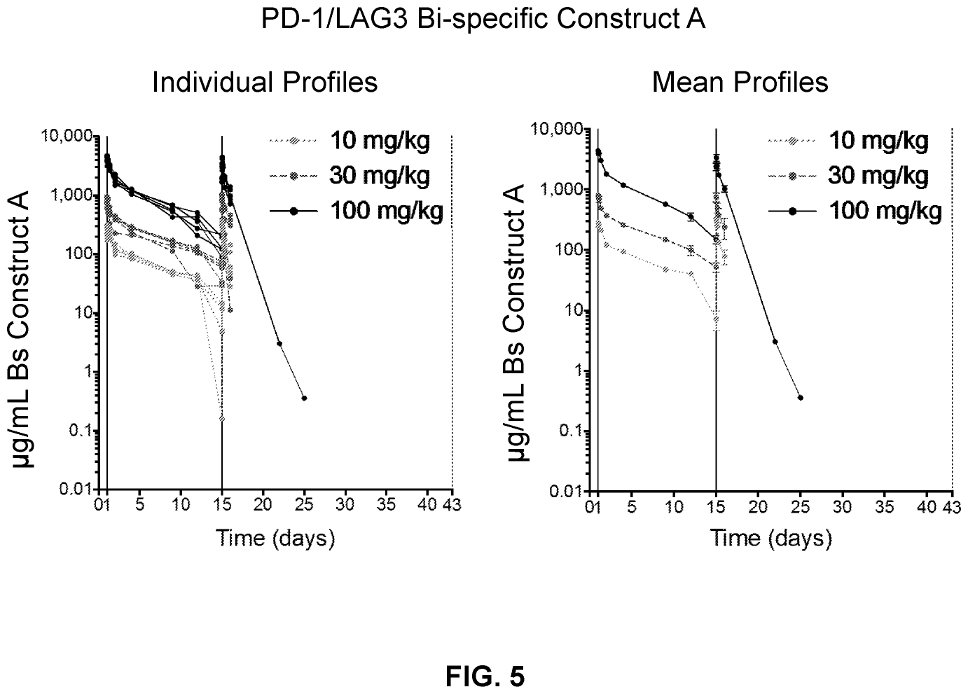

Serum levels of the bi-specific construct in all cynomolgus monkeys (n=5 animals/group) dosed at 10, 30 and 100 mg/kg (Q2W×2) at day 1 and day 15 (x-axis solid lines) are shown in FIG. 5 (left panel). Note that following day 16 (1 day post 2^{nd} dose at day 15), only 2 out of 5 animals per group were sampled for PK and ADA analysis up to day 43 (x-axis dotted line). Mean serum levels for Bi-specific Construct A (±SEM) per dose group are shown in FIG. 5 (right panel). Following day 16, only 1 of 2 animals in the 100 mg/kg group had measurable concentrations up to day 25 (FIG. 5). All other animals (n=2) in 10 and 30 mg/kg dose groups had no measurable concentrations after day 16 (FIG. 5). Rapid clearance and low serum half-life of the bi-specific construct (<4 days) in cynomolgus monkeys were observed at all 3 dose levels post 1^{st} dose up to day 8 (Table 34). Table 34 provides mean pharmacokinetic values (with standard deviation values in parentheses) for each dosing group.

TABLE 34
Pharmacokinetic Profile of Bi-specific Construct A in Cynomolgus Monkeys

| | | | | | | | Half- |
|---|---|---|---|---|---|---|---|
| | Cmax | C,ax/D | AUC₀₋₈ | AUC₀₋₈/Dose | Vz | Cl | life |
| Dose | μg/mL | μg/mL | μg * day/mL | μg * day/mL/(mg/kg) | mL/kg | mL/d/kg | days |
| 10 | 283.02 | 28.30 (3.52) | 740.74 | 74.07 (6.62) | 49.89 | 10.34 | 3.33 |
| mg/kg | (35.24) | | (66.22) | | (6.55) | (0.66) | (0.27) |
| 30 | 846.79 | 28.23 (4.58) | 2120.37 | 70.68 (10.57) | 54.49 | 10.56 | 3.58 |
| mg/kg | (137.27) | | (317.07) | | (9.80) | (1.66) | (0.37) |
| 100 | 4451.29 | 44.51 (3.12) | 9927.57 | 99.28 (6.85) | 33.41 | 8.19 | 2.84 |
| mg/kg | (312.19) | | (684.81) | | (1.98) | (0.77) | (0.27) |

Cmax = the maximum observed concentration measured after dosing
Cmax/D = Cmax divided by the administered dose
AUC₀₋₈ = the area under the concentration versus time curve from the start of dosing, including all time points up to day 8 post Dose 1, using linear/log trapezoidal method
AUC₀₋₈/D = AUC₀₋₈ divided by the administered dose
Vz = apparent volume of distributiuon during terminal phase
Cl = apparent clearance

The results of the immunogenicity analysis for PD-1/LAG3 Bi-specific Construct A indicated that all monkeys dosed with the bi-specific construct at 10, 30 and 100 mg/kg tested positive for ADA on day 15 (n=5) and day 43 (n=2) (data not shown). This suggests rapid PK clearance and low serum half-life of the bi-specific construct may be attributed to cynomolgus ADA response at all 3 dose levels even after the first dose.

This information from this study showed fast clearance of PD-1/LAG3 Bi-specific Construct A and strong anti-drug antibody (ADA) response. Based on this information, new bi-specific constructs were generated and evaluated to determine if a candidate bi-specific construct with lower clearance and ADA response could be obtained.

### Example 19: Pharmacokinetics and ADA Response of Additional PD-1/LAG3 Bi-Specific Construct in Cynomolgus Monkeys

Given the higher clearance, lower serum half-life and strong ADA response with PD-1/LAG3 Bi-specific Construct A, two additional PD-1/LAG3 constructs (“B” and “C”) were assessed for pharmacokinetic (PK) properties and anti-drug antibody (ADA) responses in cynomolgus non-human primates. Bi-specific Construct B is a three-chain bi-specific antibody that includes an anti-PD-1 scFvFc with mutations: R28T/P30D/H31S in V_{H}; V262E in C_{H}2 domain; and T350V/T366L/K392L/T394W in C_{H}3; and an anti-LAG3 Fab with mutations: V262E in C_{H}2 and T350V/L351Y/F405A/Y407V in C_{H}3. Bi-specific Construct C is a three-chain bi-specific antibody that includes an anti-PD-1 scFvFc with mutations: V262E in C_{H}2 domain; and T350V/T366L/K392L/T394W in C_{H}3, in which the scFv is grafted to a kappa light chain; and an anti-LAG3 Fab with mutations: V262E in C_{H}2 and T350V/L351Y/F405A/Y407V in C_{H}3. Both bi-specific constructs were administered as IV injection to female cynomolgus monkeys (n=2 per group) on day 1 and day 15 at a dose of 10 mg/kg. Serum samples were collected at various timepoints for PK and ADA assessments (pre-dose and following post-dose after day 1: 0.25 hour to 240 hours, and pre-dose and following post-dose after day 15: 0.25 hour to 336 hours). For PK assessments, an ELISA method using mouse anti-human IgG (BioRad; clone R10Z8E9), a monoclonal Ab specific for C_{H}2 domain of human IgG with no non-human primate species reactivity, was used as capture Ab and HRP-conjugated polyclonal goat anti-human IgG with minimal cross-reactivity to cyno IgG (Bethyl Laboratories, A80-319P) was used as detection Ab to determine concentrations of Bi-specific Constructs B and C in serum samples. The same dosing materials were spiked into cynomolgus monkey serum to generate a standard curve to determine Bi-specific Construct B and C serum concentrations.

For immunogenicity analysis, pre-1^{st} dose (day 0), pre-2^{nd} dose (day 14), day 28 and day 42 (post-dose 2) serum samples were titrated with 2-fold serial dilutions (20- to 2560-fold, 8 point titration) and tested for ADA levels using an ELISA-based immunogenicity assay. Mouse anti-human IgG (BioRad; clone R10Z8E9), a monoclonal Ab specific for C_{H}2 domain of human IgG with no non-human primate species reactivity, was coated at 2 μg/ml in carbonate/bicarbonate pH 9.6 buffer (Sigma-Aldrich, C3041) overnight at 4° C. in 96-well Nunc MaxiSorp plates. All following steps were performed at room temperature. Plates were washed with PBST buffer (PBS+0.05% Tween-20) and blocked with ELISA blocking buffer (PBS+1% BSA) for 1 hour. Serum samples were serially diluted 2-fold in diluent buffer from 10- to 1280-fold in diluent buffer (PBS, 0.5% BSA, 0.05% Tween-20, 0.35 M NaCl, 0.25% CHAPS) as 2× samples. Respective dosing Abs were diluted at 5 μg/ml in diluent buffer as 2× samples. Diluted serum samples (final 20- to 2560-fold dilutions final) and diluted test Abs (2.5 μg/ml final) were mixed in a 1:1 volume ratio and incubated for 2 hours to allow formation of ADA-Ab complexes before adding to anti-human IgG coated-plates for 1 hour. Plates were washed and HRP-conjugated goat anti-monkey IgG secondary antibody (Bethyl Laboratories, A140-202P) was diluted 1:40,000-fold in diluent buffer and added to plates for 1 hour in the dark. Plates were washed and TMB substrate (SureBlue Reserve, KPL, 53-00-03) was added for 30 min in the dark. Substrate reaction was quenched with equal volume of 1M phosphoric acid and plates read at 450 nm on M5 SpectraMax plate reader (Molecular Devices). Serum samples for each timepoint per animal titrated at various dilutions were plotted as OD fold-increase at 450 nm (OD sample/OD pre-1^{st} dose) versus the inverse of serum dilutions. ADA response is considered positive if the OD fold-increase is greater than 3× OD pre-dose for each individual animal.

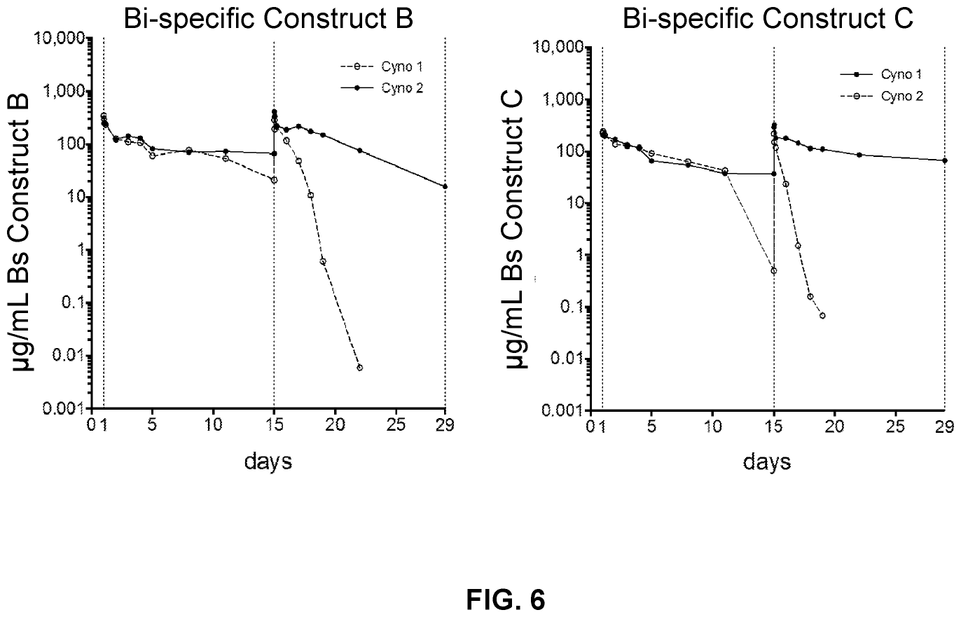

One out of 2 cynomolgus monkeys treated with PD-1/LAG3 Bi-specific Construct B (animal 2) and Bi-specific Construct C (animal 1) exhibited better PK drug profiles with 2 doses at 10 mg/kg after day 1 and day 15 (Q2W×2) (FIG. 6) compared to Bi-specific Construct A (FIG. 5). This suggests that stabilizing mutations in the 1353-G10 a-PD1 arm (e.g. R28T/P30D/H31S in V_{H}) in the a-PD-1/a-LAG3 bispecific improve antibody clearance and half-life in cynomolgus monkeys to potentially achieve sustained exposures for assessments in toxicological studies in non-human primates.

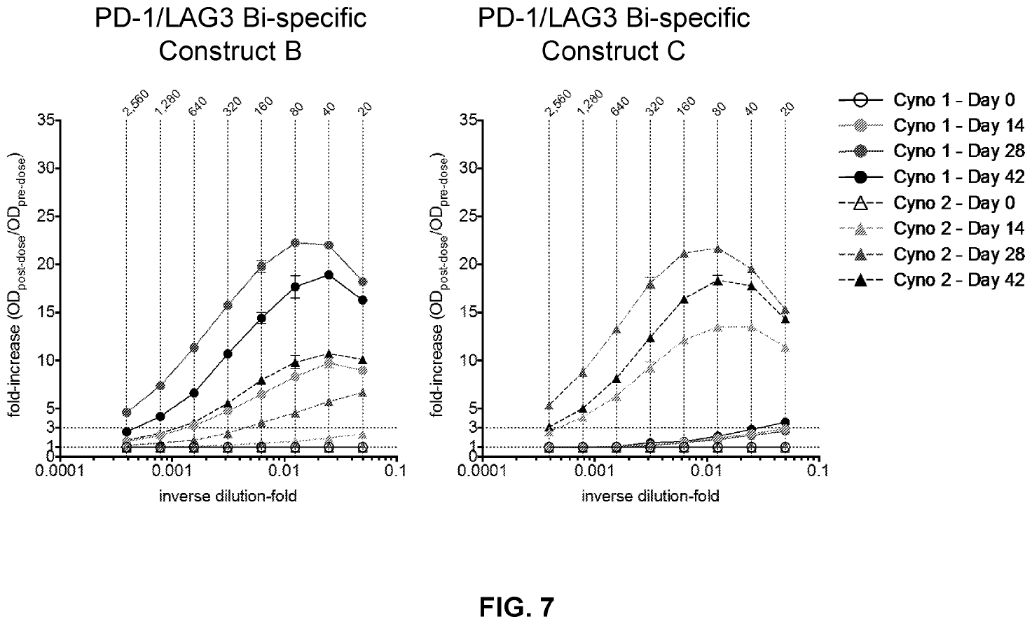

One out of 2 cynomolgus monkeys treated with PD-1/LAG3 Bi-specific Construct B (animal 2) and Bi-specific Construct C (animal 1) showed lower ADA response to the respective bispecific antibodies with 2 doses at 10 mg/kg after day 1 and day 15 (Q2W×2) (FIG. 7). The data suggests that stabilizing mutations and germ-lining mutations in the 1353-G10 a-PD1 arm (e.g. R28T/P30D/H31S in V_{H}) in the a-PD-1/a-LAG3 bispecific is less immunogenic than native a-PD1 arm in Bi-specific Construct A in cynomolgus monkey. In addition, the data indicates that using a kappa graft in the bi-specific construct (Bi-specific construct C) results in reduced immunogenicity and improved pharmacokinetics relative to Bi-specific Construct A, which includes a lambda light chain.

### Example 20: Comparison of PD-1/LAG3 Bi-Specific Constructs B and C to B-Specific Construct A

The methods described in Example 12 were used to assess the characteristics of PD-1/LAG3 Bi-specific Constructs A, B, and C.

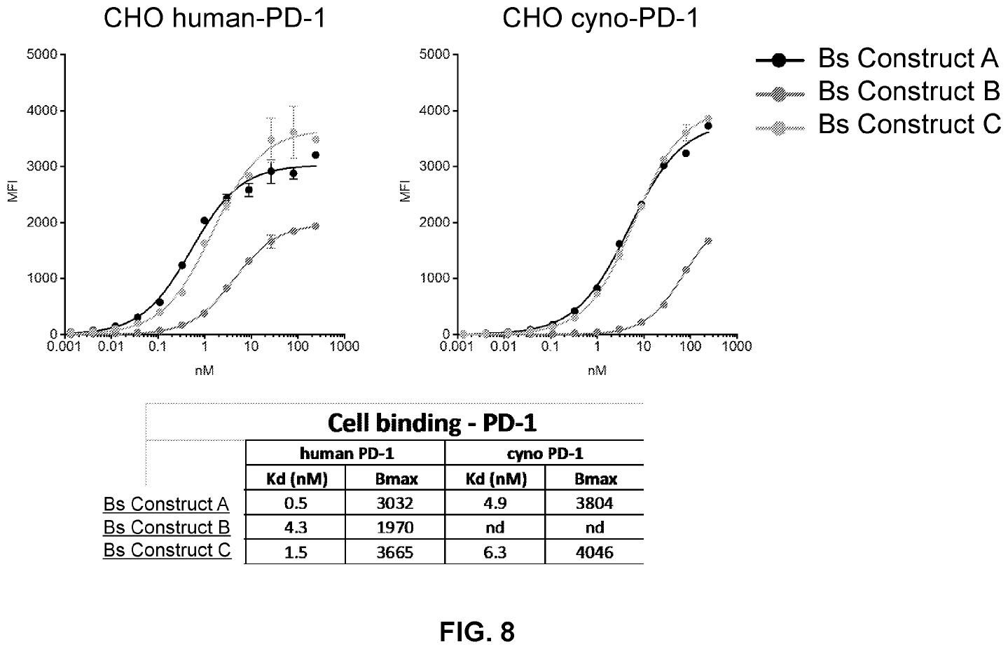

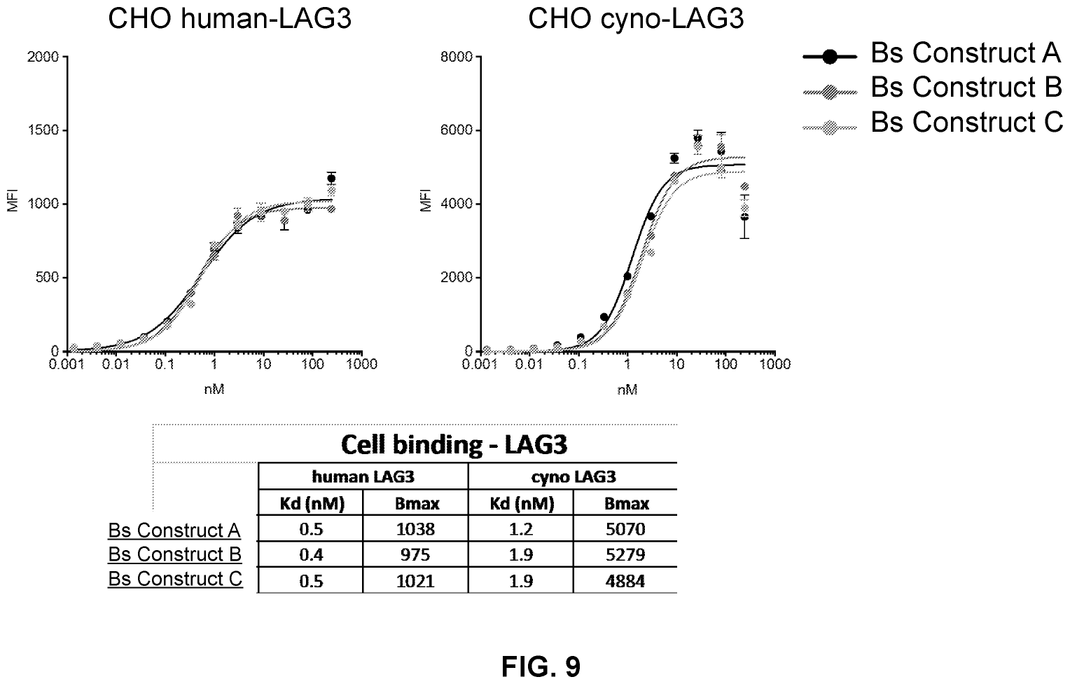

Bi-specific Constructs B and C are PD1×LAG3 bispecific antibodies with different re-engineered anti-PD1 arms relative to Bi-specific Construct A. The three constructs were compared for binding to cell-surface overexpressed human and cynomolgus PD-1 in CHO cells (FIG. 8). Both Bi-specific Constructs A and C bound to human and cynomolgus PD-1 with equivalent binding affinities. In contrast, Bi-Specific Construct B bound to human and cynomolgus PD-1 with reduced binding affinities compared to Bi-Specific Constructs A and C. All three bi-specific constructs showed similar cell binding affinities to cell-surface overexpressed human and cynomolgus LAG3 in CHO cells (FIG. 9).

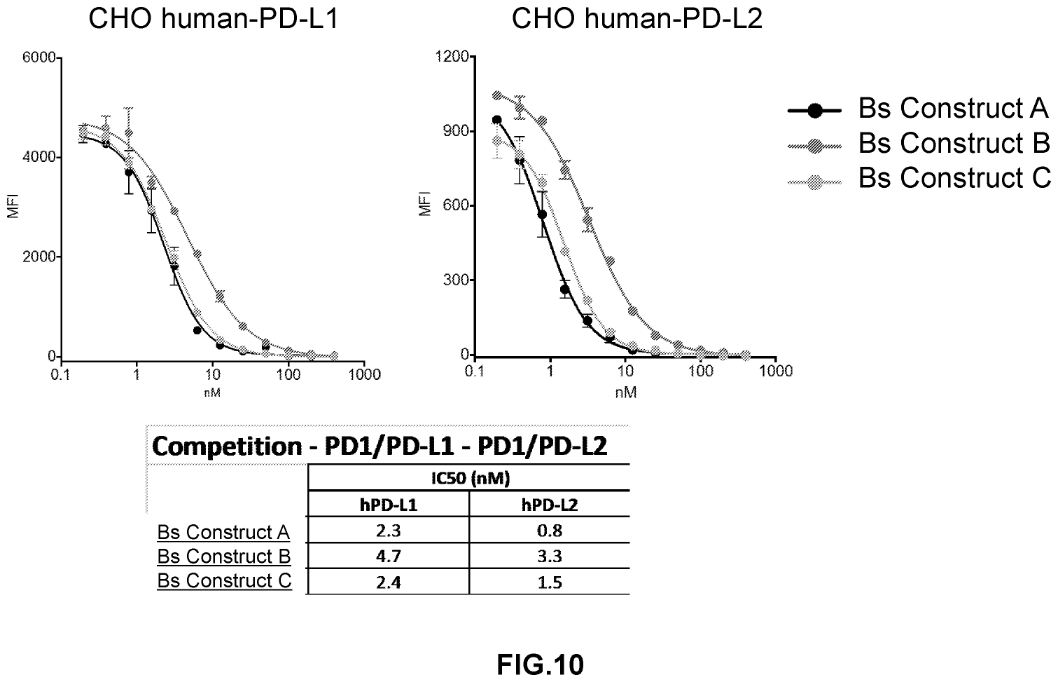

Bi-specific Constructs B and C were also compared to Bi-Specific Construct A in inhibiting recombinant human PD-1 protein binding to CHO cells overexpressing either PD-L1 or PD-L2, two cell surface ligands that bind to PD-1 (FIG. 10). Similar to PD-1 cell binding results, both Constructs A and C block recombinant PD-1 protein binding to CHO-PD-L1 and CHO-PD-L2 cells with similar IC50 values, whereas Construct B showed relative weaker inhibitory cell binding activity.

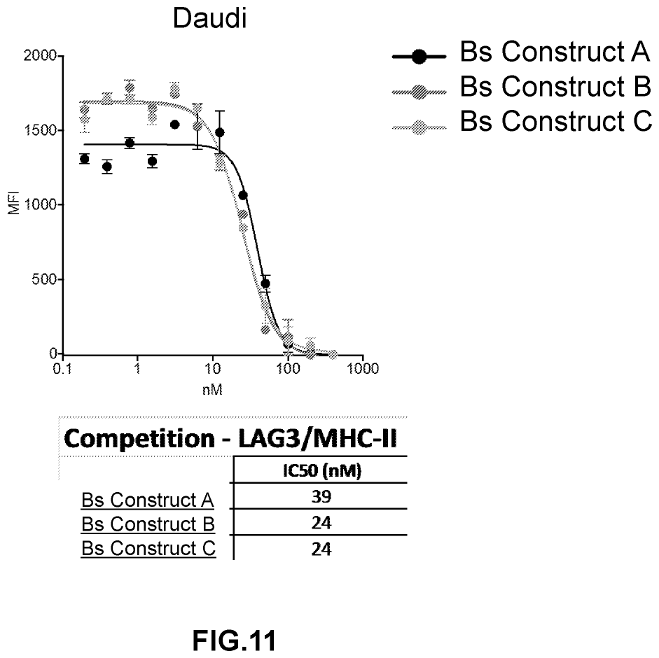

Bi-specific Constructs B and C were tested relative to Bi-Specific Construct A in inhibiting recombinant human LAG3 protein binding to Daudi cell expressing endogenous MHC-class II, a cell-surface expressed ligand that binds to LAG3 (FIG. 11). All three PD1× LAG3 bispecifics inhibited human LAG3 protein binding to Daudi cells with similar IC50 values.

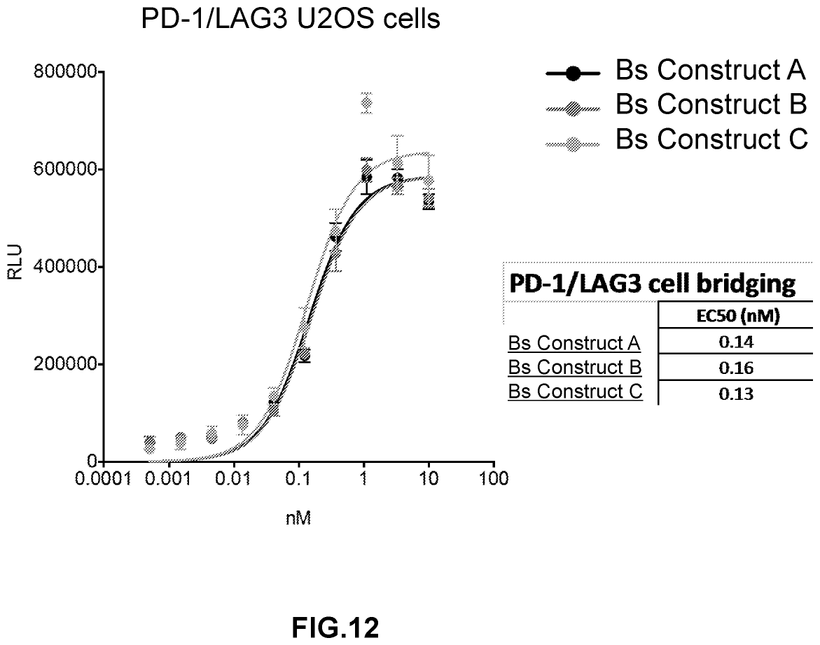

In addition, all three PD1×LAG3 bispecifics showed similar EC50 beta-galactosidase activation curves on U2OS cells co-expressing both PD-1 and LAG3 that are intracellularly fused to beta-galactosidase components in an enzyme-fragment complementation assay (FIG. 12), suggesting that all three bispecifics can bind to both PD-1 and LAG3 simultaneously on same cell.

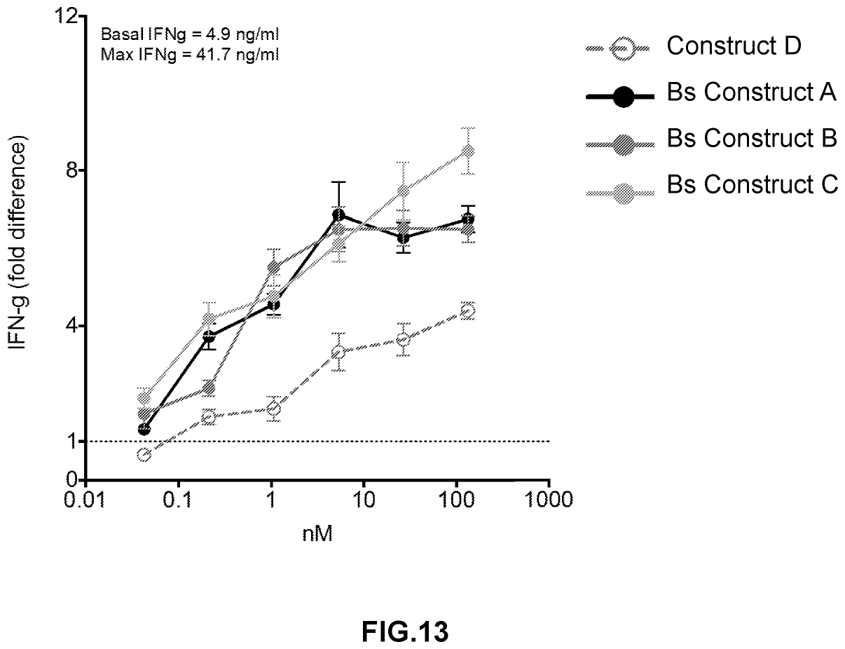

Bi-specific Constructs B and C were compared to Bi-specific Construct A and to Construct D in a PBMC CMV antigen recall functional assay (FIG. 13). Whereas Bi-specific Constructs A, B, and C are PD×LAG3 bi-specific antibodies, Construct D is a monovalent version of Bi-specific Construct A with a single a-PD-1 binding arm. All three bi-specific antibodies induced similar IFN-g secretion dose-response in a CMV-peptide treated human PBMC culture over a 5-day period compared to Construct D, the monovalent a-PD1 control antibody lacking the anti-LAG3 binding arm. Increased IFN-g secretion by all three bi-specifics suggest additive or synergistic effects in targeting both PD-1 and LAG-3 on T-cells, and Bi-specific Constructs B and C are functionally active, similar to Bi-specific Construct A on human T cells.

### Example 21: Sequences

Table 35 provides sequences referred to herein.

TABLE 35
Sequences

| SEQ ID | | | | |
|---|---|---|---|---|
| NO: | Molecule | Region | Scheme | Sequence |
| 1 | Human LAG3 | | | MWEAQFLGLLFLQPLWVAPVKPLQPGA |
| | | | | EVPVVWAQEGAPAQLPCSPTIPLQDLS |
| | | | | LLRRAGVTWQHQPDSGPPAAAPGHPLA |
| | | | | PGPHPAAPSSWGPRPRRYTVLSVGPGG |
| | | | | LRSGRLPLQPRVQLDERGRQRGDFSLW |
| | | | | LRPARRADAGEYRAAVHLRDRALSCRL |
| | | | | RLRLGQASMTASPPGSLRASDWVILNC |
| | | | | SFSRPDRPASVHWFRNRGQGRVPVRES |
| | | | | PHHHLAESFLFLPQVSPMDSGPWGCIL |
| | | | | TYRDGFNVSIMYNLTVLGLEPPTPLTV |
| | | | | YAGAGSRVGLPCRLPAGVGTRSFLTAK |
| | | | | WTPPGGGPDLLVTGDNGDFTLRLEDVS |
| | | | | QAQAGTYTCHIHLQEQQLNATVTLAII |
| | | | | TVTPKSFGSPGSLGKLLCEVTPVSGQE |
| | | | | RFVWSSLDTPSQRSFSGPWLEAQEAQL |
| | | | | LSQPWQCQLYQGERLLGAAVYFTELSS |
| | | | | PGAQRSGRAPGALPAGHLLLFLILGVL |
| | | | | SLLLLVTGAFGFHLWRRQWRPRRFSAL |
| | | | | EQGIHPPQAQSKIEELEQEPEPEPEPE |
| | | | | PEPEPEPEPEQL |
| 2 | Cynomolgus LAG3 | | | MWEAQFLGLLFLQPLWVAPVKPPQPGA |
| | | | | EISVVWAQEGAPAQLPCSPTIPLQDLS |
| | | | | LLRRAGVTWQHQPDSGPPAXAPGHPPV |
| | | | | PGHRPAAPYSWGPRPRRYTVLSVGPGG |
| | | | | LRSGRLPLQPRVQLDERGRQRGDFSLW |
| | | | | LRPARRADAGEYRATVHLRDRALSCRL |
| | | | | RLRVGQASMTASPPGSLRTSDWVILNC |
| | | | | SFSRPDRPASVHWFRSRGQGRVPVQGS |
| | | | | PHHHLAESFLFLPHVGPMDSGLWGCIL |
| | | | | TYRDGFNVSIMYNLTVLGLEPATPLTV |
| | | | | YAGAGSRVELPCRLPPAVGTQSFLTAK |
| | | | | WAPPGGGPDLLVAGDNGDFTLRLEDVS |
| | | | | QAQAGTYICHIRLQGQQLNATVTLAII |
| | | | | TVTPKSFGSPGSLGKLLCEVTPASGQE |
| | | | | HFVWSPLNTPSQRSFSGPWLEAQEAQL |
| | | | | LSQPWQCQLHQGERLLGAAVYFTELSS |
| | | | | PGAQRSGRAPGALRAGHLPLFLILGVL |
| | | | | FLLLLVTGAFGFHLWRRQWRPRRFSAL |
| | | | | EQGIHPPQAQSKIEELEQEPELEPEPE |
| | | | | LERELGPEPEPGPEPEPEQL |
| 3 | Mouse LAG3 | | | MREDLLLGFLLLGLLWEAPVVSSGPGK |
| | | | | ELPVVWAQEGAPVHLPCSLKSPNLDPN |
| | | | | FLRRGGVIWQHQPDSGQPTPIPALDLH |
| | | | | QGMPSPRQPAPGRYTVLSVAPGGLRSG |
| | | | | RQPLHPHVQLEERGLQRGDFSLWLRPA |
| | | | | LRTDAGEYHATVRLPNRALSCSLRLRV |
| | | | | GQASMIASPSGVLKLSDWVLLNCSFSR |
| | | | | PDRPVSVHWFQGQNRVPVYNSPRHFLA |
| | | | | ETFLLLPQVSPLDSGTWGCVLTYRDGF |
| | | | | NVSITYNLKVLGLEPVAPLTVYAAEGS |
| | | | | RVELPCHLPPGVGTPSLLIAKWTPPGG |
| | | | | GPELPVAGKSGNFTLHLEAVGLAQAGT |
| | | | | YTCSIHLQGQQLNATVTLAVITVTPKS |
| | | | | FGLPGSRGKLLCEVTPASGKERFVWRP |
| | | | | LNNLSRSCPGPVLEIQEARLLAERWQC |
| | | | | QLYEGQRLLGATVYAAESSSGAHSARR |
| | | | | ISGDLKGGHLVLVLILGALSLFLLVAG |
| | | | | AFGFHWWRKQLLLRRFSALEHGIQPFP |
| | | | | AQRKIEELERELETEMGQEPEPEPEPQ |
| | | | | LEPEPRQL |
| 4 | 26H10 | CDR-H1 | Chothia | GFTSSY |
| 5 | SRP1496-A03-VH | CDR-H1 | Chothia | GFNINDT |
| 6 | SRP1496-A04-VH | CDR-H1 | Chothia | GFNINDT |
| 7 | SRP1496-B08-VH | CDR-H1 | Chothia | GFNINDT |
| 8 | SRP1648-B07-VH | CDR-H1 | Chothia | GFNIADT |
| 9 | SRP1648-E02-VH | CDR-H1 | Chothia | GFNINDN |
| 10 | SRP1449-B03-VH | CDR-H1 | Chothia | GFTFSSY |
| 11 | SRP1449-F01-VH | CDR-H1 | Chothia | GFTFSSY |
| 12 | SRP1449-B07-VH | CDR-H1 | Chothia | GFTFSSY |
| 13 | 1449-G09.2-VH | CDR-H1 | Chothia | GFTFSSY |
| 14 | SRP1449-D05-VH | CDR-H1 | Chothia | GFTFRSF |
| 15 | SRP1558-F01-VH | CDR-H1 | Chothia | GFTFPDS |
| 16 | SRP1448-D09-VH | CDR-H1 | Chothia | GFTFTDS |
| 17 | SRP1558-A06-VH | CDR-H1 | Chothia | GFTFSES |
| 18 | SRP1558-E11-VH | CDR-H1 | Chothia | GFTFTSS |
| 19 | SRP1627-A02-VH | CDR-H1 | Chothia | GFNINDY |
| 20 | SRP1627-A11-VH | CDR-H1 | Chothia | GFNINDY |
| 21 | h5G11-2-VH | CDR-H1 | Chothia | GFNIKDY |
| 22 | SRP1627-B01-VH | CDR-H1 | Chothia | GFNITDL |
| 23 | 421.61.4.5G11-VH | CDR-H1 | Chothia | GFNIKDY |
| 24 | 1353-G10-wt | CDR-H1 | Chothia | GYRFPHY |
| 25 | 1353-G10 Y27D | CDR-H1 | Chothia | GDRFPHY |
| 26 | 1353-G10 R28D | CDR-H1 | Chothia | GYDFPHY |
| 27 | 1353-G10 F29D | CDR-H1 | Chothia | GYRDPHY |
| 28 | 1353-G10 P30D | CDR-H1 | Chothia | GYRFDHY |
| 29 | 1353-G10 H31D | CDR-H1 | Chothia | GYRFPDY |
| 30 | 1353-G10 Y32D | CDR-H1 | Chothia | GYRFPHD |
| 31 | 1353-G10 | CDR-H1 | Chothia | GYTFDHY |
| | R28T/P30D | | | |
| 32 | 1353-G10 | CDR-H1 | Chothia | GYDFDHY |
| | R28D/P30D | | | |
| 33 | 1353-G10 | CDR-H1 | Chothia | GYDFPDY |
| | R28D/H31D | | | |
| 34 | 1353-G10 | CDR-H1 | Chothia | GYTFPDY |
| | R28T/H31D | | | |
| 35 | 1353-G10 | CDR-H1 | Chothia | GYDFTSY |
| | R28D/P30T/H31S | | | |
| 36 | 1353-G10 | CDR-H1 | Chothia | GYTFTDY |
| | R28T/P30T/H31D | | | |
| 37 | 1353-G10 | CDR-H1 | Chothia | GYTFTSY |
| | R28T/P30T/H31S | | | |
| 38 | 1353-G10- | CDR-H1 | Chothia | GYTFDSY |
| | R28T/P30D/H31S | | | |
| 39 | 26H10 | CDR-H1 | Kabat | SYGMH |
| 40 | SRP1496-A03-VH | CDR-H1 | Kabat | DTYIH |
| 41 | SRP1496-A04-VH | CDR-H1 | Kabat | DTYIH |
| 42 | SRP1496-B08-VH | CDR-H1 | Kabat | DTYIH |
| 43 | SRP1648-B07-VH | CDR-H1 | Kabat | DTFIH |
| 44 | SRP1648-E02-VH | CDR-H1 | Kabat | DNYIH |
| 45 | SRP1449-B03-VH | CDR-H1 | Kabat | SYGMH |
| 46 | SRP1449-F01-VH | CDR-H1 | Kabat | SYGMH |
| 47 | SRP1449-B07-VH | CDR-H1 | Kabat | SYGMH |
| 48 | 1449-G09.2-VH | CDR-H1 | Kabat | SYGMH |
| 49 | SRP1449-D05-VH | CDR-H1 | Kabat | SFGMH |
| 50 | SRP1558-F01-VH | CDR-H1 | Kabat | DSSMS |
| 51 | SRP1448-D09-VH | CDR-H1 | Kabat | DSSMS |
| 52 | SRP1558-A06-VH | CDR-H1 | Kabat | ESTMS |
| 53 | SRP1558-E11-VH | CDR-H1 | Kabat | SSSMS |
| 54 | SRP1627-A02-VH | CDR-H1 | Kabat | DYFMH |
| 55 | SRP1627-A11-VH | CDR-H1 | Kabat | DYFMH |
| 56 | h5G11-2-VH | CDR-H1 | Kabat | DYYMH |
| 57 | SRP1627-B01-VH | CDR-H1 | Kabat | DLYMH |
| 58 | 421.61.4.5G11-VH | CDR-H1 | Kabat | DYYMH |
| 59 | 1353-G10-wt | CDR-H1 | Kabat | HYGIS |
| 60 | 1353-G10 H31D | CDR-H1 | Kabat | DYGIS |
| 61 | 1353-G10 Y32D | CDR-H1 | Kabat | HDGIS |
| 62 | 1353-G10G33D | CDR-H1 | Kabat | HYDIS |
| 63 | 1353-G10 S35D | CDR-H1 | Kabat | HYGID |
| 64 | 1353-G10 | CDR-H1 | Kabat | SYGIS |
| | R28D/P30T/H31S | | | |
| 65 | 1353-G10- | CDR-H1 | Kabat | SYGIS |
| | R28T/P30D/H31S | | | |
| 66 | 26H10 | CDR-H2 | Chothia | WYDGSN |
| 67 | SRP1496-A03-VH | CDR-H2 | Chothia | DPYDGA |
| 68 | SRP1496-A04-VH | CDR-H2 | Chothia | DPYDGA |
| 69 | SRP1496-B08-VH | CDR-H2 | Chothia | DPYDGA |
| 70 | SRP1648-B07-VH | CDR-H2 | Chothia | DPYDGD |
| 71 | SRP1648-E02-VH | CDR-H2 | Chothia | DPYDGF |
| 72 | SRP1449-B03-VH | CDR-H2 | Chothia | WYDASY |
| 73 | SRP1449-F01-VH | CDR-H2 | Chothia | WYDGSY |
| 74 | SRP1449-B07-VH | CDR-H2 | Chothia | WYDGSN |
| 75 | 1449-G09.2-VH | CDR-H2 | Chothia | WYDGSY |
| 76 | SRP1449-D05-VH | CDR-H2 | Chothia | WYDGSV |
| 77 | SRP1558-F01-VH | CDR-H2 | Chothia | TDNSGN |
| 78 | SRP1448-D09-VH | CDR-H2 | Chothia | TGNSGT |
| 79 | SRP1558-A06-VH | CDR-H2 | Chothia | TSDSGT |
| 80 | SRP1558-E11-VH | CDR-H2 | Chothia | SDDTGS |
| 81 | SRP1627-A02-VH | CDR-H2 | Chothia | DPWNGD |
| 82 | SRP1627-A11-VH | CDR-H2 | Chothia | DPWNGD |
| 83 | h5G11-2-VH | CDR-H2 | Chothia | DPENGD |
| 84 | SRP1627-B01-VH | CDR-H2 | Chothia | DPWNGD |
| 85 | 421.61.4.5G11-VH | CDR-H2 | Chothia | DPENGD |
| 86 | 1353-G10-wt | CDR-H2 | Chothia | SAYNGN |
| 87 | 1353-G10- | CDR-H2 | Chothia | SAYNGN |
| | R28T/P30D/H31S | | | |
| 88 | 26H10 | CDR-H2 | Kabat | VIWYDGSNKYYADSVKG |
| 89 | SRP1496-A03-VH | CDR-H2 | Kabat | IIDPYDGATDYADSVKG |
| 90 | SRP1496-A04-VH | CDR-H2 | Kabat | IIDPYDGATDYADSVKG |
| 91 | SRP1496-B08-VH | CDR-H2 | Kabat | IIDPYDGATDYADSVKG |
| 92 | SRP1648-B07-VH | CDR-H2 | Kabat | IIDPYDGDTDYADSVKG |
| 93 | SRP1648-E02-VH | CDR-H2 | Kabat | IIDPYDGFTAYADSVKG |
| 94 | SRP1449-B03-VH | CDR-H2 | Kabat | AIWYDASYKYYADSVKG |
| 95 | SRP1449-F01-VH | CDR-H2 | Kabat | VIWYDGSYKYYADSVKG |
| 96 | SRP1449-B07-VH | CDR-H2 | Kabat | VIWYDGSNKYYADSVKG |
| 97 | 1449-G09.2-VH | CDR-H2 | Kabat | VIWYDGSYKYYADSVKG |
| 98 | SRP1449-D05-VH | CDR-H2 | Kabat | VIWYDGSVKYYADSVKG |
| 99 | SRP1558-F01-VH | CDR-H2 | Kabat | VITDNSGNTDYADSVKG |
| 100 | SRP1448-D09-VH | CDR-H2 | Kabat | VITGNSGTTDYADSVKG |
| 101 | SRP1558-A06-VH | CDR-H2 | Kabat | FITSDSGTTDYADSVKG |
| 102 | SRP1558-E11-VH | CDR-H2 | Kabat | VISDDTGSTDYADSVKG |
| 103 | SRP1627-A02-VH | CDR-H2 | Kabat | RIDPWNGDTEYAPKFQG |
| 104 | SRP1627-A11-VH | CDR-H2 | Kabat | RIDPWNGDTEYAPKFQG |
| 105 | h5G11-2-VH | CDR-H2 | Kabat | WIDPENGDTEYAPKFQG |
| 106 | SRP1627-B01-VH | CDR-H2 | Kabat | RIDPWNGDTEYAPKFQG |
| 107 | 421.61.4.5G11-VH | CDR-H2 | Kabat | WIDPENGDTEYAPKFQG |
| 108 | 1353-G10-wt | CDR-H2 | Kabat | WISAYNGNTNYAQKLQG |
| 109 | 1353-G10- | CDR-H2 | Kabat | WISAYNGNTNYAQKLQG |
| | R28T/P30D/H31S | | | |
| 110 | 26H10 | CDR-H3 | | EWAVASWDYGMDV |
| 111 | SRP1496-A03-VH | CDR-H3 | | EIFG-FYWNPFDY |
| 112 | SRP1496-A04-VH | CDR-H3 | | EIFG-FYWNPFDY |
| 113 | SRP1496-B08-VH | CDR-H3 | | EIFG-FYWNPFDY |
| 114 | SRP1648-B07-VH | CDR-H3 | | EILG-FYWNPFDY |
| 115 | SRP1648-E02-VH | CDR-H3 | | ESIG-FYLNPFDY |
| 116 | SRP1449-B03-VH | CDR-H3 | | EWAVASWDYALDV |
| 117 | SRP1449-F01-VH | CDR-H3 | | ESEVASWDYGLDV |
| 118 | SRP1449-B07-VH | CDR-H3 | | EWAVSSWDYGMDV |
| 119 | 1449-G09.2-VH | CDR-H3 | | EEAPENWDYALDV |
| 120 | SRP1449-D05-VH | CDR-H3 | | EWADVSWDAGLDV |
| 121 | SRP1558-F01-VH | CDR-H3 | | VFEGGVRPYS-DY |
| 122 | SRP1448-D09-VH | CDR-H3 | | VYEGGVRPYS-DY |
| 123 | SRP1558-A06-VH | CDR-H3 | | VFEGGVRPFS-DY |
| 124 | SRP1558-E11-VH | CDR-H3 | | VDNGGVRPYS-DY |
| 125 | SRP1627-A02-VH | CDR-H3 | | -------SDALDY |
| 126 | SRP1627-A11-VH | CDR-H3 | | -------SDALDY |
| 127 | h5G11-2-VH | CDR-H3 | | -------PDALDY |
| 128 | SRP1627-B01-VH | CDR-H3 | | -------SEMVDY |
| 129 | 421.61.4.5G11-VH | CDR-H3 | | -------PDALDY |
| 130 | 1353-G10-wt | CDR-H3 | | DVDYG-T-GS-GY |
| 131 | 1353-G10- | CDR-H3 | | DVDYG-T-GS-GY |
| | R28T/P30D/H31S | | | |
| 132 | 26H10 | CDR-L1 | | RASQ----SVSSSYLA |
| 133 | SRP1449-D05-VL | CDR-L1 | | RASQ----SVSSSYLA |
| 134 | SRP1449-F01-VL | CDR-L1 | | RASR----SVSSSYLA |
| 135 | 1449-G09.2-VL | CDR-L1 | | RASQ----SVSSSYLA |
| 136 | SRP1449-B07-VL | CDR-L1 | | RASQ----SVSSSYLA |
| 137 | SRP1449-B03-VL | CDR-L1 | | RASQ----SVSSSYLA |
| 138 | SRP1558-E11-VL | CDR-L1 | | RASQ----SVSSSYLA |
| 139 | SRP1558-A06-VL | CDR-L1 | | RASQ----SVSSNPLA |
| 140 | SRP1558-F01-VL | CDR-L1 | | RASQ----SVSSGNPA |
| 141 | SRP1448-D09-VL | CDR-L1 | | RASQ----SVSSSYLA |
| 142 | trastuzumab-VL | CDR-L1 | | RASQ----DVNTA-VA |
| 143 | SRP1627-A02-VL | CDR-L1 | | KSSQSLLDSDGKTYLN |
| 144 | SRP1627-A11-VL | CDR-L1 | | KSSQSLLDSDGKTYLN |
| 145 | SRP1627-B01-VL | CDR-L1 | | KSSQSLLDSDGKTYLN |
| 146 | h5G11-2-VL | CDR-L1 | | KSSQSLLDSDGKTYLN |
| 147 | 421.61.4.5G11-VL | CDR-L1 | | KSSQSLLDSDGKTYLN |
| 148 | 1353-G10-wt | CDR-L1 | | SGDALPKQYAY |
| 149 | 1353-G10-kappa | CDR-L1 | | SGDALPKQYAY |
| 150 | 26H10 | CDR-L2 | | GASSRAT |
| 151 | SRP1449-D05-VL | CDR-L2 | | GASSRAT |
| 152 | SRP1449-F01-VL | CDR-L2 | | GASSRAT |
| 153 | 1449-G09.2-VL | CDR-L2 | | GASSRAT |
| 154 | SRP1449-B07-VL | CDR-L2 | | GASSRAT |
| 155 | SRP1449-B03-VL | CDR-L2 | | GASSRAT |
| 156 | SRP1558-E11-VL | CDR-L2 | | GASSRAT |
| 157 | SRP1558-A06-VL | CDR-L2 | | GASSRAT |
| 158 | SRP1558-F01-VL | CDR-L2 | | GASSRAT |
| 159 | SRP1448-D09-VL | CDR-L2 | | GASSRAT |
| 160 | trastuzumab-VL | CDR-L2 | | SASFLYS |
| 161 | SRP1627-A02-VL | CDR-L2 | | LVSKLDS |
| 162 | SRP1627-A11-VL | CDR-L2 | | LVSKLDS |
| 163 | SRP1627-B01-VL | CDR-L2 | | LVSKLDS |
| 164 | h5G11-2-VL | CDR-L2 | | LVSKLDS |
| 165 | 421.61.4.5G11-VL | CDR-L2 | | LVSKLDS |
| 166 | 1353-G10-wt | CDR-L2 | | KDTERPS |
| 167 | 1353-G10 K50D | CDR-L2 | | DDTERPS |
| 168 | 1353-G10T52D | CDR-L2 | | KDDERPS |
| 169 | 1353-G10 E53D | CDR-L2 | | KDTDRPS |
| 170 | 1353-G10 P55D | CDR-L2 | | KDTERDS |
| 171 | 1353-G10 S56D | CDR-L2 | | KDTERPD |
| 172 | 1353-G10-kappa | CDR-L2 | | KDTERPS |
| 173 | 26H10 | CDR-L3 | | QQYGSSPFT |
| 174 | SRP1449-D05-VL | CDR-L3 | | QQYGSTPFK |
| 175 | SRP1449-F01-VL | CDR-L3 | | QQYGSSPFT |
| 176 | 1449-G09.2-VL | CDR-L3 | | QQYGRSPFS |
| 177 | SRP1449-B07-VL | CDR-L3 | | QQYGASPFT |
| 178 | SRP1449-B03-VL | CDR-L3 | | QQYDRSPLT |
| 179 | SRP1558-E11-VL | CDR-L3 | | QQYSLAPPT |
| 180 | SRP1558-A06-VL | CDR-L3 | | QQYMAGPPT |
| 181 | SRP1558-F01-VL | CDR-L3 | | QQYTAGPPT |
| 182 | SRP1448-D09-VL | CDR-L3 | | QQDTAGPPT |
| 183 | trastuzumab-VL | CDR-L3 | | QQHYTTPPT |
| 184 | SRP1627-A02-VL | CDR-L3 | | SHGNPVPQT |
| 185 | SRP1627-A11-VL | CDR-L3 | | WHGINFPQT |
| 186 | SRP1627-B01-VL | CDR-L3 | | STYSHFPQT |
| 187 | h5G11-2-VL | CDR-L3 | | WQGSHFPQT |
| 188 | 421.61.4.5G11-VL | CDR-L3 | | WQGSHFPQT |
| 189 | 1353-G10-wt | CDR-L3 | | QSADNSITYRV |
| 190 | 1353-G10-kappa | CDR-L3 | | QSADNSITYRV |
| 191 | 26H10 | VH | | QVQLVESGGGVVQPGRSLRLSCAASGF |
| | | | | TFSSYGMHWVRQAPGKGLEWVAVIWYD |
| | | | | GSNKYYADSVKGRFTISRDNSKNTLYL |
| | | | | QMNSLRAEDTAVYYCAREWAVASWDYG |
| | | | | MDVWGQGTTVTVSS |
| 192 | SRP1496-A03-VH | VH | | EVQLVESGGGLVQPGGSLRLSCAASGF |
| | | | | NINDTYIHWVRQAPGKGLEWVGIIDPY |
| | | | | DGATDYADSVKGRFTISADTSKNTAYL |
| | | | | QMNSLRAEDTAVYYCAREIFGFYWNPF |
| | | | | DYWGQGTLVTVSS |
| 193 | SRP1496-A04-VH | VH | | EVQLVESGGGLVQPGGSLRLSCAASGFN |
| | | | | INDTYIHWVRQAPGKGLEWVGIIDPYDG |
| | | | | ATDYADSVKGRFTISADTSKNTAYLQMN |
| | | | | SLRAEDTAVYYCAREIFGFYWNPFDYWG |
| | | | | QGTLVTVSS |
| 194 | SRP1496-B08-VH | VH | | EVQLVESGGGLVQPGGSLRLSCAASGF |
| | | | | NINDTYIHWVRQAPGKGLEWVGIIDPY |
| | | | | DGATDYADSVKGRFTISADTSKNTAYL |
| | | | | QMNSLRAEDTAVYYCAREIFGFYWNPF |
| | | | | DYWGQGTLVTVSS |
| 195 | SRP1648-B07-VH | VH | | EVQLVESGGGLVQPGGSLRLSCAASGFN |
| | | | | IADTFIHWVRQAPGKGLEWVGIIDPYDG |
| | | | | DTDYADSVKGRFTISADTSKNTAYLQMN |
| | | | | SLRAEDTAVYYCAREILGFYWNPFDYWG |
| | | | | QGTLVTVSS |
| 196 | SRP1648-E02-VH | VH | | EVQLVESGGGLVQPGGSLRLSCAASGFN |
| | | | | INDNYIHWVRQAPGKGLEWVGIIDPYDG |
| | | | | FTAYADSVKGRFTISADTSKNTAYLQMN |
| | | | | SLRAEDTAVYYCARESIGFYLNPFDYWG |
| | | | | QGTLVTVSS |
| 197 | SRP1449-B03-VH | VH | | QVQLVESGGGVVQPGRSLRLSCAASGFT |
| | | | | FSSYGMHWVRQAPGKGLEWVAAIWYDAS |
| | | | | YKYYADSVKGRFTISRDNSKNTLYLQMN |
| | | | | SLRAEDTAVYYCAREWAVASWDYALDVW |
| | | | | GQGTTVTVSS |
| 198 | SRP1449-F01-VH | VH | | QVQLVESGGGVVQPGRSLRLSCAASGFT |
| | | | | FSSYGMHWVRQAPGKGLEWVAVIWYDGS |
| | | | | YKYYADSVKGRFAISRDNSKNTLYLQMN |
| | | | | SLRAEDTAVYYCARESEVASWDYGLDVW |
| | | | | GQGTTVTVSS |
| 199 | SRP1449-B07-VH | VH | | QVQLVESGGGVVQPGRSLRLSCAASGFT |
| | | | | FSSYGMHWVRQAPGKGLEWVAVIWYDGS |
| | | | | NKYYADSVKGRFTISRDNSKNTLYLQMN |
| | | | | SLRAEDTAVYYCAREWAVSSWDYGMDVW |
| | | | | GQGTTVTVSS |
| 200 | 1449-G09.2-VH | VH | | QVQLVESGGGVVQPGRSLRLSCAASGFT |
| | | | | FSSYGMHWVRQAPGKGLEWVAVIWYDGS |
| | | | | YKYYADSVKGRFTISRDNSKNTLYLQMN |
| | | | | SLRAEDTAVYYCAREEAPENWDYALDVW |
| | | | | GQGTTVTVSS |
| 201 | SRP1449-D05-VH | VH | | QVQLVESGGGVVQPGRSLRLSCAASGFT |
| | | | | FRSFGMHWVRQAPGKGLEWVAVIWYDGS |
| | | | | VKYYADSVKGRFTISRDNSKNTLYLQMN |
| | | | | SLRAEDTAVYYCAREWADVSWDAGLDVW |
| | | | | GQGTTVTVSS |
| 202 | SRP1558-F01-VH | VH | | EVQLLESGGGLVQPGGSLRLSCAASGFT |
| | | | | FPDSSMSWVRQAPGKGLEWVGVITDNSG |
| | | | | NTDYADSVKGRFTISRDNSKNTLYLQMN |
| | | | | SLRAEDTAVYYCAKVFEGGVRPYSDYWG |
| | | | | QGTLVTVSS |
| 203 | SRP1448-D09-VH | VH | | EVQLLESGGGLVQPGGSLRLSCAASGFT |
| | | | | FTDSSMSWVRQAPGKGLEWVGVITGNSG |
| | | | | TTDYADSVKGRFTISRDNSKNTLYLQMN |
| | | | | SLRAEDTAVYYCAKVYEGGVRPYSDYWG |
| | | | | QGTLVTVSS |
| 204 | SRP1558-A06-VH | VH | | EVQLLESGGGLVQPGGSLRLSCAASGFT |
| | | | | FSESTMSWVRQAPGKGLEWVGFITSDSG |
| | | | | TTDYADSVKGRFTISRDNSKNTLYLQMN |
| | | | | SLRAEDTAVYYCAKVFEGGVRPFSDYWG |
| | | | | QGTLVTVSS |
| 205 | SRP1558-E11-VH | VH | | EVQLLESGGGLVQPGGSLRLSCAASGFT |
| | | | | FTSSSMSWVRQAPGKGLEWVGVISDDTG |
| | | | | STDYADSVKGRFTISRDNSKNTLYLQMN |
| | | | | SLRAEDTAVYYCAKVDNGGVRPYSDYWG |
| | | | | QGTLVTVSS |
| 206 | SRP1627-A02-VH | VH | | QVQLVQSGAEVKKPGSSVKVSCKASGFN |
| | | | | INDYFMHWVRQAPGQGLEWIARIDPWNG |
| | | | | DTEYAPKFQGRVTITADESTSTAYMELS |
| | | | | SLRSEDTAVYYCGMSDALDYWGQGTLVT |
| | | | | VSS |
| 207 | SRP1627-A11-VH | VH | | QVQLVQSGAEVKKPGSSVKVSCKASGFN |
| | | | | INDYFMHWVRQAPGQGLEWIARIDPWNG |
| | | | | DTEYAPKFQGRVTITADESTSTAYMELS |
| | | | | SLRSEDTAVYYCGMSDALDYWGQGTLVT |
| | | | | VSS |
| 208 | h5G11-2-VH | VH | | QVQLVQSGAEVKKPGSSVKVSCKASGFN |
| | | | | IKDYYMHWVRQAPGQGLEWIAWIDPENG |
| | | | | DTEYAPKFQGRVTITADESTSTAYMELS |
| | | | | SLRSEDTAVYYCNAPDALDYWGQGTLVT |
| | | | | VSS |
| 209 | SRP1627-B01-VH | VH | | QVQLVQSGAEVKKPGSSVKVSCKASGFN |
| | | | | ITDLYMHWVRQAPGQGLEWIARIDPWNG |
| | | | | DTEYAPKFQGRATITADESTSTAYMELS |
| | | | | SLRSEDTAVYYCIASEMVDYWGQGTLVT |
| | | | | VSS |
| 210 | 421.61.4.5G11-VH | VH | | EVQLQQSGAELVRSGASVKLSCTASGFN |
| | | | | IKDYYMHWVKQRPEQGLEWIAWIDPENG |
| | | | | DTEYAPKFQGRATLTADTSSNTAYLHLS |
| | | | | SLTSEDTAVYYCNAPDALDYWGQGTSVT |
| | | | | VSS |
| 211 | 1353-G10-wt | VH | | EVQLVQSGAEVKKPGASVKVSCKASGYR |
| | | | | FPHYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 212 | 1353-G10Y27D | VH | | EVQLVQSGAEVKKPGASVKVSCKASGDR |
| | | | | FPHYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 213 | 1353-G10R28D | VH | | EVQLVQSGAEVKKPGASVKVSCKASGYD |
| | | | | FPHYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 214 | 1353-G10F29D | VH | | EVQLVQSGAEVKKPGASVKVSCKASGYR |
| | | | | DPHYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 215 | 1353-G10P30D | VH | | EVQLVQSGAEVKKPGASVKVSCKASGYR |
| | | | | FDHYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 216 | 1353-G10 H31D | VH | | EVQLVQSGAEVKKPGASVKVSCKASGYR |
| | | | | FPDYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 217 | 1353-G10 Y32D | VH | | EVQLVQSGAEVKKPGASVKVSCKASGYR |
| | | | | FPHDGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 218 | 1353-G10 G33D | VH | | EVQLVQSGAEVKKPGASVKVSCKASGYR |
| | | | | FPHYDISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 219 | 1353-G10 S35D | VH | | EVQLVQSGAEVKKPGASVKVSCKASGYR |
| | | | | FPHYGIDWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 220 | 1353-G10 | VH | | EVQLVQSGAEVKKPGASVKVSCKASGYT |
| | R28T/P30D | | | FDHYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 221 | 1353-G10 | VH | | EVQLVQSGAEVKKPGASVKVSCKASGYD |
| | R28D/P30D | | | FDHYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 222 | 1353-G10 | VH | | EVQLVQSGAEVKKPGASVKVSCKASGYD |
| | R28D/H31D | | | FPDYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 223 | 1353-G10 | VH | | EVQLVQSGAEVKKPGASVKVSCKASGYT |
| | R28T/H31D | | | FPDYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 224 | 1353-G10 | VH | | EVQLVQSGAEVKKPGASVKVSCKASGYD |
| | R28D/P30T/H31S | | | FTSYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 225 | 1353-G10 | VH | | EVQLVQSGAEVKKPGASVKVSCKASGYT |
| | R28T/P30T/H31D | | | FTDYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 226 | 1353-G10 | VH | | EVQLVQSGAEVKKPGASVKVSCKASGYT |
| | R28T/P30T/H31S | | | FTSYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 227 | 1353-G10- | VH | | EVQLVQSGAEVKKPGASVKVSCKASGYT |
| | R28T/P30D/H31S | | | FDSYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSS |
| 228 | 10B4 | VH | | QVQLQQSGAELMKPGASVKMSCKTTGYI |
| | | | | FSSYWIGWVKQRPGHGLEWIGKIFPGSG |
| | | | | SADYNENFKGKATFTVDTSSNTAYMQLS |
| | | | | SLTSEDSAVYYCARGYGNYLYFDVWGAG |
| | | | | TTVTVSS |
| 229 | 1353-A09 | VH | | EVQLVQSGAEVKKPGASVKVSCKASGYR |
| | | | | FTWYGISWVRQAPGQGLEWMGWISAYNG |
| | | | | NTNYAQKLQGRVTMTTDTSTNTAYMELR |
| | | | | SLRSDDTAVYYCARDSEYSSGSGYWGQG |
| | | | | TLVTVSS |
| 230 | 1353-C07 | VH | | EVQLVQSGAEVKKPGASVKVSCKASGYR |
| | | | | FSTFGISWVRQAPGQGLEWMGWISAYNG |
| | | | | NTNYAQKLQGRVTMTTDTSTNTAYMELR |
| | | | | SLRSDDTAVYYCARDVDYSSGSGYWGQG |
| | | | | TLVTVSS |
| 231 | 1353-E07 | VH | | EVQLVQSGAEVKKPGASVKVSCKASGYR |
| | | | | FETYGISWVRQAPGQGLEWMGWISAYNG |
| | | | | NTNYAQKLQGRVTMTTDTSTNTAYMELR |
| | | | | SLRSDDTAVYYCARDAEYSLGSGYWGQG |
| | | | | TLVTVSS |
| 232 | 1353-F09 | VH | | EVQLVQSGAEVKKPGASVKVSCKASGYR |
| | | | | FRQYGISWVRQAPGQGLEWMGWISAYNG |
| | | | | NTNYAQKLQGRVTMTTDTSTNTAYMELR |
| | | | | SLRSDDTAVYYCARDAEYGSGSGYWGQG |
| | | | | TLVTVSS |
| 233 | 1353-G08 | VH | | EVQLVQSGAEVKKPGASVKVSCKASGYR |
| | | | | FTRYGISWVRQAPGQGLEWMGWVSAHNG |
| | | | | NTNYAQKLQGRVTMTTDTSTNTAYMELR |
| | | | | SLRSDDTAVYYCARDADYGSGSGYWGQG |
| | | | | TLVTVSS |
| 234 | 1353-H08 | VH | | EVQLVQSGAEVKKPGASVKVSCKASGYR |
| | | | | FTRQGISWVRQAPGQGLEWMGWISAYNG |
| | | | | NTKYAQKLQGRVTMTTDTSTNTAYMELR |
| | | | | SLRSDDTAVYYCARDVDYGSGSGYWGQG |
| | | | | TLVTVSS |
| 235 | 1353-H09 | VH | | EVQLVQSGAEVKKPGASVKVSCKASGYR |
| | | | | FPHYGISWVRQAPGQGLEWMGWISAYNG |
| | | | | NTNYAQKLQGRVTMTTDTSTNTAYMELR |
| | | | | SLRSDDTAVYYCARDAEYGSGSGYWGQG |
| | | | | TLVTVSS |
| 236 | 1B10 | VH | | DVQLQESGPGLVKPSQSLSLTCTVTGHS |
| | | | | ITSDYAWNWIRQFPGNKLEWMGYISYSG |
| | | | | RTSYNPSLTSRISITRDTSKNQFFLQLN |
| | | | | SVTTEDTATYYCARGYALDYWGQGTSVT |
| | | | | VSS |
| 237 | 1E9 | VH | | EVKLVESGGGLVSPGGSLKLSCAASGFT |
| | | | | FSTFGMSWVRQTPEKRLEWVATISGGGS |
| | | | | DTYYPDSVQGRFIISRYNAKNNLYLQMN |
| | | | | SLRPEDTALYYCARQGYDVYSWFAYWGQ |
| | | | | GTLVTVSA |
| 238 | 4B10 | VH | | EVKLVESGGGLVKPGGSLKLSCAASGFT |
| | | | | FSTYGMSWVRQTPEKRLQWVATISGGGS |
| | | | | NTYYSDSVKGRFTISRDNAKNNLYLQMS |
| | | | | SLRSEDTALYYCARQRDSAWFASWGQGT |
| | | | | LVTVSA |
| 239 | h1E9-1 | VH | | EVQLVESGGGLVKPGGSLRLSCAASGFT |
| | | | | FSTFGMSWVRQAPGKGLEWVSTISGGGS |
| | | | | DTYYPDSVQGRFTISRDNAKNSLYLQMN |
| | | | | SLRAEDTAVYYCARQGYDVYSWFAYWGQ |
| | | | | GTLVTVSS |
| 240 | h1E9-2 | VH | | EVQLVESGGGLVKPGGSLRLSCAASGFT |
| | | | | FSTFGMSWVRQAPGKGLEWVSTISGGGS |
| | | | | DTYYPDSVQGRFTISRDNAKNSLYLQMN |
| | | | | SLRAEDTAVYYCARQGYDVYSWFAYWGQ |
| | | | | GTLVTVSS |
| 241 | h1E9-4 | VH | | EVQLVESGGGLVQPGGSLRLSCAASGFT |
| | | | | FSTFGMSWVRQAPGKGLEWVATISGGGS |
| | | | | DTYYPDSVQGRFTISRDNAKNSLYLQMN |
| | | | | SLRAEDTAVYYCARQGYDVYSWFAYWGQ |
| | | | | GTLVTVSS |
| 242 | h1E9-5 | VH | | EVQLVESGGGLVQPGGSLRLSCAASGFT |
| | | | | FSTFGMSWVRQAPGKGLEWVATISGGGS |
| | | | | DTYYPDSVQGRFTISRDNAKNSLYLQMN |
| | | | | SLRAEDTAVYYCARQGYDVYSWFAYWGQ |
| | | | | GTLVTVSS |
| 243 | h4B10-1 | VH | | EVQLVESGGGLVKPGGSLRLSCAASGFT |
| | | | | FSTYGMSWVRQAPGKGLEWVATISGGGS |
| | | | | NTYYSDSVKGRFTISRDDSKNTLYLQMN |
| | | | | SLKTEDTAVYYCARQRDSAWFASWGQGT |
| | | | | LVTVSS |
| 244 | h4B10-2 | VH | | EVQLVESGGGLVKPGGSLRLSCAASGFT |
| | | | | FSTYGMSWVRQAPGKGLEWVATISGGGS |
| | | | | NTYYSDSVKGRFTISRDDSKNTLYLQMN |
| | | | | SLKTEDTAVYYCARQRDSAWFASWGQGT |
| | | | | LVTVSS |
| 245 | h4B10-3 | VH | | EVQLVESGGGLVKPGGSLRLSCAASGFT |
| | | | | FSTYGMSWVRQAPGKGLEWVATISGGGS |
| | | | | NTYYSDSVKGRFTISRDDSKNTLYLQMN |
| | | | | SLKTEDTAVYYCARQRDSAWFASWGQGT |
| | | | | LVTVSS |
| 246 | PD1-17 | VH | | QVQLQESGPGVVKPSGTLSLTCAISGGS |
| | | | | IGSGGSIRSTRWWSWVRQSPGKGLEWIG |
| | | | | EIYHSGSTNYNPSLKSRVTISLDKSRNH |
| | | | | FSLRLNSVTAADTAVYYCARQDYGDSGD |
| | | | | WYFDLWGKGTMVTVSS |
| 247 | PD1-28 | VH | | EVQLVQSGAEVKKPGASVKVSCKASGYR |
| | | | | FTSYGISWVRQAPGQGLEWMGWISAYNG |
| | | | | NTNYAQKLQGRVTMTTDTSTNTAYMELR |
| | | | | SLRSDDTAVYYCARDADYSSGSGYWGQG |
| | | | | TLVTVSS |
| 248 | PD1-33 | VH | | QVQLVQSGAEVKKPGASVRVSCKASGYT |
| | | | | LTSYYIHWVRQAPGQGLEWMGIINPRGA |
| | | | | TISYAQKFQGRVTMTRDTSTSTVYMELR |
| | | | | NLKSEDTALYYCATAGIYGFDFDYWGRG |
| | | | | TLVTVSS |
| 249 | PD1-35 | VH | | QVQLQESGPGLVKPSQTLSLTCTVSGGS |
| | | | | ISSGAYYWSWIRQHPGKGLEWIGYIYYN |
| | | | | GNTYYNPSLRSLVTISVDASKNQFSLKL |
| | | | | SSVTAADTAVYYCARASDYVWGGYRYMD |
| | | | | AFDIWGRGTLITVSS |
| 250 | PD1-F2 | VH | | EVQLVQSGGGVVQPGRSLRLSCAASGFT |
| | | | | FSSYWCDRMSWVRQAPGKGLEWVSAISG |
| | | | | SGGSTYYADSVKGRFTISRDNSKNTLYL |
| | | | | QMNSLRAEDTAVYYCAKENWGSYFDLWG |
| | | | | QGTTVTVSS |
| 251 | 26H10 | VL | | EIVLTQSPGTLSLSPGERATLSCRASQ |
| | | | | SVSSSYLAWYQQKPGQAPRLLIYGASS |
| | | | | RATGIPDRFSGSGSGTDFTLTISRLEP |
| | | | | EDFAVYYCQQYGSSPFTFGPGTKVDIK |
| 252 | SRP1449-D05-VL | VL | | EIVLTQSPGTLSLSPGERATLSCRASQ |
| | | | | SVSSSYLAWYQQKPGQAPRLLIYGASS |
| | | | | RATGIPDRFSGSGSGTDFTLTISRLEP |
| | | | | EDFAVYYCQQYGSTPFKFGPGTKVDIK |
| 253 | SRP1449-F01-VL | VL | | EIALTQSPGTLSLSPGERATLSCRASR |
| | | | | SVSSSYLAWYQQKPGQAPRLLIYGASS |
| | | | | RATGIPDRFSGSGSGTDFTLTISRLEP |
| | | | | EDFAVYYCQQYGSSPFTFGPGTKVDIK |
| 254 | 1449-G09.2-VL | VL | | EIVLTQSPGTLSLSPGERATLSCRASQ |
| | | | | SVSSSYLAWYQQKPGQAPRLLIYGASS |
| | | | | RATGIPDRFSGSGSGTDFTLTISRLEP |
| | | | | EDFAVYYCQQYGRSPFSFGPGTKVDIK |
| 255 | SRP1449-B07-VL | VL | | EIVLTQSPGTLSLSPGERATLSCRASQ |
| | | | | SVSSSYLAWYQQKPGQAPRLLIYGASS |
| | | | | RATGIPNRFSGSGSGTDFTLTISRLEP |
| | | | | EDFAVYYCQQYGASPFTFGPGTKVDIK |
| 256 | SRP1449-B03-VL | VL | | EIVLTQSPGTMSLSPGERATLSCRASQ |
| | | | | SVSSSYLAWYQQKPGQAPRLLIYGASS |
| | | | | RATGIPDRFSGSGSGTDFTLTISRLEP |
| | | | | EDFAVYYCQQYDRSPLTFGPGTKVDIK |
| 257 | SRP1558-E11-VL | VL | | EIVLTQSPGTLSLSPGERATLSCRASQ |
| | | | | SVSSSYLAWYQQKPGQAPRLLIYGASS |
| | | | | RATGIPDRFSGSGSGTDFTLTISRLEP |
| | | | | EDFAVYYCQQYSLAPPTLGQGTKVEIK |
| 258 | SRP1558-A06-VL | VL | | EIVLTQSPGTLSLSPGERATLSCRASQ |
| | | | | SVSSNPLAWYQQKPGQAPRLLIYGASS |
| | | | | RATGIPDRFSGSGSGTDFTLTISRLEP |
| | | | | EDFAVYYCQQYMAGPPTFGQGTKVEIK |
| 259 | SRP1558-F01-VL | VL | | EIVLTQSPGTLSLSPGERATLSCRASQ |
| | | | | SVSSGNPAWYQQKPGQAPRLLIYGASS |
| | | | | RATGIPDRFSGSGSGTDFTLTISRLEP |
| | | | | XDFAVYYCQQYTAGPPTFGQGTKVEIK |
| 260 | SRP1448-D09-VL | VL | | EIVLTQSPGTLSLSPGERATLSCRASQ |
| | | | | SVSSSYLAWYQQKPGQAPRLLIYGASS |
| | | | | RATGIPDRFSGSGSGTDFTLTISRLEP |
| | | | | EDFAVYYCQQDTAGPPTFGQGTKVEIK |
| 261 | trastuzumab-VL | VL | | DIQMTQSPSSLSASVGDRVTITCRASQ |
| | | | | DVNTAVAWYQQKPGKAPKLLIYSASFL |
| | | | | YSGVPSRFSGSRSGTDFTLTISSLQPE |
| | | | | DFATYYCQQHYTTPPTFGQGTKVEIK |
| 262 | SRP1627-A02-VL | VL | | DVVMTQSPLSLPVTLGQPASISCKSSQS |
| | | | | LLDSDGKTYLNWFQQRPGQSPRRLIYLV |
| | | | | SKLDSGVPDRFSGSGSGTDFTLKISRVE |
| | | | | AEDVGVYYCSHGNPVPQTFGQGTKVEIK |
| 263 | SRP1627-A11-VL | VL | | DVVMTQSPLSLPVTLGQPASISCKSSQ |
| | | | | SLLDSDGKTYLNWFQQRPGQSPRRLIY |
| | | | | LVSKLDSGVPDRFSGSGSGTDFTLKIS |
| | | | | RVEAEDVGVYYCWHGINFPQTFGQGTK |
| | | | | VEIK |
| 264 | SRP1627-B01-VL | VL | | DVVMTQSPLSLPVTLGQPASISCKSSQ |
| | | | | SLLDSDGKTYLNWFQQRPGQSPRRLIY |
| | | | | LVSKLDSGVPDRFSGSGSGTDFTLKIS |
| | | | | RVEAEDVGVYYCSTYSHFPQTFGQGTK |
| | | | | VEIK |
| 265 | h5G11-2-VL | VL | | DVVMTQSPLSLPVTLGQPASISCKSSQ |
| | | | | SLLDSDGKTYLNWFQQRPGQSPRRLIY |
| | | | | LVSKLDSGVPDRFSGSGSGTDFTLKIS |
| | | | | RVEAEDVGVYYCWQGSHFPQTFGQGTK |
| | | | | VEIK |
| 266 | 421.61.4.5G11-VL | VL | | DVVMTQTPLTLSVTIGQIASISCKSSQ |
| | | | | SLLDSDGKTYLNWLLQRPGQSPKRLIY |
| | | | | LVSKLDSGVPDRFTGSGSGTDFTLKIS |
| | | | | RVEAEDLGVYYCWQGSHFPQTFGGGTK |
| | | | | LEIK |
| 267 | 1353-G10-wt (λ) | VL | | SYELTQPPSVSVSPGQTARITCSGDALP |
| | | | | KQYAYWYQQKPGQAPVMVIYKDTERPSG |
| | | | | IPERFSGSSSGTKVTLTISGVQAEDEAD |
| | | | | YYCQSADNSITYRVFGGGTKVTVL |
| 268 | 1353-G10 K50D (λ) | VL | | SYELTQPPSVSVSPGQTARITCSGDALP |
| | | | | KQYAYWYQQKPGQAPVMVIYDDTERPSG |
| | | | | IPERFSGSSSGTKVTLTISGVQAEDEAD |
| | | | | YYCQSADNSITYRVFGGGTKVTVL |
| 269 | 1353-G10 T52D (λ) | VL | | SYELTQPPSVSVSPGQTARITCSGDALP |
| | | | | KQYAYWYQQKPGQAPVMVIYKDDERPSG |
| | | | | IPERFSGSSSGTKVTLTISGVQAEDEAD |
| | | | | YYCQSADNSITYRVFGGGTKVTVL |
| 270 | 1353-G10 E53D (λ) | VL | | SYELTQPPSVSVSPGQTARITCSGDALP |
| | | | | KQYAYWYQQKPGQAPVMVIYKDTDRPSG |
| | | | | IPERFSGSSSGTKVTLTISGVQAEDEAD |
| | | | | YYCQSADNSITYRVFGGGTKVTVL |
| 271 | 1353-G10 P55D (λ) | VL | | SYELTQPPSVSVSPGQTARITCSGDALP |
| | | | | KQYAYWYQQKPGQAPVMVIYKDTERDSG |
| | | | | IPERFSGSSSGTKVTLTISGVQAEDEAD |
| | | | | YYCQSADNSITYRVFGGGTKVTVL |
| 272 | 1353-G10 S56D (λ) | VL | | SYELTQPPSVSVSPGQTARITCSGDALP |
| | | | | KQYAYWYQQKPGQAPVMVIYKDTERPDG |
| | | | | IPERFSGSSSGTKVTLTISGVQAEDEAD |
| | | | | YYCQSADNSITYRVFGGGTKVTVL |
| 273 | 1353-G10 VL1c (κ) | VL | | SYELTQPPSVSVSPGQTARITCSGDALP |
| | | | | KQYAYWYQQKPGQAPVMVIYKDTERPSG |
| | | | | IPERFSGSSSGTKVTLTISGVQAEDFAT |
| | | | | YYCQSADNSITYRVFGGGTKVEIK |
| 274 | 1353-G10 VL1d (κ) | VL | | SYELTQPPSVSVSPGQTARITCSGDALP |
| | | | | KQYAYWYQRKPGQAPVMVIYKDTERPSG |
| | | | | IPERFSGSSSGTKVTLTISGVQAEDFAT |
| | | | | YYCQSADNSITYRVFGGGTKVEIK |
| 275 | 1353-G10 Vk1-39 (κ) | VL | | DIQLTQSPSSLSASVGDRVTITCSGDAL |
| | | | | PKQYAYWYQQKPGKAPKLLIYKDTERPS |
| | | | | GVPSRFSGSSSGTKVTLTISSLQPEDFA |
| | | | | TYYCQSADNSITYRVFGGGTKVEIK |
| 276 | 1353-G10 Vk3-20 (κ) | VL | | EIVLTQSPGTLSLSPGERATLSCSGDAL |
| | | | | PKQYAYWYQQKPGQAPRLLIYKDTERPS |
| | | | | GIPDRFSGSSSGTKVTLTISRLEPEDFA |
| | | | | VYYCQSADNSITYRVFGGGTKVEIK |
| 277 | 10B4 | VL | | NIVMTQTPKFLLVSAGDRITITCKASQS |
| | | | | VSDDVAWYQQKPGQSPKLLISYAFKRYI |
| | | | | GVPDRFTGSGYGTDFTFTISTVQAEDLA |
| | | | | VYFCQQNYNSPYTFGGGTKLELKR |
| 278 | 1353-A09 | VL | | SYELTQPPSVSVSPGQTARITCSGDALT |
| | | | | TQYAYWYQQKPGQAPVMVIYKDTERPSG |
| | | | | IPERFSGSSSGTKVTLTISGVQAEDEAD |
| | | | | YYCQSADNSITYRVFGGGTKVTVL |
| 279 | 1353-C07 | VL | | SYELTQPPSVSVSPGQTARITCSGDALS |
| | | | | EQYAYWYQQKPGQAPVMVIYKDTERPSG |
| | | | | IPERFSGSSSGTKVTLTISGVQAEDEAD |
| | | | | YYCQSADNSITYRVFGGGTKVTVL |
| 280 | 1353-E07 | VL | | SYELTQPPSVSVSPGQTARITCSGDALP |
| | | | | KQYAYWYQQKPGQAPVMVIYKDTERPSG |
| | | | | IPERFSGSSSGTKVTLTISGVQAEDEAD |
| | | | | YYCQSADNSITYRVFGGGTKVTVL |
| 281 | 1353-F09 | VL | | SYELTQPPSVSVSPGQTARITCSGDALP |
| | | | | KQYAYWYQQKPGQAPVMVLYKDTERPSG |
| | | | | IPERFSGSSSGTKVTLTISGVQAEDEAD |
| | | | | YYCQSADNSITYRVFGGGTKVTVL |
| 282 | 1353-G08 | VL | | SYELTQPPSVSVSPGQTARITCSGDALP |
| | | | | MQYGYWYQQKPGQAPVMVIYKDTERPSG |
| | | | | IPERFSGSSSGTKVTLTISGVQAEDEAD |
| | | | | YYCQSADNSITYRVFGGGTKVTVL |
| 283 | 1353-H08 | VL | | SYELTQPPSVSVSPGQTARITCSGDALP |
| | | | | KQYAYWYQQKPGQAPVMVIYKDTERPSG |
| | | | | IPERFSGSSSGTKVTLTISGVQAEDEAD |
| | | | | YYCQSADNSITYRVFGGGTKVTVL |
| 284 | 1353-H09 | VL | | SYELTQPPSVSVSPGQTARITCSGDALP |
| | | | | KQYAYWYQQKPGQAPVMVIYKDTERPSG |
| | | | | IPERFSGSSSGTKVTLTISGVQAEDEAD |
| | | | | YYCQSADNSITYRVFGGGTKVTVL |
| 285 | 1B10 | VL | | QIVLSQSPAILSASPGEKVTMTCRTSSS |
| | | | | VNYMHWFQQKPGSSPKPWIYATSKLASG |
| | | | | VPARFSGSGSGTSYSLTISRVEAEDAAT |
| | | | | YFCQQWISDPWTFGGGTKLEIK |
| 286 | 1E9 | VL | | DIILTQSPASLAVSLGQRAAISCRASES |
| | | | | VDNSGISFMSWFQQKPGQPPKLLIYTAS |
| | | | | NQGSGVPARFSGSGSGTEFSLNIHPMEE |
| | | | | DDTAMYFCQQSKEVPWTFGGGTKLEIR |
| 287 | 4B10 | VL | | DIVLTQSPASLAVSLGQRATISCRASEN |
| | | | | VDDYGVSFMNWFQQKPGQPPKLLIYPAS |
| | | | | NQGSGVPARFSGSGSGTDFSLNIHPMEE |
| | | | | DDTAMYFCQQSKEVPWTFGGGTKLEIK |
| 288 | h1E9-1 | VL | | DIQLTQSPSFLSASVGDRVTITCRASES |
| | | | | VDNSGISFMSWYQQKPGKAPKLLIYTAS |
| | | | | NQGSGVPSRFSGSGSGTEFTLTISSLQP |
| | | | | EDFATYYCQQSKEVPWTFGQGTKVEIK |
| 289 | h1E9-2 | VL | | EIVLTQSPATLSLSPGERATLSCRASES |
| | | | | VDNSGISFMSWYQQKPGQAPRLLIYTAS |
| | | | | NQGSGIPARFSGSGSGTDFTLTISSLEP |
| | | | | EDFAVYYCQQSKEVPWTFGQGTKVEIK |
| 290 | h1E9-4 | VL | | DIQLTQSPSFLSASVGDRVTITCRASES |
| | | | | VDNSGISFMSWYQQKPGKAPKLLIYTAS |
| | | | | NQGSGVPSRFSGSGSGTEFTLTISSLQP |
| | | | | EDFATYYCQQSKEVPWTFGQGTKVEIK |
| 291 | h1E9-5 | VL | | EIVLTQSPATLSLSPGERATLSCRASES |
| | | | | VDNSGISFMSWYQQKPGQAPRLLIYTAS |
| | | | | NQGSGIPARFSGSGSGTDFTLTISSLEP |
| | | | | EDFAVYYCQQSKEVPWTFGQGTKVEIK |
| 292 | h4B10-1 | VL | | EIVLTQSPATLSLSPGERATLSCRASEN |
| | | | | VDDYGVSFMNWYQQKPGQAPRLLIYPAS |
| | | | | NQGSGIPARFSGSGSGTDFTLTISSLEP |
| | | | | EDFAVYYCQQSKEVPWTFGQGTKVEIK |
| 293 | h4B10-2 | VL | | EIVLTQSPGTLSLSPGERATLSCRASEN |
| | | | | VDDYGVSFMNWYQQKPGQAPRLLIYPAS |
| | | | | NQGSGIPDRFSGSGSGTDFTLTISRLEP |
| | | | | EDFAVYYCQQSKEVPWTFGQGTKVEIK |
| 294 | h4B10-3 | VL | | DIVMTQSPDSLAVSLGERATINCRASEN |
| | | | | VDDYGVSFMNWYQQKPGQPPKLLIYPAS |
| | | | | NQGSGVPDRFSGSGSGTDFTLTISSLQA |
| | | | | EDVAVYYCQQSKEVPWTFGGGTKLEIK |
| 295 | PD1-17 | VL | | NFMLTQPHSVSESPGKTVTISCTRSSGS |
| | | | | IASNSVQWYQQRPGSSPTTVIYEDNQRP |
| | | | | SGVPDRFSGSIDSSSNSASLTVSGLKTE |
| | | | | DEADYYCQSSDSSAVVFGSGTKLTVL |
| 296 | PD1-28 | VL | | SYELTQPPSVSVSPGQTARITCSGDALP |
| | | | | KQYAYWYQQKPGQAPVMVIYKDTERPSG |
| | | | | IPERFSGSSSGTKVTLTISGVQAEDEAD |
| | | | | YYCQSADNSITYRVFGGGTKVTVL |
| 297 | PD1-33 | VL | | QSALTQPASVSGSPGQSITISCTGTSND |
| | | | | VGGYNYVSWYQHHPGKAPKLIIYDVTNR |
| | | | | PSGVSDRFSGSKSGNTASLTISGLLAED |
| | | | | EGDYYCSSYTIVTNFEVLFGGGTKLTV |
| 298 | PD1-35 | VL | | QSVLTQPPSASGTPGQRVTISCSGSNSN |
| | | | | IGSNSVNWYQQLPGTAPKLLIYGNNQRP |
| | | | | SGVPDRFSGSKSGTSASLAISGLQSENE |
| | | | | ADYYCAAWDDSLNGPVFGRGTKVTVL |
| 299 | PD1-F2 | VL | | DIVMTQSPSTLSASVGDRVTITCRASQG |
| | | | | ISSWLAWYQQKPGRAPKVLIYKASTLES |
| | | | | GVPSRFSGSGSGTDFTLTISSLQPEDFA |
| | | | | TYYCQQSYSTPWTFGQGTKLEIK |
| 300 | Human IgG1 HC | | | ASTKGPSVFPLAPSSKSTSGGTAALGCL |
| | Constant | | | VKDYFPEPVTVSWNSGALTSGVHTFPAV |
| | | | | LQSSGLYSLSSVVTVPSSSLGTQTYICN |
| | | | | VNHKPSNTKVDKKVEPKSCDKTHTCPPC |
| | | | | PAPELLGGPSVFLFPPKPKDTLMISRTP |
| | | | | EVTCVVVDVSHEDPEVKFNWYVDGVEVH |
| | | | | NAKTKPREEQYNSTYRVVSVLTVLHQDW |
| | | | | LNGKEYKCKVSNKALPAPIEKTISKAKG |
| | | | | QPREPQVYTLPPSREEMTKNQVSLTCLV |
| | | | | KGFYPSDIAVEWESNGQPENNYKTTPPV |
| | | | | LDSDGSFFLYSKLTVDKSRWQQGNVFSC |
| | | | | SVMHEALHNHYTQKSLSLSPGK |
| 301 | Human IgG LC | | | RTVAAPSVFIFPPSDEQLKSGTASVVCL |
| | Constant Ckappa | | | LNNFYPREAKVQWKVDNALQSGNSQESV |
| | | | | TEQDSKDSTYSLSSTLTLSKADYEKHKV |
| | | | | YACEVTHQGLSSPVTKSFNRGEC |
| 302 | Mouse IgG1 HC | | | AKTTPPSVYPLAPGSAAQTNSMVTLGCL |
| | Constant | | | VKGYFPEPVTVTWNSGSLSSGVHTFPAV |
| | | | | LQSDLYTLSSSVTVPSSTWPSETVTCNV |
| | | | | AHPASSTKVDKKIVPRDCGCKPCICTVP |
| | | | | EVSSVFIFPPKPKDVLTITLTPKVTCVV |
| | | | | VDISKDDPEVQFSWFVDDVEVHTAQTQP |
| | | | | REEQFNSTFRSVSELPIMHQDWLNGKEF |
| | | | | KCRVNSAAFPAPIEKTISKTKGRPKAPQ |
| | | | | VYTIPPPKEQMAKDKVSLTCMITDFFPE |
| | | | | DITVEWQWNGQPAENYKNTQPIMDTDGS |
| | | | | YFVYSKLNVQKSNWEAGNTFTCSVLHEG |
| | | | | LHNHHTEKSLSHSPG |
| 303 | Mouse IgG LC | | | RADAAPTVSIFPPSSEQLTSGGASVVCF |
| | Constant Ckappa | | | LNNFYPKDINVKWKIDGSERQNGVLNSW |
| | | | | TDQDSKDSTYSMSSTLTLTKDEYERHNS |
| | | | | YTCEATHKTSTSPIVKSFNRNEC |
| 304 | Kappa LC | | | HMTVAAPSVFIFPPSDEQLKSGTASVVC |
| | | | | LLNNFYPREAKVQWKVDNALQSGNSQES |
| | | | | VTEQDSKDSTYSLSSTLTLSKADYEKHK |
| | | | | VYACEVTHQGLSSPVTKSFNRGEC |
| 305 | Lambda LC | | | GQPKAAPSVTLFPPSSEELQANKATLVC |
| | | | | LISDFYPGAVTVAWKADSSPVKAGVETT |
| | | | | TPSKQSNNKYAASSYLSLTPEQWKSHRS |
| | | | | YSCQVTHEGSTVEKTVAPTECS |
| 306 | IgG1 Fc from scFv-Fc | | | AAGSDQEPKSSDKTHTCPPCSAPELLGG |
| | | | | SSVFLFPPKPKDTLMISRTPEVTCVVVD |
| | | | | VSHEDPEVKFNWYVDGVEVHNAKTKPRE |
| | | | | EQYNSTYRVVSVLTVLHQDWLNGKEYKC |
| | | | | KVSNKALPAPIEKTISKAKGQPREPQVY |
| | | | | TLPPSRDELTKNQVSLTCLVKGFYPSDI |
| | | | | AVEWESNGQPENNYKTTPPVLDSDGSFF |
| | | | | LYSKLTVDKSRWQQGNVFSCSVMHEALH |
| | | | | NHYTQKSLSLSPGKGSGDYKDDDDKGSG |
| 307 | FlagHis Tag | | | GSGDYKDDDDKGSGHHHHHH |
| 308 | Linker (GGGGS)₃ | | | GGGGSGGGGSGGGGS |
| 309 | Linker (GGGGS)₄ | | | GGGGSGGGGSGGGGSGGGGS |
| 310 | Linker (GGGGS)₅ | | | GGGGSGGGGSGGGGSGGGGSGGGGS |
| 311 | Linker (GGGGS)₆ | | | GGGGSGGGGSGGGGSGGGGSGGGGSGGG |
| | | | | GS |
| 312 | Linker | | | AAGSDQEPKSS |
| 313 | Linker | | | AAGSDQ |
| 314 | Linker | | | APGPSAPSHRSLPSRAFG |
| 315 | Linker-hinge | | | AAGSDQEPKSSDKTHTCPPCP |
| 316 | Hinge-wt | | | DKTHTCPPCP |
| 317 | 26H10 | scFv | | QVQLVESGGGVVQPGRSLRLSCAASGF |
| | | | | TFSSYGMHWVRQAPGKGLEWVAVIWYD |
| | | | | GSNKYYADSVKGRFTISRDNSKNTLYL |
| | | | | QMNSLRAEDTAVYYCAREWAVASWDYG |
| | | | | MDVWGQGTTVTVSSGGGGSGGGGSGGG |
| | | | | GSEIVLTQSPGTLSLSPGERATLSCRA |
| | | | | SQSVSSSYLAWYQQKPGQAPRLLIYGA |
| | | | | SSRATGIPDRFSGSGSGTDFTLTISRL |
| | | | | EPEDFAVYYCQQYGSSPFTFGPGTKVD |
| | | | | IK |
| 318 | hPD-1 | | | MQIPQAPWPVVWAVLQLGWRPGWFLDSP |
| | | | | DRPWNPPTFSPALLVVTEGDNATFTCSF |
| | | | | SNTSESFVLNWYRMSPSNQTDKLAAFPE |
| | | | | DRSQPGQDCRFRVTQLPNGRDFHMSVVR |
| | | | | ARRNDSGTYLCGAISLAPKAQIKESLRA |
| | | | | ELRVTERRAEVPTAHPSPSPRPAGQFQT |
| | | | | LVVGVVGGLLGSLVLLVWVLAVICSRAA |
| | | | | RGTIGARRTGQPLKEDPSAVPVFSVDYG |
| | | | | ELDFQWREKTPEPPVPCVPEQTEYATIV |
| | | | | FPSGMGTSSPARRGSADGPRSAQPLRPE |
| | | | | DGHCSWPL |
| 319 | murine PD-1 | | | MWVRQVPWSFTWAVLQLSWQSGWLLEVP |
| | | | | NGPWRSLTFYPAWLTVSEGANATFTCSL |
| | | | | SNWSEDLMLNWNRLSPSNQTEKQAAFCN |
| | | | | GLSQPVQDARFQIIQLPNRHDFHMNILD |
| | | | | TRRNDSGIYLCGAISLHPKAKIEESPGA |
| | | | | ELVVTERILETSTRYPSPSPKPEGRFQG |
| | | | | MVIGIMSALVGIPVLLLLAWALAVFCST |
| | | | | SMSEARGAGSKDDTLKEEPSAAPVPSVA |
| | | | | YEELDFQGREKTPELPTACVHTEYATIV |
| | | | | FTEGLGASAMGRRGSADGLQGPRPPRHE |
| | | | | DGHCSWPL |
| 320 | cyno PD-1 | | | MWVRQVPWSFTWAVLQLSWQSGWLLEVP |
| | | | | NGPWRSLTFYPAWLTVSEGANATFTCSL |
| | | | | SNWSEDLMLNWNRLSPSNQTEKQAAFCN |
| | | | | GLSQPVQDARFQIIQLPNRHDFHMNILD |
| | | | | TRRNDSGIYLCGAISLHPKAKIEESPGA |
| | | | | ELVVTERILETSTRYPSPSPKPEGRFQG |
| | | | | MVIGIMSALVGIPVLLLLAWALAVFCST |
| | | | | SMSEARGAGSKDDTLKEEPSAAPVPSVA |
| | | | | YEELDFQGREKTPELPTACVHTEYATIV |
| | | | | FTEGLGASAMGRRGSADGLQGPRPPRHE |
| | | | | DGHCSWPL |
| 321 | CH₂—CH₃, Fc-knob | | | APELLGGPSVFLFPPKPKDTLMISRTPE |
| | | | | VTCVVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPSREEMTKNQVSLWCLVK |
| | | | | GFYPSDIAVEWESNGQPENNYKTTPPVL |
| | | | | DSDGSFFLYSKLTVDKSRWQQGNVFSCS |
| | | | | VMHEALHNHYTQKSLSLSPGK |
| 322 | CH₂—CH₃, | | | APELLGGPSVFLFPPKPKDTLMISRTPE |
| | Fc-knob-V262E | | | VTCEVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPSREEMTKNQVSLWCLVK |
| | | | | GFYPSDIAVEWESNGQPENNYKTTPPVL |
| | | | | DSDGSFFLYSKLTVDKSRWQQGNVFSCS |
| | | | | VMHEALHNHYTQKSLSLSPGK |
| 323 | CH₂—CH₃, | | | APELLGGPSVFLFPPKPKDTLMISRTPE |
| | Fc-knob-V264S | | | VTCVVSDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPSREEMTKNQVSLWCLVK |
| | | | | GFYPSDIAVEWESNGQPENNYKTTPPVL |
| | | | | DSDGSFFLYSKLTVDKSRWQQGNVFSCS |
| | | | | VMHEALHNHYTQKSLSLSPGK |
| 324 | CH₂—CH₃, | | | APELLGGPSVFLFPPKPKDTLMISRTPE |
| | Fc-knob-D399C | | | VTCVVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPSRDELTKNQVSLWCLVK |
| | | | | GFYPSDIAVEWESNGQPENNYKTTPPVL |
| | | | | CSDGSFFLYSKLTVDKSRWQQGNVFSCS |
| | | | | VMHEALHNHYTQKSLSLSPGK |
| 325 | CH₂—CH₃, | | | APELLGGPSVFLFPPKPKDTLMISRTPE |
| | Fc-knob-S354C | | | VTCVVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPCRDELTKNQVSLWCLVK |
| | | | | GFYPSDIAVEWESNGQPENNYKTTPPVL |
| | | | | DSDGSFFLYSKLTVDKSRWQQGNVFSCS |
| | | | | VMHEALHNHYTQKSLSLSPGK |
| 326 | CH₂—CH₃, Fc-hole | | | APELLGGPSVFLFPPKPKDTLMISRTPE |
| | | | | VTCVVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPSREEMTKNQVSLSCAVK |
| | | | | GFYPSDIAVEWESNGQPENNYKTTPPVL |
| | | | | DSDGSFFLVSKLTVDKSRWQQGNVFSCS |
| | | | | VMHEALHNHYTQKSLSLSPGK |
| 327 | CH₂—CH₃, | | | APELLGGPSVFLFPPKPKDTLMISRTPE |
| | Fc-hole-V262E | | | VTCEVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPSREEMTKNQVSLSCAVK |
| | | | | GFYPSDIAVEWESNGQPENNYKTTPPVL |
| | | | | DSDGSFFLVSKLTVDKSRWQQGNVFSCS |
| | | | | VMHEALHNHYTQKSLSLSPGK |
| 328 | CH₂—CH₃, | | | APELLGGPSVFLFPPKPKDTLMISRTPE |
| | Fc-hole-V264S | | | VTCVVSDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPSREEMTKNQVSLSCAVK |
| | | | | GFYPSDIAVEWESNGQPENNYKTTPPVL |
| | | | | DSDGSFFLVSKLTVDKSRWQQGNVFSCS |
| | | | | VMHEALHNHYTQKSLSLSPGK |
| 329 | CH₂—CH₃, | | | APELLGGPSVFLFPPKPKDTLMISRTPE |
| | Fc-hole-Y349C | | | VTCVVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVCTLPPSRDELTKNQVSLSCAVK |
| | | | | GFYPSDIAVEWESNGQPENNYKTTPPVL |
| | | | | DSDGSFFLVSKLTVDKSRWQQGNVFSCS |
| | | | | VMHEALHNHYTQKSLSLSPGK |
| 330 | CH₂—CH₃, | | | APELLGGPSVFLFPPKPKDTLMISRTPE |
| | Fc-hole-K392C | | | VTCVVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYTLPPSRDELTKNQVSLSCAVK |
| | | | | GFYPSDIAVEWESNGQPENNYCTTPPVL |
| | | | | DSDGSFFLVSKLTVDKSRWQQGNVFSCS |
| | | | | VMHEALHNHYTQKSLSLSPGK |
| 331 | Fc-zwA | | | APELLGGPSVFLFPPKPKDTLMISRTPE |
| | | | | VTCVVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYVYPPSREEMTKNQVSLTCLVK |
| | | | | GFYPSDIAVEWESNGQPENNYKTTPPVL |
| | | | | DSDGSFALVSKLTVDKSRWQQGNVFSCS |
| | | | | VMHEALHNHYTQKSLSLSPGK |
| 332 | Fc-zwA-V262E | | | APELLGGPSVFLFPPKPKDTLMISRTPE |
| | | | | VTCEVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYVYPPSREEMTKNQVSLTCLVK |
| | | | | GFYPSDIAVEWESNGQPENNYKTTPPVL |
| | | | | DSDGSFALVSKLTVDKSRWQQGNVFSCS |
| | | | | VMHEALHNHYTQKSLSLSPGK |
| 333 | Fc-zwA-V264S | | | APELLGGPSVFLFPPKPKDTLMISRTPE |
| | | | | VTCVVSDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYVYPPSREEMTKNQVSLTCLVK |
| | | | | GFYPSDIAVEWESNGQPENNYKTTPPVL |
| | | | | DSDGSFALVSKLTVDKSRWQQGNVFSCS |
| | | | | VMHEALHNHYTQKSLSLSPGK |
| 334 | Fc-zwB | | | APELLGGPSVFLFPPKPKDTLMISRTPE |
| | | | | VTCVVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYVLPPSREEMTKNQVSLLCLVK |
| | | | | GFYPSDIAVEWESNGQPENNYLTWPPVL |
| | | | | DSDGSFFLYSKLTVDKSRWQQGNVFSCS |
| | | | | VMHEALHNHYTQKSLSLSPGK |
| 335 | Fc-zwB-V262E | | | APELLGGPSVFLFPPKPKDTLMISRTPE |
| | | | | VTCEVVDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYVLPPSREEMTKNQVSLLCLVK |
| | | | | GFYPSDIAVEWESNGQPENNYLTWPPVL |
| | | | | DSDGSFFLYSKLTVDKSRWQQGNVFSCS |
| | | | | VMHEALHNHYTQKSLSLSPGK |
| 336 | Fc-zwB-V264S | | | APELLGGPSVFLFPPKPKDTLMISRTPE |
| | | | | VTCVVSDVSHEDPEVKFNWYVDGVEVHN |
| | | | | AKTKPREEQYNSTYRVVSVLTVLHQDWL |
| | | | | NGKEYKCKVSNKALPAPIEKTISKAKGQ |
| | | | | PREPQVYVLPPSREEMTKNQVSLLCLVK |
| | | | | GFYPSDIAVEWESNGQPENNYLTWPPVL |
| | | | | DSDGSFFLYSKLTVDKSRWQQGNVFSCS |
| | | | | VMHEALHNHYTQKSLSLSPGK |
| 337 | hinge-C_{H}2-C_{H}3-zwA | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYVYPPSREEMTK |
| | | | | NQVSLTCLVKGFYPSDIAVEWESNGQPE |
| | | | | NNYKTTPPVLDSDGSFALVSKLTVDKSR |
| | | | | WQQGNVFSCSVMHEALHNHYTQKSLSLS |
| | | | | PGK |
| 338 | hinge-C_{H}2-C_{H}3-zwA | | | DKTHTCPPCPAPELLGGPSVFLFPPKPK |
| | V262E | | | DTLMISRTPEVTCEVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYVYPPSREEMTK |
| | | | | NQVSLTCLVKGFYPSDIAVEWESNGQPE |
| | | | | NNYKTTPPVLDSDGSFALVSKLTVDKSR |
| | | | | WQQGNVFSCSVMHEALHNHYTQKSLSLS |
| | | | | PGK |
| 339 | hinge-C_{H}2-C_{H}3-zwA | | | DKTHTCPPCPAPELLGGPSVFLFPPKPK |
| | V264S | | | DTLMISRTPEVTCVVSDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYVYPPSREEMTK |
| | | | | NQVSLTCLVKGFYPSDIAVEWESNGQPE |
| | | | | NNYKTTPPVLDSDGSFALVSKLTVDKSR |
| | | | | WQQGNVFSCSVMHEALHNHYTQKSLSLS |
| | | | | PGK |
| 340 | hinge-C_{H}2-C_{H}3-zwB | | | DKTHTCPPCPAPELLGGPSVFLFPPKPK |
| | | | | DTLMISRTPEVTCVVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYVLPPSREEMTK |
| | | | | NQVSLLCLVKGFYPSDIAVEWESNGQPE |
| | | | | NNYLTWPPVLDSDGSFFLYSKLTVDKSR |
| | | | | WQQGNVFSCSVMHEALHNHYTQKSLSLS |
| | | | | PGK |
| 341 | hinge-C_{H}2-C_{H}3-zwB | | | DKTHTCPPCPAPELLGGPSVFLFPPKPK |
| | V262E | | | DTLMISRTPEVTCEVVDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYVLPPSREEMTK |
| | | | | NQVSLLCLVKGFYPSDIAVEWESNGQPE |
| | | | | NNYLTWPPVLDSDGSFFLYSKLTVDKSR |
| | | | | WQQGNVFSCSVMHEALHNHYTQKSLSLS |
| | | | | PGK |
| 342 | hinge-C_{H}2-C_{H}3-zwB | | | DKTHTCPPCPAPELLGGPSVFLFPPKPK |
| | V264S | | | DTLMISRTPEVTCVVSDVSHEDPEVKFN |
| | | | | WYVDGVEVHNAKTKPREEQYNSTYRVVS |
| | | | | VLTVLHQDWLNGKEYKCKVSNKALPAPI |
| | | | | EKTISKAKGQPREPQVYVLPPSREEMTK |
| | | | | NQVSLLCLVKGFYPSDIAVEWESNGQPE |
| | | | | NNYLTWPPVLDSDGSFFLYSKLTVDKSR |
| | | | | WQQGNVFSCSVMHEALHNHYTQKSLSLS |
| | | | | PGK |
| 343 | C_{H}1-(a)1 | | | ASTKGPSVFPEAPSSKSTSGGTAALGCL |
| | | | | VTDYFPEPVTVSWNSGALTSGVHTFPAV |
| | | | | LESSGLYSLSSVVTVPSSSLGTQTYICN |
| | | | | VNHKPSNTKVDKKVEPKSC |
| 344 | C_{H}1-(b)1 | | | ASTKGPSVFPRAPSSKSTSGGTAALGCL |
| | | | | VKDYFPEPVTVSWNSGALTSGVHTFPAV |
| | | | | LQSSGLYKLSSVVTVPSSSLGTQTYICN |
| | | | | VNHKPSNTKVDKKVEPKSC |
| 345 | C_{H}1-(c)1 | | | ASTKGPSVFPLAPSSKSTSGGTAWLGCE |
| | | | | VTDYFPEPVTVSWNSGALTSGVHTFPAV |
| | | | | LESSGLYSLSSVVTVPSSSLGTQTYICN |
| | | | | VNHKPSNTKVDKKVEPKSC |
| 346 | C_{H}1-(d)1 | | | ASTKGPSVFPLAPSSKSTSGGTAALGCE |
| | | | | VTDYFPEPVTVSWNSGALTSGVHTFPAV |
| | | | | LESSGLYSLSSVVTVPSSSLGTQTYICN |
| | | | | VNHKPSNTKVDKKVEPKSC |
| 347 | C_{H}1-(e)(f)1 | | | ASTKGPSVFPLAPSSKSTSGGTAALGCL |
| | | | | VKGYFPEPVTVSWNSGALTSGVHTFPAV |
| | | | | LKSSGLYSLSSVVTVPSSSLGTQTYICN |
| | | | | VNHKPSNTKVDKKVEPKSC |
| 348 | C_{H}1-(a)2 | | | ASTKGPSVFPLAPSSKSTSGGTAALGCL |
| | | | | VKDYFPEPVTVSWNSGALTSGVHTFPAV |
| | | | | LQSSGLYSLKSVVTVPSSSLGTQTYICN |
| | | | | VNHKPSNTKVDKKVEPKSC |
| 349 | C_{H}1-(b)2 | | | ASTKGPSVFPLAPSSKSTSGGTAALGCE |
| | | | | VTDYFPEPVTVSWNSGALTSGVHTFPAV |
| | | | | LESSGLYSLSSVVTVPSSSLGTQTYICN |
| | | | | VNHKPSNTKVDKKVEPKSC |
| 350 | C_{H}1-(c)2 | | | ASTKGPSVFPLAPSSKSTSGGTAALGCL |
| | | | | VKDYFPEPVTVSWNSGALTSGVHTFPAV |
| | | | | LKSSGLYSLSSVVTVPSSSLGTQTYICN |
| | | | | VNHKPSNTKVDKKVEPKSC |
| 351 | C_{H}1-(d)2 | | | ASTKGPSVFPLAPSSKSTSGGTAALGCL |
| | | | | VKDYFPEPVTVSWNSGALTSGVRTFPAV |
| | | | | LKSSGLYSLSSVVTVPSSSLGTQTYICN |
| | | | | VNHKPSNTKVDKKVEPKSC |
| 352 | C_{H}1-(e)(f)2 | | | ASTKGPSVFPLAPSSKSTSGGTAALGCE |
| | | | | VTDYFPEPVTVSWNSGALTSGVHTFPAV |
| | | | | LQSSGLYSLSSVVTVPSSSLGTQTYICN |
| | | | | VNHKPSNTKVDKKVEPKSC |
| 353 | Ck-(a)1 | | | RTVAAPSVFIFPPSDEQLKSGTARVGCL |
| | | | | LNNFYPREAKVQWKVDNALQSGNSQESV |
| | | | | TEQDSKDSTYSLRSTLTLSKADYEKHKV |
| | | | | YACEVTHQGLSSPVTKSFNRGEC |
| 354 | Ck-(b)1 | | | RTVAAPSVFIFPPSDEQLKSGTASVGCL |
| | | | | LNNFYPREAKVQWKVDNALQSGNSQESV |
| | | | | TEQDSKDSTYSLDSELTLSKADYEKHKV |
| | | | | YACEVTHQGLSSPVTKSFNRGEC |
| 355 | Ck-(c)1 | | | RTVAAPSVAIFPPSDERLKSGTASVVCV |
| | | | | LNNFYPREAKVQWKVDNALQSGNSQESV |
| | | | | TEQDSKDSTYSLSSRLTLSKADYEKHKV |
| | | | | YACEVTHQGLSSPVTKSFNRGEC |
| 356 | Ck-(d)1 | | | RTVAAPSVFIFPPSDERLKSGTASVVCL |
| | | | | LNNFYPREAKVQWKVDNALQSGNSKESV |
| | | | | TEQDSKDSTYSLSSRLTLSKADYEKHKV |
| | | | | YACEVTHQGLSSPVTKSFNRGEC |
| 357 | Ck-(e)(f)1 | | | RTVAAPSVFIFPPSDEELKSGTASVVCL |
| | | | | LNNFYPREAKVQWKVDNALQSGNSEESV |
| | | | | TEQDSKDSTYSLSSTLELSKADYEKHKV |
| | | | | YACEVTHQGLSSPVTKSFNRGEC |
| 358 | Cl-(a)2 | | | GQPKAAPSVTLFPPSSEELQANKATLVC |
| | | | | LISDFYPGAVTVAWKADSSPVKAGVETT |
| | | | | TPSKQSNNKYAAESELSLTPEQWKSHRS |
| | | | | YSCQVTHEGSTVEKTVAPTECS |
| 359 | Cl-(b)2 | | | GQPKAAPSVTLFPPSSEQLQANKARLVC |
| | | | | LISDFYPGAVTVAWKADSSPVKAGVETT |
| | | | | TPSKQSNNKYAASSYLSLTPEQWKSHRS |
| | | | | YSCQVTHEGSTVEKTVAPTECS |
| 360 | Ck-(c)2 | | | RTVAAPSVFIFPPSDEELKSGTASVVCW |
| | | | | LNNFYPREAKVQWKVDNALQSGNSEESV |
| | | | | TEQDSKDSTYSLSSTLELSKADYEKHKV |
| | | | | YACEVTHQGLSSPVTKSFNRGEC |
| 361 | Ck-(d)2 | | | RTVAAPSVFIFPPSDEELKSGTASVVCL |
| | | | | LNNFYPREAKVQWKVDNALQSGNSEESV |
| | | | | TEQDSKDSTYSLSSTLELSKADYEKHKV |
| | | | | YACEVTHQGLSSPVTKSFNRGEC |
| 362 | Ck-(e)2 | | | RTVAAPSVFIFPPSDERLKSGTASVVCL |
| | | | | LNNFYPREAKVQWKVDNALQSGNSKESV |
| | | | | TEQDSKDSTYSLSSRLTLSKADYEKHKV |
| | | | | YACEVTHQGLSSPVTKSFNRGEC |
| 363 | Ck-(f)2 | | | RTVAAPSVFIFPPSDERLKSGTASVVCL |
| | | | | LNNFYPREAKVQWKVDNALQSGNSQESV |
| | | | | TEQDSKDSTYSLSSTLTLSKADYEKHKV |
| | | | | YACEVTHQGLSSPVTKSFNRGEC |
| 364 | aPD-1-1353- | scFv | | EVQLVQSGAEVKKPGASVKVSCKASGYT |
| | G10_R28T/P30D/H31S_Vk1- | | | FDSYGISWVRQAPGQGLEWMGWISAYNG |
| | 39_LC | | | NTNYAQKLQGRVTMTTDTSTNTAYMELR |
| | | | | SLRSDDTAVYYCARDVDYGTGSGYWGQG |
| | | | | TLVTVSSGGGGSGGGGSGGGGSDIQLTQ |
| | | | | SPSSLSASVGDRVTITCSGDALPKQYAY |
| | | | | WYQQKPGKAPKLLIYKDTERPSGVPSRF |
| | | | | SGSSSGTKVTLTISSLQPEDFATYYCQS |
| | | | | ADNSITYRVFGGGTKVEIK |
| 365 | aPD1 1353-G10 | scFv | | EVQLVQSGAEVKKPGASVKVSCKASGYT |
| | scFvFc zwB | | | FDSYGISWVRQAPGQGLEWMGWISAYNG |
| | R28T/P30D/H31S | | | NTNYAQKLQGRVTMTTDTSTNTAYMELR |
| | | | | SLRSDDTAVYYCARDVDYGTGSGYWGQG |
| | | | | TLVTVSSGGGGSGGGGSGGGGSSYELTQ |
| | | | | PPSVSVSPGQTARITCSGDALPKQYAYW |
| | | | | YQQKPGQAPVMVIYKDTERPSGIPERFS |
| | | | | GSSSGTKVTLTISGVQAEDEADYYCQSA |
| | | | | DNSITYRVFGGGTKVTVL |
| 366 | aPD1 1353-G10 | scFv | | EVQLVQSGAEVKKPGASVKVSCKASGYR |
| | Vk1-39 | | | FPHYGISWVRQAPGQGLEWMGWISAYNG |
| | | | | NTNYAQKLQGRVTMTTDTSTNTAYMELR |
| | | | | SLRSDDTAVYYCARDVDYGTGSGYWGQG |
| | | | | TLVTVSSGGGGSGGGGSGGGGSDIQLTQ |
| | | | | SPSSLSASVGDRVTITCSGDALPKQYAY |
| | | | | WYQQKPGKAPKLLIYKDTERPSGVPSRF |
| | | | | SGSSSGTKVTLTISSLQPEDFATYYCQS |
| | | | | ADNSITYRVFGGGTKVEIK |
| 367 | 1353-G10 | scFv-Fc | | MEVQLVQSGAEVKKPGASVKVSCKASGY |
| | | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSGGGGSGGGGSGGGGSSYELT |
| | | | | QPPSVSVSPGQTARITCSGDALPKQYAY |
| | | | | WYQQKPGQAPVMVIYKDTERPSGIPERF |
| | | | | SGSSSGTKVTLTISGVQAEDEADYYCQS |
| | | | | ADNSITYRVFGGGTKVTVLAAGSDQEPK |
| | | | | KLAAGSDQEPKSSDKTHTCPPCSAPELL |
| | | | | GGSSVFLFPPKPKDTLMISRTPEVTCVV |
| | | | | VDVSHEDPEVKFNWYVDGVEVHNAKTKP |
| | | | | REEQYNSTYRVVSVLTVLHQDWLNGKEY |
| | | | | KCKVSNKALPAPIEKTISKAKGQPREPQ |
| | | | | VYTLPPSRDELTKNQVSLTCLVKGFYPS |
| | | | | DIAVEWESNGQPENNYKTTPPVLDSDGS |
| | | | | FFLYSKLTVDKSRWQQGNVFSCSVMHEA |
| | | | | LHNHYTQKSLSLSPGKGSGDYKDDDDKG |
| | | | | SGHHHHHH |
| 368 | 1353-G10 scFv-Fc | scFv-Fc | | MEVQLVQSGAEVKKPGASVKVSCKASGY |
| | hole | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSGGGGSGGGGSGGGGSSYELT |
| | | | | QPPSVSVSPGQTARITCSGDALPKQYAY |
| | | | | WYQQKPGQAPVMVIYKDTERPSGIPERF |
| | | | | SGSSSGTKVTLTISGVQAEDEADYYCQS |
| | | | | ADNSITYRVFGGGTKVTVLAAGSDQEPK |
| | | | | SSDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYTLPPSRDEL |
| | | | | TKNQVSLSCAVKGFYPSDIAVEWESNGQ |
| | | | | PENNYKTTPPVLDSDGSFFLVSKLTVDK |
| | | | | SRWQQGNVFSCSVMHEALHNHYTQKSLS |
| | | | | LSPGK |
| 369 | 1353-G10 scFv-Fc | scFv-Fc | | MEVQLVQSGAEVKKPGASVKVSCKASGY |
| | knob | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSGGGGSGGGGSGGGGSSYELT |
| | | | | QPPSVSVSPGQTARITCSGDALPKQYAY |
| | | | | WYQQKPGQAPVMVIYKDTERPSGIPERF |
| | | | | SGSSSGTKVTLTISGVQAEDEADYYCQS |
| | | | | ADNSITYRVFGGGTKVTVLAAGSDQEPK |
| | | | | SSDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYTLPPSRDEL |
| | | | | TKNQVSLWCLVKGFYPSDIAVEWESNGQ |
| | | | | PENNYKTTPPVLDSDGSFFLYSKLTVDK |
| | | | | SRWQQGNVFSCSVMHEALHNHYTQKSLS |
| | | | | LSPGK |
| 370 | 1353-G10 scFv-Fc | scFv-Fc | | MEVQLVQSGAEVKKPGASVKVSCKASGY |
| | zwA | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSGGGGSGGGGSGGGGSSYELT |
| | | | | QPPSVSVSPGQTARITCSGDALPKQYAY |
| | | | | WYQQKPGQAPVMVIYKDTERPSGIPERF |
| | | | | SGSSSGTKVTLTISGVQAEDEADYYCQS |
| | | | | ADNSITYRVFGGGTKVTVLAAGSDQEPK |
| | | | | SSDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYVYPPSREEM |
| | | | | TKNQVSLTCLVKGFYPSDIAVEWESNGQ |
| | | | | PENNYKTTPPVLDSDGSFALVSKLTVDK |
| | | | | SRWQQGNVFSCSVMHEALHNHYTQKSLS |
| | | | | LSPGK |
| 371 | 1449-G09.2 scFv-Fc | scFv-Fc | | MQVQLVESGGGVVQPGRSLRLSCAASGF |
| | knob | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSAPGPSAPSHRSLPSRAF |
| | | | | GEIVLTQSPGTLSLSPGERATLSCRASQ |
| | | | | SVSSSYLAWYQQKPGQAPRLLIYGASSR |
| | | | | ATGIPDRFSGSGSGTDFTLTISRLEPED |
| | | | | FAVYYCQQYGRSPFSFGPGTKVDIKAAG |
| | | | | SDQEPKSSDKTHTCPPCPAPELLGGPSV |
| | | | | FLFPPKPKDTLMISRTPEVTCVVVDVSH |
| | | | | EDPEVKFNWYVDGVEVHNAKTKPREEQY |
| | | | | NSTYRVVSVLTVLHQDWLNGKEYKCKVS |
| | | | | NKALPAPIEKTISKAKGQPREPQVYTLP |
| | | | | PSRDELTKNQVSLWCLVKGFYPSDIAVE |
| | | | | WESNGQPENNYKTTPPVLDSDGSFFLYS |
| | | | | KLTVDKSRWQQGNVFSCSVMHEALHNHY |
| | | | | TQKSLSLSPGK |
| 372 | 1449-G09.2 HC hole | HC | | MQVQLVESGGGVVQPGRSLRLSCAASGF |
| | | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSASTKGPSVFPLAPSSKS |
| | | | | TSGGTAALGCLVKDYFPEPVTVSWNSGA |
| | | | | LTSGVHTFPAVLQSSGLYSLSSVVTVPS |
| | | | | SSLGTQTYICNVNHKPSNTKVDKKVEPK |
| | | | | SCDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYTLPPSREEM |
| | | | | TKNQVSLSCAVKGFYPSDIAVEWESNGQ |
| | | | | PENNYKTTPPVLDSDGSFFLVSKLTVDK |
| | | | | SRWQQGNVFSCSVMHEALHNHYTQKSLS |
| | | | | LSPGK |
| 373 | 1449-G09.2 HC zwB | HC | | MQVQLVESGGGVVQPGRSLRLSCAASGF |
| | | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSASTKGPSVFPLAPSSKS |
| | | | | TSGGTAALGCLVKDYFPEPVTVSWNSGA |
| | | | | LTSGVHTFPAVLQSSGLYSLSSVVTVPS |
| | | | | SSLGTQTYICNVNHKPSNTKVDKKVEPK |
| | | | | SCDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYVLPPSREEM |
| | | | | TKNQVSLLCLVKGFYPSDIAVEWESNGQ |
| | | | | PENNYLTWPPVLDSDGSFFLYSKLTVDK |
| | | | | SRWQQGNVFSCSVMHEALHNHYTQKSLS |
| | | | | LSPGK |
| 374 | 1449-G09.2 HC1a | HC | | MQVQLVESGGGVVQPGRSLRLSCAASGF |
| | zwA | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSASTKGPSVFPEAPSSKS |
| | | | | TSGGTAALGCLVTDYFPEPVTVSWNSGA |
| | | | | LTSGVHTFPAVLESSGLYSLSSVVTVPS |
| | | | | SSLGTQTYICNVNHKPSNTKVDKKVEPK |
| | | | | SCDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYVYPPSREEM |
| | | | | TKNQVSLTCLVKGFYPSDIAVEWESNGQ |
| | | | | PENNYKTTPPVLDSDGSFALVSKLTVDK |
| | | | | SRWQQGNVFSCSVMHEALHNHYTQKSLS |
| | | | | LSPGK |
| 375 | 1449-G09.2 HC2a | HC | | MEVQLVQSGAEVKKPGASVKVSCKASGY |
| | zwB | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSASTKGPSVFPLAPSSKSTSG |
| | | | | GTAALGCLVKDYFPEPVTVSWNSGALTS |
| | | | | GVHTFPAVLQSSGLYSLKSVVTVPSSSL |
| | | | | GTQTYICNVNHKPSNTKVDKKVEPKSCD |
| | | | | KTHTCPPCPAPELLGGPSVFLFPPKPKD |
| | | | | TLMISRTPEVTCVVVDVSHEDPEVKFNW |
| | | | | YVDGVEVHNAKTKPREEQYNSTYRVVSV |
| | | | | LTVLHQDWLNGKEYKCKVSNKALPAPIE |
| | | | | KTISKAKGQPREPQVYVLPPSREEMTKN |
| | | | | QVSLLCLVKGFYPSDIAVEWESNGQPEN |
| | | | | NYLTWPPVLDSDGSFFLYSKLTVDKSRW |
| | | | | QQGNVFSCSVMHEALHNHYTQKSLSLSP |
| | | | | GK |
| 376 | 1449-G09.2 LC | LC | | MEIVLTQSPGTLSLSPGERATLSCRASQ |
| | | | | SVSSSYLAWYQQKPGQAPRLLIYGASSR |
| | | | | ATGIPDRFSGSGSGTDFTLTISRLEPED |
| | | | | FAVYYCQQYGRSPFSFGPGTKVDIKRTV |
| | | | | AAPSVFIFPPSDEQLKSGTASVVCLLNN |
| | | | | FYPREAKVQWKVDNALQSGNSQESVTEQ |
| | | | | DSKDSTYSLSSTLTLSKADYEKHKVYAC |
| | | | | EVTHQGLSSPVTKSFNRGEC |
| 3771 | 1449-G09.2 LC1a | LC | | MEIVLTQSPGTLSLSPGERATLSCRASQ |
| | | | | SVSSSYLAWYQQKPGQAPRLLIYGASSR |
| | | | | ATGIPDRFSGSGSGTDFTLTISRLEPED |
| | | | | FAVYYCQQYGRSPFSFGPGTKVDIKRTV |
| | | | | AAPSVFIFPPSDEQLKSGTARVGCLLNN |
| | | | | FYPREAKVQWKVDNALQSGNSQESVTEQ |
| | | | | DSKDSTYSLRSTLTLSKADYEKHKVYAC |
| | | | | EVTHQGLSSPVTKSFNRGEC |
| 378 | 1449-G09.2 LC2a | LC | | MSYELTQPPSVSVSPGQTARITCSGDAL |
| | | | | PKQYAYWYQQKPGQAPVMVIYKDTERPS |
| | | | | GIPERFSGSSSGTKVTLTISGVQAEDEA |
| | | | | DYYCQSADNSITYRVFGGGTKVTVLGQP |
| | | | | KAAPSVTLFPPSSEELQANKATLVCLIS |
| | | | | DFYPGAVTVAWKADSSPVKAGVETTTPS |
| | | | | KQSNNKYAAESELSLTPEQWKSHRSYSC |
| | | | | QVTHEGSTVEKTVAPTECS |
| 379 | LAG3 scFvFc | scFv | | QVQLVESGGGVVQPGRSLRLSCAASGF |
| | | | | TFSSYGMHWVRQAPGKGLEWVAVIWYD |
| | | | | GSYKYYADSVKGRFTISRDNSKNTLYL |
| | | | | QMNSLRAEDTAVYYCAREEAPENWDYA |
| | | | | LDVWGQGTTVTVSSGGGGSGGGGSGGG |
| | | | | GSEIVLTQSPGTLSLSPGERATLSCRA |
| | | | | SQSVSSSYLAWYQQKPGQKVDIK |
| 380 | 1449-G09.2 scFv-Fc | scFv-Fc | | MQVQLVESGGGVVQPGRSLRLSCAASGF |
| | hole | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSAPGPSAPSHRSLPSRAF |
| | | | | GEIVLTQSPGTLSLSPGERATLSCRASQ |
| | | | | SVSSSYLAWYQQKPGQAPRLLIYGASSR |
| | | | | ATGIPDRFSGSGSGTDFTLTISRLEPED |
| | | | | FAVYYCQQYGRSPFSFGPGTKVDIKAAG |
| | | | | SDQEPKSSDKTHTCPPCSAPELLGGSSV |
| | | | | FLFPPKPKDTLMISRTPEVTCVVVDVSH |
| | | | | EDPEVKFNWYVDGVEVHNAKTKPREEQY |
| | | | | NSTYRVVSVLTVLHQDWLNGKEYKCKVS |
| | | | | NKALPAPIEKTISKAKGQPREPQVYTLP |
| | | | | PSRDELTKNQVSLSCAVKGFYPSDIAVE |
| | | | | WESNGQPENNYKTTPPVLDSDGSFFLVS |
| | | | | KLTVDKSRWQQGNVFSCSVMHEALHNHY |
| | | | | TQKSLSLSPGK |
| 381 | Anti-PD1 1353-G10 | HC | | MEVQLVQSGAEVKKPGASVKVSCKASGY |
| | HC knob | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSASTKGPSVFPLAPSSKSTSG |
| | | | | GTAALGCLVKDYFPEPVTVSWNSGALTS |
| | | | | GVHTFPAVLQSSGLYSLSSVVTVPSSSL |
| | | | | GTQTYICNVNHKPSNTKVDKKVEPKSCD |
| | | | | KTHTCPPCPAPELLGGPSVFLFPPKPKD |
| | | | | TLMISRTPEVTCVVVDVSHEDPEVKFNW |
| | | | | YVDGVEVHNAKTKPREEQYNSTYRVVSV |
| | | | | LTVLHQDWLNGKEYKCKVSNKALPAPIE |
| | | | | KTISKAKGQPREPQVYTLPPSREEMTKN |
| | | | | QVSLWCLVKGFYPSDIAVEWESNGQPEN |
| | | | | NYKTTPPVLDSDGSFFLYSKLTVDKSRW |
| | | | | QQGNVFSCSVMHEALHNHYTQKSLSLSP |
| | | | | GK |
| 382 | Anti-PD1 1353-G10 | HC | | MEVQLVQSGAEVKKPGASVKVSCKASGY |
| | HC hole | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSASTKGPSVFPLAPSSKSTSG |
| | | | | GTAALGCLVKDYFPEPVTVSWNSGALTS |
| | | | | GVHTFPAVLQSSGLYSLSSVVTVPSSSL |
| | | | | GTQTYICNVNHKPSNTKVDKKVEPKSCD |
| | | | | KTHTCPPCPAPELLGGPSVFLFPPKPKD |
| | | | | TLMISRTPEVTCVVVDVSHEDPEVKFNW |
| | | | | YVDGVEVHNAKTKPREEQYNSTYRVVSV |
| | | | | LTVLHQDWLNGKEYKCKVSNKALPAPIE |
| | | | | KTISKAKGQPREPQVYTLPPSREEMTKN |
| | | | | QVSLSCAVKGFYPSDIAVEWESNGQPEN |
| | | | | NYKTTPPVLDSDGSFFLVSKLTVDKSRW |
| | | | | QQGNVFSCSVMHEALHNHYTQKSLSLSP |
| | | | | GK |
| 383 | Anti-PD1 1353-G10 | LC | | MSYELTQPPSVSVSPGQTARITCSGDAL |
| | LC | | | PKQYAYWYQQKPGQAPVMVIYKDTERPS |
| | | | | GIPERFSGSSSGTKVTLTISGVQAEDEA |
| | | | | DYYCQSADNSITYRVFGGGTKVTVLGQP |
| | | | | KAAPSVTLFPPSSEELQANKATLVCLIS |
| | | | | DFYPGAVTVAWKADSSPVKAGVETTTPS |
| | | | | KQSNNKYAASSYLSLTPEQWKSHRSYSC |
| | | | | QVTHEGSTVEKTVAPTECS |
| 384 | Anti-LAG3 1449- | scFv-Fc | | MQVQLVESGGGVVQPGRSLRLSCAASGF |
| | G09.2 scFvFc hole | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSAPGPSAPSHRSLPSRAF |
| | | | | GEIVLTQSPGTLSLSPGERATLSCRASQ |
| | | | | SVSSSYLAWYQQKPGQAPRLLIYGASSR |
| | | | | ATGIPDRFSGSGSGTDFTLTISRLEPED |
| | | | | FAVYYCQQYGRSPFSFGPGTKVDIKAAG |
| | | | | SDQEPKSSDKTHTCPPCSAPELLGGSSV |
| | | | | FLFPPKPKDTLMISRTPEVTCVVVDVSH |
| | | | | EDPEVKFNWYVDGVEVHNAKTKPREEQY |
| | | | | NSTYRVVSVLTVLHQDWLNGKEYKCKVS |
| | | | | NKALPAPIEKTISKAKGQPREPQVYTLP |
| | | | | PSRDELTKNQVSLSCAVKGFYPSDIAVE |
| | | | | WESNGQPENNYKTTPPVLDSDGSFFLVS |
| | | | | KLTVDKSRWQQGNVFSCSVMHEALHNHY |
| | | | | TQKSLSLSPGK |
| 385 | C_{H}1-wt | | | ASTKGPSVFPLAPSSKSTSGGTAALGCL |
| | | | | VKDYFPEPVTVSWNSGALTSGVHTFPAV |
| | | | | LQSSGLYSLSSVVTVPSSSLGTQTYICN |
| | | | | VNHKPSNTKVDKKVEPKSC |
| 386 | C_{K}-wt | | | RTVAAPSVFIFPPSDEQLKSGTASVVCL |
| | | | | LNNFYPREAKVQWKVDNALQSGNSQESV |
| | | | | TEQDSKDSTYSLSSTLTLSKADYEKHKV |
| | | | | YACEVTHQGLSSPVTKSFNRGEC |
| 387 | Cλ-wt | | | GQPKAAPSVTLFPPSSEELQANKATLVC |
| | | | | LISDFYPGAVTVAWKADSSPVKAGVETT |
| | | | | TPSKQSNNKYAASSYLSLTPEQWKSHRS |
| | | | | YSCQVTHEGSTVEKTVAPTECS |
| 388 | 1353-G10 scFv-Fc | scFv-Fc | | MEVQLVQSGAEVKKPGASVKVSCKASGY |
| | zwB | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSGGGGSGGGGSGGGGSSYELT |
| | | | | QPPSVSVSPGQTARITCSGDALPKQYAY |
| | | | | WYQQKPGQAPVMVIYKDTERPSGIPERF |
| | | | | SGSSSGTKVTLTISGVQAEDEADYYCQS |
| | | | | ADNSITYRVFGGGTKVTVLAAGSDQEPK |
| | | | | SSDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYVLPPSREEM |
| | | | | TKNQVSLLCLVKGFYPSDIAVEWESNGQ |
| | | | | PENNYLTWPPVLDSDGSFFLYSKLTVDK |
| | | | | SRWQQGNVFSCSVMHEALHNHYTQKSLS |
| | | | | LSPGK |
| 389 | 1353-G10 HC zwA | HC | | MEVQLVQSGAEVKKPGASVKVSCKASGY |
| | | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSASTKGPSVFPLAPSSKSTSG |
| | | | | GTAALGCLVKDYFPEPVTVSWNSGALTS |
| | | | | GVHTFPAVLQSSGLYSLSSVVTVPSSSL |
| | | | | GTQTYICNVNHKPSNTKVDKKVEPKSCD |
| | | | | KTHTCPPCPAPELLGGPSVFLFPPKPKD |
| | | | | TLMISRTPEVTCVVVDVSHEDPEVKFNW |
| | | | | YVDGVEVHNAKTKPREEQYNSTYRVVSV |
| | | | | LTVLHQDWLNGKEYKCKVSNKALPAPIE |
| | | | | KTISKAKGQPREPQVYVYPPSREEMTKN |
| | | | | QVSLTCLVKGFYPSDIAVEWESNGQPEN |
| | | | | NYKTTPPVLDSDGSFALVSKLTVDKSRW |
| | | | | QQGNVFSCSVMHEALHNHYTQKSLSLSP |
| | | | | GK |
| 390 | 1353-G10 HC zwB | HC | | MEVQLVQSGAEVKKPGASVKVSCKASGY |
| | | | | RFPHYGISWVRQAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSASTKGPSVFPLAPSSKSTSG |
| | | | | GTAALGCLVKDYFPEPVTVSWNSGALTS |
| | | | | GVHTFPAVLQSSGLYSLSSVVTVPSSSL |
| | | | | GTQTYICNVNHKPSNTKVDKKVEPKSCD |
| | | | | KTHTCPPCPAPELLGGPSVFLFPPKPKD |
| | | | | TLMISRTPEVTCVVVDVSHEDPEVKFNW |
| | | | | YVDGVEVHNAKTKPREEQYNSTYRVVSV |
| | | | | LTVLHQDWLNGKEYKCKVSNKALPAPIE |
| | | | | KTISKAKGQPREPQVYVLPPSREEMTKN |
| | | | | QVSLLCLVKGFYPSDIAVEWESNGQPEN |
| | | | | NYLTWPPVLDSDGSFFLYSKLTVDKSRW |
| | | | | QQGNVFSCSVMHEALHNHYTQKSLSLSP |
| | | | | GK |
| 391 | 1449-G09.2 scFv-Fc | scFv-Fc | | MQVQLVESGGGVVQPGRSLRLSCAASGF |
| | zwA | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSAPGPSAPSHRSLPSRAF |
| | | | | GEIVLTQSPGTLSLSPGERATLSCRASQ |
| | | | | SVSSSYLAWYQQKPGQAPRLLIYGASSR |
| | | | | ATGIPDRFSGSGSGTDFTLTISRLEPED |
| | | | | FAVYYCQQYGRSPFSFGPGTKVDIKAAG |
| | | | | SDQEPKSSDKTHTCPPCPAPELLGGPSV |
| | | | | FLFPPKPKDTLMISRTPEVTCVVVDVSH |
| | | | | EDPEVKFNWYVDGVEVHNAKTKPREEQY |
| | | | | NSTYRVVSVLTVLHQDWLNGKEYKCKVS |
| | | | | NKALPAPIEKTISKAKGQPREPQVYVYP |
| | | | | PSREEMTKNQVSLTCLVKGFYPSDIAVE |
| | | | | WESNGQPENNYKTTPPVLDSDGSFALVS |
| | | | | KLTVDKSRWQQGNVFSCSVMHEALHNHY |
| | | | | TQKSLSLSPGK |
| 392 | 1449-G09.2 scFv-Fc | scFv-Fc | | MQVQLVESGGGVVQPGRSLRLSCAASGF |
| | zwB | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSAPGPSAPSHRSLPSRAF |
| | | | | GEIVLTQSPGTLSLSPGERATLSCRASQ |
| | | | | SVSSSYLAWYQQKPGQAPRLLIYGASSR |
| | | | | ATGIPDRFSGSGSGTDFTLTISRLEPED |
| | | | | FAVYYCQQYGRSPFSFGPGTKVDIKAAG |
| | | | | SDQEPKSSDKTHTCPPCPAPELLGGPSV |
| | | | | FLFPPKPKDTLMISRTPEVTCVVVDVSH |
| | | | | EDPEVKFNWYVDGVEVHNAKTKPREEQY |
| | | | | NSTYRVVSVLTVLHQDWLNGKEYKCKVS |
| | | | | NKALPAPIEKTISKAKGQPREPQVYVLP |
| | | | | PSREEMTKNQVSLLCLVKGFYPSDIAVE |
| | | | | WESNGQPENNYLTWPPVLDSDGSFFLYS |
| | | | | KLTVDKSRWQQGNVFSCSVMHEALHNHY |
| | | | | TQKSLSLSPGK |
| 393 | 1449-G09.2 HC zwA | HC | | MQVQLVESGGGVVQPGRSLRLSCAASGF |
| | | | | TFSSYGMHWVRQAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSASTKGPSVFPLAPSSKS |
| | | | | TSGGTAALGCLVKDYFPEPVTVSWNSGA |
| | | | | LTSGVHTFPAVLQSSGLYSLSSVVTVPS |
| | | | | SSLGTQTYICNVNHKPSNTKVDKKVEPK |
| | | | | SCDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYVYPPSREEM |
| | | | | TKNQVSLTCLVKGFYPSDIAVEWESNGQ |
| | | | | PENNYKTTPPVLDSDGSFALVSKLTVDK |
| | | | | SRWQQGNVFSCSVMHEALHNHYTQKSLS |
| | | | | LSPGK |
| 394 | 1353-G10 LC1c | LC | | MSYELTQPPSVSVSPGQTARITCSGDAL |
| | | | | PKQYAYWYQQKPGQAPVMVIYKDTERPS |
| | | | | GIPERFSGSSSGTKVTLTISGVQAEDFA |
| | | | | TYYCQSADNSITYRVFGGGTKVEIKRTV |
| | | | | AAPSVAIFPPSDERLKSGTASVVCVLNN |
| | | | | FYPREAKVQWKVDNALQSGNSQESVTEQ |
| | | | | DSKDSTYSLSSRLTLSKADYEKHKVYAC |
| | | | | EVTHQGLSSPVTKSFNRGEC |
| 395 | 1353-G10 LC1d | LC | | MSYELTQPPSVSVSPGQTARITCSGDAL |
| | | | | PKQYAYWYQRKPGQAPVMVIYKDTERPS |
| | | | | GIPERFSGSSSGTKVTLTISGVQAEDFA |
| | | | | TYYCQSADNSITYRVFGGGTKVEIKRTV |
| | | | | AAPSVFIFPPSDERLKSGTASVVCLLNN |
| | | | | FYPREAKVQWKVDNALQSGNSKESVTEQ |
| | | | | DSKDSTYSLSSRLTLSKADYEKHKVYAC |
| | | | | EVTHQGLSSPVTKSFNRGEC |
| 396 | 1449-G09.2 LC2d | LC | | MEIVLTQSPGTLSLSPGERATLSCRASQ |
| | | | | SVSSSYLAWYQEKPGQAPRLLIYGASSR |
| | | | | ATGIPDRFSGSGSGTDFTLTISRLEPED |
| | | | | FAVYYCQQYGRSPFSFGPGTKVDIKRTV |
| | | | | AAPSVFIFPPSDEELKSGTASVVCLLNN |
| | | | | FYPREAKVQWKVDNALQSGNSEESVTEQ |
| | | | | DSKDSTYSLSSTLELSKADYEKHKVYAC |
| | | | | EVTHQGLSSPVTKSFNRGEC |
| 397 | 1353-G10 HC1d zwB | HC | | MEVQLVQSGAEVKKPGASVKVSCKASGY |
| | | | | RFPHYGISWVREAPGQGLEWMGWISAYN |
| | | | | GNTNYAQKLQGRVTMTTDTSTNTAYMEL |
| | | | | RSLRSDDTAVYYCARDVDYGTGSGYWGQ |
| | | | | GTLVTVSSASTKGPSVFPLAPSSKSTSG |
| | | | | GTAALGCEVTDYFPEPVTVSWNSGALTS |
| | | | | GVHTFPAVLESSGLYSLSSVVTVPSSSL |
| | | | | GTQTYICNVNHKPSNTKVDKKVEPKSCD |
| | | | | KTHTCPPCPAPELLGGPSVFLFPPKPKD |
| | | | | TLMISRTPEVTCVVVDVSHEDPEVKFNW |
| | | | | YVDGVEVHNAKTKPREEQYNSTYRVVSV |
| | | | | LTVLHQDWLNGKEYKCKVSNKALPAPIE |
| | | | | KTISKAKGQPREPQVYVLPPSREEMTKN |
| | | | | QVSLLCLVKGFYPSDIAVEWESNGQPEN |
| | | | | NYLTWPPVLDSDGSFFLYSKLTVDKSRW |
| | | | | QQGNVFSCSVMHEALHNHYTQKSLSLSP |
| | | | | GK |
| 398 | 1449-G09.2 HC2d | HC | | MQVQLVESGGGVVQPGRSLRLSCAASGF |
| | zwA | | | TFSSYGMHWVRRAPGKGLEWVAVIWYDG |
| | | | | SYKYYADSVKGRFTISRDNSKNTLYLQM |
| | | | | NSLRAEDTAVYYCAREEAPENWDYALDV |
| | | | | WGQGTTVTVSSASTKGPSVFPLAPSSKS |
| | | | | TSGGTAALGCLVKDYFPEPVTVSWNSGA |
| | | | | LTSGVRTFPAVLKSSGLYSLSSVVTVPS |
| | | | | SSLGTQTYICNVNHKPSNTKVDKKVEPK |
| | | | | SCDKTHTCPPCPAPELLGGPSVFLFPPK |
| | | | | PKDTLMISRTPEVTCVVVDVSHEDPEVK |
| | | | | FNWYVDGVEVHNAKTKPREEQYNSTYRV |
| | | | | VSVLTVLHQDWLNGKEYKCKVSNKALPA |
| | | | | PIEKTISKAKGQPREPQVYVYPPSREEM |
| | | | | TKNQVSLTCLVKGFYPSDIAVEWESNGQ |
| | | | | PENNYKTTPPVLDSDGSFALVSKLTVDK |
| | | | | SRWQQGNVFSCSVMHEALHNHYTQKSLS |
| | | | | LSPGK |

### EQUIVALENTS

The disclosure set forth above may encompass multiple distinct inventions with independent utility. Although each of these inventions has been disclosed in its preferred form(s), the specific embodiments thereof as disclosed and illustrated herein are not to be considered in a limiting sense, because numerous variations are possible. The subject matter of the inventions includes all novel and nonobvious combinations and subcombinations of the various elements, features, functions, and/or properties disclosed herein. The following claims particularly point out certain combinations and subcombinations regarded as novel and nonobvious. Inventions embodied in other combinations and subcombinations of features, functions, elements, and/or properties may be claimed in this application, in applications claiming priority from this application, or in related applications. Such claims, whether directed to a different invention or to the same invention, and whether broader, narrower, equal, or different in scope in comparison to the original claims, also are regarded as included within the subject matter of the inventions of the present disclosure.

What is claimed is:

1. A bi-specific antibody or antigen-binding construct, comprising a first binding domain that specifically binds to LAG3, and a second binding domain that specifically binds to PD-1, wherein:
i. the first binding domain comprises a CDR-H3 sequence selected from SEQ ID NOs: 110-129; and
ii. the second binding domain comprises a CDR-H3 sequence selected from the group consisting of: SEQ ID NOs: 130-131 and the CDR-H3 sequences of the V_{H} sequences SEQ ID NOs: 212-250.

2. The bi-specific antibody or antigen binding construct of claim 1, wherein the first binding domain comprises a Chothia CDR-H2 sequence selected from SEQ ID NOs: 66-85.

3. The bi-specific antibody or antigen binding construct of claim 1, wherein the first binding domain comprises a Chothia CDR-H1 sequence selected from SEQ ID NOs: 4-23.

4. The bi-specific antibody or antigen binding construct of claim 1, wherein the first binding domain comprises a Kabat CDR-H2 sequence selected from SEQ ID NOs: 88-107.

5. The bi-specific antibody or antigen binding construct of claim 1, wherein the first binding domain comprises a Kabat CDR-H1 sequence selected from SEQ ID NOs: 39-58.

6. The bi-specific antibody or antigen binding construct of claim 1, wherein the first binding domain comprises a CDR-L3 sequence selected from SEQ ID NOs: 173-188.

7. The bi-specific antibody or antigen binding construct of claim 1, wherein the first binding domain comprises a CDR-L2 sequence selected from SEQ ID NOs: 150-165.

8. The bi-specific antibody or antigen binding construct of claim 1, wherein the first binding domain comprises a CDR-L1 sequence selected from SEQ ID NOs: 132-147.

9. The bi-specific antibody or antigen binding construct of claim 1, wherein the first binding domain comprises:
i. a V_{H} comprising: a CDR-H1 comprising one or more of SEQ ID NOs: 7 and 42; a CDR-H2 comprising one or more of SEQ ID NOs: 69 and 91; and a CDR-H3 comprising SEQ ID NO: 113;
ii. a V_{H} comprising: a CDR-H1 comprising one or more of SEQ ID NOs: 4 and 39; a CDR-H2 comprising one or more of SEQ ID NOs: 66 and 88; and a CDR-H3 comprising SEQ ID NO: 110;
iii. a V_{H} comprising: a CDR-H1 comprising one or more of SEQ ID NOs: 5 and 40; a CDR-H2 comprising one or more of SEQ ID NOs: 67 and 89; and a CDR-H3 comprising SEQ ID NO: 111;
iv. a V_{H} comprising: a CDR-H1 comprising one or more of SEQ ID NOs: 6 and 41; a CDR-H2 comprising one or more of SEQ ID NOs: 68 and 90; and a CDR-H3 comprising SEQ ID NO: 112;
v. a V_{H} comprising: a CDR-H1 comprising one or more of SEQ ID NOs: 8 and 43; a CDR-H2 comprising one or more of SEQ ID NOs: 70 and 92; and a CDR-H3 comprising SEQ ID NO: 114;
vi. a V_{H} comprising: a CDR-H1 comprising one or more of SEQ ID NOs: 9 and 44; a CDR-H2 comprising one or more of SEQ ID NOs: 71 and 93; and a CDR-H3 comprising SEQ ID NO: 115;
vii. a V_{H} comprising: a CDR-H1 comprising one or more of SEQ ID NOs: 10 and 45; a CDR-H2 comprising one or more of SEQ ID NOs: 72 and 94; and a CDR-H3 comprising SEQ ID NO: 116;
viii. a V_{H} comprising: a CDR-H1 comprising one or more of SEQ ID NOs: 11 and 46; a CDR-H2 comprising one or more of SEQ ID NOs: 73 and 95; and a CDR-H3 comprising SEQ ID NO: 117;
ix. a V_{H} comprising: a CDR-H1 comprising one or more of SEQ ID NOs: 12 and 47; a CDR-H2 comprising one or more of SEQ ID NOs: 74 and 96; and a CDR-H3 comprising SEQ ID NO: 118;
x. a V_{H} comprising: a CDR-H1 comprising one or more of SEQ ID NOs: 13 and 48; a CDR-H2 comprising one or more of SEQ ID NOs: 75 and 97; and a CDR-H3 comprising SEQ ID NO: 119;
xi. a V_{H} comprising: a CDR-H1 comprising one or more of SEQ ID NOs: 14 and 49; a CDR-H2 comprising one or more of SEQ ID NOs: 76 and 98; and a CDR-H3 comprising SEQ ID NO: 120;
xii. a V_{H} comprising: a CDR-H1 comprising one or more of SEQ ID NOs: 15 and 50; a CDR-H2 comprising one or more of SEQ ID NOs: 77 and 99; and a CDR-H3 comprising SEQ ID NO: 121;
xiii. a V_{H} comprising: a CDR-H1 comprising one or more of SEQ ID NOs: 16 and 51; a CDR-H2 comprising one or more of SEQ ID NOs: 78 and 100; and a CDR-H3 comprising SEQ ID NO: 122;
xiv. a V_{H} comprising: a CDR-H1 comprising one or more of SEQ ID NOs: 17 and 52; a CDR-H2 comprising one or more of SEQ ID NOs: 79 and 101; and a CDR-H3 comprising SEQ ID NO: 123;
xv. a V_{H} comprising: a CDR-H1 comprising one or more of SEQ ID NOs: 18 and 53; a CDR-H2 comprising one or more of SEQ ID NOs: 80 and 102; and a CDR-H3 comprising SEQ ID NO: 124;
xvi. a V_{H} comprising: a CDR-H1 comprising one or more of SEQ ID NOs: 19 and 54; a CDR-H2 comprising one or more of SEQ ID NOs: 81 and 103; and a CDR-H3 comprising SEQ ID NO: 125;
xvii. a V_{H} comprising: a CDR-H1 comprising one or more of SEQ ID NOs: 20 and 55; a CDR-H2 comprising one or more of SEQ ID NOs: 82 and 104; and a CDR-H3 comprising SEQ ID NO: 126;
xviii. a V_{H} comprising: a CDR-H1 comprising one or more of SEQ ID NOs: 21 and 56; a CDR-H2 comprising one or more of SEQ ID NOs: 83 and 105; and a CDR-H3 comprising SEQ ID NO: 127;
xix. a V_{H} comprising: a CDR-H1 comprising one or more of SEQ ID NOs: 22 and 57; a CDR-H2 comprising one or more of SEQ ID NOs: 84 and 106; and a CDR-H3 comprising SEQ ID NO: 128; or
xx. a V_{H} comprising: a CDR-H1 comprising one or more of SEQ ID NOs: 23 and 58; a CDR-H2 comprising one or more of SEQ ID NOs: 85 and 107; and a CDR-H3 comprising SEQ ID NO: 129.

10. The bi-specific antibody or antigen binding construct of claim 1, wherein the first binding domain comprises a V_{H} selected from SEQ ID NOs: 191-210.

11. The bi-specific antibody or antigen binding construct of claim 9, wherein the first binding domain comprises:
i. a V_{L} comprising: a CDR-L1 comprising SEQ ID NO: 135; a CDR-L2 comprising SEQ ID NO: 153; and a CDR-L3 comprising SEQ ID NO: 176;
ii. a V_{L} comprising: a CDR-L1 comprising SEQ ID NO: 132; a CDR-L2 comprising SEQ ID NO: 150; and a CDR-L3 comprising SEQ ID NO: 173;
iii. a V_{L} comprising: a CDR-L1 comprising SEQ ID NO: 133; a CDR-L2 comprising SEQ ID NO: 151; and a CDR-L3 comprising SEQ ID NO: 174;
iv. a V_{L} comprising: a CDR-L1 comprising SEQ ID NO: 134; a CDR-L2 comprising SEQ ID NO: 152; and a CDR-L3 comprising SEQ ID NO: 175;
v. a V_{L} comprising: a CDR-L1 comprising SEQ ID NO: 136; a CDR-L2 comprising SEQ ID NO: 154; and a CDR-L3 comprising SEQ ID NO: 177;
vi. a V_{L} comprising: a CDR-L1 comprising SEQ ID NO: 137; a CDR-L2 comprising SEQ ID NO: 155; and a CDR-L3 comprising SEQ ID NO: 178;
vii. a V_{L} comprising: a CDR-L1 comprising SEQ ID NO: 138; a CDR-L2 comprising SEQ ID NO: 156; and a CDR-L3 comprising SEQ ID NO: 179;
viii. a V_{L} comprising: a CDR-L1 comprising SEQ ID NO: 139; a CDR-L2 comprising SEQ ID NO: 157; and a CDR-L3 comprising SEQ ID NO: 180;
ix. a V_{L} comprising: a CDR-L1 comprising SEQ ID NO: 140; a CDR-L2 comprising SEQ ID NO: 158; and a CDR-L3 comprising SEQ ID NO: 181;
x. a V_{L} comprising: a CDR-L1 comprising SEQ ID NO: 141; a CDR-L2 comprising SEQ ID NO: 159; and a CDR-L3 comprising SEQ ID NO: 182;
xi. a V_{L} comprising: a CDR-L1 comprising SEQ ID NO: 142; a CDR-L2 comprising SEQ ID NO: 160; and a CDR-L3 comprising SEQ ID NO: 183;
xii. a V_{L} comprising: a CDR-L1 comprising SEQ ID NO: 143; a CDR-L2 comprising SEQ ID NO: 161; and a CDR-L3 comprising SEQ ID NO: 184;
xiii. a V_{L} comprising: a CDR-L1 comprising SEQ ID NO: 144; a CDR-L2 comprising SEQ ID NO: 162; and a CDR-L3 comprising SEQ ID NO: 185;
xiv. a V_{L} comprising: a CDR-L1 comprising SEQ ID NO: 145; a CDR-L2 comprising SEQ ID NO: 163; and a CDR-L3 comprising SEQ ID NO: 186;
xv. a V_{L} comprising: a CDR-L1 comprising SEQ ID NO: 146; a CDR-L2 comprising SEQ ID NO: 164; and a CDR-L3 comprising SEQ ID NO: 187; or
xvi. a V_{L} comprising: a CDR-L1 comprising SEQ ID NO: 147; a CDR-L2 comprising SEQ ID NO: 165; and a CDR-L3 comprising SEQ ID NO: 188.

12. The bi-specific antibody or antigen binding construct of claim 1, wherein the first binding domain comprises a V_{L} sequence selected from SEQ ID NOs: 251-266.

13. The bi-specific antibody or antigen binding construct of claim 1, wherein the first binding domain comprises:
i. the V_{H} region SEQ ID NO: 200, or a variant thereof, and the V_{L} region SEQ ID NO: 254, or a variant thereof;
ii. the V_{H} region is SEQ ID NO: 191, or a variant thereof, and the V_{L} region is SEQ ID NO: 251, or a variant thereof;
iii. the V_{H} region is SEQ ID NO: 192, or a variant thereof, and the V_{L} region is SEQ ID NO: 261, or a variant thereof;
iv. the V_{H} region is SEQ ID NO: 193, or a variant thereof, and the V_{L} region is SEQ ID NO: 261, or a variant thereof;
v. the V_{H} region is SEQ ID NO: 194, or a variant thereof, and the V_{L} region is SEQ ID NO: 261, or a variant thereof;
vi. the V_{H} region is SEQ ID NO: 195, or a variant thereof, and the V_{L} region is SEQ ID NO: 261, or a variant thereof;
vii. the V_{H} region is SEQ ID NO: 196, or a variant thereof, and the V_{L} region is SEQ ID NO: 261, or a variant thereof;
viii. the V_{H} region is SEQ ID NO: 197, or a variant thereof, and the V_{L} region is SEQ ID NO: 256, or a variant thereof;
ix. the V_{H} region is SEQ ID NO: 198, or a variant thereof, and the V_{L} region is SEQ ID NO: 253, or a variant thereof;
x. the V_{H} region is SEQ ID NO: 199, or a variant thereof, and the V_{L} region is SEQ ID NO: 255, or a variant thereof;
xi. the V_{H} region is SEQ ID NO: 201, or a variant thereof, and the V_{L} region is SEQ ID NO: 252, or a variant thereof;
xii. the V_{H} region is SEQ ID NO: 202, or a variant thereof, and the V_{L} region is SEQ ID NO: 259, or a variant thereof;
xiii. the V_{H} region is SEQ ID NO: 203, or a variant thereof, and the V_{L} region is SEQ ID NO: 260, or a variant thereof;
xiv. the V_{H} region is SEQ ID NO: 204, or a variant thereof, and the V_{L} region is SEQ ID NO: 258, or a variant thereof;
xv. the V_{H} region is SEQ ID NO: 205, or a variant thereof, and the V_{L} region is SEQ ID NO: 257, or a variant thereof;
xvi. the V_{H} region is SEQ ID NO: 206, or a variant thereof, and the V_{L} region is SEQ ID NO: 262, or a variant thereof;
xvii. the V_{H} region is SEQ ID NO: 207, or a variant thereof, and the V_{L} region is SEQ ID NO: 263, or a variant thereof;
xviii. the V_{H} region is SEQ ID NO: 208, or a variant thereof, and the V_{L} region is SEQ ID NO: 265, or a variant thereof;
xix. the V_{H} region is SEQ ID NO: 209, or a variant thereof, and the V_{L} region is SEQ ID NO: 264, or a variant thereof; or
xx. the V_{H} region is SEQ ID NO: 210, or a variant thereof, and the V_{L} region is SEQ ID NO: 266, or a variant thereof.

14.-21. (canceled)

22. The bi-specific antibody or antigen binding construct of claim 13, wherein the second binding domain comprises:
i. a V_{H} comprising: a CDR-H1 comprising one or more of SEQ ID NOs: 38 and 65; a CDR-H2 comprising one or more of SEQ ID NOs: 87 and 109; and a CDR-H3 comprising SEQ ID NO: 131;
ii. a V_{H} comprising: a CDR-H1 comprising one or more of SEQ ID NOs: 24 and 59; a CDR-H2 comprising one or more of SEQ ID NOs: 86 and 108; and a CDR-H3 comprising SEQ ID NO: 130;
iii. a V_{H} comprising: a CDR-H1 comprising one or more of SEQ ID NOs: 25 and 59; a CDR-H2 comprising one or more of SEQ ID NOs: 86 and 108; and a CDR-H3 comprising SEQ ID NO: 130;
iv. a V_{H} comprising: a CDR-H1 comprising one or more of SEQ ID NOs: 26 and 59; a CDR-H2 comprising one or more of SEQ ID NOs: 86 and 108; and a CDR-H3 comprising SEQ ID NO: 130;
v. a V_{H} comprising: a CDR-H1 comprising one or more of SEQ ID NOs: 27 and 59; a CDR-H2 comprising one or more of SEQ ID NOs: 86 and 108; and a CDR-H3 comprising SEQ ID NO: 130;
vi. a V_{H} comprising: a CDR-H1 comprising one or more of SEQ ID NOs: 28 and 59; a CDR-H2 comprising one or more of SEQ ID NOs: 86 and 108; and a CDR-H3 comprising SEQ ID NO: 130;
vii. a V_{H} comprising: a CDR-H1 comprising one or more of SEQ ID NOs: 29 and 60; a CDR-H2 comprising one or more of SEQ ID NOs: 86 and 108; and a CDR-H3 comprising SEQ ID NO: 130;
viii. a V_{H} comprising: a CDR-H1 comprising one or more of SEQ ID NOs: 30 and 61; a CDR-H2 comprising one or more of SEQ ID NOs: 86 and 108; and a CDR-H3 comprising SEQ ID NO: 130;
ix. a V_{H} comprising: a CDR-H1 comprising one or more of SEQ ID NOs: 24 and 62; a CDR-H2 comprising one or more of SEQ ID NOs: 86 and 108; and a CDR-H3 comprising SEQ ID NO: 130;
x. a V_{H} comprising: a CDR-H1 comprising one or more of SEQ ID NOs: 24 and 63; a CDR-H2 comprising one or more of SEQ ID NOs: 86 and 108; and a CDR-H3 comprising SEQ ID NO: 130;
xi. a V_{H} comprising: a CDR-H1 comprising one or more of SEQ ID NOs: 31 and 59; a CDR-H2 comprising one or more of SEQ ID NOs: 86 and 108; and a CDR-H3 comprising SEQ ID NO: 130;
xii. a V_{H} comprising: a CDR-H1 comprising one or more of SEQ ID NOs: 32 and 59; a CDR-H2 comprising one or more of SEQ ID NOs: 86 and 108; and a CDR-H3 comprising SEQ ID NO: 130;
xiii. a V_{H} comprising: a CDR-H1 comprising one or more of SEQ ID NOs: 33 and 60; a CDR-H2 comprising one or more of SEQ ID NOs: 86 and 108; and a CDR-H3 comprising SEQ ID NO: 130;
xiv. a V_{H} comprising: a CDR-H1 comprising one or more of SEQ ID NOs: 34 and 60; a CDR-H2 comprising one or more of SEQ ID NOs: 86 and 108; and a CDR-H3 comprising SEQ ID NO: 130;
xv. a V_{H} comprising: a CDR-H1 comprising one or more of SEQ ID NOs: 35 and 64; a CDR-H2 comprising one or more of SEQ ID NOs: 86 and 108; and a CDR-H3 comprising SEQ ID NO: 130;
xvi. a V_{H} comprising: a CDR-H1 comprising one or more of SEQ ID NOs: 36 and 60; a CDR-H2 comprising one or more of SEQ ID NOs: 86 and 108; and a CDR-H3 comprising SEQ ID NO: 130;
xvii. a V_{H} comprising: a CDR-H1 comprising one or more of SEQ ID NOs: 37 and 64; a CDR-H2 comprising one or more of SEQ ID NOs: 86 and 108; and a CDR-H3 comprising SEQ ID NO: 130;
xviii. a V_{H} comprising: a CDR-H1 comprising a Chothia and/or Kabat CDR-H1 sequence of SEQ ID NO: 228; a CDR-H2 comprising a Chothia and/or Kabat CDR-H2 sequence of SEQ ID NO: 228; and a CDR-H3 comprising a CDR-H3 sequence of SEQ ID NO: 228;
xix. a V_{H} comprising: a CDR-H1 comprising a Chothia and/or Kabat CDR-H1 sequence of SEQ ID NO: 229; a CDR-H2 comprising a Chothia and/or Kabat CDR-H2 sequence of SEQ ID NO: 229; and a CDR-H3 comprising a CDR-H3 sequence of SEQ ID NO: 229;
xx. a V_{H} comprising: a CDR-H1 comprising a Chothia and/or Kabat CDR-H1 sequence of SEQ ID NO: 230; a CDR-H2 comprising a Chothia and/or Kabat CDR-H2 sequence of SEQ ID NO: 230; and a CDR-H3 comprising a CDR-H3 sequence of SEQ ID NO: 230;
xxi. a V_{H} comprising: a CDR-H1 comprising a Chothia and/or Kabat CDR-H1 sequence of SEQ ID NO: 231; a CDR-H2 comprising a Chothia and/or Kabat CDR-H2 sequence of SEQ ID NO: 231; and a CDR-H3 comprising a CDR-H3 sequence of SEQ ID NO: 231;
xxii. a V_{H} comprising: a CDR-H1 comprising a Chothia and/or Kabat CDR-H1 sequence of SEQ ID NO: 232; a CDR-H2 comprising a Chothia and/or Kabat CDR-H2 sequence of SEQ ID NO: 232; and a CDR-H3 comprising a CDR-H3 sequence of SEQ ID NO: 232;
xxiii. a V_{H} comprising: a CDR-H1 comprising a Chothia and/or Kabat CDR-H1 sequence of SEQ ID NO: 233; a CDR-H2 comprising a Chothia and/or Kabat CDR-H2 sequence of SEQ ID NO: 233; and a CDR-H3 comprising a CDR-H3 sequence of SEQ ID NO: 233;
xxiv. a V_{H} comprising: a CDR-H1 comprising a Chothia and/or Kabat CDR-H1 sequence of SEQ ID NO: 234; a CDR-H2 comprising a Chothia and/or Kabat CDR-H2 sequence of SEQ ID NO: 234; and a CDR-H3 comprising a CDR-H3 sequence of SEQ ID NO: 234;
xxv. a V_{H} comprising: a CDR-H1 comprising a Chothia and/or Kabat CDR-H1 sequence of SEQ ID NO: 235; a CDR-H2 comprising a Chothia and/or Kabat CDR-H2 sequence of SEQ ID NO: 235; and a CDR-H3 comprising a CDR-H3 sequence of SEQ ID NO: 235;
xxvi. a V_{H} comprising: a CDR-H1 comprising a Chothia and/or Kabat CDR-H1 sequence of SEQ ID NO: 236; a CDR-H2 comprising a Chothia and/or Kabat CDR-H2 sequence of SEQ ID NO: 236; and a CDR-H3 comprising a CDR-H3 sequence of SEQ ID NO: 236;
xxvii. a V_{H} comprising: a CDR-H1 comprising a Chothia and/or Kabat CDR-H1 sequence of SEQ ID NO: 237; a CDR-H2 comprising a Chothia and/or Kabat CDR-H2 sequence of SEQ ID NO: 237; and a CDR-H3 comprising a CDR-H3 sequence of SEQ ID NO: 237;
xxviii. a V_{H} comprising: a CDR-H1 comprising a Chothia and/or Kabat CDR-H1 sequence of SEQ ID NO: 238; a CDR-H2 comprising a Chothia and/or Kabat CDR-H2 sequence of SEQ ID NO: 238; and a CDR-H3 comprising a CDR-H3 sequence of SEQ ID NO: 238;
xxix. a V_{H} comprising: a CDR-H1 comprising a Chothia and/or Kabat CDR-H1 sequence of SEQ ID NO: 239; a CDR-H2 comprising a Chothia and/or Kabat CDR-H2 sequence of SEQ ID NO: 239; and a CDR-H3 comprising a CDR-H3 sequence of SEQ ID NO: 239;
xxx. a V_{H} comprising: a CDR-H1 comprising a Chothia and/or Kabat CDR-H1 sequence of SEQ ID NO: 240; a CDR-H2 comprising a Chothia and/or Kabat CDR-H2 sequence of SEQ ID NO: 240; and a CDR-H3 comprising a CDR-H3 sequence of SEQ ID NO: 240;
xxxi. a V_{H} comprising: a CDR-H1 comprising a Chothia and/or Kabat CDR-H1 sequence of SEQ ID NO: 241; a CDR-H2 comprising a Chothia and/or Kabat CDR-H2 sequence of SEQ ID NO: 241; and a CDR-H3 comprising a CDR-H3 sequence of SEQ ID NO: 241;
xxxii. a V_{H} comprising: a CDR-H1 comprising a Chothia and/or Kabat CDR-H1 sequence of SEQ ID NO: 242; a CDR-H2 comprising a Chothia and/or Kabat CDR-H2 sequence of SEQ ID NO: 242; and a CDR-H3 comprising a CDR-H3 sequence of SEQ ID NO: 242;
xxxiii. a V_{H} comprising: a CDR-H1 comprising a Chothia and/or Kabat CDR-H1 sequence of SEQ ID NO: 243; a CDR-H2 comprising a Chothia and/or Kabat CDR-H2 sequence of SEQ ID NO: 243; and a CDR-H3 comprising a CDR-H3 sequence of SEQ ID NO: 243;
xxxiv. a V_{H} comprising: a CDR-H1 comprising a Chothia and/or Kabat CDR-H1 sequence of SEQ ID NO: 244; a CDR-H2 comprising a Chothia and/or Kabat CDR-H2 sequence of SEQ ID NO: 244; and a CDR-H3 comprising a CDR-H3 sequence of SEQ ID NO: 244;
xxxv. a V_{H} comprising: a CDR-H1 comprising a Chothia and/or Kabat CDR-H1 sequence of SEQ ID NO: 245; a CDR-H2 comprising a Chothia and/or Kabat CDR-H2 sequence of SEQ ID NO: 245; and a CDR-H3 comprising a CDR-H3 sequence of SEQ ID NO: 245;
xxxvi. a V_{H} comprising: a CDR-H1 comprising a Chothia and/or Kabat CDR-H1 sequence of SEQ ID NO: 246; a CDR-H2 comprising a Chothia and/or Kabat CDR-H2 sequence of SEQ ID NO: 246; and a CDR-H3 comprising a CDR-H3 sequence of SEQ ID NO: 246;
xxxvii. a V_{H} comprising: a CDR-H1 comprising a Chothia and/or Kabat CDR-H1 sequence of SEQ ID NO: 247; a CDR-H2 comprising a Chothia and/or Kabat CDR-H2 sequence of SEQ ID NO: 247; and a CDR-H3 comprising a CDR-H3 sequence of SEQ ID NO: 247;
xxxviii. a V_{H} comprising: a CDR-H1 comprising a Chothia and/or Kabat CDR-H1 sequence of SEQ ID NO: 248; a CDR-H2 comprising a Chothia and/or Kabat CDR-H2 sequence of SEQ ID NO: 248; and a CDR-H3 comprising a CDR-H3 sequence of SEQ ID NO: 248;
xxxix. a V_{H} comprising: a CDR-H1 comprising a Chothia and/or Kabat CDR-H1 sequence of SEQ ID NO: 249; a CDR-H2 comprising a Chothia and/or Kabat CDR-H2 sequence of SEQ ID NO: 249; and a CDR-H3 comprising a CDR-H3 sequence of SEQ ID NO: 249; or
xl. a V_{H} comprising: a CDR-H1 comprising a Chothia and/or Kabat CDR-H1 sequence of SEQ ID NO: 250; a CDR-H2 comprising a Chothia and/or Kabat CDR-H2 sequence of SEQ ID NO: 250; and a CDR-H3 comprising a CDR-H3 sequence of SEQ ID NO: 250.

23. The bi-specific antibody or antigen binding construct of claim 1, wherein the second binding domain comprises a V_{H} selected from SEQ ID NOs: 211-250.

24. The bi-specific antibody or antigen binding construct of claim 22, wherein the second binding domain comprises:
i. a V_{L} comprising: a CDR-L1 comprising SEQ ID NO: 149; a CDR-L2 comprising SEQ ID NO: 172; and a CDR-L3 comprising SEQ ID NO: 190;
ii. a V_{L} comprising: a CDR-L1 comprising SEQ ID NO: 148; a CDR-L2 comprising SEQ ID NO: 166; and a CDR-L3 comprising SEQ ID NO: 189;
iii. a V_{L} comprising: a CDR-L1 comprising SEQ ID NO: 148; a CDR-L2 comprising SEQ ID NO: 167; and a CDR-L3 comprising SEQ ID NO: 189;
iv. a V_{L} comprising: a CDR-L1 comprising SEQ ID NO: 148; a CDR-L2 comprising SEQ ID NO: 168; and a CDR-L3 comprising SEQ ID NO: 189;
v. a V_{L} comprising: a CDR-L1 comprising SEQ ID NO: 148; a CDR-L2 comprising SEQ ID NO: 169; and a CDR-L3 comprising SEQ ID NO: 189;
vi. a V_{L} comprising: a CDR-L1 comprising SEQ ID NO: 148; a CDR-L2 comprising SEQ ID NO: 170; and a CDR-L3 comprising SEQ ID NO: 189;
vii. a V_{L} comprising: a CDR-L1 comprising SEQ ID NO: 148; a CDR-L2 comprising SEQ ID NO: 171; and a CDR-L3 comprising SEQ ID NO: 189;
ix. a V_{L} comprising: a CDR-L1 comprising a CDR-L1 sequence of SEQ ID NO: 277; a CDR-L2 comprising a CDR-L2 sequence of SEQ ID NO: 277; and a CDR-L3 comprising a CDR-L3 sequence of SEQ ID NO: 277;
x. a V_{L} comprising: a CDR-L1 comprising a CDR-L1 sequence of SEQ ID NO: 278; a CDR-L2 comprising a CDR-L2 sequence of SEQ ID NO: 278; and a CDR-L3 comprising a CDR-L3 sequence of SEQ ID NO: 278;
xi. a V_{L} comprising: a CDR-L1 comprising a CDR-L1 sequence of SEQ ID NO: 279; a CDR-L2 comprising a CDR-L2 sequence of SEQ ID NO: 279; and a CDR-L3 comprising a CDR-L3 sequence of SEQ ID NO: 279;
xii. a V_{L} comprising: a CDR-L1 comprising a CDR-L1 sequence of SEQ ID NO: 280; a CDR-L2 comprising a CDR-L2 sequence of SEQ ID NO: 280; and a CDR-L3 comprising a CDR-L3 sequence of SEQ ID NO: 280;
xiii. a V_{L} comprising: a CDR-L1 comprising a CDR-L1 sequence of SEQ ID NO: 281; a CDR-L2 comprising a CDR-L2 sequence of SEQ ID NO: 281; and a CDR-L3 comprising a CDR-L3 sequence of SEQ ID NO: 281;
xiv. a V_{L} comprising: a CDR-L1 comprising a CDR-L1 sequence of SEQ ID NO: 282; a CDR-L2 comprising a CDR-L2 sequence of SEQ ID NO: 282; and a CDR-L3 comprising a CDR-L3 sequence of SEQ ID NO: 282;
xv. a V_{L} comprising: a CDR-L1 comprising a CDR-L1 sequence of SEQ ID NO: 283; a CDR-L2 comprising a CDR-L2 sequence of SEQ ID NO: 283; and a CDR-L3 comprising a CDR-L3 sequence of SEQ ID NO: 283;
xvi. a V_{L} comprising: a CDR-L1 comprising a CDR-L1 sequence of SEQ ID NO: 284; a CDR-L2 comprising a CDR-L2 sequence of SEQ ID NO: 284; and a CDR-L3 comprising a CDR-L3 sequence of SEQ ID NO: 284;
xvii. a V_{L} comprising: a CDR-L1 comprising a CDR-L1 sequence of SEQ ID NO: 285; a CDR-L2 comprising a CDR-L2 sequence of SEQ ID NO: 285; and a CDR-L3 comprising a CDR-L3 sequence of SEQ ID NO: 285;
xviii. a V_{L} comprising: a CDR-L1 comprising a CDR-L1 sequence of SEQ ID NO: 286; a CDR-L2 comprising a CDR-L2 sequence of SEQ ID NO: 286; and a CDR-L3 comprising a CDR-L3 sequence of SEQ ID NO: 286;
xix. a V_{L} comprising: a CDR-L1 comprising a CDR-L1 sequence of SEQ ID NO: 287; a CDR-L2 comprising a CDR-L2 sequence of SEQ ID NO: 287; and a CDR-L3 comprising a CDR-L3 sequence of SEQ ID NO: 287;
xx. a V_{L} comprising: a CDR-L1 comprising a CDR-L1 sequence of SEQ ID NO: 288; a CDR-L2 comprising a CDR-L2 sequence of SEQ ID NO: 288; and a CDR-L3 comprising a CDR-L3 sequence of SEQ ID NO: 288;
xxi. a V_{L} comprising: a CDR-L1 comprising a CDR-L1 sequence of SEQ ID NO: 289; a CDR-L2 comprising a CDR-L2 sequence of SEQ ID NO: 289; and a CDR-L3 comprising a CDR-L3 sequence of SEQ ID NO: 289;
xxii. a V_{L} comprising: a CDR-L1 comprising a CDR-L1 sequence of SEQ ID NO: 290; a CDR-L2 comprising a CDR-L2 sequence of SEQ ID NO: 290; and a CDR-L3 comprising a CDR-L3 sequence of SEQ ID NO: 290;
xxiii. a V_{L} comprising: a CDR-L1 comprising a CDR-L1 sequence of SEQ ID NO: 291; a CDR-L2 comprising a CDR-L2 sequence of SEQ ID NO: 291; and a CDR-L3 comprising a CDR-L3 sequence of SEQ ID NO: 291;
xxiv. a V_{L} comprising: a CDR-L1 comprising a CDR-L1 sequence of SEQ ID NO: 292; a CDR-L2 comprising a CDR-L2 sequence of SEQ ID NO: 292; and a CDR-L3 comprising a CDR-L3 sequence of SEQ ID NO: 292;
xxv. a V_{L} comprising: a CDR-L1 comprising a CDR-L1 sequence of SEQ ID NO: 293; a CDR-L2 comprising a CDR-L2 sequence of SEQ ID NO: 293; and a CDR-L3 comprising a CDR-L3 sequence of SEQ ID NO: 293;
xxvi. a V_{L} comprising: a CDR-L1 comprising a CDR-L1 sequence of SEQ ID NO: 294; a CDR-L2 comprising a CDR-L2 sequence of SEQ ID NO: 294; and a CDR-L3 comprising a CDR-L3 sequence of SEQ ID NO: 294;
xxvii. a V_{L} comprising: a CDR-L1 comprising a CDR-L1 sequence of SEQ ID NO: 295; a CDR-L2 comprising a CDR-L2 sequence of SEQ ID NO: 295; and a CDR-L3 comprising a CDR-L3 sequence of SEQ ID NO: 295;
xxviii. a V_{L} comprising: a CDR-L1 comprising a CDR-L1 sequence of SEQ ID NO: 296; a CDR-L2 comprising a CDR-L2 sequence of SEQ ID NO: 296; and a CDR-L3 comprising a CDR-L3 sequence of SEQ ID NO: 296;
xxix. a V_{L} comprising: a CDR-L1 comprising a CDR-L1 sequence of SEQ ID NO: 297; a CDR-L2 comprising a CDR-L2 sequence of SEQ ID NO: 297; and a CDR-L3 comprising a CDR-L3 sequence of SEQ ID NO: 297;
xxx. a V_{L} comprising: a CDR-L1 comprising a CDR-L1 sequence of SEQ ID NO: 298; a CDR-L2 comprising a CDR-L2 sequence of SEQ ID NO: 298; and a CDR-L3 comprising a CDR-L3 sequence of SEQ ID NO: 298; or
xxxi. a V_{L} comprising: a CDR-L1 comprising a CDR-L1 sequence of SEQ ID NO: 299; a CDR-L2 comprising a CDR-L2 sequence of SEQ ID NO: 299; and a CDR-L3 comprising a CDR-L3 sequence of SEQ ID NO: 299.

25. The bi-specific antibody or antigen binding construct of claim 13, wherein the second binding domain comprises a V_{H} sequence selected from SEQ ID NOs: 211-227 and a V_{L} sequence selected from SEQ ID NOs: 267-276.

26. The bi-specific antibody or antigen binding construct of claim 25, wherein the second binding domain comprises:
i. the V_{H} region is SEQ ID NO: 227 and the V_{L} region is SEQ ID NO: 275;
ii. the V_{H} region is SEQ ID NO: 211 and the V_{L} region is SEQ ID NO: 275;
iii. the V_{H} region is SEQ ID NO: 227 and the V_{L} region is SEQ ID NO: 267;
iv. the V_{H} region is SEQ ID NO: 211 and the V_{L} region is SEQ ID NO: 267;
v. the V_{H} region is SEQ ID NO: 228 and the V_{L} region is SEQ ID NO: 277;
vi. the V_{H} region is SEQ ID NO: 229 and the V_{L} region is SEQ ID NO: 278;
vii. the V_{H} region is SEQ ID NO: 230 and the V_{L} region is SEQ ID NO: 279;
viii. the V_{H} region is SEQ ID NO: 231 and the V_{L} region is SEQ ID NO: 280;
ix. the V_{H} region is SEQ ID NO: 232 and the V_{L} region is SEQ ID NO: 281;
x. the V_{H} region is SEQ ID NO: 233 and the V_{L} region is SEQ ID NO: 282;
xi. the V_{H} region is SEQ ID NO: 234 and the V_{L} region is SEQ ID NO: 283;
xii. the V_{H} region is SEQ ID NO: 235 and the V_{L} region is SEQ ID NO: 284;
xiii. the V_{H} region is SEQ ID NO: 236 and the V_{L} region is SEQ ID NO: 285;
xiv. the V_{H} region is SEQ ID NO: 237 and the V_{L} region is SEQ ID NO: 286;
xv. the V_{H} region is SEQ ID NO: 238 and the V_{L} region is SEQ ID NO: 287;
xvi. the V_{H} region is SEQ ID NO: 239 and the V_{L} region is SEQ ID NO: 288;
xvii. the V_{H} region is SEQ ID NO: 240 and the V_{L} region is SEQ ID NO: 289;
xviii. the V_{H} region is SEQ ID NO: 241 and the V_{L} region is SEQ ID NO: 290;
xix. the V_{H} region is SEQ ID NO: 242 and the V_{L} region is SEQ ID NO: 291;
xx. the V_{H} region is SEQ ID NO: 243 and the V_{L} region is SEQ ID NO: 292;
xxi. the V_{H} region is SEQ ID NO: 244 and the V_{L} region is SEQ ID NO: 293;
xxii. the V_{H} region is SEQ ID NO: 245 and the V_{L} region is SEQ ID NO: 294;
xxiii. the V_{H} region is SEQ ID NO: 246 and the V_{L} region is SEQ ID NO: 295;
xxiv. the V_{H} region is SEQ ID NO: 247 and the V_{L} region is SEQ ID NO: 296;
xxv. the V_{H} region is SEQ ID NO: 248 and the V_{L} region is SEQ ID NO: 297;
xxvi. the V_{H} region is SEQ ID NO: 249 and the V_{L} region is SEQ ID NO: 298; or
xxvii. the V_{H} region is SEQ ID NO: 250 and the V_{L} region is SEQ ID NO: 299.

27.-28. (canceled)

29. The bi-specific antibody or antigen binding construct of claim 1, wherein the antibody comprises at least one constant region domain.

30. (canceled)

31. The bi-specific antibody or antigen binding construct of claim 1, wherein the bi-specific antibody or antigen binding construct is humanized or human.

32. The bi-specific antibody or antigen binding construct of claim 1, wherein the bi-specific antibody or antigen binding construct is aglycosylated.

33.-39. (canceled)

40. The bi-specific antibody or antigen binding construct of claim 1, wherein the first binding domain has a kₐ of about 5.02×10⁴ M⁻¹×sec⁻¹ to about 5.31×10⁷ M⁻¹×sec⁻¹ when associating with human LAG3 at a temperature of 25° C.

41.-42. (canceled)

43. The bi-specific antibody or antigen binding construct of claim 1, wherein the first binding domain specifically binds cynomolgus LAG3.

44.-46. (canceled)

47. The bi-specific antibody or antigen binding construct of claim 1, wherein the second binding domain specifically binds one or more of murine PD-1 and cynomolgus PD-1.

48.-54. (canceled)

55. The bi-specific antibody or antigen binding construct of claim 1, comprising:
i. a first antigen-binding polypeptide comprising:
i. a first V_{H} comprising a sequence selected from SEQ ID NOs: 211-227;
ii. a first V_{L} comprising a sequence selected from SEQ ID NOs: 267-276;
iii. a linker-hinge sequence comprising SEQ ID NO: 315; and
iv. a CH₂-CH₃ region comprising a sequence selected from SEQ ID NOs: 321-325; and
ii. a second antigen-binding polypeptide comprising:
i. a second V_{H} comprising a sequence selected from SEQ ID NOs: 191-210;
ii. a linker hinge sequence comprising SEQ ID NO: 316;
iii. a CH₂-CH₃ region comprising a sequence selected from SEQ ID NOs: 326-330;
iv. a second V_{L} comprising a sequence selected from SEQ ID NOs: 251-266; and
v. a C_{L} comprising SEQ ID NO: 386.

56.-72. (canceled)

73. An isolated antibody that specifically binds to LAG3, wherein the antibody comprises:
i. a V_{H} comprising SEQ ID NO: 200;
ii. a CH₁ region comprising a sequence selected from SEQ ID NOs: 343-352;
iii. a linker sequence comprising SEQ ID NO: 316; and
iv. a CH₂-CH₃ region comprising a sequence selected from SEQ ID NOs: 331-336.

74.-75. (canceled)

76. The antibody of claim 73, wherein the antibody is a monoclonal antibody.

77. The antibody of claim 73, wherein the antibody is an IgA, an IgD, an IgE, an IgG, or an IgM.

78. The antibody of claim 73, wherein the antibody is humanized or human.

79. The antibody of claim 73, wherein the antibody is aglycosylated.

80.-85. (canceled)

86. A kit comprising an antibody of claim 1, and instructions for use of the antibody.

87.-88. (canceled)

89. A polynucleotide encoding an antibody of claim 73.

90. A vector comprising the polynucleotide of claim 89.

91. A recombinant host cell comprising the vector of claim 90.

92.-94. (canceled)

95. A pharmaceutical composition comprising the antibody of claim 1 and a pharmaceutically acceptable carrier.

96. A method of treating or preventing a disease or condition in a subject in need thereof, comprising administering to the subject an effective amount of an antibody of claim 1, or a pharmaceutical composition of claim 95.

97. A method of diagnosing a disease or condition in a subject in need thereof, comprising administering to the subject an effective amount of an antibody of claim 1, or a pharmaceutical composition of claim 95.

98. (canceled)
